(19) United States
(12) Patent Application Publication
(10) Pub. No.: US 2003/0228618 A1
(43) Pub. Date: Dec. 11, 2003

(54) Methods and systems for identifying naturally occurring antisense transcripts and methods, kits and arrays utilizing same

(76) Inventors: Erez Levanon, Petach Tikva (IL); Sarah Pollock, Tel Aviv (IL); Sergey Nemzer, Ramat Gan (IL); Avi Shoshan, Kiryat Gat (IL); Rami Khosravi, Herzlia (IL); Shira Walach, Hod Hasharon (IL); Zurit Levine, Herzlia (IL); Jeanne Bernstein, Kfar Yona (IL); Dvir Dahari, Tel Aviv (IL); Alon Wasserman, Tel Aviv (IL); Galit Rotman, Herzlia (IL)

Correspondence Address: G.E. EHRLICH (1995) LTD., c/o ANTHONY CASTORINA, 2001JEFFERSON DAVIS HIGHWAY, SUITE 207, ARLINGTON, VA, 22202, US

(21) Appl. No.: 10/441,281

(22) Filed: May 20, 2003

Related U.S. Application Data
(63) Continuation-in-part of application No. PCT/IL02/00904, filed on Nov. 11, 2002.
Application No. PCT/IL02/00904 is a continuation-in-part of application No. 10/201,605, filed on Jul. 24, 2002.
Application No. 10/201,605 is a continuation-in-part of application No. 09/993,398, filed on Nov. 26, 2001.
Application No. 09/993,398 is a continuation-in-part of application No. 09/907,923, filed on Jul. 18, 2001, now abandoned.
Application No. 09/907,923 is a continuation-in-part of application No. 09/785,439, filed on Feb. 20, 2001, now abandoned.
Application No. 09/785,439 is a continuation-in-part of application No. 09/732,938, filed on Dec. 11, 2000, now abandoned.
(63) Continuation-in-part of application No. 09/718,407, filed on Nov. 24, 2000, now abandoned.

(51) Int. Cl. C12Q 1/68; G06F 19/00; G01N 33/48; G01N 33/50

(52) U.S. Cl. 435/6; 702/20

(57) ABSTRACT

A method of identifying putative naturally occurring antisense transcripts is provided. The method is effected by (a) computationally aligning a first database including sense-oriented polynucleotide sequences with a second database including expressed polynucleotide sequences; and (b) identifying expressed polynucleotide sequences from the second database being capable of forming a duplex with at least one sense-oriented polynucleotide sequence of the first database, thereby identifying putative naturally occurring antisense transcripts.

This is a continuation-in-part of PCT Patent Application No. IL02/00904, filed Nov. 11, 2002, which claims priority from U.S. patent application Ser. No. 10/201,605, filed Jul. 24, 2002, which is a continuation-in-part of U.S. patent application Ser. No. 09/993,398, filed Nov. 26, 2001, which is a continuation-in-part of U.S. patent application Ser. No. 09/907,923, filed Jul. 18, 2001, which is a continuation-in-part of U.S. patent application Ser. No. 09/785,439, filed Feb. 20, 2001, which is a continuation-in-part of U.S. patent application Ser. No. 09/732,938, filed Dec. 11, 2000. This Application also claims the benefit of priority from U.S. patent application Ser. No. 09/718,407, filed Nov. 24, 2000.

### BACKGROUND AND FIELD OF THE INVENTION

The present invention relates to the field of naturally occurring, antisense transcripts. More particularly, the present invention relates to methods of identifying naturally occurring antisense transcripts, databases storing polynucleotide sequences encoding identified naturally occurring antisense transcripts, oligonucleotides derived therefrom and methods and kits utilizing same.

Naturally occurring antisense RNA transcripts are endogenous transcripts, which exhibit complementarity to sense transcripts of which are typically of a known function. It has been established that these endogenous antisense transcripts play an important role in regulating prokaryotic gene expression and are increasingly implicated as involved in eukaryotic gene regulation.

Cis-encoded antisense transcripts are encoded by the same locus as the sense transcripts and are transcribed from strand of DNA opposite to that encoding the sense transcript; as such, cis encoded antisense transcripts are typically completely complementary with a portion of the sense transcript.

Trans-encoded antisense transcripts are by contrast, transcripts, which are encoded on a different locus and as such, may display only partial complementarity with a sense transcript.

Natural antisense RNAs were first described in prokaryote studies, which suggested that such transcripts play a role in gene expression regulation. Prokaryotic antisense transcripts are widely distributed and are involved in the control of numerous biological functions including transposition, plasmid replication, incompatibility and conjugation. In prokaryotes, antisense transcripts are typically involved in down-regulation of sense transcript expression, although involvement in positive regulation was also suggested [reviewed in Wagner E G. and Simons R W. (1994) Annu. Rev. Microbiol. 48:713-742].

The first example of transcription from both strands of eukaryotic DNA was illustrated in human and mouse mitochondrial genes [Anderson S. et al. (1981) Nature 290:457-465 and Bibb M J. et al. (1981) Cell 26:167-180]. Since then, examples of antisense transcripts have been documented in a variety of organisms including viruses, slime molds, insects, amphibians and birds as well as mammals. It is thought that these antisense RNAs are involved in extremely diverse biological functions, such as, hormonal response, control of proliferation, development, structure, viral replication and others. Some antisense RNAs are conserved between species suggesting that these antisense RNAs are not fortuitous but rather play an important role in gene expression regulation [Kidny M S. et al. (1987) Mol. Cell Biol. 7:2857-2862, Nepveu A. and Marcu K B. (1986) EMBO J. 5:2859-2865 and Bentley D L. et al. (1986) Nature 321:702-706].

Antisense transcripts can also encode proteins. Examples for protein encoding antisense transcripts include rev-ErbAx [Lazar M A. (1989) Mol. Cell. Biol. 9:1128-1136], gfg [Kimelman D. et al. (1989) Cell 59:687-696] and n-cym [Armstrong B C. et al. (1992) Cell Growth Differ. 3:385-390]. Such antisense transcripts typically include a distinct open reading frame (ORF) and polyadenylation signal for cytoplasm transportation.

However, it is believed that most antisense transcripts play a role in gene expression regulation. This assumption is mostly based on spatial and/or temporal distributions of sense and antisense transcripts. Indeed, tissue distribution studies suggest that high levels of sense and antisense transcripts rarely occur together, as was exemplified for the dopa decarboxylase transcripts in Drosophila [Spencer C A. et al. (1986) Nature 322:279-281]. Additional studies demonstrated that changes in sense gene expression correlate with presence of antisense RNA. Furthermore, an inverse relationship between levels of accumulation of sense and antisense transcripts such as has been reported for α1 (I) collagen transcripts in chondrocytes under chemotherapy has also been reported [Farrell C M. And Lukens L N. (1995) J. Biol. Chem. 270:3400-3408]. However, it will be appreciated that mutual expression of sense and their corresponding antisense transcripts is also reported and may involve a different mechanism of regulation.

Evidence for involvement of antisense-mediated gene regulation in the development of pathologies has also been presented. For example, endogenous antisense transcripts may be involved in regulation of the expression levels of the tumor suppressor gene WT1 observed in Wilm's tumors [Eccles M R. et al. (1994) Oncogene 9:2059-2063].

Natural antisense regulation of gene expression can be effected via one of several mechanisms.

Nuclear Regulation

Nuclear regulation can be effected via several gene-processing pathways [reviewed in Vanhee-Brosollet C. and Vaquero C. (1998) Gene 211:1-9]

dsRNA-mediated DNA methylation—complementation between endogenous sense transcripts and antisense transcripts of sequences as short as 30 bp may initiate DNA-methylation, a well-established phenomenon in a number of organisms [Sharp A. (2001) Genes Dev. 15:485-490]. Methylation can be directed to different portions of an encoding region of the gene or to the promoter region. DNA methylation results in complete suppression of transcription probably by recruitment of histone deacetylases.

Transcriptional regulation—in which case antisense transcription hampers sense transcription. Such interference may involve the collision of two transcription complexes. Alternatively, interference may result from competition on an essential rate limiting transcription factor resulting in premature termination or in reduced elongation of transcription, the transcripts with the highest rate of transcription being predominant.

Post-transcriptional nuclear regulation—involves antisense intervention of either maturation and/or transport of the sense transcript to the cytoplasm. Alternatively, antisense transcripts displaying similar structural features to sense transcripts can bind proteins expected to interact with their sense counterparts, thereby depriving sense messengers from proteins necessary for their function.

Cytoplasmic Regulation

Messenger stability—double stranded RNA may affect messenger stability via “RNA interference”, which involves short segments of double stranded RNA (dsRNA) homologous in sequence to the silenced gene. These undersized segments, which are generated by a ribonuclease III cleavage of longer dsRNAs, can guide a single stranded target mRNA, via base pairing, to a multisubunit complex which participates in the degradation of the target mRNA. Alternatively, messenger stability may be affected by RNA degradation, which is mediated by double stranded RNA-directed Rnases.

Translation—masking the 3′ untranslated region (UTR) and the polyA tail of the sense transcript is believed to modulate translation efficiency probably via direct or indirect interaction between 3′-proximal elements and upstream sequences or structures [reviewed in Jackson R J. And Standart N. (1990) Cell 62:15-24].

Realizing the fundamental role antisense transcripts play in regulating sense transcription, stability and function, resulted in a number of attempts to systematically identify natural antisense transcripts. Accordingly, differential approaches were taken for exploring non-coding antisense RNA transcripts and antisense transcripts including an ORF. Although the latter carries ORF consensus parameters, uncovering antisense data from general sequence databases has proven to be a complicated task, as many of these sequences include an evolutionary conserved secondary structure rather than a conserved primary sequence, therefore primary sequence alignment methods are often not very effective. Indeed, only a few attempts have been tried to date with only limited success.

Maziel's group [Chen J H. et al. (1990) Comput. Applic. Biosci. 6:7-18 and Le S Y. et al (1990) Human Genome Initiative and DNA Recombination Vol. 1:127-136] has experimented with methods that look for regions of a genome with predicted RNA structures that are significantly more stable thermodynamically than random sequence of the same base composition. Although this approach detected a few highly structured non-coding RNAs, as well as few cis-regulatory structures, it appears that it is of limited use for large-scale applications.

Another approach examined coding dense genomes, having suspicious-looking large regions with little or no coding potential termed “gray holes” [Olivas W M. et al. (1997) Nucleic acids Res. 25:4619-4625]. Fifty nine gray holes were tested in the yeast genome. Northern analysis detected distinct transcripts from 15 of the gray holes. Only one transcript appeared to be a non-coding antisense transcript illustrating the low efficiency of this method.

There is thus a widely recognized need for, and it would be highly advantageous to have, methods of systematically identifying novel naturally occurring antisense molecules and methods of artificially generating and using same for detecting, quantifying and/or regulating sense transcripts, such as for example, mRNA transcripts associated with a pathological state.

### SUMMARY OF THE INVENTION

According to one aspect of the present invention there is provided a method of identifying putative naturally occurring antisense transcripts, the method comprising: (a) computationally aligning a first database including sense-oriented polynucleotide sequences with a second database including expressed polynucleotide sequences; and (b) identifying expressed polynucleotide sequences from the second database being capable of forming a duplex with at least one sense-oriented polynucleotide sequence of the first database, thereby identifying putative naturally occurring antisense transcripts.

According to another aspect of the present invention there is provided a kit for quantifying at least one mRNA transcript of interest, the kit comprising at least one oligonucleotide being designed and configured so as to be complementary to a sequence region of the mRNA transcript of interest, the sequence region not being complementary with a naturally occurring antisense transcript.

According to yet another aspect of the present invention there is provided a kit for quantifying at least one mRNA transcript of interest, the kit comprising at least one pair of oligonucleotides including a first oligonucleotide capable of binding the at least one mRNA transcript of interest and a second oligonucleotide being capable of binding a naturally occurring antisense transcript complementary to the mRNA of interest.

According to still another aspect of the present invention there is provided a method of designing artificial antisense transcripts, the method comprising: (a) providing a database of naturally occurring antisense transcripts; (b) extracting from the database criteria governing structure and/or function of the naturally occurring antisense transcripts; and (c) designing the artificial antisense transcripts according to the criteria.

According to further features in preferred embodiments of the invention described below the criteria governing structure and/or function of the naturally occurring antisense transcripts are selected from the group consisting of antisense length, complementarity length, complementarity position, intron molecules, alternative splicing sites, tissue specificity, pathological abundance, chromosomal mapping, open reading frames, promoters, hairpin structures, helix structures, stem and loops, pseudoknots and tertiary interactions, guanidine and/or cytosine content, guanidine tandems, adenosine content, thermodynamic criteria, RNA duplex melting point, RNA modifications, protein-binding motifs, palindromic sequence and predicted single stranded and double stranded regions.

According to an additional aspect of the present invention there is provided a computer readable storage medium comprising a database including a plurality of sequences, wherein each sequence is of a naturally occurring antisense transcript.

According to still further features in the described preferred embodiments the database further includes information pertaining to each sequence of the naturally occurring antisense transcripts, the information is selected from the group consisting of related sense gene, antisense length, complementarity length, complementarity position, intron molecules, alternative splicing sites, tissue specificity, pathological abundance, chromosomal mapping, open reading frames, promoters, hairpin structures, helix structures, stem and loops, pseudoknots and tertiary interactions, guanidine and/or cytosine content, guanidine tandems, adenosine content, thermodynamic criteria, RNA duplex melting point, RNA modifications, protein-binding motifs, palindromic sequence and predicted single stranded and double stranded regions.

According to still further features in the described preferred embodiments the database further includes information pertaining to generation of the database and potential uses of the database.

According to yet an additional aspect of the present invention there is provided a method of generating a database of naturally occurring antisense transcripts, the method comprising: (a) computationally aligning a first database including sense-oriented polynucleotide sequences with a second database including expressed polynucleotide sequences; (b) identifying expressed polynucleotide sequences from the second database being capable of forming a duplex with at least one sense-oriented polynucleotide sequence of the first database so as to identify putative naturally occurring antisense transcripts; and (c) storing sequence information of the identified naturally occurring antisense transcripts, thereby generating the database of the naturally occurring antisense transcripts.

According to still an additional aspect of the present invention there is provided a system for generating a database of a plurality of putative naturally occurring antisense transcripts, the system comprising a processing unit, the processing unit executing a software application configured for: (a) computationally aligning a first database including sense-oriented polynucleotide sequences with a second database including expressed polynucleotide sequences; and (b) identifying expressed polynucleotide sequences from the second database being capable of forming a duplex with at least one sense-oriented polynucleotide sequence of the first database.

According to a further aspect of the present invention there is provided a method of identifying putative naturally occurring antisense transcripts, the method comprising screening a database of expressed polynucleotides sequences according to at least one sequence criterion, the at least one sequence criterion being selected to identify putative naturally occurring antisense transcripts.

According to yet a further aspect of the present invention there is provided A method of quantifying at least one mRNA of interest in a biological sample, the method comprising: (a) contacting the biological sample with at least one oligonucleotide capable of binding with the at least one mRNA of interest, wherein the at least one oligonucleotide is designed and configured so as to be complementary to a sequence region of the mRNA transcript of interest, the sequence region not being complementary with a naturally occurring antisense transcript; and (b) detecting a level of binding between the at least one mRNA of interest and the at least one oligonucleotide to thereby quantify the at least one mRNA of interest in the biological sample.

According to still a further aspect of the present invention there is provided a method of quantifying the expression potential of at least one mRNA of interest in a biological sample, the method comprising: (a) contacting the biological sample with at least one pair of oligonucleotides including a first oligonucleotide capable of binding the at least one mRNA of interest and a second oligonucleotide being capable of binding a naturally occurring antisense transcript complementary to the mRNA of interest; and (b) detecting a level of binding between the at least one mRNA of interest and the first oligonucleotide and a level of binding between the naturally occurring antisense transcript complementary to the mRNA of interest and the second oligonucleotide to thereby quantify the expression potential of the at least one mRNA of interest in the biological sample.

According to other aspect of the present invention there is provided a method of quantifying at least one naturally occurring antisense transcript of interest in a biological sample, the method comprising: (a) contacting the biological sample with at least one oligonucleotide capable of binding with the at least one naturally occurring antisense transcript of interest, wherein the at least one oligonucleotide is designed and configured so as to be complementary to a sequence region of the naturally occurring antisense transcript of interest, the sequence region not being complementary with a naturally occurring mRNA transcript; and (b) detecting a level of binding between the at least one naturally occurring antisense transcript of interest and the at least one oligonucleotide to thereby quantify the at least one naturally occurring antisense transcript of interest in the biological sample.

According to still further features in the described preferred embodiments the first database includes sequences of a type selected from the group consisting of genomic sequences, expressed sequence tags, contigs, intron sequences, complementary DNA (cDNA) sequences, pre-messenger RNA (mRNA) sequences and mRNA sequences.

According to still further features in the described preferred embodiments the second database includes sequences of a type selected from the group consisting of expressed sequence tags, contigs, complementary DNA (cDNA) sequences, pre-messenger RNA (mRNA) sequences and mRNA sequences.

According to still further features in the described preferred embodiments an average sequence length of the expressed polynucleotide sequences of the second database is selected from a range of 0.02 to 0.8 Kb.

According to still further features in the described preferred embodiments the second database is generated by: (i) providing a library of expressed polynucleotides; (ii) obtaining sequence information of the expressed polynucleotides; (iii) computationally selecting at least a portion of the expressed polynucleotides according to at least one sequence criterion; and (iv) storing the sequence information of the at least a portion of the expressed polynucleotides thereby generating the second database.

According to still further features in the described preferred embodiments the at least one sequence criterion for computationally selecting the at least a portion of the expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

According to still further features in the described preferred embodiments the step of testing the putative naturally occurring antisense transcripts for an ability to form the duplex with the at least one sense oriented polynucleotide sequence under physiological conditions.

According to still further features in the described preferred embodiments the method further comprising the step of computationally testing the putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

According to still further features in the described preferred embodiments a length of the at least one oligonucleotide is selected from a range of 15-200 nucleotides.

According to still further features in the described preferred embodiments the at least one oligonucleotide is a single stranded oligonucleotide.

According to still further features in the described preferred embodiments the at least one oligonucleotide is a double stranded oligonucleotide.

According to still further features in the described preferred embodiments a guanidine and cytosine content of the at least one oligonucleotide is at least 25%.

According to still further features in the described preferred embodiments the at least one oligonucleotide is labeled.

According to still further features in the described preferred embodiments the at least one oligonucleotide is attached to a solid substrate.

According to still further features in the described preferred embodiments the solid substrate is configured as a microarray and whereas the at least one oligonucleotide includes a plurality of oligonucleotides each attached to the microarray in a regio-specific manner.

According to still further features in the described preferred embodiments a length of each of the first and second oligonucleotides is selected from a range of 15-200 nucleotides.

According to still further features in the described preferred embodiments the first and second oligonucleotides are single stranded oligonucleotides.

According to still further features in the described preferred embodiments the first and second oligonucleotides are double stranded oligonucleotide.

According to still further features in the described preferred embodiments a guanidine and cytosine content of each of the first and second oligonucleotides is at least 25%.

According to still further features in the described preferred embodiments the first and second oligonucleotides are labeled.

According to still further features in the described preferred embodiments the first and second oligonucleotides are attached to a solid substrate.

According to still further features in the described preferred embodiments the solid substrate is configured as a microarray and whereas each of the first and second oligonucleotides includes a plurality of oligonucleotides each attached to the microarray in a regio-specific manner.

According to yet other aspect of the present invention there is provided a method of identifying a novel drug target, the method comprising: (a) determining expression level of at least one naturally occurring antisense transcript of interest in cells characterized by an abnormal phenotype; and (b) comparing the expression level of the at least one naturally occurring antisense transcript of interest in the cells characterized by an abnormal phenotype to an expression level of the at least one naturally occurring antisense transcript of interest in cells characterized by a normal phenotype, to thereby identify the novel drug target.

According to still further features in the described preferred embodiments the abnormal phenotype of the cells is selected from the group consisting of biochemical phenotype, morphological phenotype and nutritional phenotype.

According to still further features in the described preferred embodiments determining expression level of at least one naturally occurring antisense transcript of interest is effected by at least one oligonucleotide designed and configured so as to be complementary to a sequence region of the at least one naturally occurring antisense transcript of interest, the sequence region not being complementary with a naturally occurring mRNA transcript.

According to still other aspect of the present invention there is provided a method of treating or preventing a disease, condition or syndrome associated with an upregulation of a naturally occurring antisense transcript complementary to a naturally occurring mRNA transcript, the method comprising administering a therapeutically effective amount of an agent for regulating expression of the naturally occurring antisense transcript.

According to still further features in the described preferred embodiments the agent for regulating expression of the naturally occurring antisense transcript is at least one oligonucleotide designed and configured so as to hybridize to a sequence region of the at least one naturally occurring antisense transcript.

According to still further features in the described preferred embodiments the at least one oligonucleotide is a ribozyme.

According to still further features in the described preferred embodiments the at least one oligonucleotide is a sense transcript.

According to a supplementary aspect of the present invention there is provided a method of diagnosing a disease, condition or syndrome associated with a substandard expression ratio of an mRNA of interest over a naturally occurring antisense transcript complementary to the mRNA of interest, the method comprising: (a) quantifying expression level of the mRNA of interest and the naturally occurring antisense transcript complementary to the mRNA of interest; (b) calculating the expression ratio of the mRNA of interest over the naturally occurring antisense transcript complementary to the mRNA of interest, thereby diagnosing the disease, condition or syndrome.

The present invention successfully addresses the shortcomings of the presently known configurations by providing a novel approach for identifying naturally occurring antisense transcripts, methods of designing artificial antisense transcripts according to information derived therefrom and methods and kits using naturally occurring and synthetic antisense transcripts.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates EST alignment along genomic DNA, generated according to the teachings of the present invention. Alignment results identify two strand groups of transcripts i.e., sense transcripts and antisense transcripts with an indicated sequence overlap.

FIG. 2 illustrates a system designed and configured for generating a database of naturally occurring antisense sequences generated according to the teachings of the present invention.

FIG. 3 illustrates a remote configuration of the system described in FIG. 2.

FIGS. 4a-k are sequence alignments of overlapping regions of selected naturally occurring antisense and sense sequence pairs identified according to the teachings of the present invention.

FIGS. 5a-g are sequence alignments of overlapping regions of selected naturally occurring antisense and sense sequence pairs identified according to the teachings of the present invention.

FIG. 6 schematically illustrates two transcription products of 53BP1 gene (red and green) and their corresponding partial complementary antisense transcripts of the 76p gene (blue). Numbers in parenthesis indicate length of sequence complementation. Schematic location of strand-specific RNA probes used for northern blotting of sense (53BP1, Riboprobe#1) and antisense (76p, Riboprobe#2) transcripts is shown.

FIG. 7 is an autoradiogram of a northern blot analysis depicting cellular distribution and expression levels of 53BP1 transcripts. Arrows on the right indicate the molecular weight of the identified 53BP1 transcripts relative to the migration of 28S and 18S ribosomal RNA subunits. |Numbers on the left denote the size of molecular weight markers in Kb.

FIG. 8 is an autoradiogram of a northern blot analysis depicting cellular distribution and expression levels of 76p transcripts. Arrows on the right indicate the molecular weight of the identified 76p transcripts relative to the migration of 28S and 18S ribosomal RNA subunits. |Numbers on the left denote the size of molecular weight markers in Kb.

FIG. 9 is an autoradiogram of a northern blot analysis depicting tissue distribution and expression levels of 76p transcripts. Arrows on the right indicate the molecular weight of the identified 76p transcripts. Numbers on the left denote the migration of molecular weight marker in Kb.

FIG. 10 illustrates the genomic organization of the 53BP1 gene and 76p gene, as elucidated from the RT-PCR analysis presented in the Examples section hereinbelow. Black arrows indicate the location of the primers used for RT-PCR analysis. Asterisks denote stop codons.

FIG. 11 schematically illustrates two transcription products of CIDE-B gene and their corresponding partial complementary antisense transcript of the BLTR2 gene. Schematic location of the strand-specific 430 nucleotide RNA probe used for northern analysis of sense (CIDE-B) and antisense (BLTR2) transcripts is shown. Dashed rectangles indicate the predicted coding sequence of the transcripts.

FIG. 12 is an autoradiogram of a northern blot analysis depicting cellular distribution and expression levels of BLTR2 transcripts. Arrows on the right indicate the molecular weight of the identified BLTR2 transcripts relative to the migration of 28S and 18S ribosomal RNA subunits. Numbers on the left denote the size of molecular weight markers in Kb.

FIG. 13 shows autoradiogram of a northern blot analysis depicting cellular distribution and expression levels of CIDE-B transcripts. Arrows on the right indicate the molecular weight of the identified CIDE-B transcripts relatively to the migration of 28S and 18S ribosomal RNA subunits. Numbers on the left denote the migration size of molecular weight markers in Kb.

FIG. 14 schematically illustrates a transcription product of APAF-1 gene and its corresponding partial complementary antisense transcripts of the EB-1 gene. Schematic location of the strand-specific 366 nucleotide RNA probe used for northern analysis of sense (APAF-1) and antisense (EB-1) transcripts is shown. Asterisks indicate the predicted coding sequence borders of the transcripts.

FIGS. 15a-b are autoradiograms of northern blot analyses depicting cellular distribution and expression levels of EB-1 (FIG. 15a) and APAF-1 transcripts (FIG. 15b). Numbers on the left denote the size of molecular weight marker in Kb.

FIG. 16 schematically illustrates a transcription product of the MINK-2 gene and its corresponding partial complementary antisense transcript of the AchR-ε gene. Schematic location of the strand-specific 280 nucleotide RNA probe used for northern analysis of sense (Mink-2) and antisense (AchR-ε) transcripts is shown.

FIGS. 17a-b are autoradiograms of northern blot analyses depicting cellular distribution and expression levels of AchR-ε antisense transcripts (FIG. 17a) and the sense complementary transcript of Mink-2 (FIG. 17b). Arrows on the right denote the migration of molecular weight markers in Kb.

FIG. 18 schematically illustrates a transcription product of Cyclin-E2 gene and its corresponding partial complementary antisense transcript. Schematic location of strand-specific RNA probes used for northern blotting of sense (Riboprobe#1) and antisense (Riboprobe#2) transcripts is shown.

FIGS. 19a-b are autoradiograms of northern blot analyses depicting cellular distribution and expression levels of Cyclin E2 antisense transcript (FIG. 19a) and the sense complementary transcript (FIG. 19b). Arrows on the left denote the migration of molecular weight markers in Kb.

FIG. 20 illustrates results from RT-PCR analysis of the expression patterns of CIDE-B transcript and its complementary naturally occurring antisense transcript following concentration dependent induction of apoptosis. Lanes: (1) 50 μM etoposide; (2) 100 μM etoposide; (3) 250 μM etoposide; (4) 500 μM etoposide; (5) 10 nM staurosporine; (6) 100 nM staurosporine; (7) 250 nM staurosporine; (8) 1000 nM staurosporine; (9) untreated cells (UT).

FIGS. 21a-c are results of RT-PCR analyses depicting expression patterns of AchRε and its naturally occurring antisense transcript following time-dependent induction of differentiation. FIG. 21a illustrates the position of riboprobes used for reverse transcription reaction. FIG. 21b shows the reciprocal expression pattern of sense and antisense transcripts (indicated by arrows). FIG. 21c shows the expression pattern of the antisense transcript alone.

FIGS. 22a-j illustrate results of northern blot analysis of sense/antisense clusters revealing positive signals for sense/antisense genes in the microarray analysis. Diagrams describing genomic organization of the relevant region for each of the sense/antisense clusters are included above the autoradiograms, and regions of overlap (including GenBank accession number) from which the strand-specific riboprobes were derived are included. Sense-antisense pair numbers are as they appear in the microarray (as depicted in Table_S2 on the attached CD-ROM2 and in conversion Table 6). FIG. 22a reveals expression patterns of randomly selected sequence pair number 235, denoted as Rand_{—}235 in Table 6. Similarly, FIG. 22b corresponds to pair number 173, FIG. 22c to pair number 248, FIG. 22d to pair number 6, FIG. 22e to pair number 216, FIG. 22f to pair number 239, FIG. 22g to pair number 202, FIG. 22h to pair number 114, FIG. 22i to pair number 188, and FIG. 22j to pair number 223. Eight pairs (FIGS. 22a-h) evaluated revealed positive signals for both sense and antisense expression, while two (FIGS. 22i-j) revealed a positive signal for only one of the genes, with the counterpart being a known RefSeq mRNA.

FIG. 23 is a Table depicting expression patterns in various cell lines and tissues as probed with a subset of 264 pairs from the putative sense/antisense dataset of the present invention. The pairs are denoted by the pair number and described in Table_S1 of CD-ROM2. “C” and “AC” denote the two counterpart probes. Expression was also verified for positive controls, including the ubiquitously expressed genes gapdh, actin, hsp70 and gnb211 in various concentrations, and 11 previously documented sense/antisense pairs. Expression thresholds were verified and indicated as “+”, if the probe passed the threshold in at least one cell line or tissue or “−”, if the probe did not pass the threshold in all experiments. In cases where both the sense and the antisense oligo passed the expression threshold, the antisense was declared “verified”. In cases where only one of the probes passed the expression threshold, but the other probe was fully contained within a known mRNA deposited in GenBank, the antisense was declared “indirectly verified”. Normalization for microarray signals was conducted as described in the methods section. Rji ratios were obtained for each cell line/tissue assessed. Cases of flagged-out spots for which there was no information were marked “−1.00”. Data represent values of the two reciprocal experiments.

### DESCRIPTION OF THE PREFERRED EMBODIMENTS

The present invention is of methods of identifying naturally occurring antisense transcripts, which can be used in kits and methods for quantifying gene expression levels. Specifically, the antisense molecules and related oligonucleotides generated according to information derived therefrom of the present invention can be used to detect, quantify, or specifically regulate antisense and respective sense transcripts thereby enabling detection and treatment of a wide range of disorders.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings described in the Examples section. The invention is capable of other embodiments or of being practiced or carried out in various ways. Also, it is to be understood that the phraseology and terminology employed herein is for the purpose of description and should not be regarded as limiting.

Terminology

As used herein, the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

The term “antisense” refers to a complementary strand of an mRNA transcript e.g., antisense RNA.

The phrase “naturally occurring antisense transcripts” refers to RNA transcripts encoded from an antisense strand of the DNA. These endogenous transcript exhibit at least partial complementarity to mRNA transcripts transcribed from the sense strand of a DNA, also termed sense transcripts. cis-encoded naturally occurring antisense transcripts are transcribed from the same locus as the sense transcripts. trans-encoded antisense transcripts are transcribed from a different locus than the respective sense transcripts.

The phrase “antisense strand” or “anticoding strand” refers to a strand of DNA, which serves as a template for mRNA transcription and as such is complementary to the mRNA transcript formed.

The phrase “sense strand” or “coding strand” refers to the strand of DNA, which is identical to the mRNA transcript formed.

The phrase “complementary DNA” (cDNA) refers to the double stranded or single stranded DNA molecule, which is synthesized from a messenger RNA template.

The phrase “sense oriented polynucleotides” refers to polynucleotide sequences of a complementary or genomic DNA. Such polynucleotide sequences can be from exon regions, in which case they can encode mRNAs or portions thereof, or from intron regions, in which case they typically do not encode mRNA or portions thereof.

The term “contig” refers to a series of overlapping sequences with sufficient identity to create a longer contiguous sequence.

The term “cluster” refers to a plurality of contigs all derived, with a high degree of probability, from a single gene. Clusters are generally formed based upon a specified degree of homology and overlap (e.g., a stringency). The different contigs in a cluster do not typically represent the entire sequence of the gene, rather the gene may comprise one or more unknown intervening sequences between the defined contigs.

The phrase “open reading frame” (ORF) refers to a nucleotide sequence, which could potentially be translated into a polypeptide. Such a stretch of sequence is uninterrupted by a stop codon. An ORF that represents the coding sequence for a full protein begins with an ATG “start” codon and terminates with one of the three “stop” codons. For the purposes of this application, an ORF may be any part of a coding sequence, with or without start and/or stop codons. For an ORF to be considered as a good candidate for coding for a bona fide cellular protein, a minimum size requirement is often set, for example, a stretch of DNA that would code for a protein of 50 amino acids or more. An ORF is not usually considered an equivalent to a gene or locus until a phenotype is associated with a mutation in the ORF, an mRNA transcript for a gene product generated from the ORF's DNA has been detected, and/or the ORF's protein product has been identified.

The term “annotation” refers to a functional or structural description of a sequence, which may include identifying attributes such as locus name, poly(A)/poly(T) tail and/or signal, key words, Medline references and orientation cloning data.

Naturally occurring antisense molecules can play a role in sense transcription stability and function (e.g. translation). To date, most, if not all of the information relating to naturally occurring antisense transcripts was obtained by either low efficiency computational approaches (described hereinabove) or by approaches utilizing RNase protection assays, northern blot analysis, strand-specific RT PCR, subtractive hybridization, differential plaque hybridization, affinity chromatography, electrospray mass spectrometry and the like. These methods, though highly reliable, are extremely laborious, time consuming and are directed at individual target transcripts. As such, current approaches for uncovering antisense transcripts can be used to detect a negligible portion of the number of naturally occurring antisense molecules thought to exist.

As described hereinunder and in the Examples section, which follows, the present invention provides a novel approach for systematically identifying naturally occurring antisense molecules.

Aside from large scale applicability, the present method can be used to identify naturally occurring antisense molecules even in cases where the antisense transcriptional unit is localized to an intron of an expressed gene or to a different locus than the complementary sense encoding gene (e.g., trans-encoded antisense), or in cases where the antisense molecule lacks an open reading frame or appreciable complementarity to known sense molecules. Antisense transcripts uncovered according to the teachings of the present invention can be used for detecting and accurately quantifying respective sense counterparts as well as for sensibly designing artificial antisense molecules suitable for down-regulation of sense counterparts.

Thus, according to one aspect of the present invention there is provided a method of identifying putative naturally occurring antisense transcripts.

The method according to this aspect of the present invention is effected by the following steps.

First, sense-oriented polynucleotide sequences of a first database are computationally aligned with expressed polynucleotide sequences of a second database.

Following computational alignment, expressed polynucleotide sequences are analyzed according to one or more criteria for their ability to hybridize or form a duplex or partial complementation with the sense-oriented polynucleotide sequences (further detailed hereinbelow and in the Examples section which follows).

Expressed polynucleotide sequences which are capable of forming a duplex with sense oriented sequences are considered as putative naturally occurring antisense molecules and as such can be stored in a database which can be generated by a suitable computing platform.

Final confirmation of computationally obtained putative naturally occurring antisense molecules can be effected either computationally or preferably by using suitable laboratorial methodologies, based on nucleotide hybridization including RNase protection assay, subtractive hybridization, differential plaque hybridization, affinity chromatography, electrospray mass spectrometry, northern analysis, RT-PCR and the like (for further details see the Examples section).

Information derived from the sequence, sense position and other structure characteristics of the naturally occurring antisense transcripts identified according to the teachings of the present invention can be used to quantify respective sense transcripts of interest or to generate corresponding artificial antisense polynucleotides, which can be packed in diagnostic or therapeutic kits and implemented in various therapeutic and diagnostic methods.

Expressed polynucleotide sequences used as a potential source for identifying naturally occurring antisense transcripts according to this aspect of the present invention are preferably libraries of expressed messenger RNA [i.e., expressed sequence tags (EST), cDNA clones, contigs, pre-mRNA, etc.] obtained from tissue or cell-line preparations which can include genomic and/or cDNA sequence.

Expressed polynucleotide sequences, according to this aspect of the present invention can be retrieved from pre-existing publicly available databases (i.e., GenBank database maintained by the National Center for Biotechnology Information (NCBI), part of the National Library of Medicine, and the TIGR database maintained by The Institute for Genomic Research) or private databases (i.e., the LifeSeq.™ and PathoSeq.™ databases available from Incyte Pharmaceuticals, Inc. of Palo Alto, Calif.).

Alternatively, the sequence database of the expressed polynucleotide sequences utilized by the present invention can be generated from sequence libraries (e.g., cDNA libraries, EST libraries, mRNA libraries and others). cDNA libraries are suitable sources for expressed sequence information.

Generating a sequence database in such a case is typically effected by tissue or cell sample preparation, RNA isolation, cDNA library construction and sequencing.

It will be appreciated that such cDNA libraries can be constructed from RNA isolated from whole organisms, tissues, tissue sections, or cell populations. Libraries can also be constructed from tissue reflecting a particular pathological or physiological state. Of particular interest are libraries constructed from sources associated with certain disease states, including malignant, neoplastic, hyperplastic tissues and the like.

Once raw sequence data is obtained, sequences are selected and preferably annotated before stored in a database. Selection proceeds according to one or more sequence criterion, which will be further detailed hereinunder. The editing, annotation and selection process is divided into two stages of processing. One stage comprises removal of repetitive, redundant or non-informative and contaminant sequences. The second stage involves selection of suitable candidates of putative naturally occurring antisense sequences.

The following section describes the different selection criteria which can be used for sequence filtering.

Vector contamination—“chops” vector elements and linker motifs used for the process of cloning from desired expressed nucleotide sequences. This selection can be effected by screening manually updated databases of sequences included in commonly used expression or cloning vectors.

Contaminating sequences—includes sequences which are derived from an undesired source. Such sequences can be recognized by their nucleotide distribution and/or by homology searches such as alignment searches using any sequence alignment algorithm such as BLAST (Basic Local Alignment Search Tool, available through www.ncbi.nlm.nih.gov/BLAST) or the Smith-Waterman algorithm. Other contaminating sequences may include sequences exhibiting high occurrence of di-nucleotide distribution mostly related to sequencing artifacts and ribosomal RNA sequences.

Repetitive elements and low complexity sequences—eliminates or masks expressed sequences comprising known repetitive elements (ALU, L1 etc.) and low complexity sequences (i.e., a di- or tri-nucleotide repeat). Such elimination is preferably effected by comparison with database of known repetitive elements. It will be appreciated that this type of selection is mostly species specific. Masking of low complexity sequences can be effected by substituting an N (i.e., an inert character) for the actual nucleotide (i.e., G, A, T, or C). Masking of low complexity sequences facilitates further computational analysis and maintains the spacing of the molecule.

Sequence length—preferred expressed sequences are of a length between 20-2000, preferably 20-1000, more preferably 20-500, most preferably 20-300 base pairs.

Sequence annotation—expressed sequences retrieved from external databases, i.e., GenBank, oftentimes include an annotation which indicates direction of the sequencing of the insert clone (i.e., 5′ or 3′ direction). Sequence annotation, though “noisy” by nature due to multiple entries from various sources; artifacts taking place during directional cloning and incidence of palindromic eight-cutter restriction sites located at the end of the sequence, can serve as an important tool for deducing strand identity using dedicated computer software which are further discussed hereinunder

Intron splice site consensus sequence intron splice site sharing—intron sequences nearly always begin with a di-nucleotide sequence of GT (“splice donor”) and end with an AG (“splice acceptor”) preceded by a pyrimidine-rich tract. This consensus sequence is part of the signal for splicing. Intron splice site consensus sequence on the complementary strand (e.g., antisense strand) begins with CT and ends with AC. Thus, combined with genomic data, expressed sequences having a GT . . . AG can be considered as sense-oriented sequences, while a CT . . . AC pattern is considered as an antisense oriented sequence. This selection criterion is very stringent since only negligible portions of introns have a CT . . . AC pattern. Sequences that share a similar splicing pattern, as deduced by alignment to genomic data, may be considered as having the same sense orientation, also termed herein as “intron sharing”. It will be appreciated by one skilled in the art that using these selection criteria requires a careful and accurate alignment of expressed sequences to genomic sequence.

Poly(A) tails and Poly(T) heads—most eukaryotic mRNA molecules contain a poly-adenylation [poly(A)] tail at their 3′ end. This poly(A) tail is not encoded by DNA. Therefore an expressed sequence which has a poly(A) tail can be considered as sense oriented. Similarly, poly(T) heads, which are not encoded from a genomic sequence indicate that a sequence is of the opposite direction, namely antisense oriented. Notably, genomically encoded Poly(A) tails and poly(T) heads provide no information as to the sequence orientation.

Poly(A) signal—some mature mRNA transcripts contain internal AAUAAA sequence. This internal sequence is part of an endonuclease cleavage signal. Following cleavage by the endonuclease, a poly(A) polymerase adds about 250 A residues to the 3′ end of the transcript. Hence, expressed sequences containing a poly(A) signal can be considered as sense oriented.

Rare restriction site used for cloning—for example, eight cutter endonucleases which cleave 8-mer palindromic sequences and are characterized by a low frequency of cutting often used in genome mapping and EST library preparations (e.g., NotI. Commercially available from Promega: www.promega.com). Therefore, when a cluster of overlapping expressed sequences is characterized by a portion of sequences starting with a digestion site and another portion ending with the same, these sequences may be considered as encoded from the same strand. However, any endonuclease capable of digesting a palindromic sequence (i.e., XhoI, SalI, PacI etc.) may also affect distorted sequence clustering, therefore strand orientation is preferably effected using other parameters as well.

Sequence overlap—sequences that completely overlap are considered to have the same strand orientation.

The above described parameters are used individually or in combination to analyze the expressed polynucleotide sequences so as to select anti-sense oriented sequences.

Selection can be effected on the basis of a single criterion or several criteria considered individually or in combination.

In cases where several criteria are examined, a scoring system e.g., a scoring matrix, is preferably used.

Since in some cases identifying an intron splicing consensus site may be more important than both sequence annotation and NotI alignment, while in others, detection of poly(A) tails and poly(T) heads might be the most significant criterion, the use of a scoring matrix in which each criterion is weighted enables one to select qualified antisense transcripts.

Such a scoring matrix can list the various expressed polynucleotide sequences across the X-axis of the matrix while each criterion can be listed on the Y-axis of the matrix. Criteria include both a predetermined range of values from which a single value is selected from each sequence, and a weight. Each sequence is scored at each criterion according to its value and the weight of the criterion.

When using such a scoring matrix the scores of each criterion of a specific sequence are summed and the results are analyzed.

Expressed sequences which exhibit a total score greater than a particular stringency threshold are grouped as members of either a sense-oriented sequence set or antisense-oriented sequence set; the higher the score the more stringent the criteria of grouping.

It will be appreciated that the above described analysis can take place prior to computational alignment to sense oriented sequences, i.e., during the process of editing the expressed sequence database which is described hereinabove. Alternatively, selection can take place following computational alignment, thus further facilitating identification of proper duplex formation between the sense oriented polynucleotide sequences and expressed polynucleotide sequences.

Genomic DNA or a portion thereof is preferably used as sense-oriented sequence data according to this aspect of the present invention. It is conceivable that the present invention can determine sense orientation and antisense orientation of a database of expressed sequences simply by computationally aligning the sequences of the expressed database onto the genome, and finding whether two complementary expressed sequences hybridize to the genome (e.g., virtually generate a double stranded portion thereof). Such two overlapping sequences constitute sense and naturally occurring antisense transcripts.

Utilizing genomic DNA as a sense oriented template is preferred for the following reasons: (i) identifying trans-encoded antisense transcripts; (ii) analyzing intron splice consensus site and intron sharing; (iii) omitting genomically encoded poly(A) and poly(T) sequences; and (iv) analyzing sequences encompassing eight-cutter restriction sites.

Computational alignment of expressed polynucleotide sequences to the sense-oriented polynucleotide sequences (e.g., genomic sense sequences) can be effected using any commercially available alignment software, including sequence alignment tools utilizing algorithm such as BLAST (Basic Local Alignment Search Tool, available through www.ncbi.nlm.nih.gov/BLAST) or Smith-Waterman.

Assembly software is preferably used according to this aspect of the present invention. Such software is of high value when complete genomic information is unavailable or when handling large amounts of expressed sequence data. A number of commonly used computer software fragment read assemblers capable of forming clusters of expressed sequences are now available. These packages include but are not limited to, The TIGR Assembler [Sutton G. et al. (1995) Genome Science and Technology 1:9-19], GAP [Bonfield J K. et al. (1995) Nucleic Acids Res. 23:4992-4999], CAP2 [Huang X. et al. (1996) Genomics 33:21-31], The Genome Construction Manager [Laurence C B. Et al. (1994) Genomics 23:192-201], Bio Image Sequence Assembly Manager, SeqMan [Swindell S R. and Plasterer J N. (1997) Methods Mol. Biol. 70:75-89], LEADS and GenCarta (Compugen Ltd. Israel).

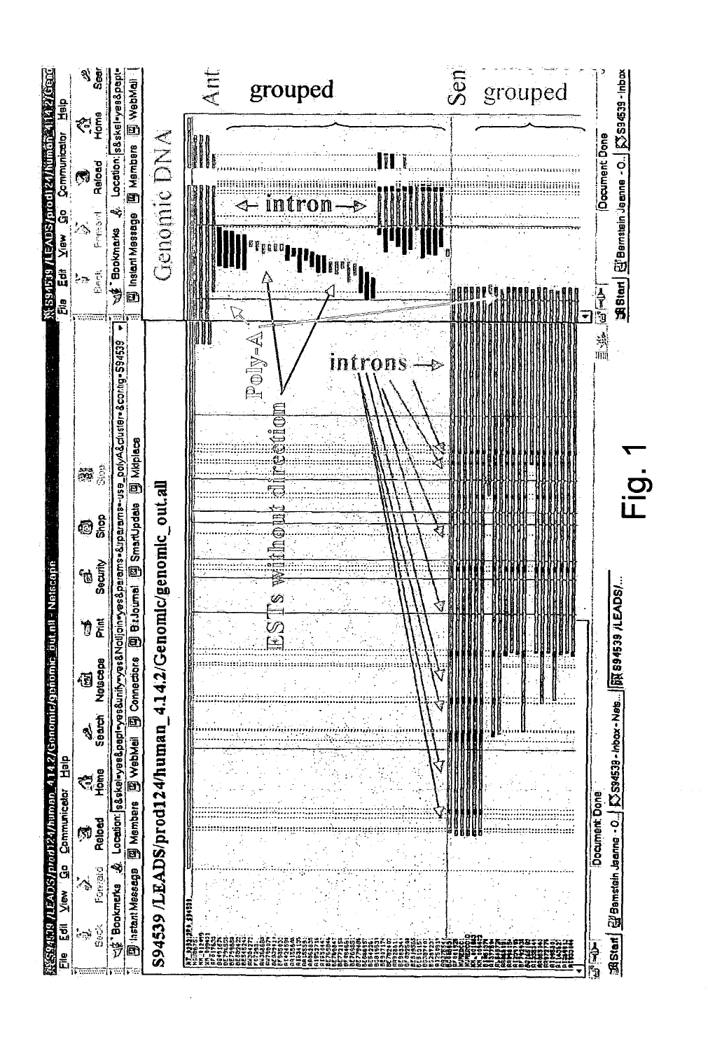

Computer assembly and alignment programs can be modified to incorporate sequence criteria for determining sense or antisense orientation of expressed nucleotide sequences, as described hereinabove. Thereby, avoiding deliberate inversion of sequences during the assembly process, while ignoring the natural orientation of the sequences (i.e., sense or antisense orientation). FIG. 1 illustrates results of expressed sequence assembly against genomic data and final distinction between sense oriented transcripts and antisense oriented transcripts of a single gene.

Following a proper alignment of expressed sequences to sense oriented polynucleotide sequences, duplexes are identified. The term “duplex” is used herein to indicate that a sequence identified according to this aspect of the present invention is complementary to a sense-oriented polynucleotide sequence. Complementation may be to a portion of the sense sequence, i.e., a region thereof, or alternatively, to two or more non-contiguous regions, which may be separated by one or more nucleotides on the sense strand.

The formation of sense-antisense duplexes does not require 100% complementation nor does it require participation of the entire sense/antisense transcript sequence. The sense or antisense transcripts can have a secondary structure (e.g., stem and loop) generated by intra-sequence hybridization which can prevent specific sequence regions in the sense or antisense transcripts from participating in duplex formation. Thus, the antisense of the sequence identified, according to this aspect of the present invention can be complementary to its sense counterparts in several regions, which are not necessarily close to each other when the sense transcript is in linear form.

Although any length of sequence overlap can generate a duplex, overlaps of at least 5 preferably 20 more preferably 30 even more preferably 40 bp are considered more indicative of true sense-antisense duplex formation.

The method of uncovering putative antisense transcripts of the present invention is preferably carried out using a dedicated computational system.

Thus, according to another aspect of the present invention and as illustrated in FIG. 2, there is provided a system for generating a database of putative naturally occurring antisense sequences which system is referred to hereinunder as system 10.

System 10 includes a processing unit 12, which executes a software application designed and configured for aligning sense oriented polynucleotide sequences with expressed polynucleotide sequences and identifying expressed polynucleotide sequences which are capable of forming a duplex with the sense oriented polynucleotide sequences, thereby recognizing putative naturally occurring antisense transcripts. System 10 may also include a user input interface 14 (e.g., a keyboard and/or a mouse) for inputting database or database related information, and a user output interface 16 (e.g., a monitor) for providing database information to a user.

System 10 preferably stores sequence information of the putative antisense transcripts identified thereby on a computer readable media such as a magnetic, optico-magnetic or optical disk to thereby generate a database of putative antisense transcript sequences. Such a database further includes information pertaining to database generation (e.g., source library), parameters used for selecting polynucleotide sequences, putative uses of the stored sequences, and various other annotations and references which relate to the stored sequences or respective sense transcripts.

System 10 of the present invention may be used by a user to query the stored database of sequences, to retrieve nucleotide sequences stored therein or to generate polynucleotide sequences from user inputted sequences.

System 10 can be any computing platform known in the art including but not limited to, a personal computer, a work station, a mainframe and the like.

The database generated and stored by system 10 can be accessed by an on-site user of system 10, or by a remote user communicating with system 10.

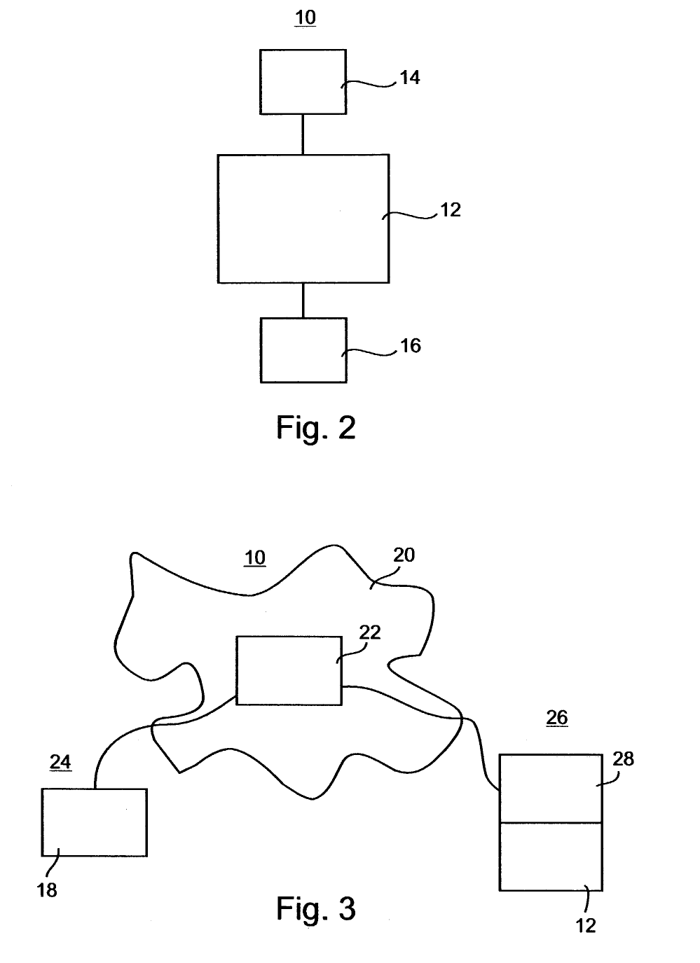

As illustrated in FIG. 3, communication between a remote user 18 and processing unit 12 is preferably effected via a communication network 20. Communication network 20 can be any private or public communication network including, but not limited to, a standard or cellular telephony network, a computer network such as the Internet or intranet, a satellite network or any combination thereof.

As illustrated in FIG. 3, communication network 20 includes one or more communication servers 22 (one shown in FIG. 3) which serves for communicating data pertaining to the polypeptide of interest between remote user 18 and processing unit 12.

It will be appreciated that existing computer networks such as the Internet can provide the infrastructure and technology necessary for supporting data communication between any number of sites 24 and remote analysis sites 26.

For example, using a computer operating a Web browser application and the World Wide Web, any expressed polynucleotide sequence of interest can be “uploaded” by user 18 onto a Web site maintained by a database server 28. Following uploading, database server 28 which serves as processing unit 12 can be instructed by the user to processes the polynucleotide as is described hereinabove.

Following such processing, which can be performed in real time, nucleic acid sequence results can be displayed at the web site maintained by database server 28 and/or communicated back to site 24, via for example, e-mail communication.

Thus, using the Internet, a remote configuration of system 10 can provide polynucleotide sequence analysis services to a plurality of sites 24 (one shown in FIG. 3).

It will be appreciated that this configuration of system 10 of the present invention is especially advantageous in cases where polypeptide analysis can not be effected on-site. For example, laboratories, which lack the equipment necessary for executing the analysis or lack the necessary skills to operate it.

Thus, data extracted from the database of naturally occurring antisense transcripts of the present invention is of high value for designing oligonucleotides suitable for transcript detection and quantification and for sensibly designing artificial antisense oligonucleotides for down-regulation and elimination of a transcript of interest or changing the balance between sense and complementary antisense transcripts. The possibility of up-regulating a transcript of interest using naturally occurring antisense based-oligonucleotides generated according to the teachings of the present invention is also realized. In addition, data extracted from the database of naturally occurring antisense transcripts may also be used for assessing endogenous double stranded-RNA also termed interfering RNA, which may distort gene-expression due to either RNA-degradation, DNA-methylation, polycomb mediated suppression etc. (for details see the Background section hereinabove).

Antisense technology is based upon the pairing of an artificially designed antisense oligonucleotide, with a target nucleic acid. The use of antisense technology requires a complementarity of the antisense nucleotide sequence to a target zone of an mRNA target sequence that will effect inhibition of gene expression [reviewed in Stein C A. and Cohen J S. (1988) Cancer Res. 48:2659-68]. Based on empiric experience it was shown that the success of antisense technology relies on: (i) cellular uptake; (ii) stability of artificial antisense molecules under physiological conditions (i.e., cellular pH, endonucleases etc.); (iii) complementation between the oligonucleotide and a single stranded target sequence (i.e., tertiary structure of target RNA will not form a good target); (iv) binding specificity of antisense oligonucleotide so as not to compete with other RNA binders (e.g. proteins) to thereby maintain an effective antisense concentration.

Various attempts to employ antisense technology while considering the above discussed limitations included using large amounts of oligonucleotides to overcome cellular uptake and environmental barriers and chemically modified antisense nucleotide compositions, for obtaining higher level of cellular stability. However, even in case where uptake difficulties are traversed, the step of target identification (i.e., RNA-target sequence region) continues to be the major bottleneck for successful implementation of antisense technology.

U.S. Pat. No. 6,183,966 discloses a method and an apparatus for ranking nucleic acid sequences based on stability of nucleic acid oligomer sequence binding interactions to select sequence zones for antisense targeting. This method however systematic, relies on thermodynamic analyses combined with numerous predictions which cannot be considered empirically accurate and reliable.

Thus according to another aspect of the present invention there is provided a method of designing artificial antisense transcripts.

The method according to this aspect of the present invention is effected by the following steps.

First, structural and/or functional parameters pertaining to naturally occurring antisense transcripts are extracted/deduced from a database such as the one described hereinabove. These parameters may be generally deduced from all sequences stored in the database, or extracted from specific antisense sequences or preferably groups of antisense sequences.

Second, artificial antisense molecules of interest are designed according to the extracted parameters.

Such parameters may be divided into three groups, topographical parameters, functional parameters and structural parameters.

Topographical parameters—(i) position of sequence overlap on the sense transcript (i.e., coding region, 5′UTR, 3′UTR); (ii) position of the sequence overlap on the antisense transcript (end overlap, middle overlap, full overlap). (iii) length of overall sequence overlap; (iv) continuity or discontinuity of sequence overlap.

Structural parameters—pertains to both sense and antisense transcripts (i) tertiary structure (i.e., hairpin, helix, stem and loop, pseudoknot, and the like); (ii) single stranded versus double stranded regions; (iii) GC content; (iv) tandem Gs; (v) adenosine/inosine content; (vi) thermodynamic stability of tertiary structures; (vii) duplex melting point; (viii) methylations and other RNA modifications; (ix) RNA-protein interactions; and (x) transcript length.

Functional parameters—(i) alternative splicing; (ii) tissue expression; (iii) pathology specific expression; (iv) antisense promoters; (v) intron content; (vi) open reading frame in antisense transcript.

These parameters can be used individually or in combination, in which case, each parameter is preferably weighted according to its importance. Due to the multi-factorial design of artificial antisense transcripts according to this aspect of the present invention, employing a scoring system (described hereinabove) is preferably used to simplify and increase the accuracy of the process.

Synthetic antisense oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this aspect of the present invention are those having a length selected from a range of 10 to about 200 bases preferably 15-150 bases, more preferably 20-100 bases, most preferably 20-50 bases.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistance to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. ,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374. Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

The present invention also includes antisense molecules, which are chimeric molecules. “Chimeric” antisense molecules”, are oligonucleotides, which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids. An example for such include RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense molecules of the present invention may be formed as composite structures of two or more oligonucleotides, modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

Finally, chimeric oligonucleotides of the present invention can comprise a ribozyme sequence. Ribozymes are being increasingly used for the sequence-specific inhibition of gene expression by the cleavage of mRNAs. Several ribozyme sequences can be fused to the oligonucleotides of the present invention. These sequences include but are not limited ANGIOZYME specifically inhibiting formation of the VEGF-R (Vascular Endothelial Growth Factor receptor), a key component in the angiogenesis pathway, and HEPTAZYME, a ribozyme designed to selectively destroy Hepatitis C Virus (HCV) RNA, (Ribozyme Pharmaceuticals, Incorporated—WEB home page).

The oligonucleotides generated according to the teachings of the present invention can be used for both diagnostic and therapeutic purposes. For example, oligonucleotides of the present invention can be used to diagnose and treat a variety of diseases or pathological conditions associated with an abnormal expression (i.e., up-regulation or down-regulation) of at least one mRNA molecule of interest, including but not limited to diabetes, autoimmune diseases, Parkinson, Alzheimer' disease, HIV, malaria, cholera, influenza, rabies, diphtheria, breast cancer, colon cancer, cervical cancer, melanoma, lung cancer, ovarian cancer, pancreatic cancer, prostate cancer, lymphomas, leukemias and the like and any other diseases (see Example 8 of the Examples section) which are associated with aberrant expression of multiple mRNAs (i.e., sense and/or antisense) or with unregulated formation of endogenous double stranded RNA complexes.

Present-day mRNA-based diagnostic assays utilize oligonucleotide probes which are complementary to one or more regions of the mRNA to be quantitated. Such probes are designed while considering interspecies sequence variation, sequence length, GC content etc. However design of such prior art probes (i.e., riboprobes or deoxyriboprobes) does not take into consideration the presence of antisense transcripts which can effect probe binding efficiency. Discounting antisense presence can lead to inaccurate diagnosis, which is oftentimes followed by an erroneous treatment protocol.

The present invention provides an mRNA-detection/quantification assay, which is devoid of this limitation.

Thus, according to an additional aspect of the present invention there is provided a method of quantifying at least one mRNA of interest in a biological sample.

As used herein, the phrase “biological sample” refers to any sample derived from biological tissues or fluids, including blood (serum or plasma), sputum, pleural effusions, urine, biopsy specimens, isolated cells and/or cell membrane preparation. Methods of obtaining tissue biopsies and body fluids from mammals are well known in the art.

The method of this aspect of the present invention is effected by contacting mRNA from a cell type or within a cell with one or more oligonucleotides that hybridizes efficiently with a sequence region of an mRNA transcript which is not complementary with a naturally occurring antisense transcript.

In addition to the limitation described above, prior art diagnostic/detection assays also fail to consider the effect of antisense transcription on the protein expression levels of a gene of interest. It stands to reason that presence of antisense transcripts in a biological sample can substantially reduce the resultant protein levels translated from a complementary sense transcript. Consistently, diseases which are associated with endogenous dsRNA complexes, are also very difficult to detect and moreover to treat, due to insufficient sequence data pertaining to duplex forming transcripts.

Thus, for accurate quantification of gene expression, both the sense and antisense levels must be quantified and/or their respective expression ratio must be determined.

By contacting a biological sample with one or more pairs of oligonucleotides, where one oligonucleotide is capable of hybridizing with the mRNA of interest and the second oligonucleotide is capable of hybridizing with a naturally occurring antisense transcript which is complementary with the mRNA of interest such accurate quantification can be effected.

Contacting the oligonucleotides of the present invention with the biological sample is effected by stringent, moderate or mild hybridization (as used in any polynucleotide hybridization assay such as northern blot, dot blot, RNase protection assay, RT-PCR and the like). Wherein stringent hybridization is effected by a hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 1-1.5° C. below the Tm, final wash solution of 3 M TMACI, 0.01 M sodium phosphate (pH 6.8), 1 mM EDTA (pH 7.6), 0.5% SDS at 1-1.5° C. below the Tm; moderate hybridization is effected by a hybridization solution of 6×SSC and 0.1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 2-2.5° C. below the Tm, final wash solution of 3 M TMACI, 0.01 M sodium phosphate (pH 6.8), 1 mM EDTA (pH 7.6), 0.5% SDS at 1-1.5° C. below the Tm, final wash solution of 6×SSC, and final wash at 22° C.; whereas mild hybridization is effected by a hybridization solution of 6×SSC and 1% SDS or 3 M TMACI, 0.01 M sodium phosphate (pH 6.8), 1 mM EDTA (pH 7.6), 0.5% SDS, 100 mg/ml denatured salmon sperm DNA and 0.1% nonfat dried milk, hybridization temperature of 37° C., final wash solution of 6×SSC and final wash at 22° C.

The oligonucleotides of the present invention can be attached to a solid substrate, which may consist of a particulate solid phase such as nylon filters, glass slides or silicon chips [Schena et al. (1995) Science 270:467-470].

In a particular embodiment, oligonucleotides of the present invention can be attached to a solid substrate, which is designed as a microarray. Microarrays are known in the art and consist of a surface to which probes that correspond in sequence to gene products (e.g., cDNAs, mRNAs, cRNAs, polypeptides, and fragments thereof), can be specifically hybridized or bound at a known position (regiospecificity).

Several methods for attaching the oligonucleotides to a microarray are known in the art including but not limited to glass-printing, described generally by Schena et al., 1995, Science 270:467-47, photolithographic techniques [Fodor et al. (1991) Science 251:767-773], inkjet printing, masking and the like.

In general, quantifying hybridization complexes is well known in the art and may be achieved by any one of several approaches. These approaches are generally based on the detection of a label or marker, such as any radioactive, fluorescent, biological or enzymatic tags or labels of standard use in the art. A label can be applied on either the oligonucleotide probes or nucleic acids derived from the biological sample.

The following illustrates a number of labeling methods suitable for use in the present invention. For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, FluorX (Amersham) and others [e.g., Kricka et al. (1992), Academic Press San Diego, Calif.] can be attached to the oligonucleotides. It will be appreciated that pairs of fluorophores are chosen when distinction between two emission spectra of two oligonucleotides is desired or optionally, a label other than a fluorescent label is used. For example, a radioactive label, or a pair of radioactive labels with distinct emission spectra, can be used [Zhao et al. (1995) Gene 156:207]. However, because of scattering of radioactive particles, and the consequent requirement for widely spaced binding sites, the use of fluorophores rather than radioisotopes is more preferred.

The intensity of signal produced in any of the detection methods described hereinabove may be analyzed manually or using a software application and hardware suited for such purposes.

In general, mRNA quantification is preferably effected alongside a calibration curve so as to enable accurate mRNA determination. Furthermore, quantifying transcript(s) originating from a biological sample is preferably effected by comparison to a normal sample, which sample is characterized by normal expression pattern of the examined transcript(s).

It will be appreciated that the detection method described above can also be used for quantifying at least one naturally occurring antisense transcript in a biological sample. In such a case, the oligonucleotide used for quantification is designed to hybridize with a sequence region of naturally occurring antisense transcript of interest, which is not complementary with a naturally occurring mRNA transcript.

The diagnostic assays described hereinabove can be used to accurately distinguish between absence, presence and excess expression of any transcripts of interest (e.g., sense, antisense), and to monitor their level during therapeutic intervention. These methods are also capable of diagnosing diseases associated with an improper balance or ratio between sense and antisense expression and diseases associated with endogenous dsRNA.

Further description of oligonucleotide-pair arrays is provided in Example 9 of the Examples section which follows.

As discussed hereinabove oligonucleotides of the present invention can be also used for therapeutic purposes, such as treating diseases or conditions associated with aberrant expression levels of one or more sense and/or antisense transcripts and conditions, which are associated with endogenous dsRNA such as unregulated formation of double-strand RNA (i.e., up/down-regulation).

Accumulative knowledge shows strong correlation between a variety of human diseases and mutations, over-expression and function of the protein building blocks (i.e., protein kinases, phosphatsases) and their effectors and regulators, which constitute numerous intracellular signaling pathways. For instance, inactivation of both copies of ZAP-70 or Jak-3 causes severe combined immunodeficiency and mutation of the X-linked BTK gene results in agammaglobulinemia. Many genetic disorders are also associated with mutations for example, in protein-serine kinases (PSKs) and phosphatases. The Coffin-Lowry syndrome results from inactivation of the X-linked Rsk2 gene, and myotonic dystrophy is due to decreased levels of expression of the myotonic dystrophy PSK. In addition, over-expression of ErbB2 receptor tyrosine kinase is implicated in breast and ovarian carcinoma [reviewed by Hunter T. (2000) Cell 100:1 13-127].

Given the importance of activated kinases in a variety of disorders such as cancer, it would be anticipated that phosphatases regulation would be found as tumor suppressor genes and as promising drug targets. So far this has not proved to be the case. Furthermore, a number of diseases are associated with insufficient expression of signaling molecules, including non-insulin-dependent diabetes and peripheral neuropathies.

Thus, it is conceivable that identification of naturally occurring antisense transcripts of signaling molecules participating in specified signaling pathways may serve as promising tools for both identification and particularly treatment of a variety of disorders at any gene expression level (i.e., RNA, DNA or protein).

The term “treating” refers to alleviating or diminishing a symptom associated with the disease or the condition. Preferably, treating cures, e.g., substantially eliminates, and/or substantially decreases, the symptoms associated with the diseases or conditions of the present invention.

The treatment method according to the teachings of the present invention includes administering to an individual a therapeutically effective amount of the synthetic antisense oligonucleotides of the present invention. Preferred individual subjects according to the present invention are mammals such as canines, felines, ovines, porcines, equines, bovines, humans and the like.

A therapeutically effective amount implies an amount of agent effective to prevent, alleviate or ameliorate symptoms of disease or prolong the survival of the individual being treated

The agent of the method of the present invention can be administered to an individual per se, or as part of a pharmaceutical composition where it is mixed with a pharmaceutically acceptable carrier.

As used herein a “pharmaceutical composition” refers to a composition of one or more of the agents described hereinabove, or physiologically acceptable salts or prodrugs thereof, with other chemical components. The purpose of a pharmaceutical composition is to facilitate administration of a compound to an organism.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

The pharmaceutical compositions of the present invention may employ various penetration enhancers to effect the efficient delivery of nucleic acids, particularly oligonucleotides, to the skin of animals.

Penetration enhancers may be classified as belonging to one of five broad categories, i.e., surfactants, fatty acids, bile salts, chelating agents, and non-chelating non-surfactants [Lee et al., Critical Reviews in Therapeutic Drug Carrier Systems (1991) 92] as disclosed in U.S. Pat. Nos. 6,300,132, 6,271,030, 6,277,633, 6,284,538, 6,287,860, 6,294,382, 6,277,640 and 6,258,601 each of which is herein fully incorporated by reference.

Other substances that enhance uptake of oligonucleotides at the cellular level may also be added to the pharmaceutical compositions of the present invention. For example, cationic lipids, such as lipofectin [U.S. Pat. No. 5,705,188], cationic glycerol derivatives, and polycationic molecules, such as polylysine [PCT Application WO 97/30731], are also known to enhance the cellular uptake of oligonucleotides.

Other reagents may be utilized to enhance the penetration of the administered nucleic acids, including glycols such as ethylene glycol and propylene glycol, pyrrols such as 2-pyrrol, azones, and terpenes such as limonene and menthone.

Certain pharmaceutical compositions of the present invention may also incorporate carrier compounds. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′ isothiocyano-stilbene-2,2′-disulfonic acid [Miyao et al., Antisense Res. Dev., (1995) 5:115-121; Takakura et al., Antisense & Nucl. Acid Drug Dev. (1996) 6:177-183].

In contrast to a carrier compound, an “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical excipients include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration, which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation. Aqueous suspensions may contain substances which increase the viscosity of the suspension including, for example, sodium carboxymethylcellulose, sorbitol and/or dextran. The suspension may also contain stabilizers.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50 found to be effective in in vitro and in vivo animal models. Persons of ordinary skill in the art can easily estimate dosing and repetition rates based on measured residence times and concentrations of the oligonucleotide in bodily fluids or tissues. Following successful treatment, it may be desirable to have the patient undergo maintenance therapy to prevent the recurrence of the disease state, wherein the oligonucleotide is administered in maintenance doses.

The methods of the present invention have evident utility in the diagnosis and treatment of various diseases and conditions. In addition, such methods can also be used in non-clinical applications, such as, for example, differential cloning, detection of rearrangements in DNA sequences as disclosed in U.S. Pat. No. 5,994,320, drug discovery and the like.

The oligonucleotides generated according to the teachings of the present invention can be included in a diagnostic or therapeutic kit. For example, oligonucleotides sets pertaining to specific disease related transcripts can be packaged in a one or more containers with appropriate buffers and preservatives along with suitable instructions for use and used for diagnosis or for directing therapeutic treatment.

Preferably, the containers include a label. Suitable containers include, for example, bottles, vials, syringes, and test tubes. The containers may be formed from a variety of materials such as glass or plastic.

In addition, other additives such as stabilizers, buffers, blockers and the like may also be added.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### In-Vitro Expression Substantiation of Computationally Retrieved Naturally Occurring Antisense Transcripts

In-vitro expression assays were conducted in order to validate the existence of naturally occurring antisense sequences identified according to the teachings of the present invention.

Table 1 below lists polynucleotide sequence pairs that were selected for the in-vitro expression validation assays described in examples 1-7.

TABLE 1

| | | | | | | | |
|---|---|---|---|---|---|---|---|
| | | | | Anti- | | Start of | Start of |
| | | Sense | | sense | Overlap | overlap | overlap |
| Name of sense | Sense | Length | Antisense | Length | length | sense | anti- |
| antisense pair | transcript | (nt) | transcript | (nt) | (nt) | transcript | sense |

| | | | | | | | |
|---|---|---|---|---|---|---|---|
| 53BP1_76P | 53BP1 | 10394 | 76P | 6837 | 3046 | 5463 | 2018 |
| | (SEQ ID NO:15) | | (SEQ ID NO: 16) | | | | |
| CIDEB_BLTR2(1) | CIDEB1 | 2289 | BLTR2 | 6530 | 2254 | 17 | 1 |
| | (SEQ ID NO:19) | | (SEQ ID NO:21) | | | | |
| CIDEB_BLTR2(2) | CIDEB2 | 1511 | BLTR2 | 6530 | 1410 | 1 | 1 |
| | (SEQ ID NO:20) | | | | | | |
| APAF1_EB1 | aAPAF1 | 7042 | EBIa | 1752 | 141 | 6889 | 1612 |
| | (SEQ ID NO:24) | | (SEQ ID NO: 25) | | | | |
| AChR_MINK2 | AchR | 2457 | MINK2 | 4863 | 236 | 2175 | 4853 |
| | (SEQ ID NO:29) | | (SEQ ID NO: 30) | | | | |
| M-AchR_Anti-AChR | M-AchR | 1590 | M-Anti-AchR | 2227 | 672 | 934 | 506 |
| | (SEQ ID NO:35) | | (SEQ ID NO: 36) | | | | |
| CyclinE2_Anti-CyclinE2 | CyclinE2 | 2714 | Anti-CyclinE2 | 5773 | 1855 | 565 | 2006 |
| | (SEQ ID NO:33) | | (SEQ ID NO: 34) | | | | |

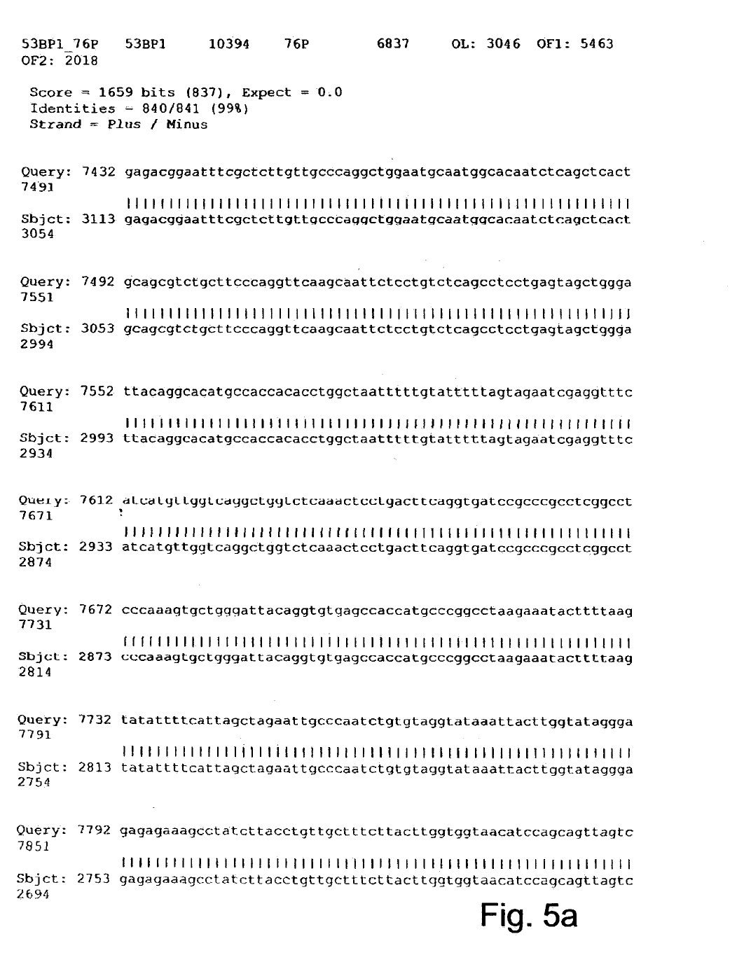

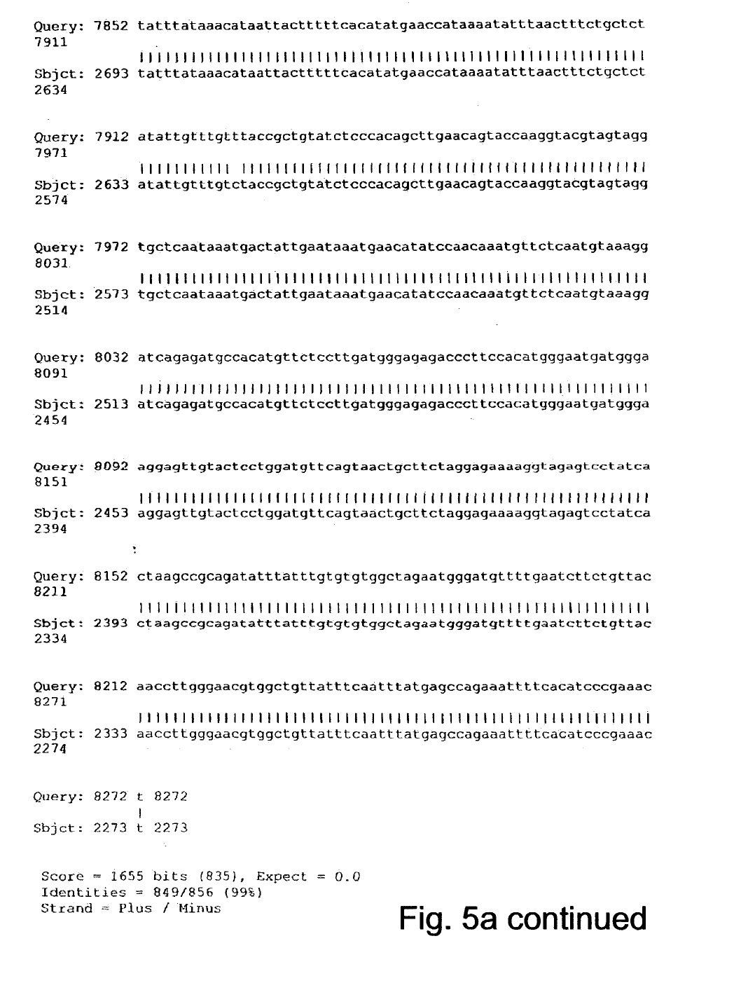

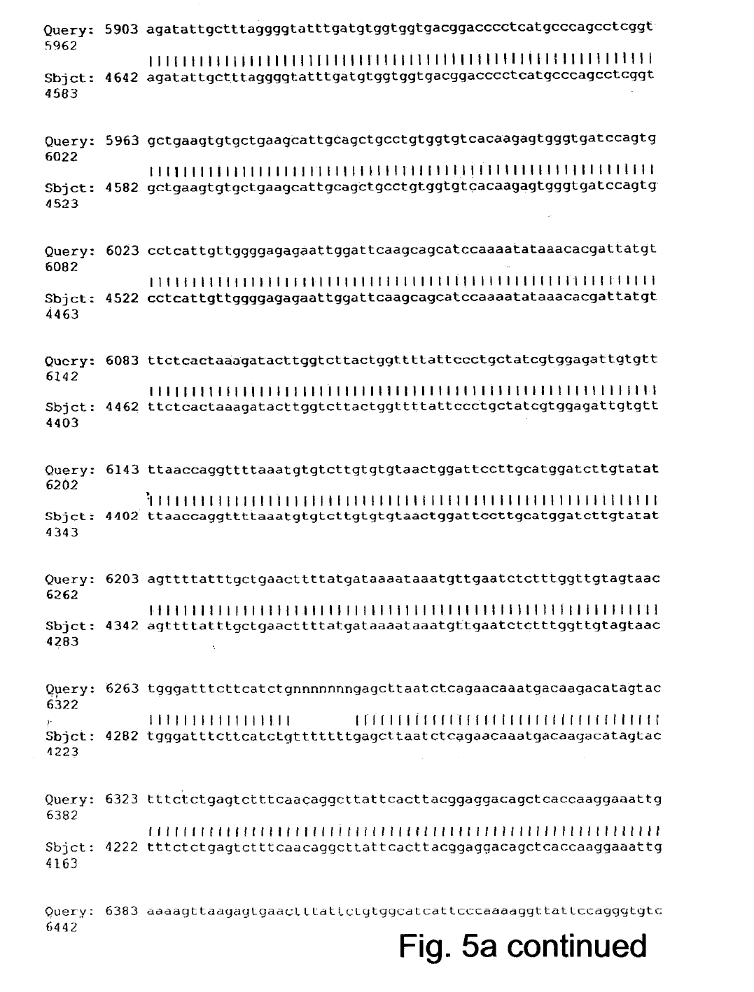

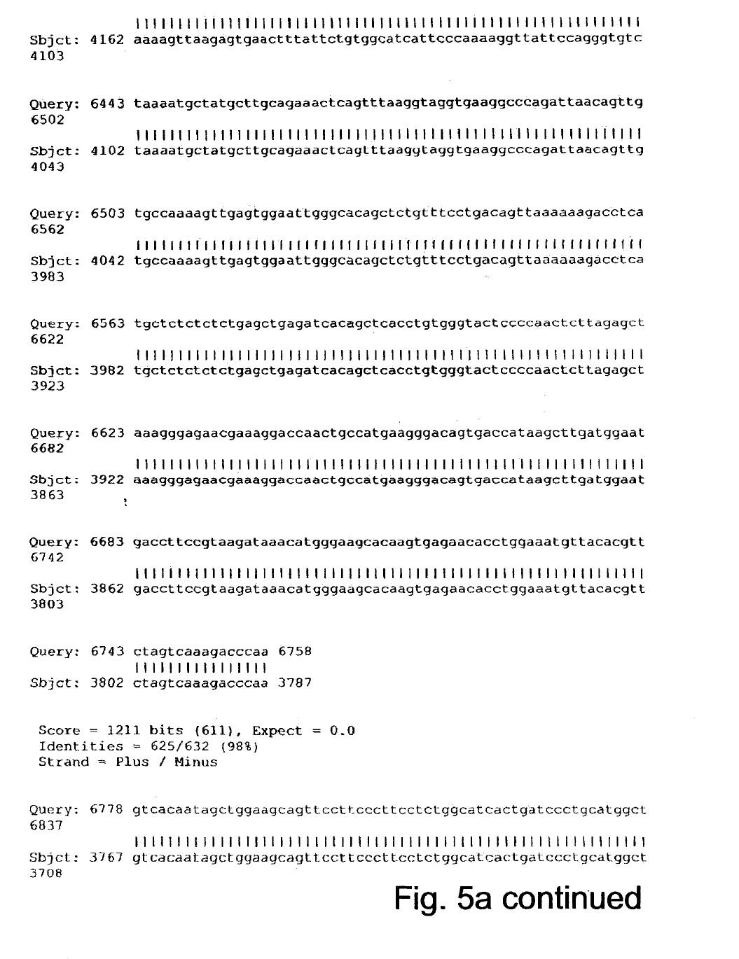

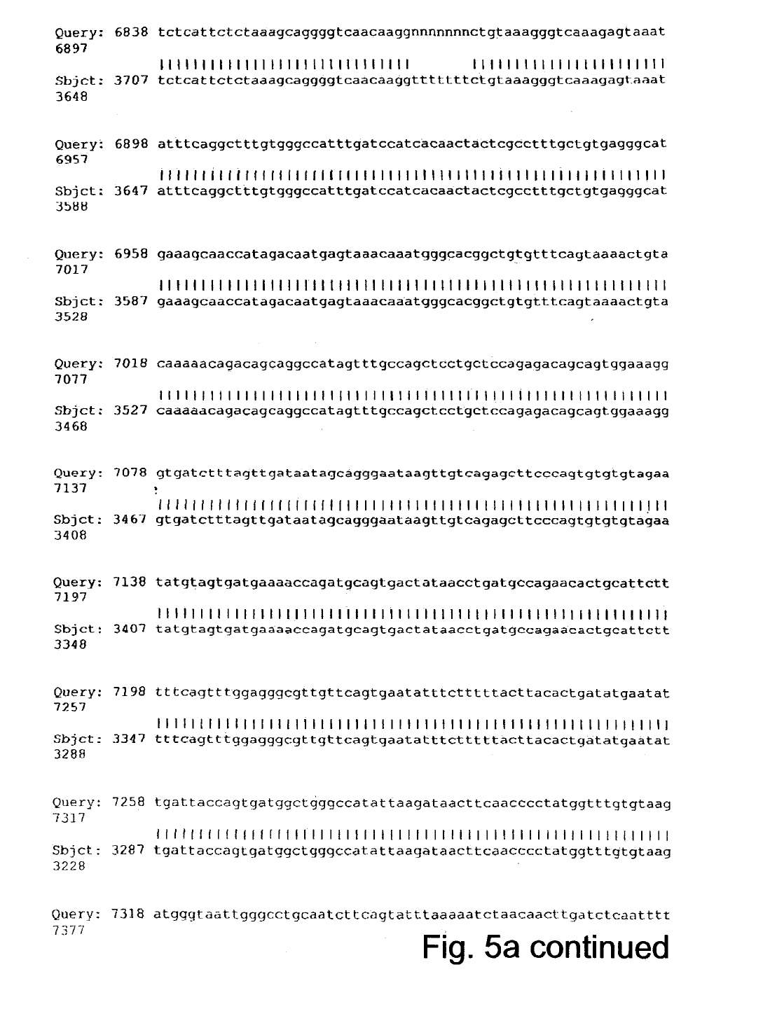

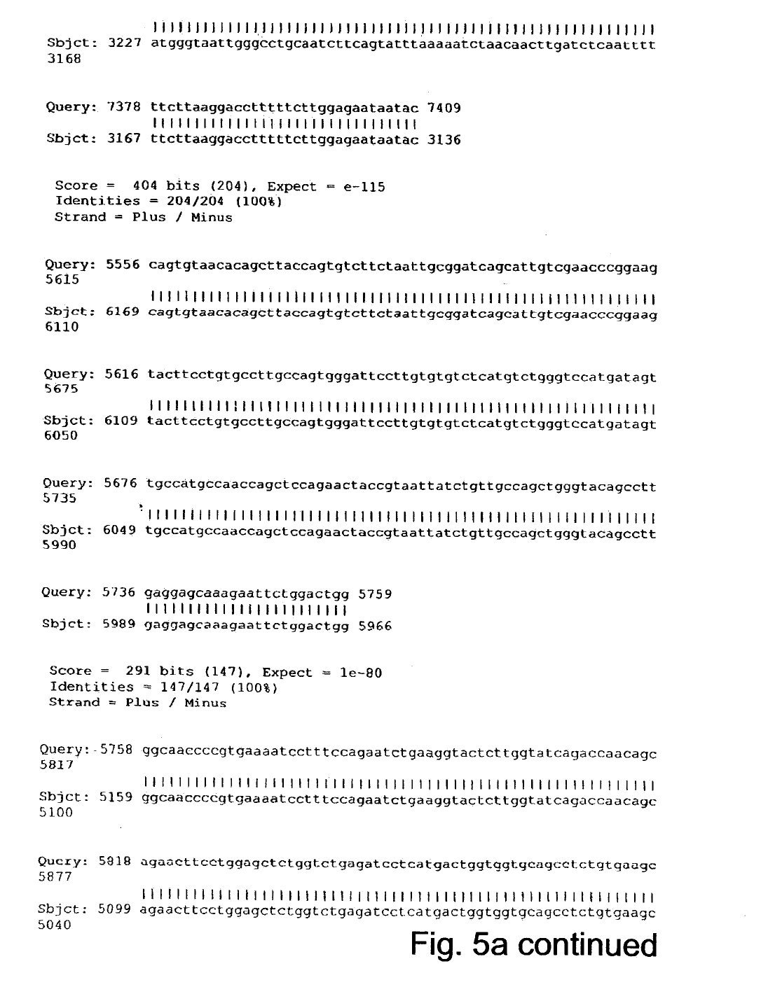

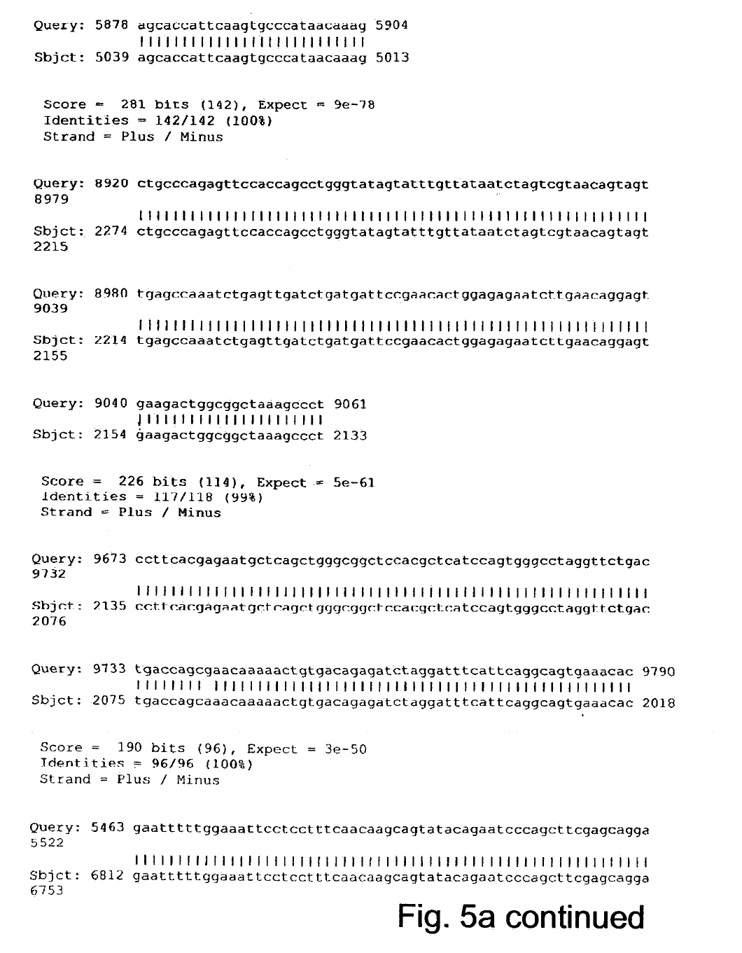

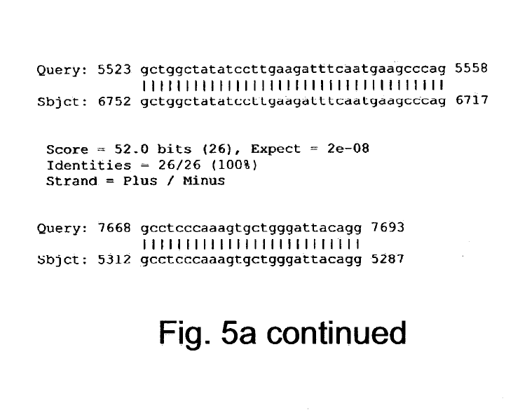

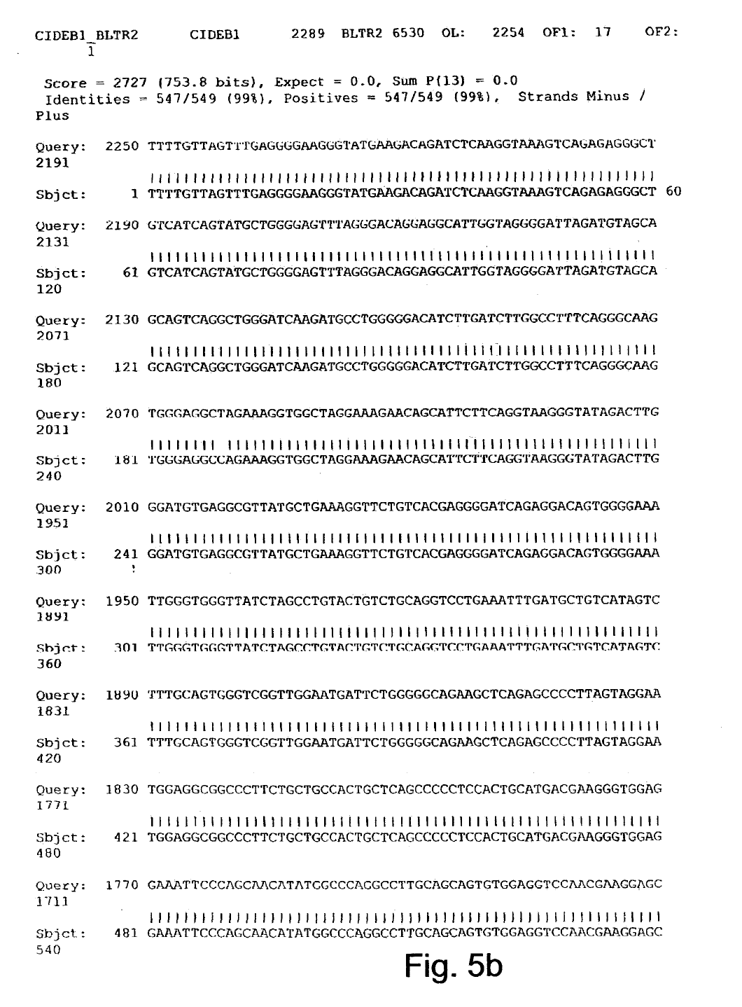

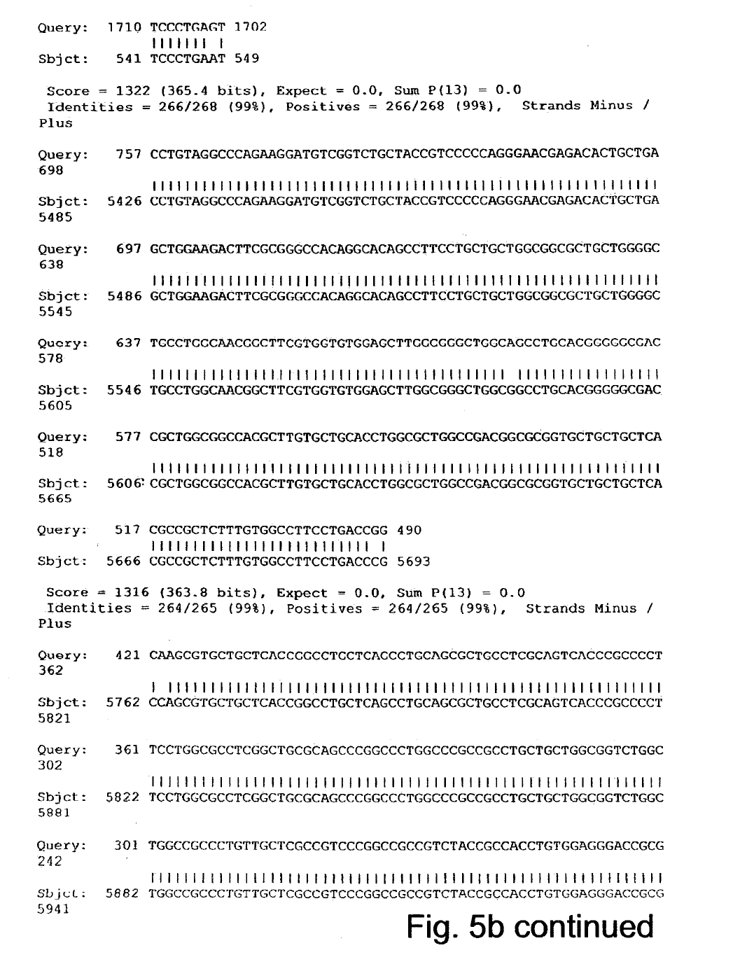

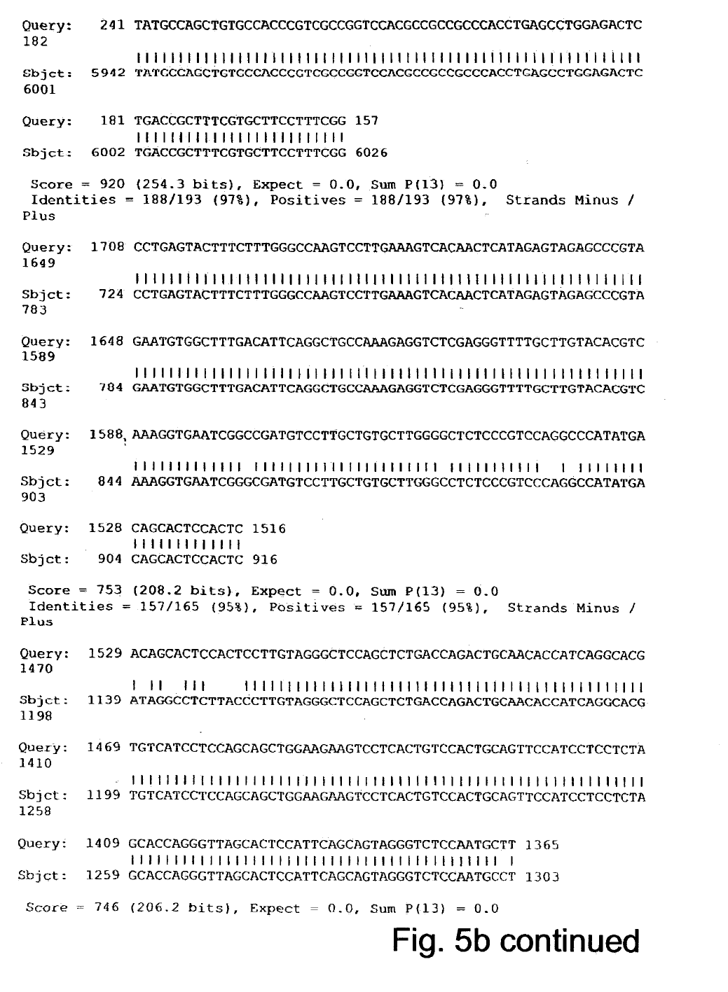

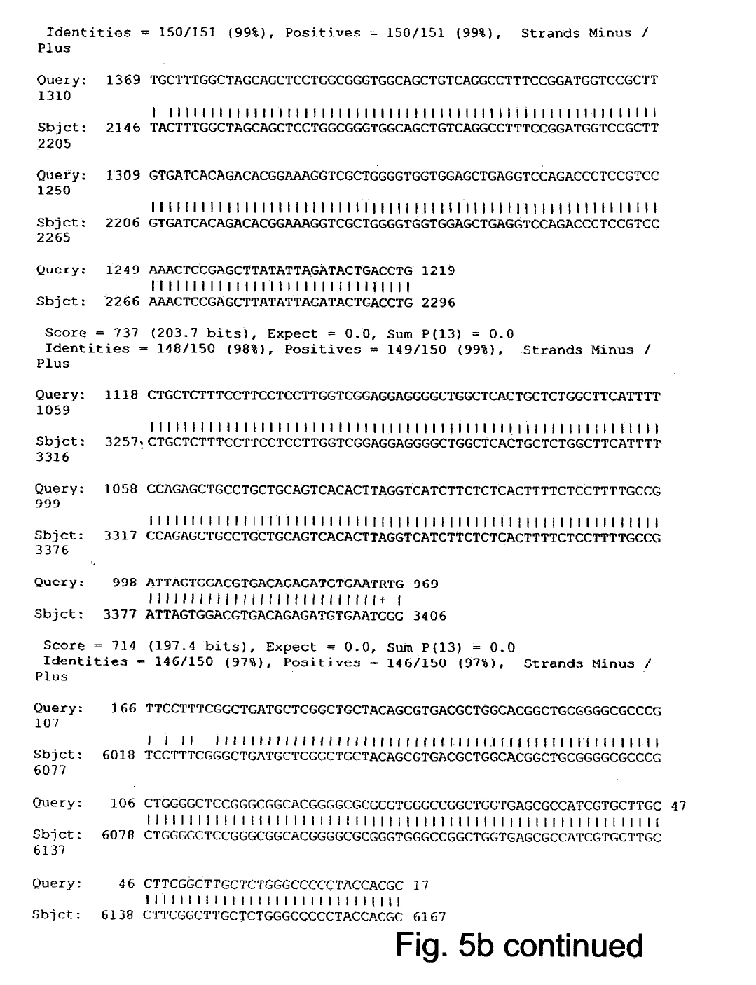

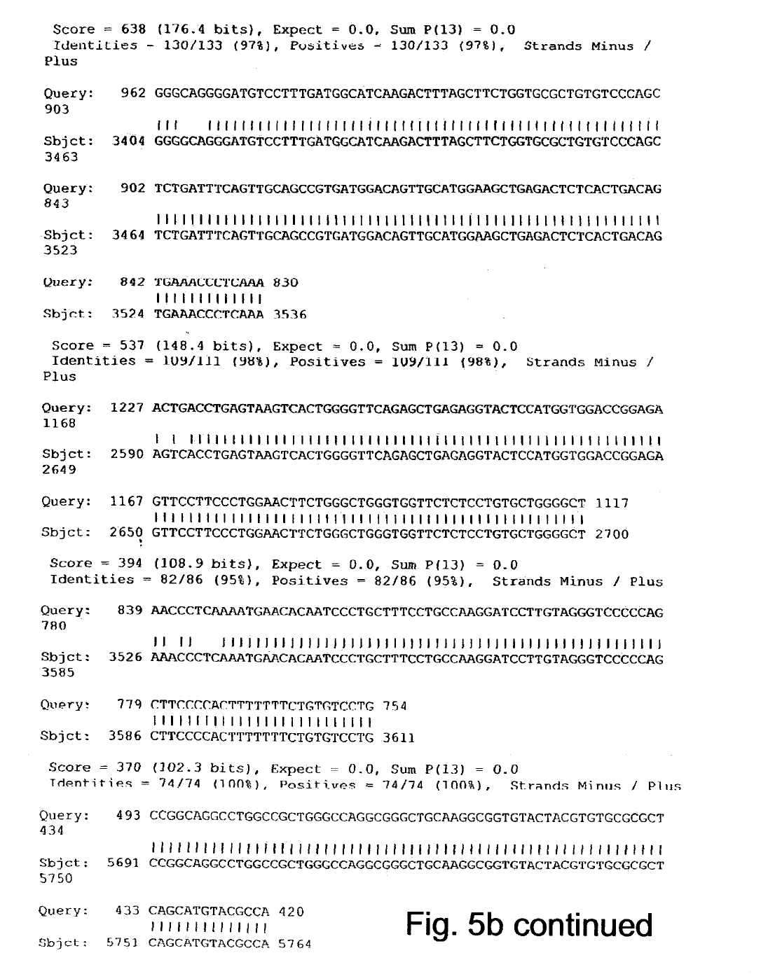

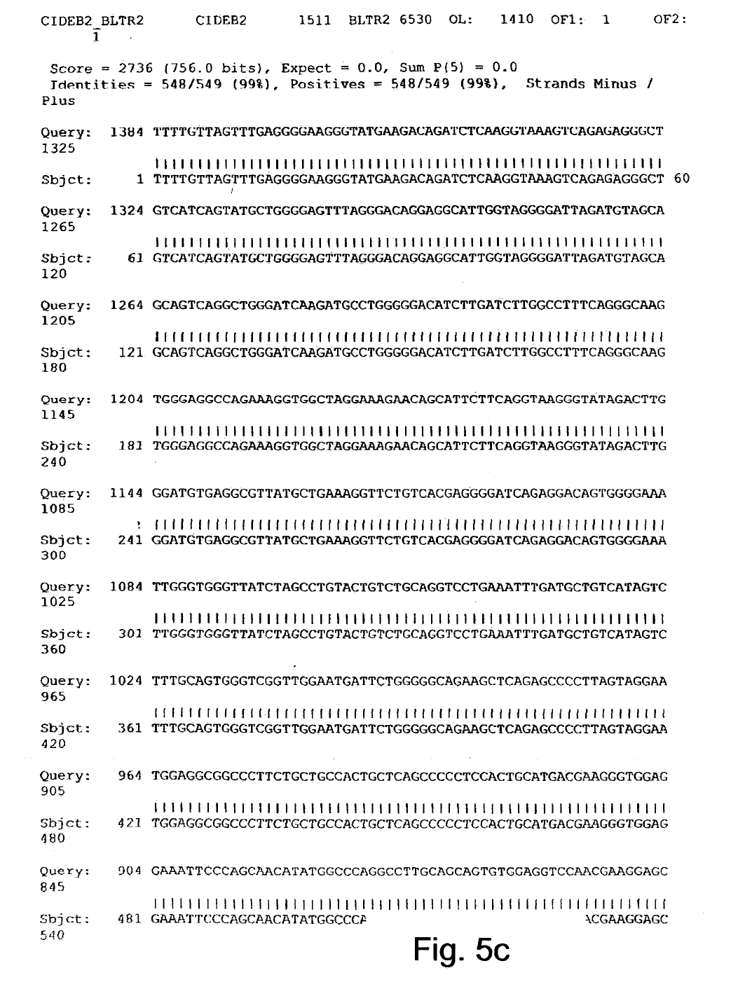

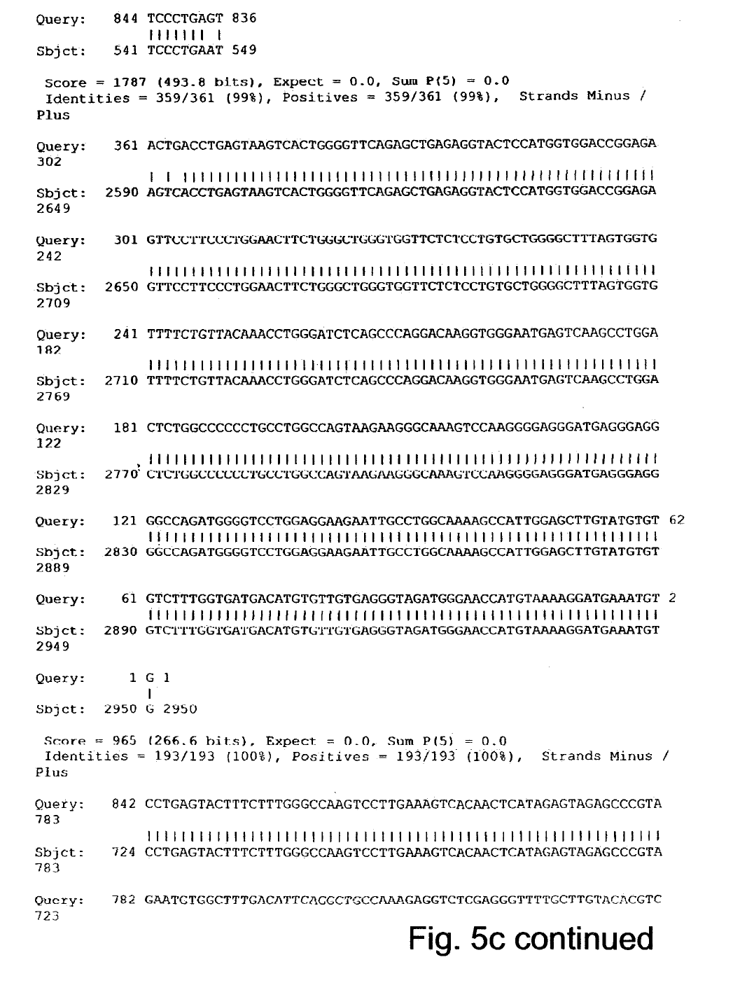

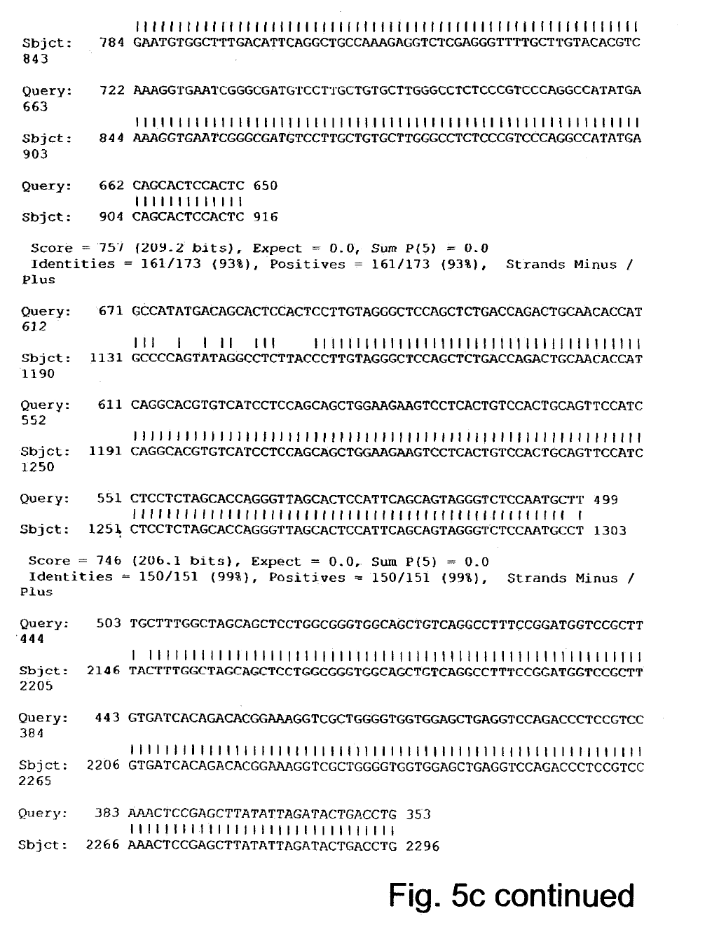

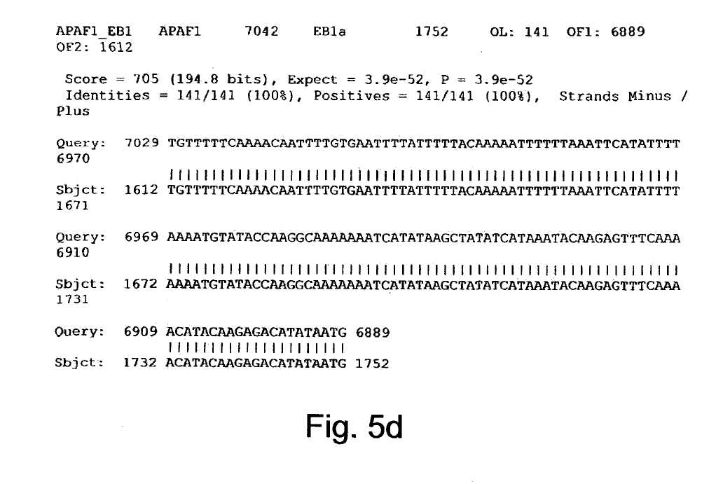

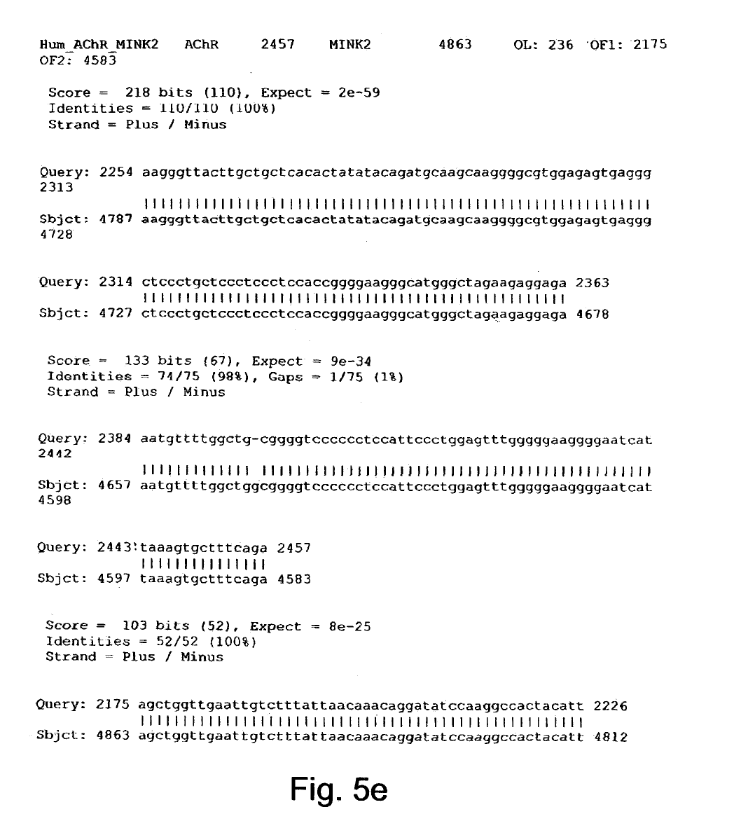

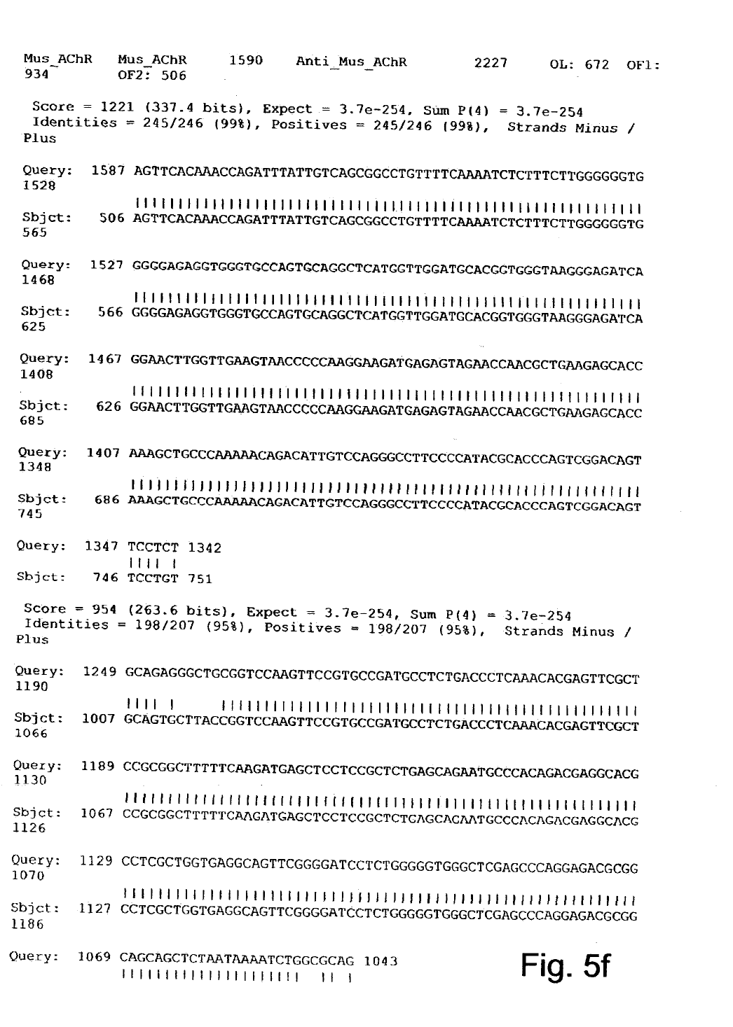

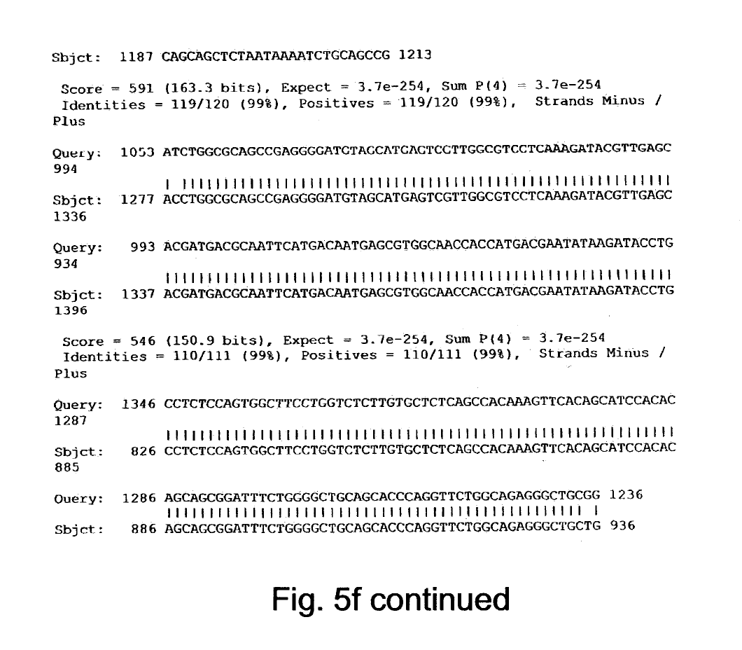

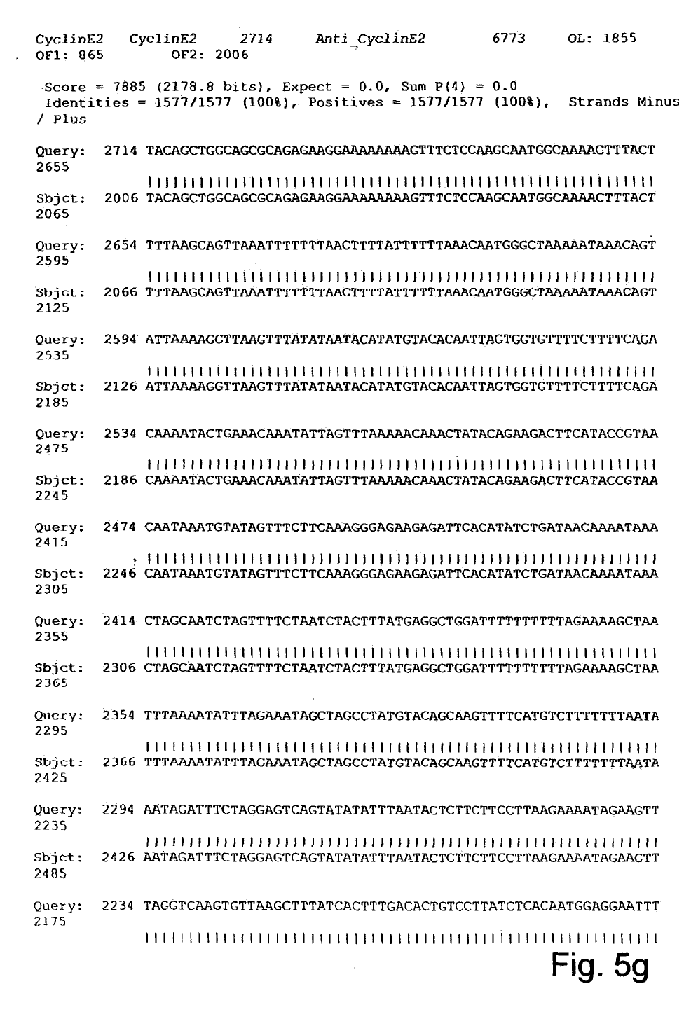

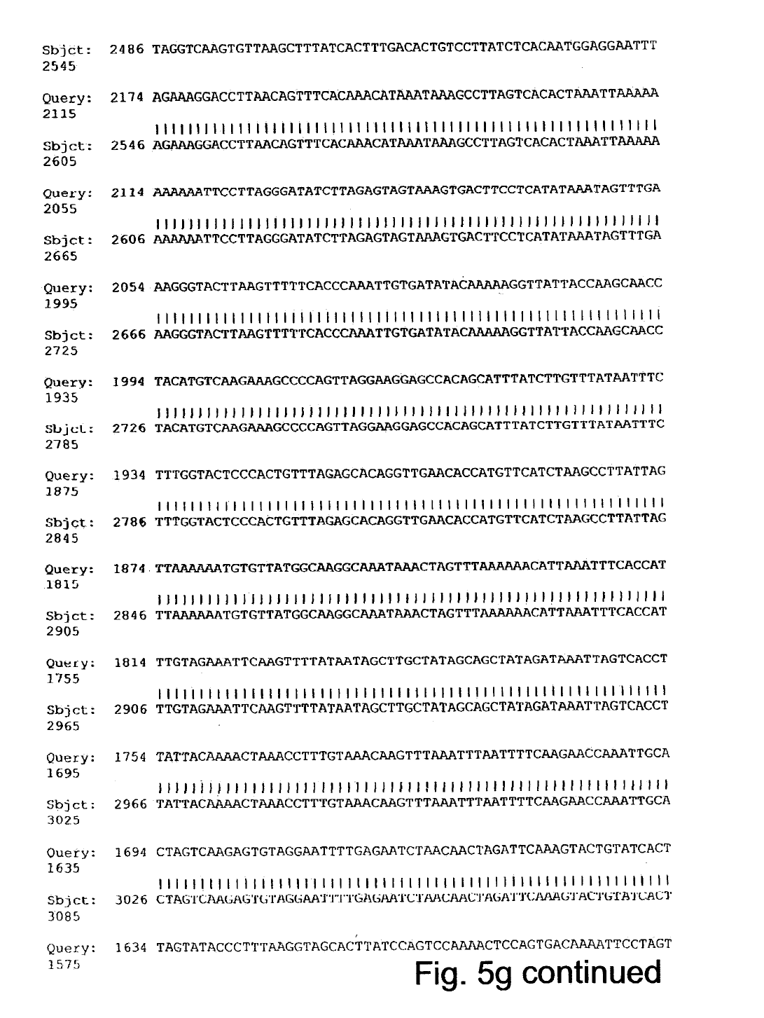

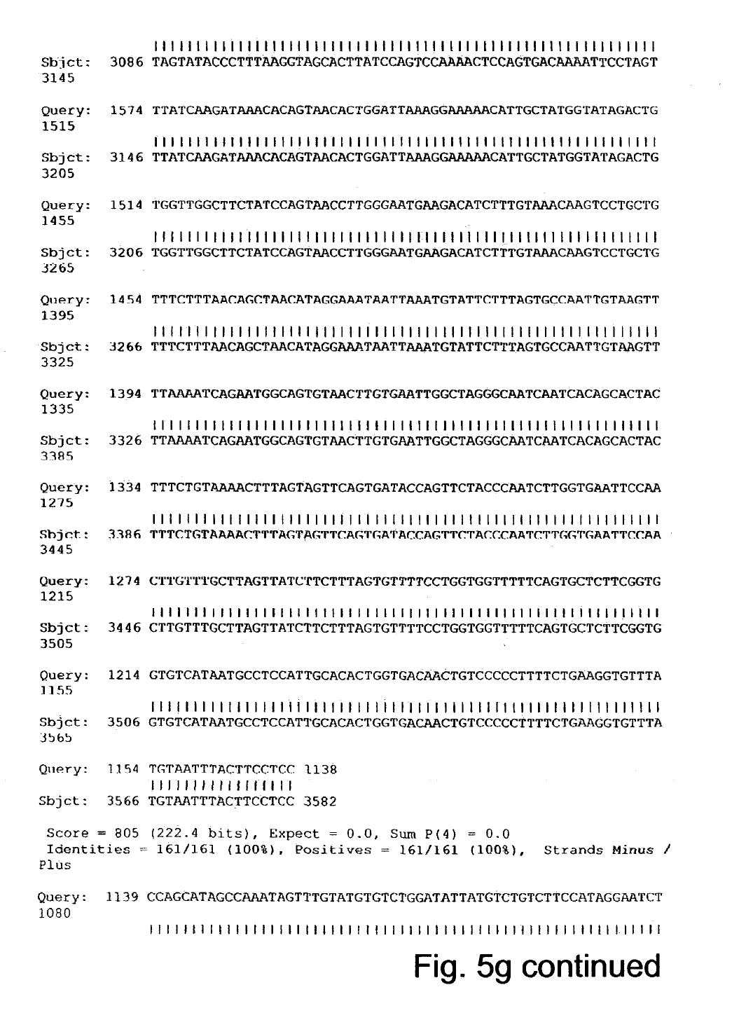

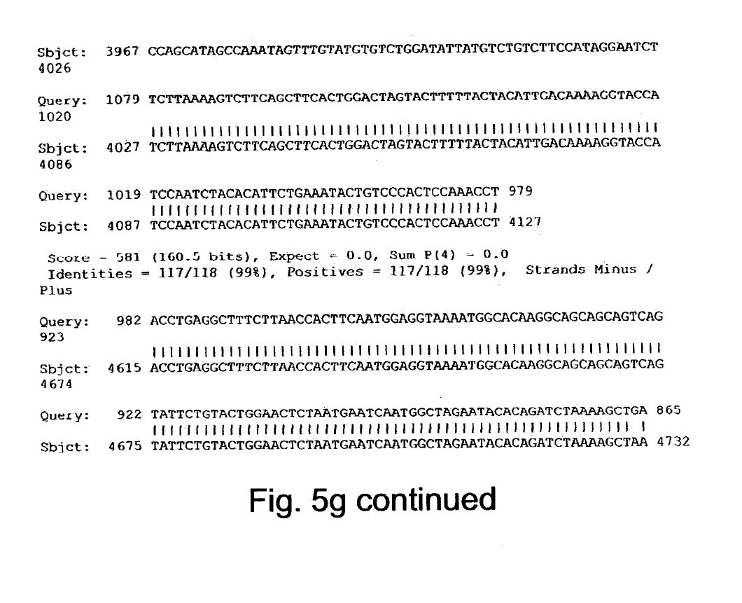

Sequence alignments of overlapping regions of each sense-antisense pair were performed using the BLAST sequence alignment algorithm (Basic Local Alignment Search Tool, available through www.ncbi.nlm.nih.gov/BLAST using the default parameters) and are exhibited in FIGS. 5a-g.

A microarray-based analysis was conducted, as well, in order to validate the existence of naturally occurring, antisense sequences identified according to the teachings of the present invention. The results are described in Example 9.

### Materials and Experimental Methods

RNA Probes Generation and Northern Analysis

RNA probes for northern analysis were generated by PCR amplification of a desired DNA fragment and cloning into Zero Blunt TOPO (Invitrogen Corp.) or pSPT18/19 vectors (Roche Ltd.). Alternatively PCR products were ligated into T7 RNA polymerase promoter-containing adaptors using the Lignscribe kit (Ambion Europe Ltd.). Corresponding RNA transcripts were synthesized using T7 RNA polymerase (Roche Ltd.) and labeled with 32P-UTP according to manufacturer's instructions. RNA probes were purified on Mini Quick Spin RNA columns.

Commercial membranes containing Poly(A)-RNA from various human tissues (2 μg RNA per lane) were obtained from Origene (OriGene Technologies Inc.) and Ambion (Ambion Inc.).

Alternatively, 2 μg of poly(A)-RNA prepared from various human cell-lines were electrophoretically separated on 1% agarose gel, and electrotransferred to Nytran SuperCharge membrane (Schleicher & Schuell) and subjected to fixing by UV radiation. Membranes were stained with methylene blue to ensure quantitative RNA transfer. Membranes were then prehybridized in a hybridization solution (UltraHyb solution Ambion Europe Ltd.) for 30 minutes at 68° C. in a rotating hybridization tube.

Hybridization solution was then supplemented with 106 cpm of labeled RNA probe per each ml of hybridization solution. Blots were hybridized for 16 hours at 68° C. in a rotating hybridization tube. Membranes were then washed twice with 2×SSC, 0.1% sodium dodecyl sulfate (SDS) and twice with 0.1% SDS at 68° C. RNA transcripts signals were detected using a phosphoimager (Molecular Dynamics, Sunnyvale Calif.).

Microarray

Oligonucleotide design—oligonucleotide design tools (1) were applied to each pair of sense/antisense genes in order to select two complementary 60-mer oligonucleotides from the region where the two genes overlap. The design criteria included the following: low cross-homology (up to 75%) to other expressed sequences in the human transcriptome; a continuous hit of no more than 17 bp to the sequence of another gene; balanced GC content (30-70%) without significant windows of local imbalance; no more than 2 palindromes with a length of 6 bp; a hit of no more than 15 bp to a repeat, vector or low-complexity region; and no long stretches of identical nucleotides.

Microarray preparation—60-mer oligonucleotides were synthesized by Sigma-Genosys (The Woodlands, TX), resuspended at 40 μM in 3×SSC, and spotted in quadruplicates on poly-L-lysine coated glass slides as detailed in the online protocol of the National Human Genome Research Institute (http://www.nhgri.nih.gov/DIR/Microarray/Protocols.pdf). To avoid local differences in the hybridization conditions, the probes selected from the overlapping regions of each sense/antisense pair were spotted in the same block, next to each other.

Human cell lines—The following cell lines utilized were purchased from ATCC (Manassas, VA): MCF7 (breast adenocarcinoma, Cat. No. HTB-22,), HeLa (cervical adenocarcinoma, Cat. No. CCL-2) HEK-293 (embryonal kidney cells, Cat. No. CRL-1573), Jurkat (acute T-cell leukemia, Cat. No. TIB-152), K-562 (chronic myclogenous leukemia, Cat. No. CCL-243), HepG2 (liver carcinoma, Cat. No. HB-8065), T24 (urinary bladder carcinoma, Cat. No. HTB-4), SK-N-DZ (neuroblastoma, Cat. No. CRL-2149), NK-92 (non-Hodgkin's lymphoma, Cat. No. CRL-2407), MG-63 (osteosarcoma, Cat. No. CRL-1427), DU 145 (prostatic carcinoma, Cat. No. HTB-81), G-361 (melanoma, Cat. No. CRL-1424), PANC-1 (pancreatic carcinoma, Cat. No. CRL-1469), ES-2 (ovary clear cell carcinoma, Cat. No. CRL-1978), Y79 (retinoblastoma, Cat. No. HTB-18), HT-29 (colorectal adenocarcinoma, Cat. No. HTB-38), H1299 (large cell lung carcinoma, Cat. No. CRL-5803), SNU1 (gastric carcinoma, Cat. No. CRL-5971), NL564 (EBV-transformed human lymphoblasts) and MCF10 (benign tumor breast cells).

RNA purification—Total RNA was extracted from the above mentioned human cell lines using TriReagent (Molecular Research Center, Cincinnati, Ohio). Poly(A)+ mRNA was purified using two cycles of the Dynabeads mRNA Purification Kit (Dynal Biotech ASA, Oslo, Norway), as per manufacturer instructions. The removal of traces of ribosomal RNA was confirmed by agarose gel electrophoresis. Poly(A)+ mRNAs from human testis, placenta, lung and brain tissue were purchased from BioChain Institute, Inc. (Hayward, Calif.). mRNAs of all cell lines described above were combined in equal quantities to obtain the reference ‘mRNA pool’.

Preparation of labeled cDNA—For each hybridization, labeled cDNA was synthesized by reverse transcription of 0.5 μg of mRNA, in the presence of 100 pmol of random 9-mers, 1 μg of oligo(dT)20, 1×RT buffer, 10 mM DTT, 3 nmol of Cy5- or Cy3-conjugated dUTP, 0.5 mM of dATP, dGTP and dCTP, and 0.2 mM dTTP, in a final volume of 40 μl (Amersham). The reaction mixture was incubated for 5 minutes at 65° C. and cooled to 42° C. 600 Units of reverse transcriptase (Superscript II, Invitrogen, Carlsbad, Calif.) and 40 U of Rnase inhibitor (RNasin Promega, Madison, Wis.) were added and the reaction was incubated for 30 minutes at 42° C. An additional 200 U of Superscript II were added and the reaction was incubated for another 15 minutes. Remaining RNA was degraded by the addition of 200 mM NaOH and 50 mM EDTA, at 65° C. for 10 minutes. The mixture was neutralized by adding half a volume of 1M Tris-HCl pH 7.5. Hybridizations were performed in duplicate using fluorescent reversal of Cy3- and CyS-labeled cDNA from test cell mRNAs and pooled mRNAs. Pairs of Cy5/Cy3-labeled cDNA samples were combined, and subsequently purified and concentrated to a final volume of 5-7 μl using a Microcon-30 (Millipore) concentrator.

Hybridization and washing conditions—Microarray slides were prehybridized with 40 μl of 5×SSC, 0.1% SDS and 1% BSA for 30 min at 42° C., washed for 2 minutes with double distilled water, then rinsed with isopropanol, and spun dried at 500 g for 3 minutes. Prior to hybridization, the labeled probe was combined with 10 μg of Cot-1 DNA, 10 μg poly(dA)80, and 4 μg yeast tRNA, in a final volume of 15 μl . The mixture was denatured at 100° C. for 3 minutes and placed on ice. Formamide (final concentration 16%), SSC (to 5× concentration) and 0.1% SDS were added to a final volume of 30 μl. The mixture was placed on the array under a glass cover slip in a tightly sealed hybridization chamber, and immersed in a water bath at 42° C., for 16 hours. Microarray slides were then washed for 4 minutes with 2×SSC, 0.1% SDS; 4 minutes with 1×SSC, 0.01% SDS; 4 minutes with 0.2×SSC and 15 seconds with 0.05×SSC and spun dry by centrifugation for 3 minutes at 500 g.

Image processing—Following hybridization, arrays were scanned using a GenePix 4000B scanner (Axon Instruments, Union City, Calif.). Scanned array images were manually inspected and areas with visible artifacts or deformities were marked. Images were processed using GenePix Pro 3.0 (www.axon.com) software.

Normalization—The intensity for each spot was calculated as its mean intensity minus the median background around the spot. The signal for each oligo was calculated as the average of intensity values of the four redundant spots of each oligo. Normalization of the oligo signals was performed at several levels as is further described below.

Normalization of blocks was carried out in order to normalize the gradient of intensities within each slide. For each block i, an Ai parameter was calculated as the average of intensities of 56 positive control spots (oligonucleotide probes for the ubiquitously expressed housekeeping genes gapdh, actin, hsp70 and gnb211, in various probe concentrations). An average A of all Ai averages was calculated. Based on this, a block normalization factor Bi was calculated for each block, as Bi=A/Ai, and applied to each spot in the block.

Normalization between slides was performed to bring all experiments to the same scale. For each experiment, the average of intensities of the 192 negative control spots on the array was set to be the 0 (zero) of the new scale. For a subset of highly signaling oligos, with intensities between the 70th and the 95th percentiles of the oligo signal distribution of the experiment, the average was arbitrarily set to be 500 in the new scale. The intensity of each oligo signal was accordingly converted to this new scale, to obtain the normalized signal. A ratio between the normalized cell-line signal and the normalized pool signal was calculated for each oligo in each experiment. To avoid misleading ratios coming from signals that were too low, the ratio Rji for oligo j in experiment i was calculated as: Rji=max [100, cell-line-signalji]/max [100, pool-signalji].

To normalize between red/green intensities in reciprocal experiments, the ratio Rjk for oligo j in cell-line k was calculated as the average of calculated ratios Rji between the two reciprocal experiments of the cell-line k. In cases where only one of the two reciprocal experiments showed an elevated or decreased ratio, while in the other the ratio was 1.0, the average Rjk was converted to 1.0.

The actual pool signal for each oligo was calculated to be the average of the normalized oligo signals in the pool channel of all experiments. A virtual pool signal was calculated as the average of the normalized oligo signals in the cell-line channel of all experiments. The virtual pool signals were found to be very close to the actual pool signals, indicating consistency in the analysis.

Threshold determination—To determine an expression threshold above, in which a normalized signal would be considered a ‘positive’ signal indicating expression, the distribution of all 16,512 normalized negative control signals and the standard deviation (neg-std-dev) were calculated. The neg-std-dev obtained was 38. An oligo j was considered ‘present’ in a cell-line k if Rjk×actual-pool-signalj≧4×neg-std-dev.

### Example 1

### Identification of 53BP1 and 76P RNA Transcripts in a Variety of Human Tissues and Cell-Lines

Background:

The tumor suppressor p53 binding protein 1 (SEQ ID NO: 15) is one of the various p53 target proteins. It binds to the DNA-binding domain of p53 and enhances p53-mediated transcriptional activation. 53BP1 is characterized by several structural motifs shared by several proteins involved in DNA repair and/or DNA damage-signaling pathways. 53BP1 becomes hyperphosphorylated and forms discrete nuclear foci in response to DNA damage induced by radiation and chemotherapy. Recent reports suggest that 53BP1 is an ataxia telangiectasia mutated (ATM) substrate that is involved early in the DNA damage-signaling pathways in mammalian cells, attributing a role to 53BP 1 in the development of various mammalian pathologies.

Results:

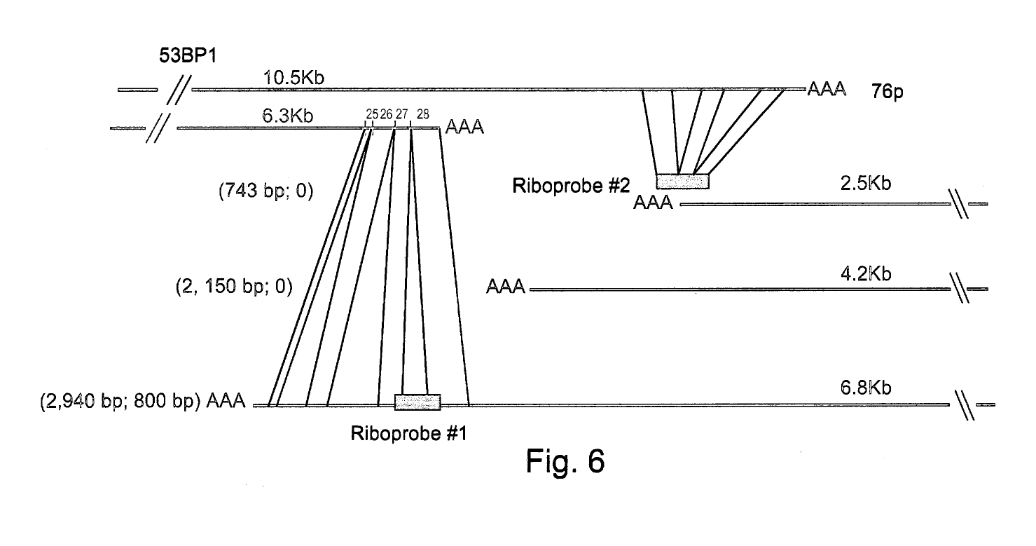

Two 53BP1 RNA sense transcripts with dissimilar 3′ UTRs were previously described [Iwabuchi K. et al. (1994) Proc. Natl. Acad. Sci. USA] and are illustrated in FIG. 6 (red and green). Leads™ assembly program modified to uncover novel antisense transcripts was used to uncover three such transcripts for the 53BP1 gene, which transcripts have different 3′ UTRs (SEQ ID NO: 16, 37 and 38) and encode the 76p gene product (Genbank accession number NM014444)(illustrated in blue).

To confirm expression of computationally retrieved antisense transcripts, two RNA-probes were generated. Schematic location of the probes used for sense and antisense validation (Riboprobe#1 and Riboprobe#2, respectively SEQ ID NO: 17 and 18, respectively) is illustrated in FIG. 6. These RNA probes were used to identify the corresponding full-length transcripts.

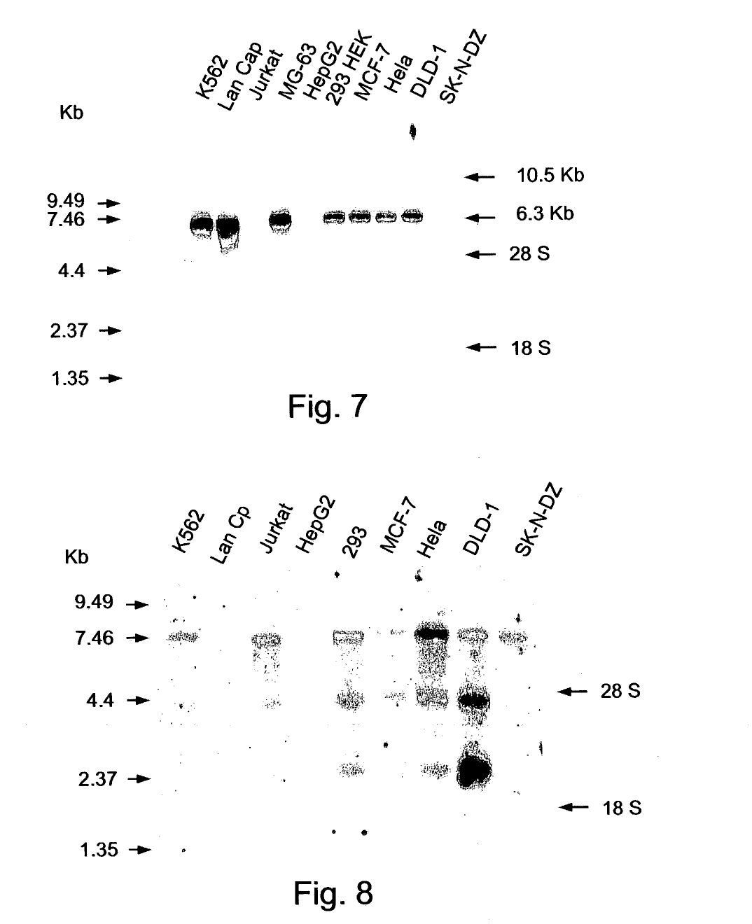

As shown in FIG. 7, Riboprobe#1 detected two transcripts of approximately 6.3 Kb and 10.5 Kb, corresponding to the sense mRNA. The absolute levels of the short messenger were rather homogeneous in all cell-lines examined. The 10.5 Kb variant exhibited a more heterogenic pattern of cellular distribution, and was mostly expressed in K562, MG-63, 293 HEK and Hela cells. In general, the longer sense transcript which is an alternatively polyadenylated variant was markedly lower expressed in the various cell lines examined.

The same membrane was used to perform northern analysis with Riboprobe#2 in order to validate expression of antisense transcripts of 53BP1. Results are shown in FIG. 8. Three variants corresponding to the 76p gene were detected in most of the cell lines: 6.8 Kb, 4.2 Kb and 2.5 Kb. Minor fluctuations of expression were observed and the largest transcript was expressed at significantly higher levels than the smaller transcripts.

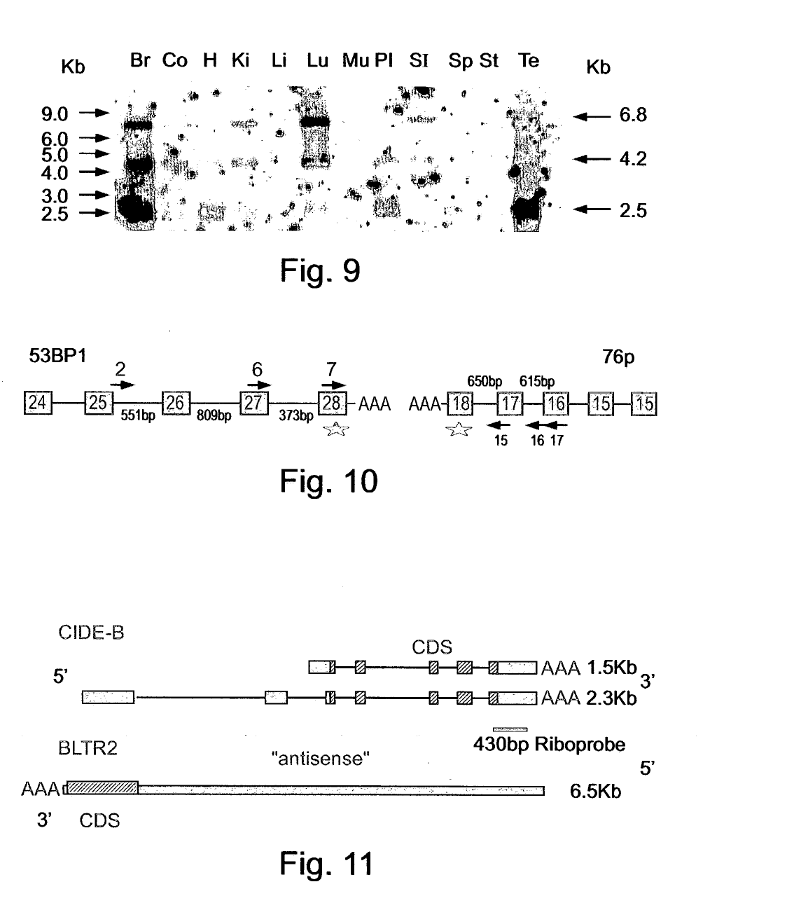

A sense strand probe was used to detect expression of the antisense transcripts in a variety of human tissues (FIG. 9). The three alternatively polyadenylated variants with different 3′ UTRs were expressed in most of the tissues. Total levels of these transcripts varied in the different tissues assayed. For example, highest level of expression for all three transcripts was observed in the brain and testis, while no expression of the 6.8 Kb and 4.2 Kb variants was detected in the spleen. Expression levels of each transcript were summarized in Table 2 below.

TABLE 2

| | Transcript Mol. Weight (Kb) | | |
|---|---|---|---|
| Tissue | 6.8 | 4.2 | 2.5 |
| brain | +++ | ++++ | ++++ |
| colon | + | ++ | + |
| heart | − | + | ++ |
| kidney | ++ | ++ | + |
| Liver | − | − | + |
| lung | ++++ | +++ | + |
| muscle | ++ | + | + |
| placenta | + | ++ | ++ |
| Small intestine. | ++ | ++ | − |
| spleen | − | − | + |
| stomach | − | − | + |
| testis | ++ | ++ | ++++ |

Reverse transcription amplification (RT-PCR) analysis was performed in order to substantiate the northern blot results. Primers were synthesized according to the scheme shown in FIG. 10 (indicated by arrows). The expected amplification products corresponded completely to the observed amplification reaction products, supporting the existence of the various 53BP1 and 76p transcription variants.

### Example 2

Identification of mRNA and Complementary Transcripts of the Cell Death Inducing DFF45-Like Effector (CIDE)-B

Background:

Cell death inducing DFF45-like effector (CIDE-B) (GenBank Accession numbers AF190901 and AF218586) is a member of a novel family of apoptosis-inducing factors that share homology with the N-terminal region of DFF, the DNA fragmentation factor. Although the molecular mechanism of CIDE-B induced apoptosis in unclear, mitochondrial localization and dimerization, both where shown to be required [Chen Z. et al. (2000) J. Biol. Chem. 275:22619-22622]. Notably, over-expression of CIDE-B in mammalian cells shows strong cell death-inducing activity, suggesting that aberrant expression of this protein may be associated with a number of mammalian pathologies [Inohara N. et al. (1998) EMBO J. 17:2526-2533].

Results:

Two sense transcript of the CIDE-B gene were previously described with different 5′ UTRs [Inohara N. et al. (1998) EMBO J. 17:2526-2533 and Lugovskoy A A. et al. (1999) Cell 99:745-755] (SEQ ID NOs: 19 and 20). Computational analysis recovered a potential elongated BLTR2 transcript (SEQ ID NO: 21), showing full complementary to the CIDE-B mRNA transcripts (FIG. 11).

Northern blot analysis was done in order to determine the distribution of the CIDE-B sense and antisense transcripts in various cell-lines. A 430 base pairs DNA fragment was selected to generate RNA probes for identification of both sense and antisense transcripts (SEQ ID NOs: 22 and 23, respectively).

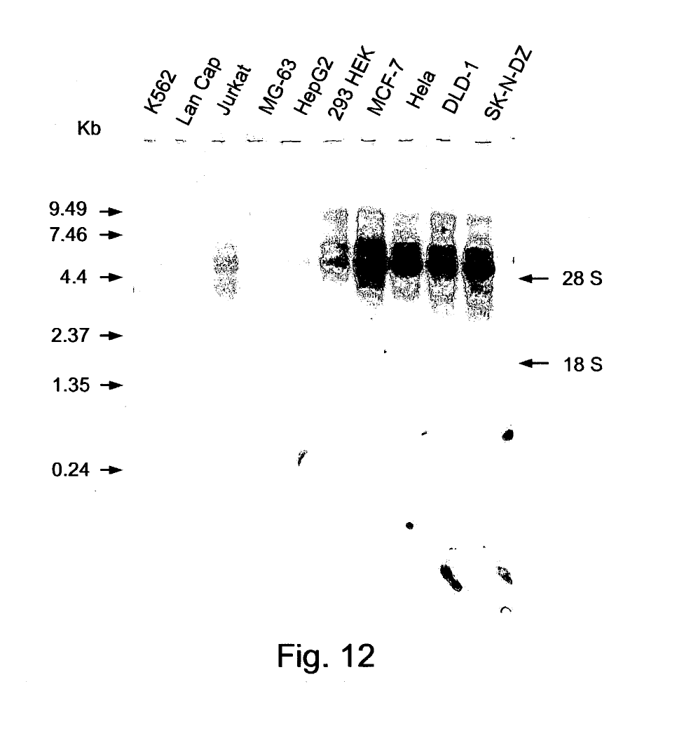

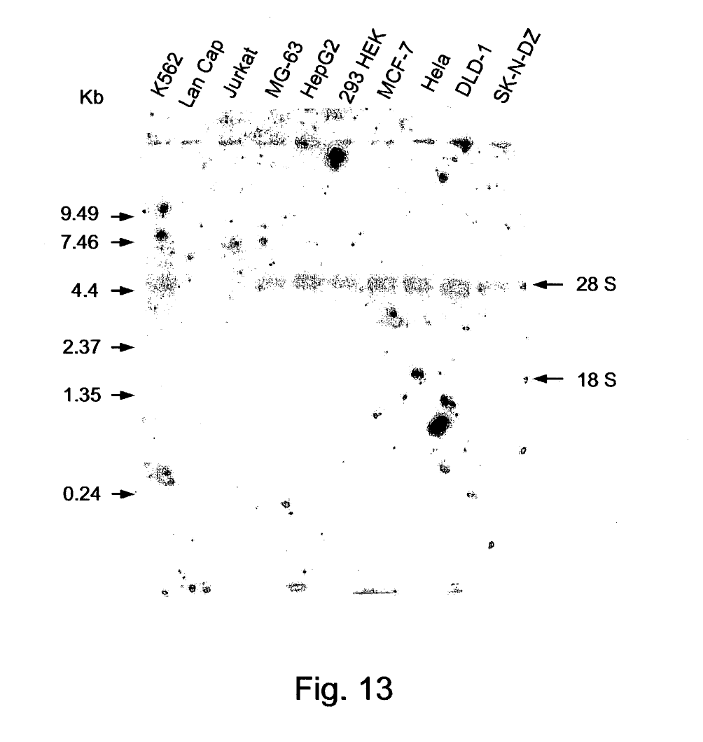

Expression of antisense mRNA transcripts was detected in various cell-lines and especially in the mammary gland adenocarcinome cell line-MCF-7 as a predominant 6.5 Kb transcript, although higher forms were also visualized (FIG. 12). Low hybridization with a CIDE-B probe was detected (FIG. 13).

Conclusion:

BLTR2 was recently identified as a putative seven-transmembrane receptor with a high homology to the Leukotriene B (4) receptor [Tryselius Y. et al. (2000) Biochem. Biophys. Res. Commun. 274:377-82]. Although the mechanism of action of BLTR2 is poorly understood, it is conceivable that BLTR2 mRNA plays a role in the regulation of CIDE-B apoptotic effector and vice versa.

### Example 3

### Identification of mRNA and Complementary Transcripts of the Apoptosis Inducing Factor APAF-1

Background:

A conserved series of events including cellular shrinkage, nuclear condensation, externalization of plasma membrane phosphatidyl serine, and oligonucleosomal DNA fragmentation characterizes apoptotic cell death. Regardless of the circumstance, induction and execution of apoptotic events require activation of caspases, a family of aspartate-specific cysteine proteinases. Caspase activation may be regulated by the mitochondrion and specifically by the apoptosome consisting of an oligomeric complex of apoptotic protease-activating factor-1 (APAF-1), cytochrome C and dATP. The apoptosome recruits and activates caspase-9, which in turn activates the executioner caspases, caspase-3 and -7. The active executioners kill the cell by proteolysis of key cellular substrates [Zou H. et al. (1999) J. Biol. Chem. 274:11549-11556]. Evasion or inactivation of the mitochondrial apoptosis pathway may contribute to oncogenesis by allowing cell proliferation. In this instance, unregulated cell proliferation may occur by inactivation of APAF-1, which has been suggested to occur via genetic loss or inhibition by HSP-70 and HSP-90. Although aberrant expression of APAF-1 was found in a variety of malignancies (including ovarian epithelial cancer), no link was found to accelerated protein degradation.

Results:

One RNA transcript has been previously described for APAF-1 [Zou H. et al. (1999) J. Biol. Chem. 274:11549-11556] (SEQ ID NO: 10) (SEQ ID NO: 24). Computational search for natural antisense transcripts has revealed two complementary transcripts for APAF-1 messenger RNA (SEQ ID NOs: 25 and 26). These antisense transcripts include an open reading frame encoding the EB-1 gene (GenBank accession numbers AF145204; AF164792). The overlap between the APAF-1 messenger RNA and the longer antisense transcript is of at least 300 nucleotides.

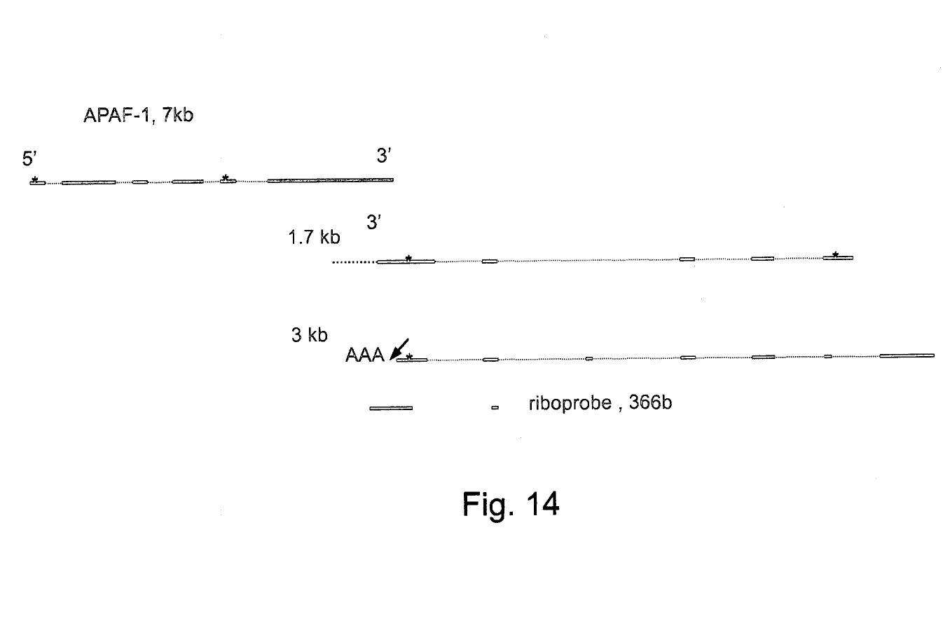

To validate expression of computationally retrieved antisense transcripts for APAF-1, as well as expression of APAF- 1 mRNA in the assayed human cell lines, RNA-probes of 366 ribonucleotides were generated (sense and antisense strands, respectively). Schematic location of the probes used for sense and antisense validation (Riboprobe#1 and Riboprobe#2, SEQ ID NOs: 27 and 28, respectively) is illustrated in FIG. 14.

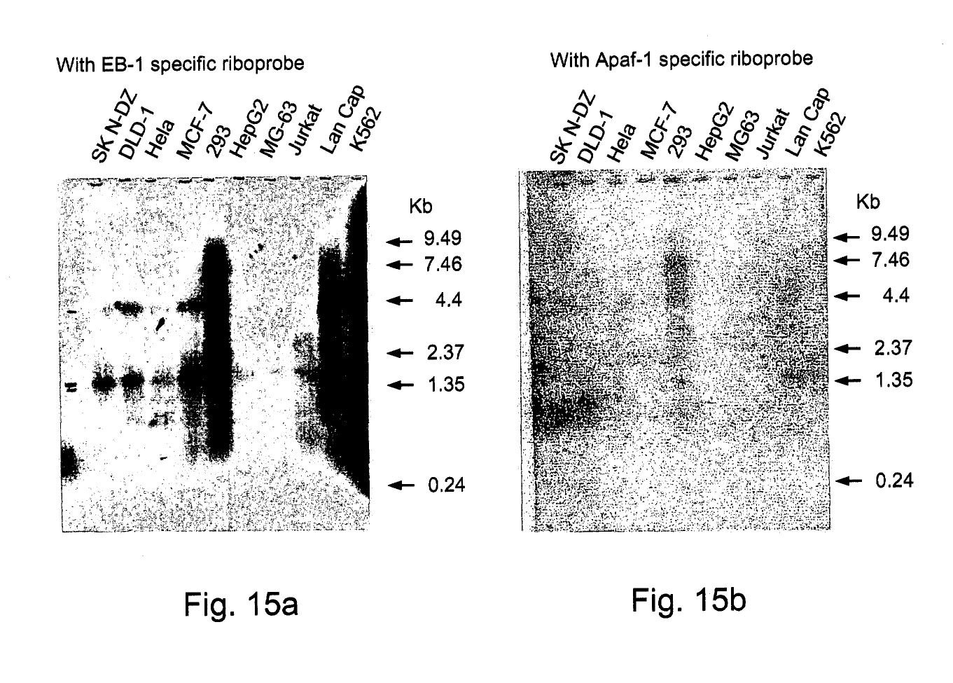

As shown in FIG. 15a, the sense RNA probe directed at visualizing the antisense transcripts, identified a clear band of 3 Kb corresponding to the long computationally retrieved antisense transcript as well as other transcripts sizing from 1 Kb to 8 Kb (FIG. 15a). Transcripts were essentially found in all cell lines but especially in 293 HEK and LN-Cap lines.

Hybridization with an RNA probe directed at visualizing the mRNA transcript of APAF-1 resulted only in a blurred patterns (FIG. 15b). However, a 7 Kb mRNA transcript consistent with APAF-1mRNA was seen in Ln Cap and 293 HEK cell lines.

Conclusion:

A reciprocal pattern of expression was observed for both APAF-1 and EB-1 transcripts, exhibiting an interesting expressional relationship between the sense and antisense transcripts suggesting antisense-mediated expression regulation.

### Example 4

### mRNA Expression of Muscle Nicotinic Acetyl-Choline Receptor ε Subunit and its Complementary MINK Transcript

Background:

The muscle nicotinic Acetylcholine Receptor ε subunit (AChRε) encodes for one of five subunits of a ligand gated ion channel receptor located at the neuromuscular synapse. AChRε is up-regulated in the postnatal period when it replaces γ subunit of the receptor [Witzamann, V. et al., (1987) FEBS Lett. 223, 104-112]. It is also up-regulated in synapse development, specifically by the trophic factor neuregulin [Martinou J. C. (1991) Pro. Natl. Acad. Sci. USA 88, 7669-7673]. In an attempt to decipher AchRε function and mechanism of regulation, computational screen for AChRε K complementary transcript was carried out.

Results:

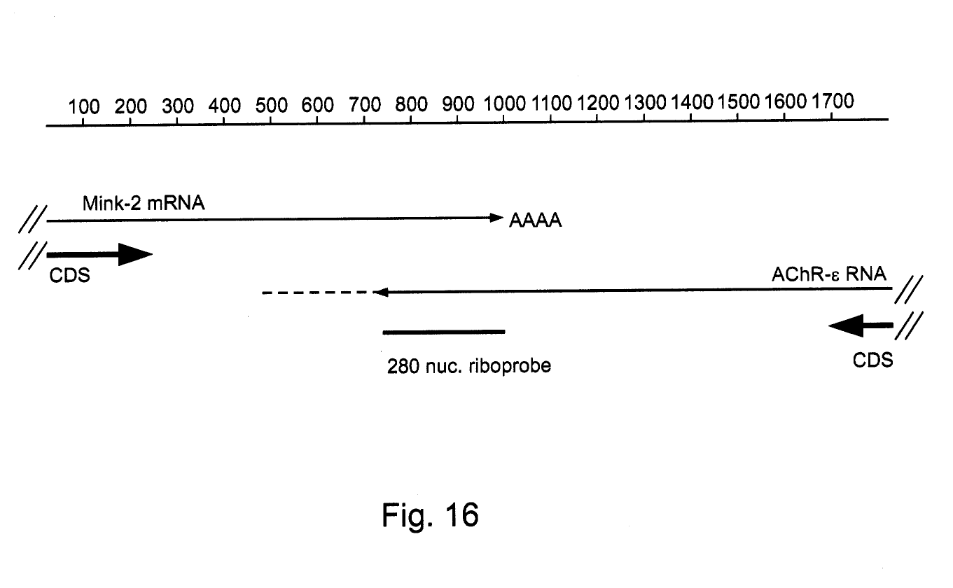

One mRNA transcript of AChRΕ gene was previously described [Beeson D. Eur. J. Biochem (1993) 215, 229-238] (SEQ ID NO: 29). Computational analysis recovered a complementary transcript belonging to Mink, a new member of the germinal center kinase (GCK) family (SEQ ID NO: 30) [Dan I. FEBS Lett. (2000) 469, 19-23] showing an overlap of at least 280 nucleotides to the AchRε mRNA, as schematically illustrated in FIG. 16.

To validate the overlap of the two genes and to learn about their tissue distribution, northern analysis of a variety of human tissues was performed. Poly(A)-RNA containing membrane was hybridized with a 280 nucleotides RNA probes, corresponding to the overlap region in either antisense or sense orientation (SEQ ID NOs: 31 and 32, respectively).

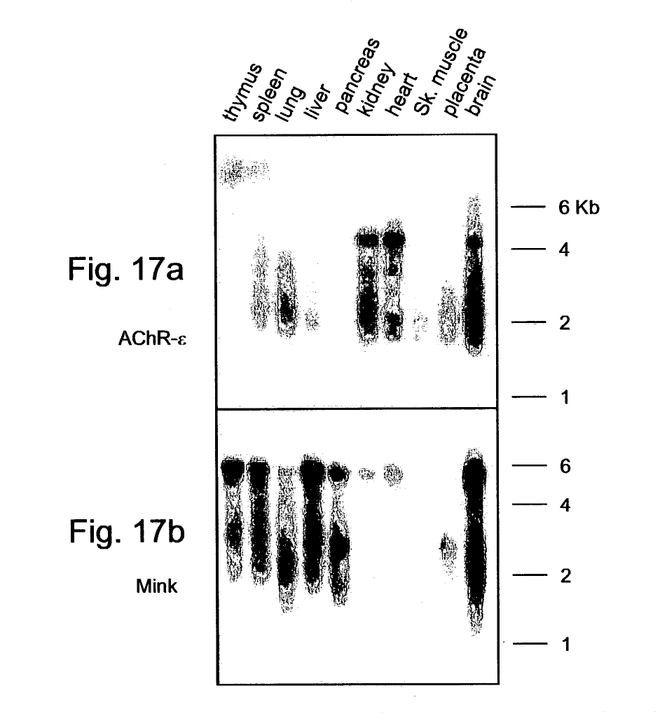

As is evident from FIG. 17a an AChRε transcript was expressed as a predominant 4 Kb band and had the highest expression in the heart, kidney and brain while surprisingly only a limited expression was observed in the skeletal muscle.

Hybridization with a MINK specific RNA probe revealed a major transcript of about 5 Kb, in accordance with previous results [Dan I. FEBS Lett. (2000) 469, 19-23] (FIG. 17b). The mRNA transcript was ubiquitously expressed with strongest expression found in brain, liver, thymus, spleen and pancreas, again in agreement with Dan I. et al.

Conclusion:

The finding that AChRε and Mink genes are antisense each to one another with a significant overlap, and the fact that the two genes are co-expressed in some tissues (eg., brain) suggest the possibility that one of them may regulate the other under certain conditions.

### Example 5

### Expression of Cyclin E2 mRNA and Complementary Transcripts in a Variety of Human Cell-Lines

Background:

The human cyclin E2 gene encodes a 404-amino-acid protein that is most closely related to cyclin E. Cyclin E2 associates with Cdk2 in a functional kinase complex that is inhibited by both p27(Kip1) and p21(Cip1). The catalytic activity associated with cyclin E2 complexes is cell cycle regulated and peaks at the G1/S transition. Overexpression of cyclin E2 in mammalian cells accelerates cell-cycle progression. Unlike cyclin E1, cyclin E2 levels are low to undetectable in nontransformed cells and increase significantly in tumor-derived cells suggesting specific mechanism of regulation.

Results:

One RNA transcript was found for cyclin E2 (SEQ ID NO: 33. Computational search for natural antisense transcripts has revealed one complementary transcript for cyclin E2 messenger RNA (SEQ ID NO: 34). The overlap between the cyclin E2 sense RNA and the antisense transcript is of at least 72 nucleotides.

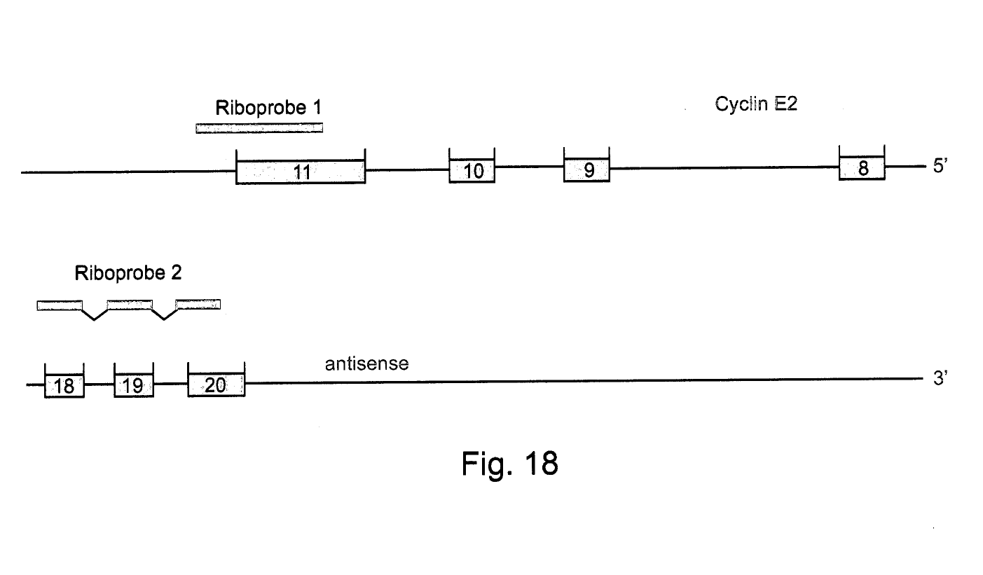

To confirm expression of the computationally retrieved antisense transcript for cyclin E2 as well as of cyclin E2 mRNA in human cell lines, two RNA-probes of 800 ribonucleotides were generated. Schematic location of the probes used for sense and antisense validation (SEQ ID NO: 44, Riboprobe#1 is illustrated in FIG. 18).

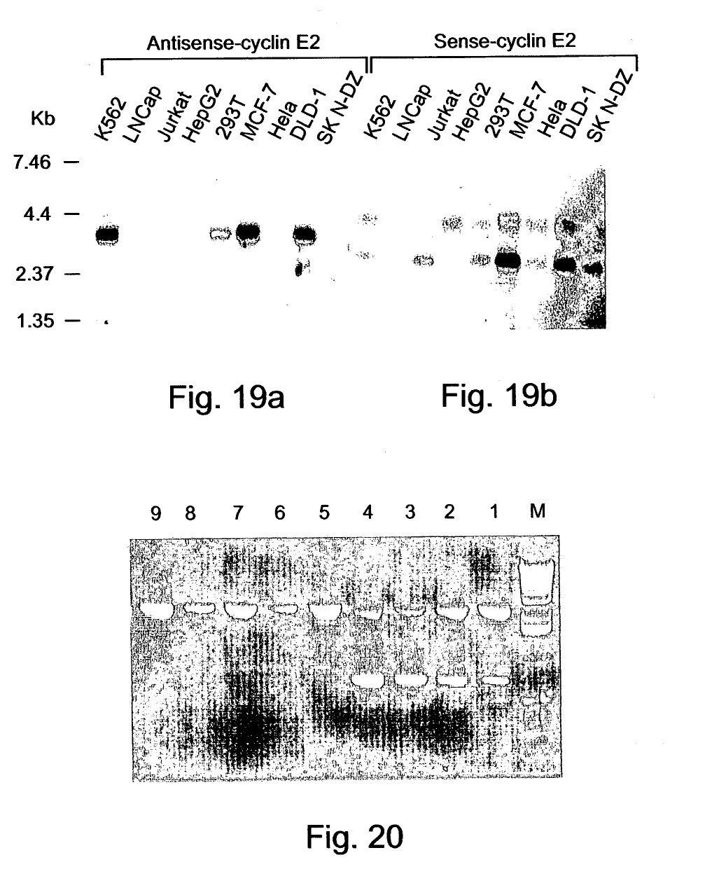

As shown in FIG. 19a, Riboprobe#1 detected two transcripts of approximately 3 Kb and 4.3 Kb. The absolute levels of the transcripts were quite heterogenic in all cell-lines examined. Both transcripts were completely absent from the Ln Cap cell line, while significantly high expression was observed in MCF-7 and DLD-1 lines, especially of the short transcript.

The same membrane was used to perform northern analysis with Riboprobe#2 in order to validate expression of antisense transcripts of cyclin E2. As is evident from FIG. 19b, an antisense transcript 3.8 Kb long was observed in most cells assayed. Significantly high pattern of expression was observed in K562, MCF-7 and DLD-1 cell lines, while only a very moderate level of expression was detected in Ln Cap and HepG2 cell lines.

### Example 6

### Co-Regulated Expression of CIDE-B and its Complementary Transcript Upon Induction of Apoptosis

The discovery of a novel naturally occurring antisense transcript to the apoptosis inducing factor, CIDE-B (see Example 2 hereinabove), suggested that the latter may be regulated by its complementary transcript, thereby establishing a novel mechanism of regulation. To address this, differential expression analysis of CIDE-B expression and its endogenous antisense transcript expression was performed following induction of apoptosis.

Materials and Methods

Induction of apoptosis and reverse transcription analysis—Monolayers of 293 cells were either left untreated (UT) or incubated with increasing concentrations of etoposide or staurosporine (Sigma IL). Twenty-four hours following addition of the drug, total RNA was extracted as described hereinabove. Purified RNA was further treated with DNaseI. A reverse transcription reaction were carried out with equivalent amounts of RNA in a final volume of 20 μl containing 100 pmol of the oligo(dT) primer, 250 ng of total RNA, 0.5 mM each of four deoxynucleoside triphosphates and 5 units of reverse transcriptase. The reaction mixture was incubated at 65° C. for 5 min, 42° C. for 50 min and 70° C. for 15 min. PCR was carried out in a final volume of 25 μl containing 12.5 pmol each of the oligonucleotide primers derived of exons 3 and 7 of CIDE-B (SEQ ID NOs: 39 and 40), 1 μl of RT solution and 1.75 units of Taq polymerase. Amplification was carried out by an initial denaturation step at 94° C. for 5 min followed by 35 cycles of [94° C. for 30 s, 68° C. for 30 s, and 68° C. for 130 min]. At the end of the PCR amplification, products were analyzed on agarose gels stained with ethidium bromide and visualized with UV light.

Results

Amplification reaction yielded two major PCR products of 740 bp and 2285 bp (FIG. 20). The small (740 bp) PCR product derived from the sense (CIDE-B) strand, whereas the larger (2285 bp) product represented an intronless antisense transcript. Evidently, an increase of sense transcript, concomitant with a decrease of antisense transcript, was observed following treatment with etoposide (lanes 1-4) as compared to untreated cells (lane 9), while no change was detected following staurosporine treatment (lanes 5-8).

These results suggest that following induction of apoptosis, antisense regulation of CIDE-B is abolished thereby allowing CIDE-B mediated apoptosis to proceed.

### Example 7

### Reciprocal Variation in Sense and Antisense Expression of Mouse Nicotinic Acetylcholine Receptor, Epsilon Subunit During Differentiation

The mouse nicotinic acetylcholine receptor, epsilon (mAchRε) subunit (SEQ ID NO: 35) has a critical function in a variety of differentiation processes. To address a novel concept of antisense regulation of AchRε-mediated differentiation, expression patterns of AchRε and its naturally occurring antisense transcript (SEQ ID NO: 36) were examined following induction of differentiation.

Materials and Methods

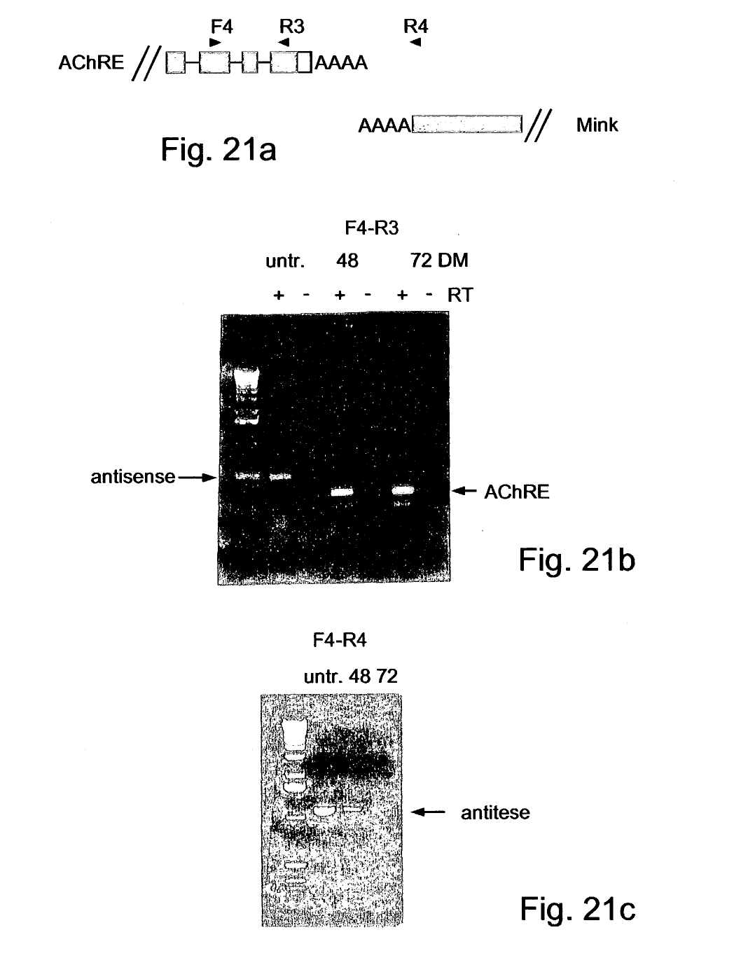

Induction of apoptosis and reverse transcription analysis—C2 mouse myoblast cells were incubated with a differentiation medium (Dulbecco's modified Eagle's medium (DMEM) including 10 μg/ml insulin and 10 μg/ml transferring) or control medium (untreated) for 48 and 72 hours. Total RNA was extracted from treated and control cells and reverse-transcribed. PCR was done using F4 and R3 primers, derived from exon numbers 10 and 12 (last exon, SEQ ID NOs: 41 and 42, respectively) of the mouse nicotinic acetylcholine receptor, epsilon subunit (mAChRε) and directed at detecting sense and antisense transcripts (see FIG. 21a).

Results

Amplification reaction showed a gradual increase in AchRε transcript expression, concomitant with the differentiation state of the cells. A second amplification product, which corresponded to an unspliced transcript was seen in untreated cells and disappeared following induction of differentiation. This fragment corresponds to a putative antisense transcript of the AchRε, and may represent an alternative 3′ UTR of the Mink gene, of which the known transcript terminates 400 bp downstream to AchRε (see Example 4). To overcome possible competition between the two transcripts, another PCR reaction was carried out using antisense specific riboprobes F4 and R4 (SEQ ID NO: 43). Reverse transcription products of this amlification reaction showed a single band which corresponded to a naturally occurring antisense transcript of the AchRε. As expected this transcript disappeared following induction of differentiation.

These results imply inverse regulation of the AchRε and its naturally occurring antisense transcript, during muscle cells differentiation from myoblasts to myotubes. Regulation may proceed, possibly through complementation of the sense and antisense transcripts to form dsRNA which can serve as a substrate for double strand RNA processing enzymes such as RNase H.

### Example 8

### A Polynucleotide Database of Sequences Corresponding to the Naturally Occurring Antisense Transcripts Identified by the Present Invention and Their Complementary Sense Sequences

Naturally occurring antisense sequences identified according to the teachings of the present invention and their corresponding sense sequences are provided in the CD-ROM1-3 enclosed herewith (file content is described in file AS_patent_data_description.doc on CD-ROM1. Generally a “seq” text file contains the actual polynucleotide sequences; a “table” file contains summarized data pertaining to each sense-antisense sequence pair; an “aligments” file contains sequence alignments of sense and antisense overlapping regions; “Table S1” and “Table S2”, further described in Example 9.

Table 3 below exemplifies the format of the Tables provided in CD-ROMs 2 and 3. Each row represents a pair of transcripts. The columns of Table 3 represent (from the left): the serial number of the pair, the name of the first transcript, its length in nucleotides, the name of the second transcript, its length in nucleotides, the number of base pairs that overlap between the two transcripts, offsets of overlap beginning at the first transcript, offsets of overlap beginning at the second transcript.

TABLE 3

| | | | | | | Start of overlap |
|---|---|---|---|---|---|---|
| | | First | | Second | Overlap | in first/ |
| Serial | First | transcript | Second | transcript | length | in second |
| No. | transcript | length (nt) | transcript | length (nt) | (nt) | transcript |
| 570_0 | AV705532_0 | 190 | Z44352_15 | 783 | OL: 52 | OF1: 1 OF2: 1 |
| | (SEQ ID NO: 1) | | (SEQ ID NO: 2) | | | |
| 570_1 | AV705532_0 | 190 | Z44352_14 | 1649 | OL: 52 | OF1: 1 OF2: 1 |
| | | | (SEQ ID NO: 3) | | | |
| 570_2 | AV705532_0 | 190 | Z44352_13 | 1861 | OL: 52 | OF1: 1 OF2: 1 |
| | | | (SEQ ID NO: 4) | | | |
| 571_0 | AW070860_0 | 214 | T81142_7 | 1934 | OL: 54 | OF1: 1 OF2: 1162 |
| | (SEQ ID NO: 5) | | (SEQ ID NO: 6) | | | |
| 571_1 | AW070860_0 | 214 | T81142_6 | 2353 | OL: 54 | OF1: 1 OF2: 1162 |
| | | | (SEQ ID NO: 7) | | | |
| 571_2 | AW070860_0 | 214 | T81142_4 | 2500 | OL: 54 | OF1: 1 OF2: 1264 |
| | | | (SEQ ID NO: 8) | | | |
| 571_3 | AW070860_0 | 214 | T81142_3 | 947 | OL: 54 | OF1: 1 OF2: 171 |
| | | | (SEQ ID NO: 9) | | | |
| 571_4 | AW070860_0 | 214 | T81142_2 | 1366 | OL: 54 | OF1: 1 OF2: 171 |
| | | | (SEQ ID NO: 10) | | | |
| 572_0 | BE046369_0 | 422 | W26553_3 | 1532 | OL: 52 | OF1: 1 OF2: 1532 |
| | (SEQ ID NO: 11) | | (SEQ ID NO: 12) | | | |
| 572_1 | BE046369_0 | 422 | W26553_2 | 1753 | OL: 52 | OF1: 1 OF2: 1753 |
| | | | (SEQ ID NO: 13) | | | |
| 572_2 | BE046369_0 | 422 | W26553_1 | 1832 | OL: 52 | OF1: 1 OF2: 1832 |
| | | | (SEQ ID NO: 14) | | | |

Pairs of transcripts are numbered, (within a contig pair, right to the underscore) that belong to a pair of contigs (numbered left to the underscore). Transcript names are arbitrary designataions.

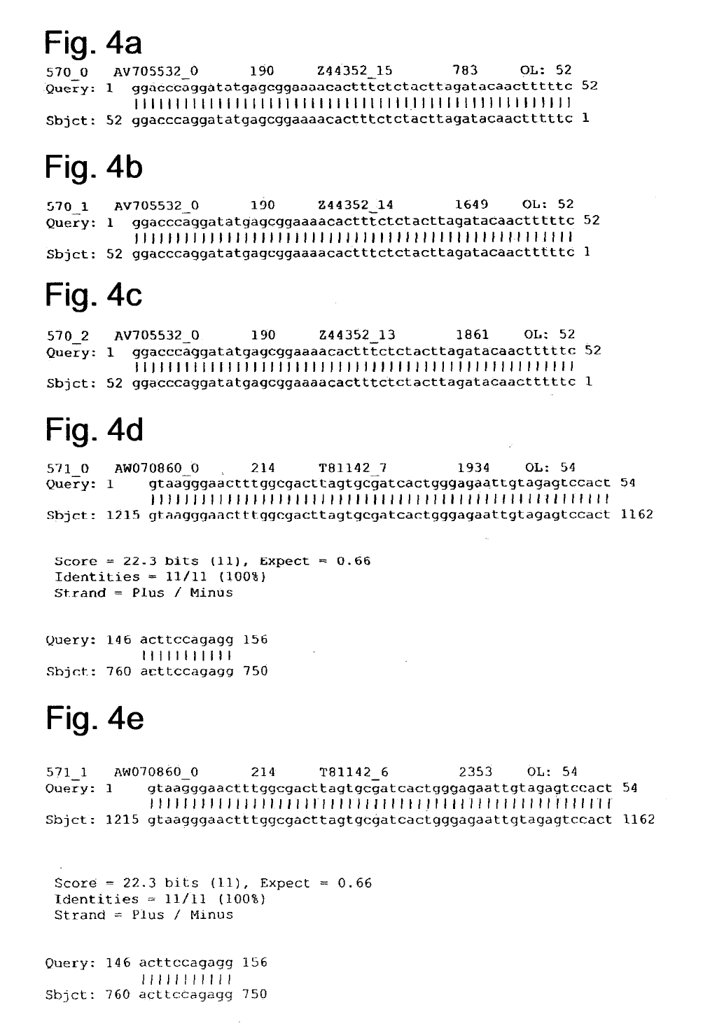

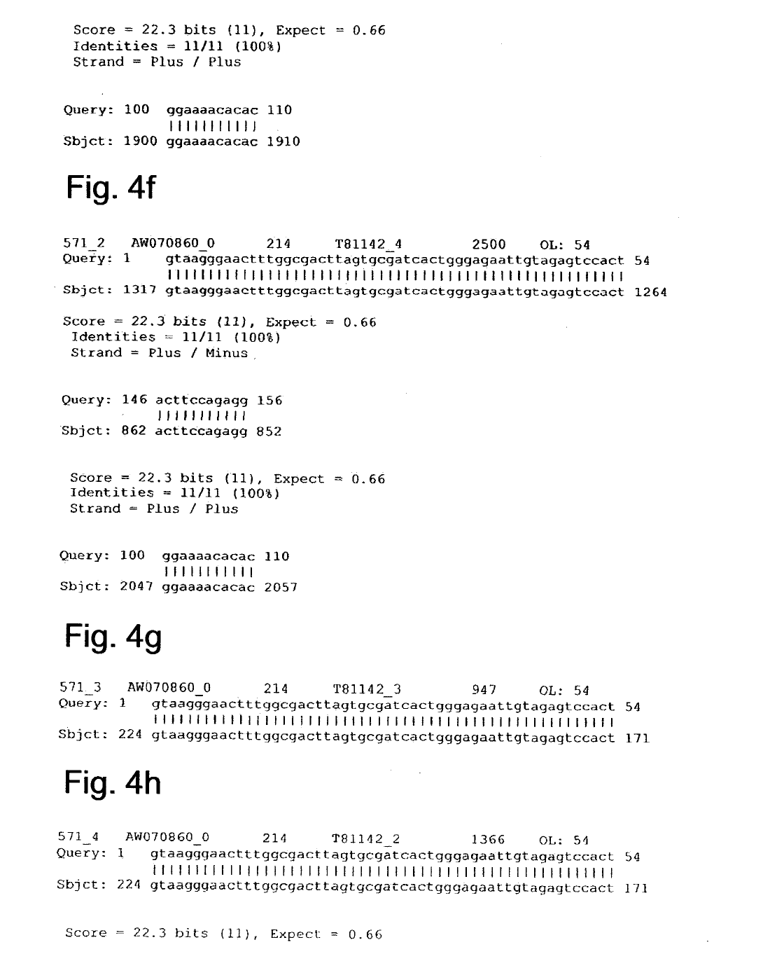

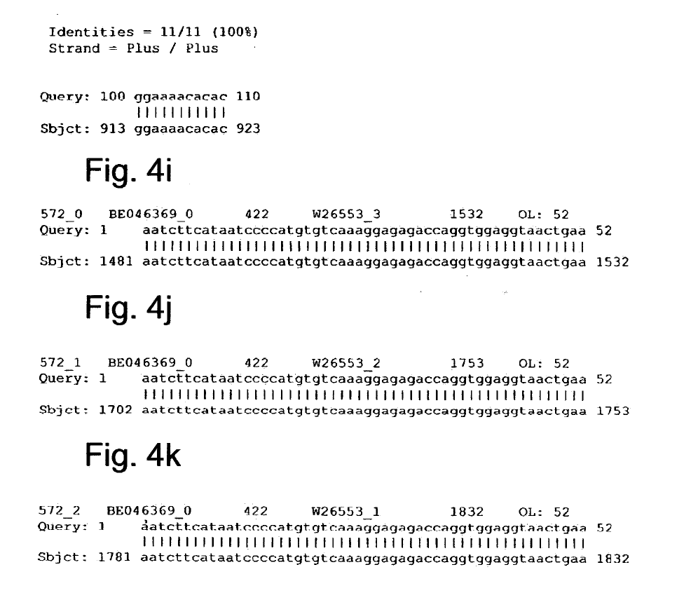

Sequence alignment of the overlapping region in each sense and antisense pair of Table 1 is demonstrated in FIG. 4a-k. Alignments were performed using the BLAST sequence alignment algorithm (Basic Local Alignment Search Tool, available through www.ncbi.nlm.nih.gov/BLAST). Interestingly, alignment profile shows high level of variability with regard to overlap lengths. It is conceivable that short overlaps are due to technical reasons associated with insufficient sequence data.

The putative naturally occurring antisense transcripts identified by the present invention and disclosed in the enclosed CD-ROMs can be used to detect and/or treat a variety of diseases, disorders or conditions, examples of which are listed hereinunder. For example, antisense transcripts or sequence information derived therefrom can be used to construct microarray kits (described in details in the preferred embodiments section) dedicated to diagnosing specific diseases, disorders or conditions.

The following sections list examples of proteins (subsection i), based on their molecular function, which participate in variety of diseases (listed in subsection ii), which diseases can be diagnosed/treated using information derived from naturally occurring antisense transcripts such as those uncovered by the present invention.

i. Molecular Function

Defense/Immunity Proteins

Information derived from proteins involved in the immune and complement systems, such as acute-phase response proteins, antimicrobial peptides, antiviral response proteins, blood coagulation factors, complement components, immunoglobulins, major histocompatibility complex antigens, and opsonins can be used to diagnose/treat diseases involving the immunological system including inflammation, autoimmune diseases, infectious diseases, as well as cancerous processes. Diseases which are manifested by non-normal coagulation processes, which may include abnormal bleeding or excessive coagulation.

Immunoglobulins

Information derived from proteins involved in the immune and complement systems including antigens and autoantigens, immunoglobulins, MHC and HLA proteins and their associated proteins can be used to diagnose/treat diseases involving the immunological system including inflammation, autoimmune diseases, infectious diseases, as well as cancerous processes.

Nucleotide Binding Proteins

Information derived from ligand binding or carrier proteins can be used to diagnose/treat diseases involving dysregulated expression, activity or localization of nucleotide binding proteins.

Nucleic Acid Binding Proteins

Information derived from proteins involved in RNA and DNA synthesis and expression regulation, such as transcription factors, RNA and DNA binding proteins, zinc fingers, helicase, isomerase, histones, nucleases, ribonucleoproteins, transcription and translation factors and others can be used to diagnose/treat diseases involving DNA or RNA binding proteins such as: helicases, isomerases, histones and nucleases, for example diseases where there is non-normal replication or transcription of DNA and RNA respectively.

RNA Polymerase II Transcription Factors

Information derived from proteins such as specific and non-specific RNA polymerase II transcription factors, enhancer binding, ligand-regulated transcription factor and general RNA polymerase II transcription factors can be used to diagnose/treat diseases involving RNA polymerase II transcription factors, for example disorders involving abnormal transcription of RNA.

RNA Binding Proteins

Information derived from RNA binding proteins involved in splicing and translation regulation, such as tRNA binding proteins, RNA helicases, double-stranded RNA and single-stranded RNA binding proteins, mRNA binding proteins, snRNA cap binding proteins, 5S RNA and 7S RNA binding proteins, poly-pyrimidine tract binding proteins, snRNA binding proteins, and AU-specific RNA binding proteins can be used to diagnose/treat diseases involving transcription and translation factors such as: helicases, isomerases, histones and nucleases, for example diseases where there is non-normal transcription, splicing, post-transcriptional processing, translation or stability of the RNA.

Chaperones

Information derived from proteins such as ribosomal chaperone, peptidylprolyl isomerase, lectin-binding chaperone, nucleosome assembly chaperone, chaperonin ATPase, cochaperone, heat shock protein, HSP70/HSP90 organizing protein, fimbrial chaperone, metallochaperone, tubulin folding, HSC70-interacting protein can be used to diagnose/treat diseases involving pathological conditions, which are associated with non-normal protein activity or structure. Binding of the products of the proteins of this family, or antibodies reactive therewith, can modulate a plurality of protein activities as well as change protein structure. Alternatively, diseases in which there is abnormal degradation of other proteins, which may cause non-normal accumulation of various proteinaceous products in cells, caused non-normal (prolonged or shortened) activity of proteins, etc.

Motor Proteins

Information derived from proteins that generate force or energy by the hydrolysis of ATP and that function in the production of intracellular movement or transportation including microfilameni motor, axonemal motor, microtubule motor, kinetochore motor (like dynein, kinesin, or myosin) can be used to diagnose/treat diseases involving un-normal chemotactic movement or motor dependent macromolecule operation such as of dynamin, which affects the regulated endocytic process.

Actin Binding Proteins

Information derived from actin binding proteins, such as actin cross-linking, actin bundling, F-actin capping, actin monomer binding, actin lateral binding, actin depolymerizing, actin monomer sequestering, actin filament severing, actin modulating, membrane associated actin binding, actin thin filament length regulation and actin polymerizing proteins can be used to diagnose/treat diseases involving cytoskeletal malformations, aberrant cellular morphology affecting extracellular interactions and dysregulated intracellular signaling.

Enzymes

Information derived from proteins possessing enzymatic activities, such as mannosylphosphate transferase, para-hydroxybenzoate:polyprenyltransferase, Rieske iron-sulfur protein, imidazoleglycerol-phosphate synthase, sphingosine hydroxylase, tRNA 2′-phosphotransferase, sterol C-24(28) reductase, C-8 sterol isomerase, C-22 sterol desaturase, C-14 sterol reductase, C-3 sterol dehydrogenase (C-4 sterol decarboxylase), 3-keto sterol reductase, C-4 methyl sterol oxidase, dihydronicotinamide riboside quinone reductase, glutamate phosphate reductase, DNA repair enzyme, telomerase, alpha-ketoacid dehydrogenase, beta-alanyl-dopamine synthase, RNA editase, aldo-keto reductase, alkylbase DNA glycosidase, glycogen debranching enzyme, dihydropterin deaminase, dihydropterin oxidase, dimethylnitrosamine demethylase, ecdysteroid UDP-glucosyl/UDP glucuronosyl transferase, glycine cleavage system, helicase, histone deacetylase, mevaldate reductase, monooxygenase, poly(ADP-ribose) glycohydrolase, pyruvate dehydrogenase, serine esterase, sterol carrier protein X-related thiolase, transposase, tyramine-beta hydroxylase, para-aminobenzoic acid (PABA) synthase, glu-tRNA(gln) amidotransferase, molybdopterin cofactor sulfurase, lanosterol 14-alpha-demethylase, aromatase, 4-hydroxybenzoate octaprenyltransferase, 7,8-dihydro-8-oxoguanine-triphosphatase, CDP-alcohol phosphotransferase, 2,5-diamino-6-(ribosylamino)-4(3H)-pyrimidonone 5′-phosphate deaminase, diphosphoinositol polyphosphate phosphohydrolase, gamma-glutamyl carboxylase, small protein conjugating enzyme, small protein activating enzyme, 1-deoxyxylulose-5-phosphate synthase, 2′-phosphotransferase, 2-octoprenyl-3-methyl-6-methoxy-1,4-benzoquinone hydroxylase, 2C-methyl-D-erythritol 2,4-cyclodiphosphate synthase, 3,4 dihydroxy-2-butanone-4-phosphate synthase, 4-amino-4-deoxychorismate lyase, 4-diphosphocytidyl-2C-methyl-D-erythritol synthase, ADP-L-glycero-D-manno-heptose synthase, D-erythro-7,8-dihydroneopterin triphosphate 2′-epimerase, N-ethylmaleimide reductase, O-antigen ligase, O-antigen polymerase, UDP-2,3-diacylglucosamine hydrolase, arsenate reductase, carnitine racemase, cobalamin [5′-phosphate] synthase, cobinamide phosphate guanylyltransferase, enterobactin synthetase, enterochelin esterase, enterochelin synthetase, glycolate oxidase, integrase, lauroyl transferase, peptidoglycan synthetase, phosphopantetheinyltransferase, phosphoglucosamine mutase, phosphoheptose isomerase, quinolinate synthase, siroheme synthase, N-acylmannosamine-6-phosphate 2-epimerase, N-acetyl-anhydromuramoyl-L-alanine amidase, carbon-phosphorous lyase, heme-copper terminal oxidase, disulfide oxidoreductase, phthalate dioxygenase reductase, sphingosine-1-phosphate lyase, molybdopterin oxidoreductase, dehydrogenase, NADPH oxidase, naringenin-chalcone synthase, N-ethylammeline chlorohydrolase, polyketide synthase, aldolase, kinase, phosphatase, CoA-ligase, oxidoreductase, transferase, hydrolase, lyase isomerase, ligase, ATPase, sulfhydryl oxidase, lipoate-protein ligase, delta-1-pyrroline-5-carboxyate synthetase, lipoic acid synthase and tRNA dihydrouridine synthase can be used to diagnose/treat diseases which can be ameliorated by modulating the activity of various enzymes which are involved both in enzymatic processes inside cells as well as in cell signaling.

Protein Serine/Threonine Kinases

Information derived from kinases, which phosphorilate serine/threonine residues, mainly involved in signal transduction, such as transmembrane receptor protein serine/threonine kinase, 3-phosphoinositide-dependent protein kinase, DNA-dependent protein kinase, G-protein-coupled receptor phosphorylating protein kinase, SNF1A/AMP-activated protein kinase, casein kinase, calmodulin regulated protein kinase, cyclic-nucleotide dependent protein kinase, cyclin-dependent protein kinase, eukaryotic translation initiation factor 2alpha kinase, galactosyltransferase-associated kinase, glycogen synthase kinase 3, protein kinase C, receptor signaling protein serine/threonine kinase, ribosomal protein S6 kinase and IkB kinase can be used to treat, or detect, respectively, diseases which may be ameliorated by a modulating kinase activity, which is one of the main signaling pathways inside cell.

Enzyme Inhibitors

Information derived from inhibitors and suppressors of other proteins and enzymes, such as inhibitors of Kinases, phosphatases, chaperones, guanylate cyclase, DNA gyrase, ribonuclease, proteasome inhibitors, diazepam-binding inhibitor, ornithine decarboxylase inhibitor GTPase inhibitors, dUTP pyrophosphatase inhibitor, phospholipase inhibitor, proteinase inhibitor, protein biosynthesis inhibitors, alpha-amylase inhibitors can be used to treat diseases in which beneficial effect may be achieved by modulating the activity of inhibitors and suppressors of proteins and enzymes.

Signal Transducers

Information derived from various signal transducers, such as activin inhibitors, receptor-associated proteins alpha-2 macroglobulin receptors, morphogens, quorum sensing signal generators, quorum sensing response regulators, receptor signaling proteins, ligands, receptors, two-component sensor molecules, two-component response regulators can be used to diagnose/treat diseases involving abnormal signal-transduction, either as a cause, or as a result of the disease.

Receptors

Information derived from various receptors, such as signal transducers, complement receptors, ligand-dependent nuclear receptors, transmembrane receptors, GPI-anchored membrane-bound receptors, various coreceptors, internalization receptors, receptors to neurotransmitters, hormones and various other effectors and ligands can be used to diagnose/treat diseases involving various receptors, including receptors to neurotransmitters, hormones and various other effectors and ligands.

Receptor Signaling Proteins

Information derived from receptor proteins involved in signal transduction, such as receptor signaling protein serine/threonine kinase, receptor signaling protein tyrosine kinase, receptor signaling protein tyrosine phosphatase, aryl hydrocarbon receptor nuclear translocator, hematopoeitin/interferon-class (D200-domain) cytokine receptor signal transducer, transmembrane receptor protein tyrosine kinase signaling protein, transmembrane receptor protein serine/threonine kinase signaling protein, receptor signaling protein serine/threonine kinase signaling protein, receptor signaling protein serine/threonine phosphatase signaling protein, small GTPase regulatory/interacting protein, receptor signaling protein tyrosine kinase signaling protein, and receptor signaling protein serine/threonine phosphatase can be used to diagnose/treat diseases involving non-normal signal transduction, either as a cause, or as a result of the disease.

Small GTPase Regulatory/Interacting Proteins

Information derived from small GTPase regulatory proteins, such as RAB escort protein, guanyl-nucleotide exchange factor, guanyl-nucleotide exchange factor adaptor, GDP-dissociation inhibitor, GTPase inhibitor, GTPase activator, guanyl-nucleotide releasing factor, GDP-dissociation stimulator, regulator of G-protein signaling, RAS interactor, RHO interactor, RAB interactor, and RAL interactor can be used to diagnose/treat diseases involving signal-transduction, typically involving G-proteases is non-normal, either as a cause, or as a result of the disease.

Ligands

Information derived from ligands such as opioid peptides, baboon receptor ligand, branchless receptor ligand, breathless receptor ligand, ephrin, frizzled receptor ligand, frizzled-2 receptor ligand, heartless receptor ligand, Notch receptor ligand, patched receptor ligand, punt receptor ligand, Ror receptor ligand, saxophone receptor ligand, SE20 receptor ligand, sevenless receptor ligand, smooth receptor ligand, thickveins receptor ligand, Toll receptor ligand, Torso receptor ligand, death receptor ligand, scavenger receptor ligand, neuroligin, integrin ligand, hormones, pheromones, growth factors and sulfonylurea receptor ligand can be used to diagnose/treat:

(a) diseases involving non-normal secretion of proteins, which may be due to non-normal presence, absence or non-normal response to normal levels of secreted proteins including hormones, neurotransmitters, and various other proteins secreted by cells to the extracellular environment;

(b) diseases which are endocrine in essence (cause or are a result of hormones), or may be ameliorated by raising, or decreasing the level of hormones and proteins;

(c) diseases which may be ameliorated by modulating the concentration or activity or interaction binding, etc. of growth factors, cytokines, interleukins, interferon and lymphokines, typically diseases such as autoimmune diseases, inflammation related disease, Graft vs. Host diseases, diseases caused by infectious agents, cancer diseases, as well as disease originating from improper concentration of growth factors causing non-normal (either excessive or too little of) growth of various tissues themselves, or causing untimely death of a desired cell population; and

(d) diseases which are manifested by non-normal development, which may be non-normal development of the organism (genetic diseases involving non-normal development of a fetus), non-normal development of a tissue (a tissue which is not properly developed) as well as cancer diseases.

Cell Adhesion Molecules

Information derived from proteins that serve as adhesion molecules between adjoining cells, such as membrane-associated protein with guanylate kinase activity, cell adhesion receptor, neuroligin, calcium-dependent cell adhesion molecule, selectin, calcium-independent cell adhesion molecule, extracellular matrix protein can be used to diagnose/treat diseases where adhesion between adjoining cells is involved, typically conditions in which the adhesion is non-normal. Typical examples of such conditions are cancer conditions in which non-normal adhesion may cause and enhance the process of metastasis. Other examples of such conditions include conditions of non-normal growth and development of various tissues in which modulation adhesion among adjoining cells can improve the condition.

Structural Proteins

Information derived from proteins involved in cell structure, such as ribosomal proteins, cell wall proteins, cytoskeletal proteins, extracellular matrix proteins, extracellular matrix glycoproteins, amyloid proteins, plasma proteins, eye lens proteins, chorion proteins (sensu Insecta), cuticle proteins (sensu Insecta), puparial glue protein (sensu Diptera), bone proteins, yolk proteins, muscle proteins, vitelline membrane proteins (sensu Insecta), peritrophic membrane proteins (sensu Insecta), and nuclear pore proteins can be used to diagnose/treat diseases involving abnormalities in cytoskeleton, including cancerous cells, and diseased cells including those which do not propagate, grow or function normally. Diseases involving non-normal sub-cellular proteins such as non-normal ribozymal proteins.

Transporter Proteins

Information derived from proteins such as amine/polyamine transporter, lipid transporter, neurotransmitter transporter, organic acid transporter, oxygen transporter, water transporter, carriers, intracellular transportes, protein transporters, ion transporters, carbohydrate transporter, polyol transporter, amino acid transporters, vitamin/cofactor transporters, siderophore transporter, drug transporter, channel/pore class transporter, group translocator, auxiliary transport proteins, Permeases, murein transporter, organic alcohol transporter, nucleobase, nucleoside and nucleotide and nucleic acid transporters can be used to diagnose/treat diseases in which abnormal transport of molecules and macromolecules such as neurotransmitters, hormones, sugar etc. leads to various pathologies.

Intracellular Transporters

Information derived from proteins that mediate the transport of molecules and macromoleules inside the cell, such as intracellular nucleoside transporter, vacuolar assembly proteins, vesicle transporters, vesicle fusion proteins, and type II protein secretors can be used to diagnose/treat diseases in which abnormal transport of molecules and macromolecules leads to various pathologies.

Ligand Binding or Carrier Proteins

Information derived from various proteins, involved in diverse biological functions, such as pyridoxal phosphate binding, carbohydrate binding, magnesium binding, amino acid binding, cyclosporin A binding, nickel binding, chlorophyll binding, biotin binding, penicillin binding, selenium binding, tocopherol binding, lipid binding, drug binding, oxygen transporter, electron transporter, steroid binding, juvenile hormone binding, retinoid binding, heavy metal binding, calcium binding, protein binding, glycosaminoglycan binding, folate binding, odorant binding, lipopolysaccharide binding, and nucleotide binding can be used to diagnose/treat diseases involving improper intracellular or extracellular accumulation or removal of small molecules such as calcium ions, improper incorporation of metals and modified amino acids (i.e., seleno-cystein), dysregulated signaling effected by improper steroid titration etc.

Electron Transporters

Information derived from ligand binding proteins or carrier proteins involved in electron transport, such as flavin-containing electron transporter, cytochromes, electron donors, electron acceptors, electron carriers and cytochrome-c oxidases can be used to diagnose/treat diseases involving dysregulated mitochondrial activity.

Calcium Binding Proteins

Information derived from calcium binding proteins, ligand binding proteins or carriers, such as diacylglycerol kinase, Calpain, calcium-dependent protein serine/threonine phosphatase, calcium sensing proteins and calcium storage proteins can be used to diagnose/treat diseases in which intracellular or extracellular calcium storage or release is improper.

Binding Proteins

Information derived from various proteins exhibiting intermediate filament binding, LIM-domain binding, LLR-domain binding, clathrin binding, ARF binding, vinculin binding, KU70 binding, troponin C binding PDZ-domain binding, SH3-domain binding, fibroblast growth factor binding, membrane-associated protein with guanylate kinase activity interacting, Wnt-protein binding, DEAD/H-box RNA helicase binding, beta-amyloid binding, myosin binding, TATA-binding protein binding DNA topoisomerase I binding, polypeptide hormone binding, RHO binding, FH1-domain binding, syntaxin-1 binding, HSC70-interacting, transcription factor binding, metarhodopsin binding, tubulin binding, JUN kinase binding, RAN protein binding, protein signal sequence binding, importin alpha export receptor, poly-glutamine tract binding, protein carrier, beta-catenin binding, protein C-terminus binding, lipoprotein binding, cytoskeletal protein binding protein, nuclear localization sequence binding, protein phosphatase 1 binding, adenylate cyclase binding, eukaryotic initiation factor 4E binding, calmodulin binding, collagen binding, insulin-like growth factor binding, lamin binding, profilin binding, tropomyosin binding, actin binding, peroxisome targeting sequence binding, SNARE binding and cyclin binding can be used to diagnose/treat diseases involving non-normal protein activity or structure. Binding of the products of the variants of this family, or antibodies reactive therewith, can modulate a plurality of protein activities as well as change protein structure.

Transcription Factor Binding Proteins

Information derived from proteins involved in transcription factors binding, RNA and DNA binding, such as transcription factors, RNA and DNA binding proteins, zinc fingers, helicase, isomerase, histones, and nucleases can be used to diagnose/treat diseases involving transcription factors binding proteins, for example diseases where there is abnormal replication or transcription of DNA and RNA respectively.

Enzyme Regulators

Information derived from enzyme regulators, such as activators of kinases, phosphatases, sphingolipids, chaperones, guanylate cyclase, tryptophan hydroxylase, proteases, phospholipases, caspases, proprotein convertase 2 activator, cyclin-dependent protein kinase 5 activator, superoxide-generating NADPH oxidase activator, sphingomyelin phosphodiesterase activator, monophenol monooxygenase activator, proteasome activator, and GTPase activator can be used to diagnose/treat diseases in which beneficial effect may be achieved by modulating the activity of activators of proteins and enzymes.

Cell Growth and/or Maintenance Proteins

Information derived from proteins involved in any biological process required for cell survival, growth and maintenance including proteins involved in cell organization and biogenesis, cell growth, cell proliferation, metabolism, cell cycle, budding, cell shape and cell size control, sporulation (sensu Saccharomyces), transport, ion homeostasis, autophagy, cell motility, chemi-mechanical coupling, membrane fusion, cell-cell fusion and stress response can be used to diagnose/treat diseases involving premature death of cells, such as degenerative diseases, for example neurodegenerative diseases or conditions associated with aging, or alternatively, diseases in which cell apoptosis is not turned on, such as cancerous diseases.

Metabolic Proteins

Information derived from proteins involved in carbohydrate metabolism, energy pathways, electron transport, nucleobase, nucleoside, nucleotide and nucleic acid metabolism, protein metabolism and modification, amino acid and derivative metabolism, protein targeting, lipid metabolism, aromatic compound metabolism, one-carbon compound metabolism, coenzymes and prosthetic group metabolism, sulfur metabolism, phosphorus metabolism, phosphate metabolism, oxygen and radical metabolism, xenobiotic metabolism, nitrogen metabolism, fat body metabolism (sensu Insecta), protein localization, catabolism, biosynthesis, toxin metabolism, methylglyoxal metabolism, cyanate metabolism, glycolate metabolism, carbon utilization, and antibiotic metabolism can be used to treat or detect diseases in which metabolism of small molecules and macromolecules such as toxins, lipids, proteins and carbohydrates is abnormal leading to various pathologies.

Channel/Pore Class Transporters

Information derived from proteins that mediate the transport of molecules and macromoleules across membranes, such as alpha-type channels, porins and pore-forming toxins can be used to diagnose/treat diseases in which the transport of molecules and macromolecules such as neurotransmitters, hormones, sugar etc. is non-normal leading to various pathologies.

Tubulin Binding Proteins

Information derived from proteins that bind tubulin, such as microtubule binding proteins can be used to diagnose/treat diseases involving abnormal tubulin activity or structure. Binding of the RNA products of the genes of this family, or antibodies reactive therewith, can modulate a plurality of tubulin activities as well as change microtubulin structure.

Kinases

Information derived from kinases such as 2-amino-4-hydroxy-6-hydroxymethyldihydropteridine pyrophosphokinase, NAD(+) kinase, acetylglutamate kinase, adenosine kinase, adenylate kinase, adenylsulfate kinase, arginine kinase, aspartate kinase, choline kinase, creatine kinase, cytidylate kinase, deoxyadenosine kinase, deoxycytidine kinase, deoxyguanosine kinase, dephospho-CoA kinase, diacylglycerol kinase, dolichol kinase, ethanolamine kinase, galactokinase, glucokinase, glutamate 5-kinase, glycerol kinase, glycerone kinase, guanylate kinase, hexokinase, homoserine kinase, hydroxyethylthiazole kinase, inositol/phosphatidylinositol kinase, ketohexokinase, mevalonate kinase, nucleoside-diphosphate kinase, pantothenate kinase, phosphoenolpyruvate carboxykinase, phosphoglycerate kinase, phosphomevalonate kinase, protein kinase, pyruvate dehydrogenase (lipoamide) kinase, pyruvate kinase, ribokinase, ribose-phosphate pyrophosphokinase, selenide,water dikinase, shikimate kinase, thiamine pyrophosphokinase, thymidine kinase, thymidylate kinase, uridine kinase, xylulokinase, 1D-myo-inositol-trisphosphate 3-kinase, phosphofructokinase, pyridoxal kinase, sphinganine kinase, riboflavin kinase, 2-dehydro-3-deoxygalactonokinase, 2-dehydro-3-deoxygluconokinase, 4-diphosphocytidyl-2C-methyl-D-erythritol kinase, GTP pyrophosphokinase, L-fuculokinase, L-ribulokinase, L-xylulokinase, isocitrate dehydrogenase (NADP+)] kinase, acetate kinase, allose kinase, carbamate kinase, cobinamide kinase, diphosphate-purine nucleoside kinase, fructokinase, glycerate kinase, hydroxymethylpyrimidine kinase, hygromycin-B kinase, inosine kinase, kanamycin kinase, phosphomethylpyrimidine kinase, phosphoribulokinase, polyphosphate kinase, propionate kinase, pyruvate,water dikinase, rhamnulokinase, tagatose-6-phosphate kinase, tetraacyldisaccharide 4′-kinase, thiamine-phosphate kinase, undecaprenol kinase, uridylate kinase, N-acylmannosamine kinase and D-erythro-sphingosine kinase can be used to diagnose/treat diseases, which may be ameliorated by a modulating kinase activity, which is one of the main signaling pathways inside cells.

Oxidoreductases

Information derived from enzymes that catalyze an oxidation-reduction reaction, including oxidoreductases acting on CH—OH, CH—CH, CH—NH2, CH—NH, NADH or NADPH, nitrogenous compounds, sulfur group of donors, heme group, hydrogen group, diphenols and related substances as donors, oxidoreductases acting on peroxide as acceptor, superoxide radicals as acceptor, oxidizing metal ions, CH2 groups, reduced ferredoxin donor, reduced flavodoxin donor, and aldehyde or oxo group of donors can be used to diagnose/treat diseases involving non-normal activity of oxidoreductases.

Transferases

Information derived from enzymes that catalyze the transfer of a chemical group, such as a phosphate or amine, from one molecule to another including transferases, transferring one-carbon groups, aldehyde or ketonic groups, acyl groups, glycosyl groups, alkyl or aryl (other than methyl) groups, nitrogenous, phosphorus-containing groups, sulfur-containing groups and lipoyltransferase, deoxycytidyl transferases can be used to diagnose/treat diseases in which the transfer of a chemical group from one molecule to another is abnormal and a beneficial effect may be achieved by modulation of such abnormal reactions.

Transferases—One-Carbon Group

Information derived from enzymes that catalyze the transfer of a single carbon from one molecule to another including methyltransferase, amidinotransferase, hydroxymethyl-, formyl- and related transferase, carboxyl- and carbamoyltransferase can be used to diagnose/treat diseases in which the transfer of a one-carbon chemical group from one molecule to another is abnormal and a beneficial effect may be achieved by modulation of such an abnormal reaction.

Transferases—Glycosyl Groups

Information derived from enzymes that catalyze the transfer of a glycosyl from one molecule to another including murein lytic endotransglycosylase E and sialyltransferase can be used to diagnose/treat diseases in which the transfer of a glycosyl chemical group from one molecule to another is abnormal and a beneficial effect may be achieved by modulation of such an abnormal reaction.

Transferases—Phosphorus-Containing Groups

Information derived from enzymes that catalyze the transfer of phosphate from one molecule to another can be used to diagnose/treat diseases in which the transfer of a phosphate group to a modulated moiety is abnormal and a beneficial effect may be achieved by modulation of such abnormal transfer.

Hydrolases

Information derived from hydrolytic enzymes acting on ester bonds, glycosyl bonds, ether bonds, carbon-nitrogen (but not peptide) bonds, acid anhydrides, acid carbon-carbon bonds, acid halide bonds, acid phosphorus-nitrogen bonds, acid sulfur-nitrogen bonds, acid carbon-phosphorus bonds and acid sulfur-sulfur bonds can be used to diagnose/treat diseases in which the hydrolytic cleavage of a covalent bond with accompanying addition of water, —H being added to one product of the cleavage and —OH to the other, is abnormal and a beneficial effect may be achieved by modulation of such an abnormal reaction.

Hydrolases, Acting on Ester Bonds

Information derived from hydrolytic enzymes, acting on ester bonds, such as nucleases, sulfuric ester hydrolase, carboxylic ester hydrolases, thiolester hydrolase, phosphoric monoester hydrolase, phosphoric diester hydrolase, triphosphoric monoester hydrolase, diphosphoric monoester hydrolase and phosphoric triester hydrolase can be used to diagnose/treat diseases in which the hydrolytic cleavage of a covalent bond with accompanying addition of water, —H being added to one product of the cleavage and —OH to the other, is abnormal and a beneficial effect may be achieved by modulation of such an abnormalreaction.

Carboxylic Ester Hydrolases

Information derived from hydrolytic enzymes, acting on carboxylic ester bonds, such as N-acetylglucosaminylphosphatidylinositol deacetylase, 2-acetyl-1-alkylglycerophosphocholine esterase, aminoacyl-tRNA hydrolase, arylesterase, carboxylesterase, cholinesterase, gluconolactonase, sterol esterase, acetylesterase, carboxymethylenebutenolidase, protein-glutamate methylesterase, and lipase, 6-phosphogluconolactonase can be used to diagnose/treat diseases which the hydrolytic cleavage of a covalent bond with accompanying addition of water, —H being added to one product of the cleavage and —OH to the other, is abnormal and a beneficial effect may be achieved by modulation of such an abnormal reaction.

Phosphoric Monoester Hydrolases

Information derived from hydrolytic enzymes acting on ester bonds, such as nuclease, sulfuric ester hydrolase, carboxylic ester hydrolase, thiolester hydrolase, phosphoric monoester hydrolase, phosphoric diester hydrolase, triphosphoric monoester hydrolase, diphosphoric monoester hydrolase and phosphoric triester hydrolase can be used to diagnose/treat diseases in which the hydrolytic cleavage of a covalent bond with accompanying addition of water, —H being added to one product of the cleavage and —OH to the other, is abnormal and a beneficial effect may be achieved by modulation of such an abnormal reaction.

Hydrolases Acting on Glycosyl Bonds

Information derived from hydrolytic enzymes that act on glycosyl bonds, such as hydrolases hydrolyzing N-glycosyl compounds and S-glycosyl compounds, O-glycosyl compounds can be used to diagnose/treat diseases in which the bydrolase-related activities are abnormal.

Hydrolases Acting on Acid Anhydrides

Information derived from hydrolytic enzymes which act on acid anhydrides, such as phosphorus-containing anhydrides, sulfonyl-containing anhydrides, and hydrolases catalysing transmembrane movement of substances, and involved in cellular and subcellular movement can be used to diagnose/treat diseases in which the hydrolase-related activities are abnormal.

Lyases

Information derived from enzymes that catalyze the formation of double bonds by removing chemical groups from a substrate without hydrolysis or catalyze the addition of chemical groups to double bonds including carbon-carbon lyases, carbon-oxygen lyases, carbon-nitrogen lyases, carbon-sulfur lyases, carbon-halide lyases, phosphorus-oxygen lyases, and other lyases can be used to diagnose/treat diseases in which lyase activity, expression or localization is abnormal.

Ligases

Information derived from enzymes that catalyze the linkage of two molecules, generally utilizing ATP as the energy donor can be used to diagnose/treat diseases in which the joining together of two molecules in an energy-dependent process is abnormal and a beneficial effect may be achieved by modulation of such an abnormal reaction.

Ligases Catalyzing Carbon-Oxygen Bonds

Information derived from enzymes that catalyze the linkage between carbon and oxygen, such as ligase forming aminoacyl-tRNA and related compounds can be used to diagnose/treat diseases in which the linkage between carbon and oxygen in an energy-dependent process is abnormal and a beneficial effect may be achieved by modulation of such an abnormal reaction.

ATPases

Information derived from enzymes such as plasma membrane cation-transporting ATPase, ATP-binding cassette (ABC) transporter, magnesium-ATPase, hydrogen-/sodium-translocating ATPase, arsenite-transporting ATPase, protein-transporting ATPase, DNA translocase, and P-type ATPase can be used to diagnose/treat diseases associated with abnormal activity of an ATP hydrolyzing enzyme.

ii. Diseases

Various types of diseases can be diagnosed/treated using the teachings of the present invention.

Inflammatory Diseases

Examples of inflamatory diseases Include, but are not limited to, chronic inflammatory diseases and acute inflammatory diseases.

Inflammatory Diseases Associated With Hypersensitivity

Examples of hypersensitivity include, but are not limited to, Types I-IV hypersensitivity, immediate hypersensitivity, antibody mediated hypersensitivity, immune complex mediated hypersensitivity, T lymphocyte mediated hypersensitivity and DTH.

An example of type I or immediate hypersensitivity is asthma. Examples of type II hypersensitivity include, but are not limited to, rheumatoid diseases, rheumatoid autoimmune diseases, rheumatoid arthritis (Krenn V. et al., Histol Histopathol 2000 Jul;15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998;17 (1-2):49), sclerosis, systemic sclerosis (Renaudineau Y. et al., Clin Diagn Lab Immunol. 1999 Mar;6 (2):156); Chan O T. et al., Immunol Rev 1999 Jun;169:107), glandular diseases, glandular autoimmune diseases, pancreatic autoimmune diseases, diabetes, Type I diabetes (Zimmet P. Diabetes Res Clin Pract 1996 Oct;34 Suppl:S125), thyroid diseases, autoimmune thyroid diseases, Graves' disease (Orgiazzi J. Endocrinol Metab Clin North Am 2000 Jun;29 (2):339), thyroiditis, spontaneous autoimmune thyroiditis (Braley-Mullen H. and Yu S, J Immunol 2000 Dec 15;165 (12):7262), Hashimoto's thyroiditis (Toyoda N. et al., Nippon Rinsho 1999 Aug;57 (8):1810), myxedema, idiopathic myxedema (Mitsuma T. Nippon Rinsho. 1999 Aug;57 (8):1759); autoimmune reproductive diseases, ovarian diseases, ovarian autoimmunity (Garza K M. et al., J Reprod Immunol 1998 Feb;37 (2):87), autoimmune anti-sperm infertility (Diekman A B. et al., Am J Reprod Immunol. 2000 Mar;43 (3):134), repeated fetal loss (Tincani A. et al., Lupus 1998;7 Suppl 2:S107-9), neurodegenerative diseases, neurological diseases, neurological autoimmune diseases, multiple sclerosis (Cross A H. et al., J Neuroimmunol 2001 Jan 1; 112 (1-2):1), Alzheimer's disease (Oron L. et al., J Neural Transm Suppl. 1997;49:77), myasthenia gravis (Infante A J. And Kraig E, Int Rev Immunol 1999;18 (1-2):83), motor neuropathies (Kornberg A J. J Clin Neurosci. 2000 May;7 (3):191), Guillain-Barre syndrome, neuropathies and autoimmune neuropathies (Kusunoki S. Am J Med Sci. 2000 Apr;319 (4):234), myasthenic diseases, Lambert-Eaton myasthenic syndrome (Takamori M. Am J Med Sci. 2000 Apr;319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathics, autoimmune polyendocrinopathies (Antoine J C. and Honnorat J. Rev Neurol (Paris) 2000 Jan;156 (1):23); neuropathies, dysimmune neuropathies (Nobile-Orazio E. et al., Electroencephalogr Clin Neurophysiol Suppl 1999;50:419); neuromyotonia, acquired neuromyotonia, arthrogryposis multiplex congenita (Vincent A. et al., Ann N Y Acad Sci. 1998 May 13;841:482), cardiovascular diseases, cardiovascular autoimmune diseases, atherosclerosis (Matsuura E. et al., Lupus. 1998;7 Suppl 2:S135), myocardial infarction (Vaarala O. Lupus. 1998;7 Suppl 2:S132), thrombosis (Tincani A. et al., Lupus 1998;7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug 25;112 (15-16):660); anti-factor VIII autoimmune disease (Lacroix-Desmazes S. et al., Semin Thromb Hemost.2000;26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May;151 (3):178); antiphospholipid syndrome (Flamholz R. et al., J Clin Apheresis 1999;14 (4):171); heart failure, agonist-like beta-adrenoceptor antibodies in heart failure (Wallukat G. et al., Am J Cardiol. 1999 Jun 17;83 (12A):75H), thrombocytopenic purpura (Moccia F. Ann Ital Med Int. 1999 Apr-Jun;14 (2):114); hemolytic anemia, autoimmune hemolytic anemia (Efremov D G. et al., Leuk Lymphoma 1998 Jan;28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 Jan;23 (1):16), celiac disease (Landau Y E. and Shoenfeld Y. Harefuah 2000 Jan 16;138 (2):122), autoimmune diseases of the musculature, myositis, autoimmune myositis, Sjogren's syndrome (Feist E. et al., Int Arch Allergy Immunol 2000 Sep;123 (1):92); smooth muscle autoimmune disease (Zauli D. et al., Biomed Pharmacother 1999 Jun;53 (5-6):234), hepatic diseases, hepatic autoimmune diseases, autoimmune hepatitis (Manns M P. J Hepatol 2000 Aug;33 (2):326) and primary biliary cirrhosis (Strassburg C P. et al., Eur J Gastroenterol Hepatol. 1999 Jun;11 (6):595).

Examples of type IV or T cell mediated hypersensitivity, include, but are not limited to, rheumatoid diseases, rheumatoid arthritis (Tisch R, McDevitt H O. Proc Natl Acad Sci USA 1994 Jan 18;91 (2):437), systemic diseases, systemic autoimmune diseases, systemic lupus erythematosus (Datta S K., Lupus 1998;7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 Mar;92 (1):77); ovarian diseases (Garza K M. et al., J Reprod Immunol 1998 Feb;37 (2):87), prostatitis, autoimmune prostatitis (Alexander R B. et al., Urology 1997 Dec;50 (6):893), polyglandular syndrome, autoimmune polyglandular syndrome, Type I autoimmune polyglandular syndrome (Hara T. et al., Blood. 1991 Mar 1;77 (5):1127), neurological diseases, autoimmune neurological diseases, multiple sclerosis, neuritis, optic neuritis (Soderstrom M. et al., J Neurol Neurosurg Psychiatry 1994 May;57 (5):544), myasthenia gravis (Oshima M. et al., Eur J Immunol 1990 Dec;20 (12):2563), stiff-man syndrome (Hiemstra H S. et al., Proc Natl Acad Sci USA 2001 Mar 27;98 (7):3988), cardiovascular diseases, cardiac autoimmunity in Chagas' disease (Cunha-Neto E. et al., J Clin Invest 1996 Oct 15;98 (8):1709), autoimmune thrombocytopenic purpura (Semple J W. et al., Blood 1996 May 15;87 (10):4245), anti-helper T lymphocyte autoimmunity (Caporossi A P. et al, Viral Immunol 1998;11 (1):9), hemolytic anemia (Sallah S. et al., Ann Hematol 1997 Mar;74 (3):139), hepatic diseases, hepatic autoimmune diseases, hepatitis, chronic active hepatitis (Franco A. et al., Clin Immunol Immunopathol 1990 Mar;54 (3):382), biliary cirrhosis, primary biliary cirrhosis (Jones D E. Clin Sci (Colch) 1996 Nov;91 (5):551), nephric diseases, nephric autoimmune diseases, nephritis, interstitial nephritis (Kelly C J. J Am Soc Nephrol 1990 Aug;1 (2):140), connective tissue diseases, ear diseases, autoimmune connective tissue diseases, autoimmnune ear disease (Yoo T J. et al., Cell Immunol 1994 Aug;157 (1):249), disease of the inner ear (Gloddek B. et al., Ann N Y Acad Sci 1997 Dec 29;830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Autoimmune Diseases

Examples of autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998;7 Suppl 2:S135), myocardial infarction (Vaarala O. Lupus. 1998;7 Suppl 2:S132), thrombosis (Tincani A. et al., Lupus 1998;7 Suppl 2:S107-9), Wegener's granulomatosis, Takayasu's arteritis, Kawasaki syndrome (Praprotnik S. et al., Wien Kin Wochenschr 2000 Aug 25;112 (15-16):660), anti-factor VIII autoimmune disease (Lacroix-Desmazes S. et al., Semin Thromb Hemost.2000;26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May;151 (3):178), antiphospholipid syndrome (Flamholz R. et al., J Clin Apheresis 1999;14 (4):171), antibody-induced heart failure (Wallukat G. et al., Am J Cardiol. 1999 Jun 17;83 (12A):75H), thrombocytopenic purpura (Moccia F. Ann Ital Med Int. 1999 Apr-Jun;14 (2):114; Semple J W. et al., Blood 1996 May 15;87 (10):4245), autoimmune hemolytic anemia (Efremov D G. et al., Leuk Lymphoma 1998 Jan;28 (3-4):285; Sallah S. et al., Ann Hematol 1997 Mar;74 (3):139), cardiac autoimmunity in Chagas' disease (Cunha-Neto E. et al., J Clin Invest 1996 Oct 15;98 (8):1709) and anti-helper T lymphocyte autoimmunity (Caporossi A P. et al., Viral Immunol 1998;11 (1):9).

Examples of autoimmune rheumatoid diseases include, but are not limited to rheumatoid arthritis (Krenn V. et al., Histol Histopathol 2000 Jul; 15 (3):791; Tisch R, McDevitt H O. Proc Natl Acad Sci units SA 1994 Jan 18;91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 Oct;34 Suppl:S125), autoimmune thyroid diseases, Graves' disease (Orgiazzi J. Endocrinol Metab Clin North Am 2000 Jun;29 (2):339; Sakata S. et al., Mol Cell Endocrinol 1993 Mar;92 (1):77), spontaneous autoimmune thyroiditis (Braley-Mullen H. and Yu S, J Immunol 2000 Dec 15;165 (12):7262), Hashimoto's thyroiditis (Toyoda N. et al., Nippon Rinsho 1999 Aug;57 (8):1810), idiopathic myxedema (Mitsuma T. Nippon Rinsho. 1999 Aug;57 (8):1759), ovarian autoimmunity (Garza K M. et al., J Reprod Immunol 1998 Feb;37 (2):87), autoimmune anti-sperm infertility (Dickman A B. et al., Am J Reprod Immunol. 2000 Mar;43 (3):134), autoimmune prostatitis (Alexander R B. et al., Urology 1997 Dec;50 (6):893) and Type I autoimmune polyglandular syndrome (Hara T. et al., Blood. 1991 Mar 1;77 (5):1127).

Examples of autoimmune gastrointestinal diseases include, but are not limited to, chronic inflammatory intestinal diseases (Garcia Herola A. et al., Gastroenterol Hepatol. 2000 Jan;23 (1):16), celiac disease (Landau Y E. and Shoenfeld Y. Harefuah 2000 Jan 16;138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 Mar;54 (3):382), primary biliary cirrhosis (Jones D E. Clin Sci (Colch) 1996 Nov;91 (5):551; Strassburg C P. et al., Eur J Gastroenterol Hepatol. 1999 Jun;11 (6):595) and autoimmune hepatitis (Manns M P. J Hepatol 2000 Aug;33 (2):326).

Examples of autoimmune neurological diseases include, but are not limited to, multiple sclerosis (Cross A H. et al., J Neuroimmunol 2001 Jan 1;112 (1-2):1), Alzheimer's disease (Qron L. et al., J Neural Transm Suppl. 1997;49:77), myasthenia gravis (Infante A J. And Kraig E, Int Rev Immunol 1999;18 (1-2):83; Oshima M. et al., Eur J Immunol 1990 Dec;20 (12):2563), neuropathies, motor neuropathies (Kornberg A J. J Clin Neurosci. 2000 May;7 (3):191); Guillain-Barre syndrome and autoimmune neuropathies (Kusunoki S. Am J Med Sci. 2000 Apr;319 (4):234), myasthenia, Lambert-Eaton myasthenic syndrome (Takamori M. Am J Med Sci. 2000 Apr;319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar 27;98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 Jan;156 (1):23); dysimmune neuropathies (Nobile-Orazio E. et al., Electroencephalogr Clin Neurophysiol Suppl 1999;50:419); acquired neuromyotonia, arthrogryposis multiplex congenita (Vincent A. et al., Ann N Y Acad Sci. 1998 May 13;841:482), neuritis, optic neuritis (Soderstrom M. et al., J Neurol Neurosurg Psychiatry 1994 May;57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 Sep;123 (1):92) and smooth muscle autoimmune disease (Zauli D. et al., Biomed Pharmacother 1999 Jun;53 (5-6):234).

Examples of autoimmune nephric diseases include, but are not limited to, nephritis and autoimmune interstitial nephritis (Kelly C J. J Am Soc Nephrol 1990 Aug;1 (2):140).

Examples of autoimmune diseases related to reproduction include, but are not limited to, repeated fetal loss (Tincani A. et al., Lupus 1998;7 Suppl 2:S107-9).

Examples of autoimmune connective tissue diseases include, but are not limited to, ear diseases, autoimmune ear diseases (Yoo T J. et al., Cell Immunol 1994 Aug;157 (1):249) and autoimmune diseases of the inner ear (Gloddek B. et al., Ann N Y Acad Sci 1997 Dec 29;830:266).

Examples of autoimmune systemic diseases include, but are not limited to, systemic lupus erythematosus (Erikson J. et al., Immunol Res 1998;17 (1-2):49) and systemic sclerosis (Renaudineau Y. et al., Clin Diagn Lab Immunol. 1999 Mar;6 (2):156); Chan O T. et al., Immunol Rev 1999 Jun; 169: 107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

### Example 9

### Microarray Analysis Based Validation of the Antisense Dataset

A microarray-based analysis using oligonucleotide probes that hybridize to the target in a strand-specific manner, was conducted in order to experimentally validate the predicted antisense/sense pairs of the database. Two complementary 60-mer oligonucleotide probes derived from the predicted overlap region of the sense/antisense pairs, were designed. Single 60-mer oligonucleotides were previously shown to offer reliability and sensitivity for detecting specific transcripts (T. R. Hughes, et al., Nature Biotech. 19, 342 (2001).) Initially only pairs of clusters with an overlap greater than 60 bases (2,464 pairs agree with this restriction) were selected for array construction. The overlap region of each antisense pair was then verified for the presence of 60-mer oligonucleotides that matched a set of standards, such as minimal sequence similarity elsewhere in the human genome, uniform GC-content and Tm, and absence of palindromic sequences, in order to maximize the hybridization specificity. Oligonucleotide probes meeting the criteria set forth were identified for 1,211 sense/antisense pairs and a random sample of 264 pairs, which constitutes roughly one-tenth of the original dataset of 2667 sense/antisense cluster pairs, was selected for analysis by Microarrays (Table_S1 on CD-ROM2, an excerpt of which is shown in Table 5 below). In this sample, the proportion of each of the nine subgroups depicted in Table 4 is similar to that of the original dataset, indicating a good representation of the various subgroups.

TABLE 4

| mRNA/ | No cluster | 1 cluster | 2 clusters | Total |
|---|---|---|---|---|
| Splicing | w introns | w | w intron(s) | |
| | | intron(s) | | |
| No cluster w mRNA | 48 | 132 | 197 | 377 (14%) |
| 1 cluster w mRNA | 17 | 490 | 1039 | 1546 (58%) |
| 2 clusters w mRNA | 1 | 85 | 658 | 744 (28%) |
| Total | 66 (2.5%) | 707 (26%) | 1894 (71%) | 2667 (100%) |

Table represents the proportion of sense/antisense clusters in the dataset of 2667 that contain: 1) a known mRNA and 2) expressed sequences spanning at least one intron, in one of the two clusters, in both clusters or in none of the clusters.

Table 5 below is an excerpt of Table_S1 provided on CD-ROM2; Table 5 exemplifies five of the putative sense/antisense pairs that were selected for microarray analysis. The first column provides the pair number. The next two columns provide the accession numbers of representative expressed sequences from the overlapping region of the sense and the antisense genes, respectively. The two columns identified by the “RNA” header provide the accession numbers of known mRNAs in the sense and antisense clusters (if available), and the last two columns provide the GenBank descriptions of these mRNAs.

TABLE 5

| | sense seq. | antisense | RNA | RNA | description | description |
|---|---|---|---|---|---|---|
| | from over- | seq. from | in | in | of RNA | of RNA |
| Pair | lapping | overlapping | sense | a-sense | in sense | in antisense |
| no. | region | region | cluster | cluster | cluster | cluster |
| 235 | NM_{—} | NM_{—} | NM_{—} | NM_{—} | Homo sapiens | Homo sapiens |
| | 6227 | 308 | 6227 | 308 | phospholipid | protective protein for |
| | | | | | transfer protein | beta-galactosidase |
| | | | | | (PLTP), mRNA | (galactosialidosis) |
| | | | | | #DV L26232.1 | (PPGB), mRNA |
| 237 | NM_{—} | NM_ | NM_ | NM_ | Homo sapiens | Homo sapiens |
| | 4703 | 2532 | 4703 | 2532 | rabaptin-5 | nucleoporin 88kD |
| | | | | | (RAB5EP), mRNA | (NUP88) mRNA |
| | | | | | #DV X91141.1 | #DV Y08612.2 |
| 217 | NM_{—} | AV | NM_ | NM_ | Homo sapiens | Homo sapiens ATP- |
| | 14885 | 723808 | 14885 | 2940 | anaphase-promoting | binding cassette, |
| | | | | | complex 10 | sub-family E |
| | | | | | (APC 10) mRNA. | (OABP), member 1 |
| | | | | | #DV AL080090.1 | (ABCE1), mRNA. |
| 209 | BC | BG | NM_ | NM_ | Homo sapiens | Homo sapiens |
| | 8865 | 717574 | 32231 | 3099 | hypothetical protein | sorting nexin 1 |
| | | | | | FLJ22875 | (SNX1), mRNA. |
| | | | | | (FLJ22875), mRNA | #DV U53225.1 |
| 196 | BE | AL | NM_ | NM_ | Homo sapiens | Homo sapiens |
| | 885605 | 527611 | 17832 | 3640 | hypothetical protein | inhibitor of kappa |
| | | | | | FLJ20457 | light polypeptide gene |
| | | | | | (FLJ20457), mRNA | enhancer in B-cells, |
| | | | | | | kinase complex- |
| | | | | | | associated protein |
| | | | | | | (IKBKAP), mRNA |

Microarrays were constructed by spotting each of the 264 pairs of oligonucleotide probes onto treated glass slides in quadruplicates. The two counterpart oligonucleotide probes of each pair were spotted next to each other to ensure similar hybridization conditions.

As positive controls, each of the blocks contained oligonucleotides spotted at various concentrations for four ubiquitously expressed housekeeping genes: guanine nucleotide binding protein beta polypeptide 2-like 1 (gnb211, HUMMHBA123, NM_{—}006098), heat shock 70 kD protein 10 (hsp70, HSHSC70CDS0, NM_{—}006597), beta actin (actin, ACTB, NM_{—}001101), and glyceraldehyde-3-phosphate dehydrogenase (gapdh, NM_{—}002046).

Two random oligonucleotides were used as negative controls. These computer-generated arbitrary sequences displayed no alignment to human genome sequences but had the same physical characteristics as the other oligonucleotide probes. In addition, 22 probes for 11 previously documented sense/antisense pairs were also analyzed in the Microarrays (entries Pair no. “known 1”-“known 11” on Table_S1 of CD-ROM2).

The Microarrays were hybridized with poly(A)+ RNAs obtained from 19 human cell lines representing a variety of tissues and four normal human tissues (see General Materials and Methods section above). Each poly(A)+ RNA was reverse transcribed by priming with oligo(dT) and random nonamers, and engineered to incorporate a fluorescent marker. A pool containing an equal mix of the RNAs from all cell lines was also transcribed and used as a reference target. The resulting fluorescently-labeled cDNAs were combined and hybridized to the oligonucleotide Microarrays.

The experiments were performed in duplicate and utilized a fluorescent reversal of the Cy3- and Cy5-labelled cDNA. Stringent hybridization conditions were utilized in order to minimize the appearance of false positive signals, despite the possibility of compromised detection of low abundance transcripts.

The raw data was normalized at several levels; within each slide, between reciprocal slides, and globally between slides (see General Materials and Methods section above). Non-specific levels of hybridization were estimated from the negative controls. The threshold for significant positive signals resulting from authentic hybridization was set at 4 standard deviations of the mean normalized signals for the negative controls. Processed data was presented as normalized signal intensity and as normalized signal ratios (Table_S2 on CD-ROM2).

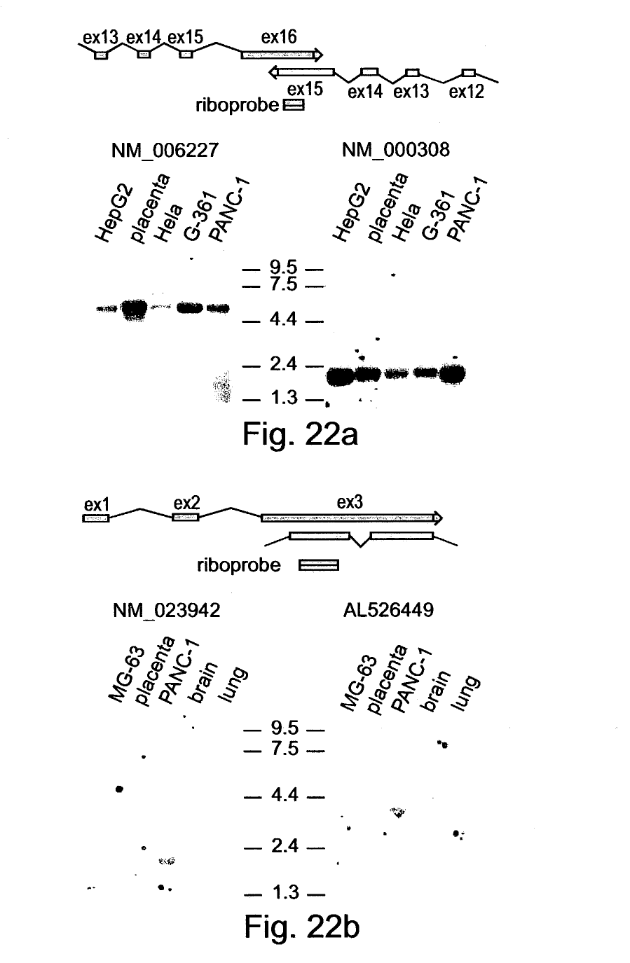

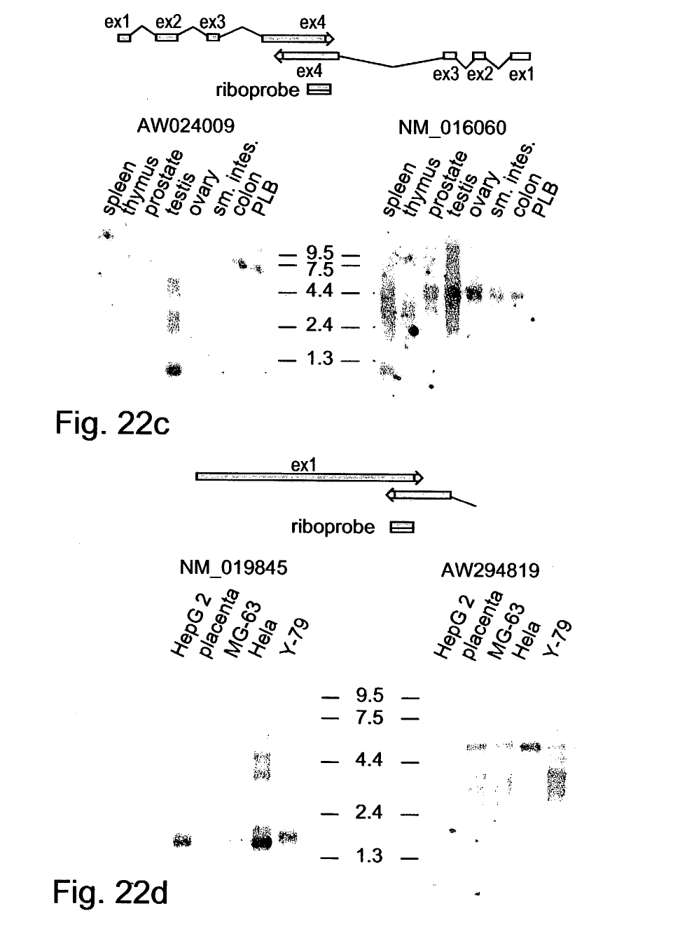

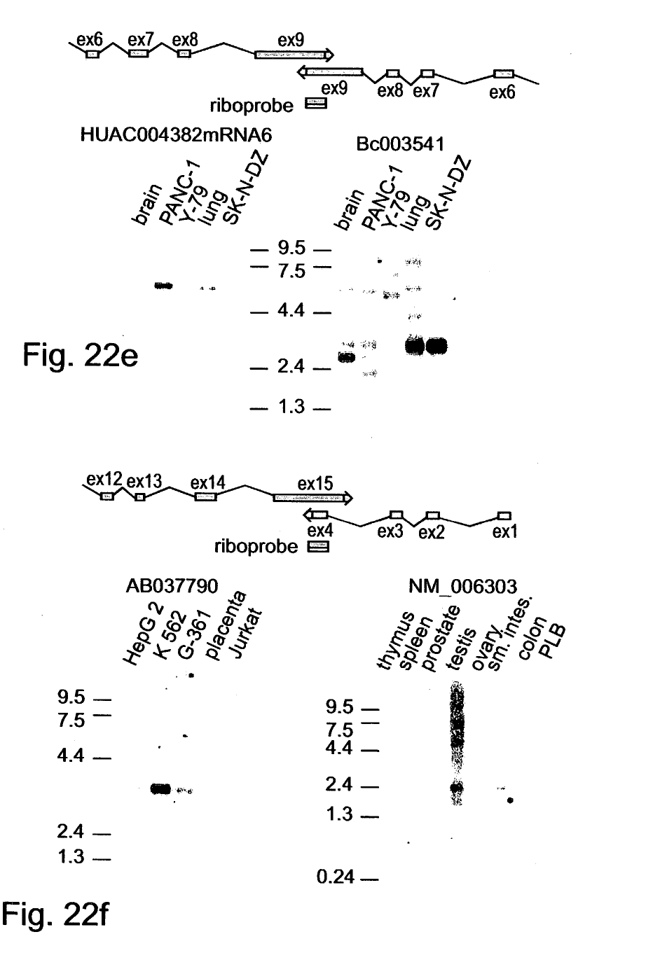

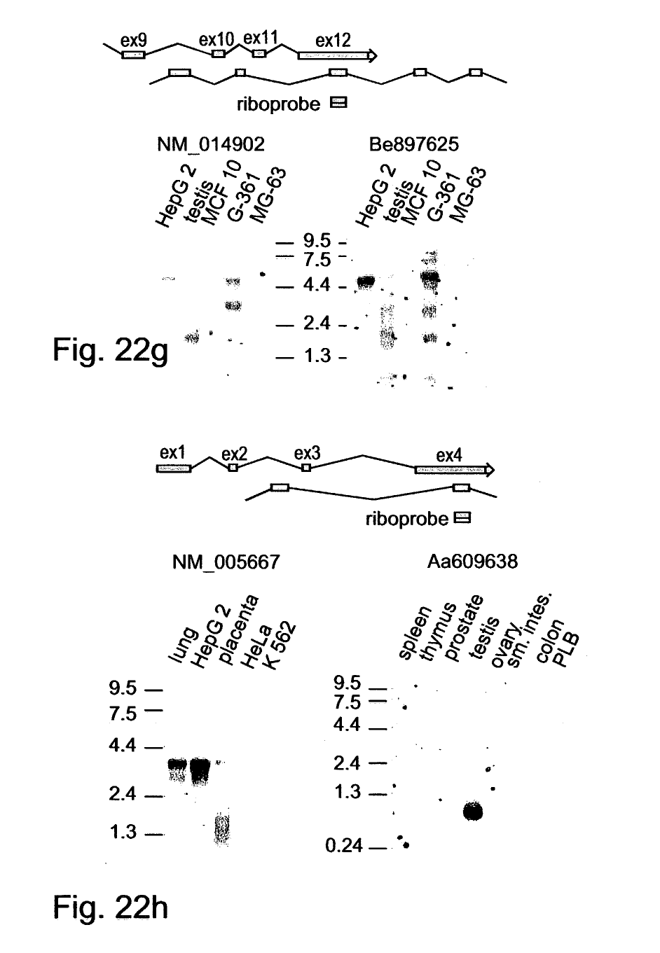

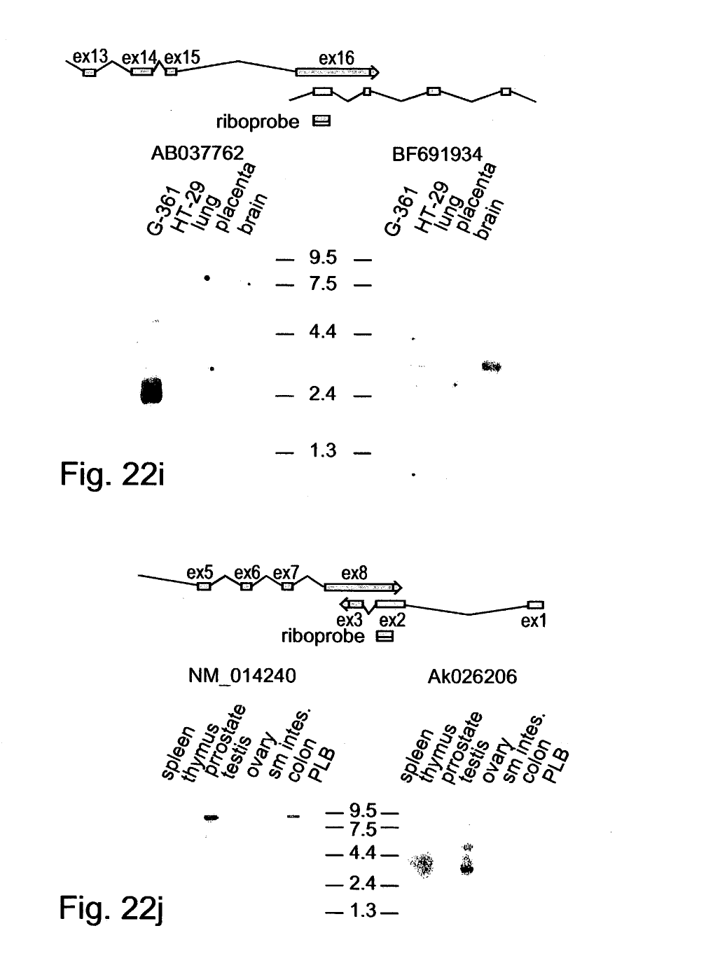

To further substantiate array results, several pairs of oligonucleotides were also utilized in Northern blot analysis. FIGS. 22a-j illustrate results of such northern blot analysis. FIG. 22a reveals expression patterns of randomly selected sequence pair number 235, denoted as Rand_{—}235 in Table 6. Similarly, FIG. 22b corresponds to pair number 173, FIG. 22c to pair number 248, FIG. 22d to pair number 6, FIG. 22e to pair number 216, FIG. 22f to pair number 239, FIG. 22g to pair number 202, FIG. 22h to pair number 114, FIG. 22i to pair number 188, and FIG. 22j to pair number 223. Eight pairs (FIGS. 22a-h) evaluated revealed positive signals for both sense and antisense expression, while two (FIGS. 22i-j) revealed a positive signal for only one of the genes, with the counterpart being a known RefSeq mRNA.

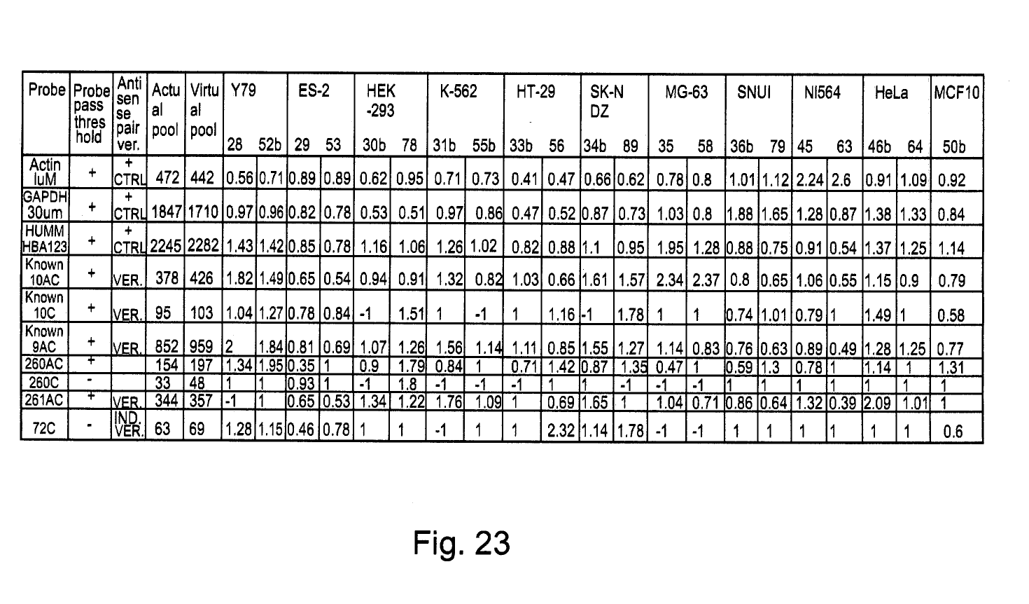

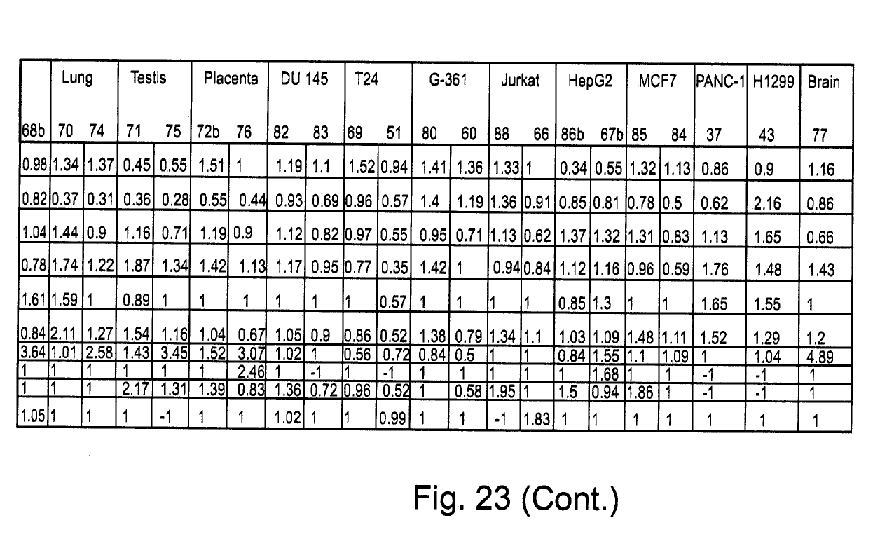

FIG. 23 represents an excerpt of Table_S2 (provided in CD-ROM2) which summarizes the results obtained utilizing the array generated according to the teachings of the present invention. Expression thresholds were verified and indicated and normalization for microarray signals was conducted as described above. Rji ratios were obtained for each cell line/tissue assessed.

Taken cumulatively, the data presented herein revealed positive signals for both sense and antisense transcripts in 65 cluster pairs. In another 47 cases, significant hybridization signals were detected for antisense sequences with known counterpart sense transcripts, i.e. RefSeq mRNAs, which did not give clear hybridization signals on the Microarrays. Thus, 42.5% (112 cases) of the 264 represented on the Microarrays, yielded detectable antisense transcription.

The conversion table, assigning the respective serial number as it appears in the “table_{—}125” file of CD-ROM2 and “table_{—}133” file of CD-ROM 3 enclosed herewith, is shown in Table 6 below.

TABLE 6

| Rand_# | Serial No | Rand_# | Serial No | Rand_# | Serial No |
|---|---|---|---|---|---|
| Rand_1 | 2326 | Rand_179 | 3266 | Rand_258 | 3807 |
| Rand_10 | 3647 | Rand_18 | 3073 | Rand_259 | 2621 |
| Rand_100 | 2758 | Rand_180 | 1794 | Rand_26 | 4009 |
| Rand_101 | 1595 | Rand_181 | 1585 | Rand_27 | 3393 |
| Rand_102 | 3686 | Rand_182 | 3554 | Rand_28 | 3589 |
| Rand_103 | 2331 | Rand_183 | 3377 | Rand_29 | 1837 |
| Rand_104 | 3496 | Rand_184 | 3466 | Rand_3 | 3046 |
| Rand_105 | 3134 | Rand_185 | 3159 | Rand_30 | 3297 |
| Rand_106 | 1339 | Rand_186 | 1413 | Rand_31 | 3692 |
| Rand_107 | 908 | Rand_187 | 3645 | Rand_32 | 707 2376 |
| Rand_108 | 2929 | Rand_188 | 3880 | Rand_33 | 2052 |
| Rand_109 | 2537 | Rand_189 | 3009 | Rand_34 | 1904 |
| Rand_11 | 2806 | Rand_19 | 3641 | Rand_35 | 3718 |
| Rand_110 | 3594 | Rand_190 | 2549 | Rand_36 | 3898 |
| Rand_111 | 2819 | Rand_191 | 2874 | Rand_37 | 1821 |
| Rand_112 | 3019 | Rand_192 | 2515 | Rand_38 | 3092 |
| Rand_113 | 3815 | Rand_193 | 3914 | Rand_39 | 3262 |
| Rand_114 | 2606 | Rand_194 | 2751 | Rand_4 | 3558 |
| Rand_115 | 1662 | Rand_195 | 2091 | Rand_40 | 2474 |
| Rand_116 | 2171 | Rand_196 | 1966 | Rand_41 | 3568 |
| Rand_117 | 2539 | Rand_197 | 3778 | Rand_42 | 864 |
| Rand_118 | 2802 | Rand_198 | 3877 | Rand_43 | 1864 |
| Rand_119 | 2761 | Rand_199 | 2248 | Rand_44 | 3045 |
| Rand_12 | 1947 | Rand_2 | 3172 | Rand_45 | 2854 |
| Rand_120 | 3228 | Rand_20 | 2360 | Rand_46 | 3852 |
| Rand_121 | 2076 | Rand_200 | 2064 | Rand_47 | 3096 |
| Rand_122 | 1835 | Rand_201 | 3597 | Rand_48 | 1987 |
| Rand_123 | 3029 | Rand_202 | 2826 | Rand_49 | 2893 |
| Rand_124 | 2898 | Rand_203 | 2388 | Rand_5 | 2060 |
| Rand_125 | 1568 | Rand_204 | 3889 | Rand_50 | 1058 |
| Rand_126 | 2456 | Rand_205 | 2211 | Rand_51 | 3560 |
| Rand_127 | 2019 | Rand_206 | 3512 | Rand_52 | 2604 |
| Rand_128 | 2346 | Rand_207 | 3452 | Rand_53 | 3397 |
| Rand_129 | 2460 | Rand_208 | 3886 | Rand_54 | 2040 |
| Rand_13 | 2429 | Rand_209 | 1600 | Rand_55 | 3784 |
| Rand_130 | 3374 | Rand_21 | 2952 | Rand_56 | 3659 |
| Rand_131 | 3292 | Rand_210 | 2432 | Rand_57 | 2005 2688 |
| Rand_132 | 3259 | Rand_211 | 1651 3968 | Rand_58 | 3187 |
| Rand_133 | 3591 | Rand_212 | 3074 | Rand_59 | 1350 |
| Rand_134 | 3340 | Rand_213 | 2341 | Rand_6 | 2202 |
| Rand_135 | 1958 | Rand_214 | 1984 | Rand_60 | 3183 |
| Rand_136 | 2274 | Rand_215 | 2803 | Rand_61 | 2275 |
| Rand_137 | 3527 | Rand_216 | 3806 | Rand_62 | 3882 |
| Rand_138 | 1533 | Rand_217 | 2186 | Rand_63 | 1044 3899 |
| Rand_139 | 2622 | Rand_218 | 857 | Rand_64 | 2811 |
| Rand_14 | 2058 | Rand_219 | 1744 | Rand_65 | 3232 |
| Rand_140 | 2578 | Rand_22 | 2285 | Rand_66 | 3242 |
| Rand_141 | 3492 | Rand_220 | 2977 | Rand_67 | 34 112 2727 |
| Rand_142 | 3928 | Rand_221 | 3863 | Rand_68 | 3909 |
| Rand_143 | 2282 3790 | Rand_222 | 2846 | Rand_69 | 4016 |
| Rand_144 | 2820 | Rand_223 | 3986 | Rand_7 | 2337 |
| Rand_145 | 1329 | Rand_224 | 579 3688 | Rand_70 | 2101 3707 |
| Rand_146 | 1783 | Rand_225 | 3984 | Rand_71 | 3703 |
| Rand_147 | 1527 | Rand_226 | 2889 | Rand_72 | 3477 |
| Rand_148 | 2662 | Rand_227 | 3869 | Rand_73 | 2437 |
| Rand_149 | 2031 | Rand_228 | 3994 | Rand_74 | 3808 |
| Rand_15 | 2677 | Rand_229 | 3818 | Rand_75 | 3905 |
| Rand_150 | 1303 1659 | Rand_23 | 3890 | Rand_76 | 1138 2194 |
| Rand_151 | 1767 | Rand_230 | 3152 | Rand_77 | 819 |
| Rand_152 | 3378 | Rand_231 | 3445 | Rand_78 | 3704 |
| Rand_153 | 984 | Rand_232 | 3663 | Rand_79 | 2309 |
| Rand_154 | 3759 | Rand_233 | 3410 | Rand_8 | 3441 |
| Rand_155 | 2046 | Rand_234 | 1112 | Rand_80 | 1219 |
| Rand_156 | 2528 | Rand_235 | 3918 | Rand_81 | 1416 |
| Rand_157 | 283 1798 | Rand_236 | 2316 | Rand_82 | 1543 |
| | 2048 | | | | |
| Rand_158 | 3710 | Rand_237 | 3673 | Rand_83 | 3269 |
| Rand_159 | 3178 | Rand_238 | 3990 | Rand_84 | 532 732 |
| Rand_16 | 3336 | Rand_239 | 4012 | Rand_85 | 2607 |
| Rand_160 | 1645 | Rand_24 | 3250 | Rand_86 | 1867 |
| Rand_161 | 2074 3464 | Rand_240 | 2932 | Rand_87 | 627 3006 |
| Rand_162 | 3436 | Rand_241 | 3836 | Rand_88 | 2068 |
| Rand_163 | 2738 | Rand_242 | 3424 | Rand_89 | 2296 |
| Rand_164 | 2749 | Rand_243 | 3982 | Rand_9 | 3741 |
| Rand_165 | 2206 | Rand_244 | 3472 | Rand_90 | 1076 |
| Rand_166 | 1349 | Rand_245 | 2071 | Rand_91 | 3385 |
| Rand_167 | 2773 | Rand_246 | 3904 | Rand_92 | 2334 |
| Rand_168 | 3305 | Rand_247 | 2056 | Rand_93 | 2833 |
| Rand_169 | 1954 | Rand_248 | 3855 | Rand_94 | 2626 |
| Rand_17 | 3940 | Rand_249 | 2980 | Rand_95 | 3671 |
| Rand_170 | 2813 | Rand_25 | 3453 | Rand_96 | 1923 |
| Rand_171 | 3868 | Rand_250 | 3565 | Rand_97 | 1863 |
| Rand_172 | 762 1424 | Rand_251 | 2459 | Rand_98 | 3437 |
| | 3942 | | | | |
| Rand_173 | 3872 | Rand_252 | 71 3147 | Rand_99 | 3469 |
| Rand_174 | 3801 | Rand_253 | 3967 | Rand_260 | 1975 3171 |
| Rand_175 | 2547 | Rand_254 | 702 2867 | Rand_261 | 4013 |
| | | | 3088 | | |
| Rand_176 | 1251 | Rand_255 | 3156 | Rand_262 | 2418 |
| Rand_177 | 1603 | Rand_256 | 2324 2998 | Rand_263 | 2451 |
| Rand_178 | 2769 | Rand_257 | 2284 | Rand_264 | 3832 |

Rand # = the name of the pair on the chip as it appears in Table_S2 on CD-ROM2, column “Probe”; Serial No = no of the pair in the Table files on CD-ROMs 2 and 3 (could be more than one in case the antisense event was separated to more than two contigs).

The sensitivity of the experimental approach utilized, i.e. the ability to detect a given transcript, stems from a combination of the stringency used in the microarray analysis and the level of expression and tissue specificity of the RNA. This can be estimated from the positive signals obtained for 65% of the oligos representing known RefSeq mRNAs on the Microarrays. This level of detection is comparable to that obtained in other studies, such as the 58% of known exons verified using microarray analysis (D. D. Shoemaker, et al., Nature 409, 922; 2001).

Thus, the present methodology provides a level of detection for a pair of genes that is 0.65×0.65=0.42, a value supported by the detection of positive signals for both sense and antisense expression in 5 out of 11 (0.45) clusters of previously described sense/antisense pairs (Table_S2 on CD-ROM2).

Of the 264 cluster pairs analyzed in the Microarrays of the present invention, 65 clusters (0.25) showed significant signals for both sense and antisense transcripts, which is 60% of the proposed level of detection for a pair of genes (0.25/0.42). Extrapolating this figure to the predicted antisense dataset of 2667 clusters, predicts at least 1600 sense/antisense transcriptional units in the human genome.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### CD-ROM Content

The following CD-ROMs are attached herewith:

Information provided as: File name/byte size/date of creation/operating system/machine format

CD-ROM1:

1. AS_patent_data_description.doc/19,968 bytes/Nov. 15, 2001/Microsoft Windows Internet Explorer/PC.

2. seqs_{—}125/343,564,655 bytes/Nov. 15, 2001/Microsoft Windows Internet Explorer/PC.

3. seqs_{—}133/259,487,305 bytes/Apr. 8, 2003/Microsoft Windows Internet Explorer/PC.

CD-ROM2:

1. alignments_{—}125/401,093,394 bytes/Nov. 15, 2001/Microsoft Windows Internet Explorer/PC.

2. table_{—}125/14,259,430 bytes/Nov. 15, 2001/Microsoft Windows Internet Explorer/PC.

3. Table_S1.xls/81,408 bytes/Jul. 10, 2002/Microsoft Windows Microsoft Excel Worksheet/PC.

4. Table S2.xls/342,528 bytes/Jul. 10, 2002/Microsoft Windows Microsoft Excel Worksheet/PC.

CD-ROM3:

1. alignments_{—}133/465,079,370 bytes/Apr. 8, 2003/Microsoft Windows Internet Explorer/PC.

2. table_{—}133/10,998,484 bytes/Apr. 8, 2003/Microsoft Windows Internet Explorer/PC.

What is claimed is:

1. A method of identifying putative naturally occurring antisense transcripts, the method comprising:
(a) computationally aligning a first database including sense-oriented polynucleotide sequences with a second database including expressed polynucleotide sequences; and
(b) identifying expressed polynucleotide sequences from said second database being capable of forming a duplex with at least one sense-oriented polynucleotide sequence of said first database, thereby identifying putative naturally occurring antisense transcripts.

2. The method of claim 1, wherein said first database includes sequences of a type selected from the group consisting of genomic sequences, expressed sequence tags, contigs, intron sequences, complementary DNA (cDNA) sequences, pre-messenger RNA (mRNA) sequences and mRNA sequences.

3. The method of claim 1, wherein said second database includes sequences of a type selected from the group consisting of expressed sequence tags, contigs, complementary DNA (cDNA) sequences, pre-messenger RNA (mRNA) sequences and mRNA sequences.

4. The method of claim 1, wherein an average sequence length of said expressed polynucleotide sequences of said second database is selected from a range of 0.02 to 0.8 Kb.

5. The method of claim 1, wherein said second database is generated by:
(i) providing a library of expressed polynucleotides;
(ii) obtaining sequence information of said expressed polynucleotides;
(iii) computationally selecting at least a portion of said expressed polynucleotides according to at least one sequence criterion; and
(iv) storing said sequence information of said at least a portion of said expressed polynucleotides thereby generating said second database.

6. The method of claim 5, wherein said at least one sequence criterion for computationally selecting said at least a portion of said expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

7. The method of claim 1 further comprising the step of testing the putative naturally occurring antisense transcripts for an ability to form said duplex with said at least one sense oriented polynucleotide sequence under physiological conditions.

8. The method of claim 1 further comprising the step of computationally testing the putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

9. A kit for quantifying at least one mRNA transcript of interest, the kit comprising at least one oligonucleotide being designed and configured so as to be complementary to a sequence region of the mRNA transcript of interest, said sequence region not being complementary with a naturally occurring antisense transcript.

10. The kit of claim 9, wherein a length of said at least one oligonucleotide is selected from a range of 15-200 nucleotides.

11. The kit of claim 9, wherein said at least one oligonucleotide is a single stranded oligonucleotide.

12. The kit of claim 9, wherein said at least one oligonucleotide is a double stranded oligonucleotide.

13. The kit of claim 9, wherein a guanidine and cytosine content of said at least one oligonucleotide is at least 25%.

14. The kit of claim 9, wherein said at least one oligonucleotide is labeled.

15. The kit of claim 9, wherein said at least one oligonucleotide is attached to a solid substrate.

16. The kit of claim 15, wherein said solid substrate is configured as a microarray and whereas said at least one oligonucleotide includes a plurality of oligonucleotides each attached to said microarray in a regio-specific manner.

17. A kit for quantifying at least one mRNA transcript of interest, the kit comprising at least one pair of oligonucleotides including a first oligonucleotide capable of binding the at least one mRNA transcript of interest and a second oligonucleotide being capable of binding a naturally occurring antisense transcript complementary to the mRNA of interest.

18. The kit of claim 17, wherein a length of each of said first and second oligonucleotides is selected from a range of 15-200 nucleotides

19. The kit of claim 17, wherein said first and second oligonucleotides are single stranded oligonucleotides.

20. The kit of claim 17, wherein said first and second oligonucleotides are double stranded oligonucleotide.

21. The kit of claim 17, wherein a guanidine and cytosine content of each of said first and second oligonucleotides is at least 25%.

22. The kit of claim 17, wherein said first and second oligonucleotides are labeled.

23. The kit of claim 17, wherein said first and second oligonucleotides are attached to a solid substrate.

24. The kit of claim 23, wherein said solid substrate is configured as a microarray and whereas each of said first and second oligonucleotides includes a plurality of oligonucleotides each attached to said microarray in a regio-specific manner.

25. A kit for quantifying at least one naturally occurring antisense transcript of interest, the kit comprising at least one oligonucleotide being designed and configured so as to be complementary to a sequence region of the at least one naturally occurring antisense transcript of interest, said sequence region not being complementary with a naturally occurring mRNA transcript.

26. The kit of claim 25, wherein a length of said at least one oligonucleotide is selected from a range of 15-200 nucleotides.

27. The kit of claim 25, wherein said at least one oligonucleotide is a single stranded oligonucleotide.

28. The kit of claim 25, wherein said at least one oligonucleotide is a double stranded oligonucleotide.

29. The kit of claim 25, wherein a guanidine and cytosine content of said at least one oligonucleotide is at least 25%.

30. The kit of claim 25, wherein said at least one oligonucleotide is labeled.

31. The kit of claim 25, wherein said at least one oligonucleotide is attached to a solid substrate.

32. The kit of claim 31, wherein said solid substrate is configured as a microarray and whereas said at least one oligonucleotide includes a plurality of oligonucleotides each attached to said microarray in a regio-specific manner.

33. A method of designing artificial antisense transcripts, the method comprising:
(a) providing a database of naturally occurring antisense transcripts;
(b) extracting from said database criteria governing structure and/or function of said naturally occurring antisense transcripts; and
(c) designing the artificial antisense transcripts according to said criteria.

34. The method of claim 33, wherein said criteria governing structure and/or function of said naturally occurring antisense transcripts are selected from the group consisting of antisense length, complementarity length, complementarity position, intron molecules, alternative splicing sites, tissue specificity, pathological abundance, chromosomal mapping, open reading frames, promoters, hairpin structures, helix structures, stem and loops, pseudoknots and tertiary interactions, guanidine and/or cytosine content, guanidine tandems, adenosine content, thermodynamic criteria, RNA duplex melting point, RNA modifications, protein-binding motifs, palindromic sequence and predicted single stranded and double stranded regions.

35. The method of claim 33, wherein said step of providing said database of naturally occurring antisense transcripts is effected by:
(a) computationally aligning a first database including sense-oriented polynucleotide sequences with a second database including expressed polynucleotide sequences; and
(b) identifying expressed polynucleotide sequences from said second database being capable of forming a duplex with at least one sense-oriented polynucleotide sequence of said first database,
(c) storing a sequence of said expressed polynucleotide sequences identified in step (b), thereby providing said database of said naturally occurring antisense transcripts.

36. The method of claim 35, wherein said first database includes sequences of a type selected from the group consisting of genomic sequences, expressed sequence tags, contigs, intron sequences, complementary DNA (cDNA) sequences, pre-messenger RNA (mRNA) sequences and mRNA sequences.

37. The method of claim 35, wherein said second database includes sequences of a type selected from the group consisting of expressed sequence tags, contigs, complementary DNA (cDNA) sequences, pre-messenger RNA (mRNA) sequences and mRNA sequences.

38. The method of claim 35, wherein an average sequence length of said expressed polynucleotide sequences of said second database is selected from a range of 0.02 to 0.8 Kb.

39. The method of claim 35, wherein said second database is generated by:
(i) providing a library of expressed polynucleotides;
(ii) obtaining sequence information of said expressed polynucleotides;
(iii) computationally selecting at least a portion of said expressed polynucleotides according to at least one sequence criterion; and
(iv) storing said sequence information of said at least a portion of said expressed polynucleotides thereby generating said second database.

40. The method of claim 39, wherein said at least one sequence criterion for computationally selecting said at least a portion of said expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

41. The method of claim 35, further comprising the step of testing said putative naturally occurring antisense transcripts for an ability to form said duplex with said at least one sense oriented polynucleotide sequence under physiological conditions.

42. The method of claim 35 further comprising the step of computationally testing said putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

43. A computer readable storage medium comprising a database including a plurality of sequences, wherein each sequence is of a naturally occurring antisense transcript.

44. The computer readable storage medium of claim 43, wherein said database further includes information pertaining to each sequence of said naturally occurring antisense transcripts, said information is selected from the group consisting of related sense gene, antisense length, complementarity length, complementarity position, intron molecules, alternative splicing sites, tissue specificity, pathological abundance, chromosomal mapping, open reading frames, promoters, hairpin structures, helix structures, stem and loops, pseudoknots and tertiary interactions, guanidine and/or cytosine content, guanidine tandems, adenosine content, thermodynamic criteria, RNA duplex melting point, RNA modifications, protein-binding motifs, palindromic sequence and predicted single stranded and double stranded regions.

45. The computer readable storage medium of claim 43, wherein said database further includes information pertaining to generation of said database and potential uses of said database.

46. A method of generating a database of naturally occurring antisense transcripts, the method comprising:
(a) computationally aligning a first database including sense-oriented polynucleotide sequences with a second database including expressed polynucleotide sequences;
(b) identifying expressed polynucleotide sequences from said second database being capable of forming a duplex with at least one sense-oriented polynucleotide sequence of said first database so as to identify putative naturally occurring antisense transcripts; and
(c) storing sequence information of said identified naturally occurring antisense transcripts, thereby generating the database of the naturally occurring antisense transcripts.

47. The method of claim 46, wherein said first database includes sequences of a type selected from the group consisting of genomic sequences, expressed sequence tags, contigs, intron sequences, complementary DNA (cDNA) sequences, pre-messenger RNA (mRNA) sequences and mRNA sequences.

48. The method of claim 46, wherein said second database includes sequences of a type selected from the group consisting of expressed sequence tags, contigs, complementary DNA (cDNA) sequences, pre-messenger RNA (mRNA) sequences and mRNA sequences.

49. The method of claim 46, wherein an average sequence length of said expressed polynucleotide sequences of said second database is selected from a range of 0.02 to 0.8 Kb.

50. The method of claim 46, wherein said second database is generated by:
(i) providing a library of expressed polynucleotides;
(ii) obtaining sequence information of said expressed polynucleotides;
(iii) computationally selecting at least a portion of said expressed polynucleotides according to at least one sequence criterion; and
(iv) storing said sequence information of said at least a portion of said expressed polynucleotides thereby generating said second database.

51. The method of claim 50, wherein said at least one sequence criterion for computationally selecting said at least a portion of said expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

52. The method of claim 46 further comprising the step of testing the putative naturally occurring antisense transcripts for an ability to form said duplex with said at least one sense oriented polynucleotide sequence under physiological conditions.

53. The method of claim 46 further comprising the step of computationally testing the putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

54. A system for generating a database of a plurality of putative naturally occurring antisense transcripts, the system comprising a processing unit, said processing unit executing a software application configured for:
(a) computationally aligning a first database including sense-oriented polynucleotide sequences with a second database including expressed polynucleotide sequences; and
(b) identifying expressed polynucleotide sequences from said second database being capable of forming a duplex with at least one sense-oriented polynucleotide sequence of said first database.

55. The system of claim 54, wherein said first database includes sequences of a type selected from the group consisting of genomic sequences, expressed sequence tags, contigs, intron sequences, complementary DNA (cDNA) sequences, pre-messenger RNA (mRNA) sequences and mRNA sequences.

56. The system of claim 54, wherein said second database includes sequences of a type selected from the group consisting of expressed sequence tags, contigs, complementary DNA (cDNA) sequences, pre-messenger RNA (mRNA) sequences and mRNA sequences.

57. The system of claim 54, wherein an average sequence length of said expressed polynucleotide sequences of said second database is selected from a range of 0.02 to 0.8 Kb.

58. The system of claim 54, wherein said second database is generated by:
(i) providing a library of expressed polynucleotides;
(ii) obtaining sequence information of said expressed polynucleotides;
(iii) computationally selecting at least a portion of said expressed polynucleotides according to at least one sequence criterion; and
(iv) storing said sequence information of said at least a portion of said expressed polynucleotides thereby generating said second database.

59. The system of claim 58, wherein said at least one sequence criterion for computationally selecting said at least a portion of said expressed polynucleotide is selected from the group consisting of sequence length, sequence annotation, sequence information, intron splice consensus site, intron sharing, sequence overlap, rare restriction site, poly(T) head, poly(A) tail, and poly(A) signal.

60. The system of claim 54 further comprising the step of testing the putative naturally occurring antisense transcripts for an ability to form said duplex with said at least one sense oriented polynucleotide sequence under physiological conditions.

61. The system of claim 54 further comprising the step of computationally testing the putative naturally occurring antisense transcripts according to at least one criterion selected from the group consisting of sequence annotation, sequence information, intron splice consensus site, intron sharing, sequence overlap, rare restriction site, poly(T) head, poly(A) tail, and poly(A) signal.

62. A method of identifying putative naturally occurring antisense transcripts, the method comprising screening a database of expressed polynucleotides sequences according to at least one sequence criterion, said at least one sequence criterion being selected to identify putative naturally occurring antisense transcripts.

63. The method of claim 63, wherein said database includes sequences of a type selected from the group consisting of expressed sequence tags, contigs, complementary DNA (cDNA) sequences, pre-messenger RNA (mRNA) sequences and mRNA sequences.

64. The method of claim 63, wherein an average sequence length of said expressed polynucleotide sequences of said second database is selected from a range of 0.02 to 0.8 Kb.

65. The method of claim 63, wherein said at least one sequence criterion is selected from the group consisting of sequence length, sequence annotation, sequence information, intron splice consensus site, intron sharing, sequence overlap, rare restriction site, poly(T) head, poly(A) tail, and poly(A) signal.

66. The method of claim 63 further comprising the step of testing the putative naturally occurring antisense transcripts for an ability to form a duplex with at least one sense oriented polynucleotide sequence under physiological conditions.

67. A method of quantifying at least one mRNA of interest in a biological sample, the method comprising:
(a) contacting the biological sample with at least one oligonucleotide capable of binding with the at least one mRNA of interest, wherein said at least one oligonucleotide is designed and configured so as to be complementary to a sequence region of the mRNA transcript of interest, said sequence region not being complementary with a naturally occurring antisense transcript; and
(b) detecting a level of binding between the at least one mRNA of interest and said at least one oligonucleotide to thereby quantify the at least one mRNA of interest in the biological sample.

68. The method of claim 67, wherein said at least one oligonucleotide is attached to a solid substrate.

69. The method of claim 68, wherein said solid substrate is configured as a microarray and whereas said at least one oligonucleotide includes a plurality of oligonucleotides each attached to said microarray in a regio-specific manner.

70. The method of claim 67, wherein said at least one oligonucleotide is labeled and whereas step (b) is effected by quantifying said label.

71. The method of claim 67, wherein a length of said at least one oligonucleotide is selected from a range of 15-200 nucleotides.

72. The method of claim 67, wherein said at least one oligonucleotide is a single stranded oligonucleotide.

73. The method of claim 67, wherein said at least one oligonucleotide is a double stranded oligonucleotide.

74. The method of claim 67, wherein a guanidine and cytosine content of said at least one oligonucleotide is at least 25%.

75. A method of quantifying the expression potential of at least one mRNA of interest in a biological sample, the method comprising:
(a) contacting the biological sample with at least one pair of oligonucleotides including a first oligonucleotide capable of binding the at least one mRNA of interest and a second oligonucleotide being capable of binding a naturally occurring antisense transcript complementary to the mRNA of interest; and
(b) detecting a level of binding between the at least one mRNA of interest and said first oligonucleotide and a level of binding between said naturally occurring antisense transcript complementary to the mRNA of interest and said second oligonucleotide to thereby quantify the expression potential of the at least one mRNA of interest in the biological sample.

76. The method of claim 75, wherein a length of each of said first and second oligonucleotides is selected from a range of 15-200 nucleotides

77. The method of claim 75, wherein said first and second oligonucleotides are single stranded oligonucleotides.

78. The method of claim 75, wherein said first and second oligonucleotides are double stranded oligonucleotide.

79. The method of claim 75, wherein a guanidine and cytosine content of each of said first and second oligonucleotides is at least 25%.

80. The method of claim 75, wherein said first and second oligonucleotides are labeled and whereas step (b) is effected by quantifying said label.

81. The method of claim 75, wherein said first and second oligonucleotides are attached to a solid substrate.

82. The method of claim 81, wherein said solid substrate is configured as a microarray and whereas each of said first and second oligonucleotides includes a plurality of oligonucleotides each attached to said microarray in a regio-specific manner.

83. A method of quantifying at least one naturally occurring antisense transcript of interest in a biological sample, the method comprising:
(a) contacting the biological sample with at least one oligonucleotide capable of binding with the at least one naturally occurring antisense transcript of interest, wherein said at least one oligonucleotide is designed and configured so as to be complementary to a sequence region of the naturally occurring antisense transcript of interest, said sequence region not being complementary with a naturally occurring mRNA transcript; and
(b) detecting a level of binding between the at least one naturally occurring antisense transcript of interest and said at least one oligonucleotide to thereby quantify the at least one naturally occurring antisense transcript of interest in the biological sample.

84. The method of claim 83, wherein said at least one oligonucleotide is attached to a solid substrate.

85. The method of claim 84, wherein said solid substrate is configured as a microarray and whereas said at least one oligonucleotide includes a plurality of oligonucleotides each attached to said microarray in a regio-specific manner.

86. The method of claim 83, wherein said at least one oligonucleotide is labeled and whereas step (b) is effected by quantifying said label.

87. The method of claim 83, wherein a length of said at least one oligonucleotide is selected from a range of 15-200 nucleotides.

88. The method of claim 83, wherein said at least one oligonucleotide is a single stranded oligonucleotide.

89. The method of claim 83, wherein said at least one oligonucleotide is a double stranded oligonucleotide.

90. The method of claim 83, wherein a guanidine and cytosine content of said at least one oligonucleotide is at least 25%.

91. A method of identifying a novel drug target, the method comprising:
(a) determining expression level of at least one naturally occurring antisense transcript of interest in cells characterized by an abnormal phenotype; and
(b) comparing said expression level of said at least one naturally occurring antisense transcript of interest in said cells characterized by an abnormal phenotype to an expression level of said at least one naturally occurring antisense transcript of interest in cells characterized by a normal phenotype, to thereby identify the novel drug target.

92. The method of claim 91, wherein said abnormal phenotype of said cells is selected from the group consisting of biochemical phenotype, morphological phenotype and nutritional phenotype.

93. The method of claim 91, wherein said determining expression level of at least one naturally occurring antisense transcript of interest is effected by at least one oligonucleotide designed and configured so as to be complementary to a sequence region of said at least one naturally occurring antisense transcript of interest, said sequence region not being complementary with a naturally occurring mRNA transcript.

94. The method of claim 93, wherein a length of said at least one oligonucleotide is selected from a range of 15-200 nucleotides.

95. The method of claim 93, wherein said at least one oligonucleotide is a single stranded oligonucleotide.

96. The method of claim 93, wherein said at least one oligonucleotide is a double stranded oligonucleotide.

97. The method of claim 93, wherein a guanidine and cytosine content of said at least one oligonucleotide is at least 25%.

98. The method of claim 93, wherein said at least one oligonucleotide is labeled and whereas step (b) is effected by quantifying said label.

99. The method of claim 93, wherein said at least one oligonucleotide is attached to a solid substrate.

100. The method of claim 99, wherein said solid substrate is configured as a microarray and whereas said at least one oligonucleotide includes a plurality of oligonucleotides each attached to said microarray in a regio-specific manner.

101. A method of treating or preventing a disease, condition or syndrome associated with an upregulation of a naturally occurring antisense transcript complementary to a naturally occurring mRNA transcript, the method comprising administering a therapeutically effective amount of an agent for regulating expression of the naturally occurring antisense transcript.

102. The method of claim 101, wherein said agent for regulating expression of the naturally occurring antisense transcript is at least one oligonucleotide designed and configured so as to hybridize to a sequence region of said at least one naturally occurring antisense transcript.

103. The method of claim 102, wherein said at least one oligonucleotide is a ribozyme.

104. The method of claim 102, wherein said at least one oligonucleotide is a sense transcript.

105. A method of diagnosing a disease, condition or syndrome associated with a substandard expression ratio of an mRNA of interest over a naturally occurring antisense transcript complementary to the mRNA of interest, the method comprising:
(a) quantifying expression level of the mRNA of interest and the naturally occurring antisense transcript complementary to the mRNA of interest;
(b) calculating the expression ratio of the mRNA of interest over the naturally occurring antisense transcript complementary to the mRNA of interest, thereby diagnosing the disease, condition or syndrome.

106. The method of claim 105, wherein quantifying said expression level of the mRNA of interest and the naturally occurring antisense transcript complementary to the mRNA of interest is effected by at least one pair of oligonucleotides including a first oligonucleotide capable of binding the mRNA of interest and a second oligonucleotide being capable of binding the naturally occurring antisense transcript complementary to the mRNA of interest.

107. The method of claim 106, wherein a length of each of said first and second oligonucleotides is selected from a range of 15-200 nucleotides

108. The method of claim 106, wherein said first and second oligonucleotides are single stranded oligonucleotides.

109. The method of claim 106, wherein said first and second oligonucleotides are double stranded oligonucleotides.

110. The method of claim 106, wherein a guanidine and cytosine content of each of said first and second oligonucleotides is at least 25%.

111. The method of claim 106, wherein said first and second oligonucleotides are labeled.

112. The method of claim 106, wherein said first and second oligonucleotides are attached to a solid substrate.

113. The method of claim 112, wherein said solid substrate is configured as a microarray and whereas each of said first and second oligonucleotides includes a plurality of oligonucleotides each attached to said microarray in a regio-specific manner.


SEQUENCE LISTING (44 sequences)

SEQ ID NO: 1 - DNA, 190 bp, Homo sapiens
ggacccaggatatgagcggaaaacactttctctacttagatacaactttttcctgtgcgcatgcctgtaatcccagctactcaggaggctgaggcaggagaatcccttgaacccaggaggcagaggttgcggtgagccaagatctcaccattgcactccagcctgggcaataagaacaaaactccgtctc

SEQ ID NO: 2 - DNA, 783 bp, Homo sapiens
gaaaaagttgtatctaagtagagaaagtgttttccgctcatatcctgggtccacatcgaagaattcagtccttgtggatgaactgtaaacagcacccttcctctaagatgccgaagatcatagtttgtggtttttttctttcaggcggtggaagcagggcagagccgaagcagcccgctcctcaagaggccggtgcggacccaggcggtgctggaccagtcagatgtgtacacccatgtcctgtcagccttcgtggaaaagaaggtgggccgcagctttccgcctcttctggactgagaatgctcaaaacaaggaagttgctgaaaacgaggagacttcatgtgattagagtcacttgaagtgattagaatcactggagtttccttgggtgaggccctagagctggtagtttggcttctaatgctgaggcctaaagcataattgttgacgggtggttctggagcgatttgtgcaaaaccagtgaaagatgaacactgggccattttaagatggaaacaaggtgggggttggatagagagttatatgcagcctcttttgcacctcgttggtatttgtaagaccacatttttttctccctaggagatgcctcataaatttgtgatagccgtgctgatggaatacattcgttctcttaaccagtttcagattgcagtacagctatgtaactgagtaagacagggagaaatattaatccgtgagtggctcccagtaagaccatggccaaatacatcctgaagtagaatatctggaaaatttgagatt

SEQ ID NO: 3 - DNA, 1649 bp, Homo sapiens
gaaaaagttgtatctaagtagagaaagtgttttccgctcatatcctgggtccacatcgaagaattcagtccttgtggatgaactgtaaacagcacccttcctctaagatgccgaagatcatagtttgtggtttttttctttcaggcggtggaagcagggcagagccgaagcagcccgctcctcaagaggccggtgcggacccaggcggtgctggaccagtcagatgtgtacacccatgtcctgtcagccttcgtggaaaagaaggtgggccgcagctttccgcctcttctggactgagaatgctcaaaacaaggaagttgctgaaaacgaggagacttcatgtgattagagtcacttgaagtgattagaatcactggagtttccttgggtgaggccctagagctggtagtttggcttctaatgctgaggcctaaagcataattgttgacgggtggttctggagcgatttgtgcaaaaccagtgaaagatgaacactgggccattttaagatggaaacaaggtgggggttggatagagagttatatgcagcctcttttgcacctcgttggtatttgtaagaccacatttttttctccctaggagatgcctcataaatttgtgatagccgtgctgatggaatacattcgttctcttaaccagtttcagattgcagtacagccttcaaatcatctgggcccaagttaaaacagaaggaatttaaaaaaaaaacacagtcactgtcttagaagatgactcatatgctaagacaggtctgcctccctgactcagaatgctgagtgactcctgacattattagttggaatgggaagtgtaaggtcaagttggggtctttacctgcatgacgaaaccacttcttgtaatgacagacttttactgtgttggttagaatagccagtccttggggagcctctagtctgttgtagctgaatgatttggaagtgttctttcactttttacttttgtcctcagcattacctacatgaacttgttatcaaaacccttgtccagcacaacctcttttatatgctgcatcagttcctgcagtaccacgtcctcagcgactccaaacctttggcttgtctgctgttatccctagagagtttctatcctcctgctcatcagctatctctggacatgctgaaggtaactctgatgtgtgaggttttagactatggaaactaactctgttcctgttgtttgcactgacctggacttctctcccttactgctagcgactttcaacagcaaatgatgaaatagtagaagttctcctttccaaacaccaagtgttagctgccttaaggtttatccggggcattggtggccatgacaacatttctgcacgaaaatttttagatgctgcaaagcagactgaagacaacatgcttttctatacaatattccgcttttttgaacagcgaaaccagcgtttgcgagggagccccaatttcacaccaggggaacactgtgaagaacatgttgcttttttcaaacagatttttggagaccaagctctaatgaggcctacaacattctgaaatcacttgctgtttttttatataaaaatgtgtacaaagttaatttattgcattaataaagctctttaaactataaaatgttaaaaagtg

SEQ ID NO: 4 - DNA, 1861 bp, Homo sapiens
gaaaaagttgtatctaagtagagaaagtgttttccgctcatatcctgggtccacatcgaagaattcagtccttgtggatgaactgtaaacagcacccttcctctaagatgccgaagatcatagtttgtggtttttttctttcaggcggtggaagcagggcagagccgaagcagcccgctcctcaagaggccggtgcggacccaggcggtgctggaccagtcagatgtgtacacccatgtcctgtcagccttcgtggaaaagaaggtgggccgcagctttccgcctcttctggactgagaatgctcaaaacaaggaagttgctgaaaacgaggagacttcatgtgattagagtcacttgaagtgattagaatcactggagtttccttgggtgaggccctagagctggtagtttggcttctaatgctgaggcctaaagcataattgttgacgggtggttctggagcgatttgtgcaaaaccagtgaaagatgaacactgggccattttaagatggaaacaaggtgggggttggatagagagttatatgcagcctcttttgcacctcgttggtatttgtaagaccacatttttttctccctaggagatgcctcataaatttgtgatagccgtgctgatggaatacattcgttctcttaaccagtttcagattgcagtacagccttcaaatcatctgggcccaagttaaaacagaaggaatttaaaaaaaaaacacagtcactgtcttagaagatgactcatatgctaagacaggtctgcctccctgactcagaatgctgagtgactcctgacattattagttggaatgggaagtgtaaggtcaagttggggtctttacctgcatgacgaaaccacttcttgtaatgacagacttttactgtgttggttagaatagccagtccttggggagcctctagtctgttgtagctgaatgatttggaagtgttctttcactttttacttttgtcctcagcattacctacatgaacttgttatcaaaacccttgtccagcacaacctcttttatatgctgcatcagttcctgcagtaccacgtcctcagcgactccaaacctttggcttgtctgctgttatccctagagagtttctatcctcctgctcatcagctatctctggacatgctgaaggtaactctgatgtgtgaggttttagactatggaaactaactctgttcctgttgtttgcactgacctggacttctctcccttactgctagcgactttcaacagcaaatgatgaaatagtagaagttctcctttccaaacaccaagtgttagctgccttaaggtttatccggggcattggtggccatgacaacatttctgcacgaaaatttttagatgctgcaaagcagactgaagacaacatgcttttctatacaatattccgcttttttgaacagcgaaaccagcgtttgcgagggagccccaatttcacaccaggtgagaatgcaatgaaaagacttggggtaaccatagcctcaaagagtagcagagggcactggcagctggtgggcgaggaccctgggttagcatttttgtaaacaacacaatttgataacagcccacctagcccttggcccattatttgtagtagagtgaattcagtatactgacagaatctggattatgctctggaactcaccgaggaggtgtgttttgagtcaagacacatttaggacccagatcaggcacagcccatctcttatagcagatcttggaatatctcttaaagccaggaataagacggcaaatggtggctaagggttttaaagggtctggggcttattaaggtttcagttttatgaagtatacattggttgat

SEQ ID NO: 5 - DNA, 214 bp, Homo sapiens
gtaagggaactttggcgacttagtgcgatcactgggagaattgtagagtccactggagagaaagaaaaatggtcaaaaagagcccagagagttcctgggggaaaacacaccgcagcccagacctattcataactgcacagctggtacttccagaggcacatgcaccaggggcacgtggttctctttgctgacaagatttattaaaagaaaagag

SEQ ID NO: 6 - DNA, 1934 bp, Homo sapiens
aagtcaacgaaaggttccgttgtccttgaccacgtattccatcacgtaaaccttgtggagatagattattttgggctacgttactgtgacagaagccatcagacgtattggctggatcctgcaaaaacccttgctgaacacaaagaactgatcaacactggacctccatatactttgtattttggtattaaattctatgctgaagatccatgtaaacttaaagaagaaataaccagatatcagtttttcttgcaggtgaagcaagatgtccttcagggccgtctgccctgtcccgtcaacactgctgctcagctgggagcgtatgccatccagtcggagcttggagattatgacccatataaacatactgcaggatatgtatctgagtaccggtttgttcctgatcagaaggaagaacttgaagaagccatagaaaggattcataaaactctaatgggtcagattccttctgaggctgagctgaattacttgaggactgccaaatccctggagatgtatggcgttgacctccatcccgtctatggagaaaacaagtctgagtatttcttaggattaactccggttggtgttgttgtgtacaagaataaaaagcaagtggggaagtatttctggcctcggattacaaaggttcacttcaaggagactcaatttgaactcagagtactgggaaaagattgtaacgaaacctcattcttttttgaagctcggagtaaaactgcttgcaagcacctctggaagtgcagtgtggaacatcatacattttttagaatgccagaaaatgaatccaattcactgtcaagaaaactcagcaagtttggatccatacgttataagcaccgctacagtggcaggacagctttgcaaatgagccgagatctttctattcagcttccccggcctgatcagaatgtgacaagaagtcgaagcaagacttaccctaagcgaatagcacaaacacagccagctgaatcaaacaccatcagtaggataactgcaaacatggaaaatggagaaaatgaaggaacaattaaaattattgcaccttcaccagtaaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccataacagtggtgaagattcagatcttaagcaaaggaggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagtgagtgcctttcagaatcttctcaccaaagctttattagtgcttgtgagtaatccattctaattcttcaattgtgttccagacagtgctttaatttgtctttacattttaaccaaaactaggtgacagtagcgaaagaggaagaaaagtgtgcattaaagctacttattctacactataatcactatcatctcttattagccacctctttgtacttggtaggtacaagggggcttttcctgattaatgtcagttttaaaataaattcttttctgagattctcactgaaaaaat

SEQ ID NO: 7 - DNA, 2353 bp, Homo sapiens
aagtcaacgaaaggttccgttgtccttgaccacgtattccatcacgtaaaccttgtggagatagattattttgggctacgttactgtgacagaagccatcagacgtattggctggatcctgcaaaaacccttgctgaacacaaagaactgatcaacactggacctccatatactttgtattttggtattaaattctatgctgaagatccatgtaaacttaaagaagaaataaccagatatcagtttttcttgcaggtgaagcaagatgtccttcagggccgtctgccctgtcccgtcaacactgctgctcagctgggagcgtatgccatccagtcggagcttggagattatgacccatataaacatactgcaggatatgtatctgagtaccggtttgttcctgatcagaaggaagaacttgaagaagccatagaaaggattcataaaactctaatgggtcagattccttctgaggctgagctgaattacttgaggactgccaaatccctggagatgtatggcgttgacctccatcccgtctatggagaaaacaagtctgagtatttcttaggattaactccggttggtgttgttgtgtacaagaataaaaagcaagtggggaagtatttctggcctcggattacaaaggttcacttcaaggagactcaatttgaactcagagtactgggaaaagattgtaacgaaacctcattcttttttgaagctcggagtaaaactgcttgcaagcacctctggaagtgcagtgtggaacatcatacattttttagaatgccagaaaatgaatccaattcactgtcaagaaaactcagcaagtttggatccatacgttataagcaccgctacagtggcaggacagctttgcaaatgagccgagatctttctattcagcttccccggcctgatcagaatgtgacaagaagtcgaagcaagacttaccctaagcgaatagcacaaacacagccagctgaatcaaacaccatcagtaggataactgcaaacatggaaaatggagaaaatgaaggaacaattaaaattattgcaccttcaccagtaaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccataacagtggtgaagattcagatcttaagcaaaggaggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagacacaaggtgacccaatccgcatcaggcattctcattcgccacgaagttaccgccagtatcgcaggtcccagtgttcagatggggagcgatcagttctctcggaagtgaattcaaaaacagatcttgtaccaccacttccggtgacccattcttcggatgctcagggttctggggatgctacagttcatcagagaagaaatgggtctaaagatagcctgatggaagaaaaacctcagacatctacaaacaacctggctggaaaacacacagcaaaaacaataaaaactatacaagcttcccgcctcaagacagagacttgatcctgatgaagggtcaagggtaggggtgggaaggttgtgtgcgccactggtacttttgaaactgtgaaataggtatcttaattcaaatctcagacctgcaagtatttcttcagcatgagaaaatacattatcttttgcttcttttttttttttttttgagatgttatcactctgtcgcccaggctggagtgcagcggcaccgtgtcagctcaccgcagcctccacttactgggttaagcgattctcctgtctcaggctaccgagcagctgggattacaggcgtgcaccacaacacccggctaattctttttgtatttttagtagagacagggctttgccatgttggaggctggtctcgaactcctgacctcaagtgatccgcctgcctcag

SEQ ID NO: 8 - DNA, 2500 bp, Homo sapiens
gacatgggctgtttctgcgctgttccggaagaattttactgcgaagttttgctcctggatgaatccaagttaacccttaccacccagcagcagggcatcaagaagtcaacgaaaggttccgttgtccttgaccacgtattccatcacgtaaaccttgtggagatagattattttgggctacgttactgtgacagaagccatcagacgtattggctggatcctgcaaaaacccttgctgaacacaaagaactgatcaacactggacctccatatactttgtattttggtattaaattctatgctgaagatccatgtaaacttaaagaagaaataaccagatatcagtttttcttgcaggtgaagcaagatgtccttcagggccgtctgccctgtcccgtcaacactgctgctcagctgggagcgtatgccatccagtcggagcttggagattatgacccatataaacatactgcaggatatgtatctgagtaccggtttgttcctgatcagaaggaagaacttgaagaagccatagaaaggattcataaaactctaatgggtcagattccttctgaggctgagctgaattacttgaggactgccaaatccctggagatgtatggcgttgacctccatcccgtctatggagaaaacaagtctgagtatttcttaggattaactccggttggtgttgttgtgtacaagaataaaaagcaagtggggaagtatttctggcctcggattacaaaggttcacttcaaggagactcaatttgaactcagagtactgggaaaagattgtaacgaaacctcattcttttttgaagctcggagtaaaactgcttgcaagcacctctggaagtgcagtgtggaacatcatacattttttagaatgccagaaaatgaatccaattcactgtcaagaaaactcagcaagtttggatccatacgttataagcaccgctacagtggcaggacagctttgcaaatgagccgagatctttctattcagcttccccggcctgatcagaatgtgacaagaagtcgaagcaagacttaccctaagcgaatagcacaaacacagccagctgaatcaaacaccatcagtaggataactgcaaacatggaaaatggagaaaatgaaggaacaattaaaattattgcaccttcaccagtaaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccataacagtggtgaagattcagatcttaagcaaaggaggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagtgagtgcctttcagaatcttctcaccaaagctttattagtgcttgacacaaggtgacccaatccgcatcaggcattctcattcgccacgaagttaccgccagtatcgcaggtcccagtgttcagatggggagcgatcagttctctcggaagtgaattcaaaaacagatcttgtaccaccacttccggtgacccattcttcggatgctcagggttctggggatgctacagttcatcagagaagaaatgggtctaaagatagcctgatggaagaaaaacctcagacatctacaaacaacctggctggaaaacacacagcaaaaacaataaaaactatacaagcttcccgcctcaagacagagacttgatcctgatgaagggtcaagggtaggggtgggaaggttgtgtgcgccactggtacttttgaaactgtgaaataggtatcttaattcaaatctcagacctgcaagtatttcttcagcatgagaaaatacattatcttttgcttcttttttttttttttttgagatgttatcactctgtcgcccaggctggagtgcagcggcaccgtgtcagctcaccgcagcctccacttactgggttaagcgattctcctgtctcaggctaccgagcagctgggattacaggcgtgcaccacaacacccggctaattctttttgtatttttagtagagacagggctttgccatgttggaggctggtctcgaactcctgacctcaagtgatccgcctgcctcag

SEQ ID NO: 9 - DNA, 947 bp, Homo sapiens
gaaagatgatactaggtcaggaaatagcatttgaaagtcattctcatctggagggatgaagccaagataaggcggaaccagggaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccattaacagtggtgaagtttcagatcttaaggcaaagggagggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagtgagtgcctttcagaatcttctcaccaaagctttattagtgcttgtgagtaatccattctaattcttcaattgtgttccagacagtgctttaatttgtctttacattttaaccaaaactaggtgacagtagcgaaagaggaagaaaagtgtgcattaaagctacttattctacactataatcactatcatctcttattagccacctctttgtacttggtaggtacaagggggcttttcctgattaatgtcagttttaaaataaattcttttctgagattctcactgaaaaaat

SEQ ID NO: 10 - DNA, 1366 bp, Homo sapiens
gaaagatgatactaggtcaggaaatagcatttgaaagtcattctcatctggagggatgaagccaagataaggcggaaccagggaaaagctttaagaaagcaaagaatgaaaatagccctgatacccaaagaagcaaatctcatgcaccgtgggaagaaaatggcccccagagtggactctacaattctcccagtgatcgcactaagtcgccaaagttcccttacacgcgtcgccgaaacccctcctgtggaagtgacaatgattctgtacagcctgtgaggaggaggaaagcccattaacagtggtgaagtttcagatcttaaggcaaagggagggaggtcacgttcacgctgtaacaccagcagtggtagtgaatcagaaaattctaatagagaacaccggaaaaagagaaacagaatacggcaggagaatgatatggttgattcagcgcctcagtgggaagctgtattaaggagacaaaaggaaaaaaaccaagccgaccccaacagcaggcgatccagacacagatctcgttcgagaagccccgatatccaagcaaaagaagagttatggaagcacattcaaaaagaacttgtggatccatccggattgtccgaagaacaattaaaagagattccatacactaaaatagagacacaaggtgacccaatccgcatcaggcattctcattcgccacgaagttaccgccagtatcgcaggtcccagtgttcagatggggagcgatcagttctctcggaagtgaattcaaaaacagatcttgtaccaccacttccggtgacccattcttcggatgctcagggttctggggatgctacagttcatcagagaagaaatgggtctaaagatagcctgatggaagaaaaacctcagacatctacaaacaacctggctggaaaacacacagcaaaaacaataaaaactatacaagcttcccgcctcaagacagagacttgatcctgatgaagggtcaagggtaggggtgggaaggttgtgtgcgccactggtacttttgaaactgtgaaataggtatcttaattcaaatctcagacctgcaagtatttcttcagcatgagaaaatacattatcttttgcttcttttttttttttttttgagatgttatcactctgtcgcccaggctggagtgcagcggcaccgtgtcagctcaccgcagcctccacttactgggttaagcgattctcctgtctcaggctaccgagcagctgggattacaggcgtgcaccacaacacccggctaattctttttgtatttttagtagagacagggctttgccatgttggaggctggtctcgaactcctgacctcaagtgatccgcctgcctcag

SEQ ID NO: 11 - DNA, 422 bp, Homo sapiens
aatcttcataatccccatgtgtcaaaggagagaccaggtggaggtaactgaatcatgggggtggtttccccaggctgtttttgtgatagtgagtgagttctcatgagatctgatggttttataaggggctcttccctcctttgcttgtgaagaaggtgccttttttcccctttgccttctgccatgattgtaagtttcctgaggcctccccagccaagctgaactgtgagtcaattaaacctcttttcttcgtaaattacccagtcttgagcagttctttacagcagtgtgaaaacagaggaatacacccatacatgctattctctgcccagaagccagggggagcctgccattaaaatgaaagtcactccttgactcagaaccctcaaatagctttcatctcacccagaaaaaaaagaaaa

SEQ ID NO: 12 - DNA, 1532 bp, Homo sapiens
aggtttctgcacaggaatatcgagagcgtcatgaacccgagctatagagaaaggagatgaggcgtgagccaccgcacccggctgacaagtgtccttctaagaaacacacagaggagaagacacagaagaggagagcaccatgtgatggtagacacagaaattggagttctacagccacaagccaaggaactcctggagccaccaggagatggaagatgcaaagaactgattttctctcagagcctctggagggagtgtggccctggtgacaccttgattttggacttctggcctacagaaccatgcacacaggaggacttcatttcccaggtctccttgcagtgaagttgaggccatgtgactggtcttgggccaatggaatgggtgcagaagggacacagcccatttctagactcagcctgaaatgtcctccataatccttactctttctcccttcactcactggctgcaggaagctgagaattatccttggacttacataaagcattttggactttatgtaagtaacaacctgttgtattaagctactaagattttacggttgtttgttaaatcagctaaccttaaacatcctaacaactacaaatagaatacctgttactgcatacataaaaatacaaaaattagctggatgtggtcccacctgtagtcccagctactcgggaggctgaggcaggagaattgcttgaacctgggaggcggaggttgtggtgagctgagatcgcaccactgcactgcagcctgggcaacagagcaggactctatctcaaaaaaaaaacacaataaacatttcttacctactgtagtttttgtgggtcaggaatctgggagcagcttagttggatgatttctgctcacagtgttttatgaggttgcagtcaagatgttggctggggctgtagtcatctggagatttaactacggctggaggatccacttcaccatggttcactcacctggtgctggttgctggcaggaaatttcagctcttctcttatatggatctcttcacagattgcttgagtgtcctcaccgtatggtgactggcttcctttacagaaatcagttgaagggaatgggcaagtaagaaacagcaatgctttttatgacctagtcctgaagttccccaccattacttatgttcattggaagccagttgctaaggagagcctgcactcaaagattggggaaatagactttatctttcaaagtgttgaagaatttgcagacgtattttaaaaccaccacacaatccatcaacacatcatgtcggctctattcttgaaatagatccagaatttgaccacttttcaccatctccattgctattacccagatctaatcaacaccatcacttgcctggactagagatttcctcctcactgggctctctgcttctatctttagcccattgctatgatttggctgtgtccccacccaaaatctcatcttgaattataatcttcataatccccatgtgtcaaaggagagaccaggtggaggtaactgaa

SEQ ID NO: 13 - DNA, 1753 bp, Homo sapiens
tttcttagggtttttttttgagttggagcctcgctctgtcccccaggctggagtgcagtgatgtgatctcggctcactgcaacctctgcctcccaggttcaagtgattctcctgcctcagcctccctagtagctgcgactacaggcatgtgccaccatgcctggctaacgttttgtatttttgagtagagacagggtttcaccatgttggccaggctattctcgaactcctgacctcaagtgatccacctgcctcggcttcccaaagtttctgggattacaggcgtgagccaccgcacccggctgacaagtgtccttctaagaaacacacagaggagaagacacagaagaggagagcaccatgtgatggtagacacagaaattggagttctacagccacaagccaaggaactcctggagccaccaggagatggaagatgcaaagaactgattttctctcagagcctctggagggagtgtggccctggtgacaccttgattttggacttctggcctacagaaccatgcacacaggaggacttcatttcccaggtctccttgcagtgaagttgaggccatgtgactggtcttgggccaatggaatgggtgcagaagggacacagcccatttctagactcagcctgaaatgtcctccataatccttactctttctcccttcactcactggctgcaggaagctgagaattatccttggacttacataaagcattttggactttatgtaagtaacaacctgttgtattaagctactaagattttacggttgtttgttaaatcagctaaccttaaacatcctaacaactacaaatagaatacctgttactgcatacataaaaatacaaaaattagctggatgtggtcccacctgtagtcccagctactcgggaggctgaggcaggagaattgcttgaacctgggaggcggaggttgtggtgagctgagatcgcaccactgcactgcagcctgggcaacagagcaggactctatctcaaaaaaaaaacacaataaacatttcttacctactgtagtttttgtgggtcaggaatctgggagcagcttagttggatgatttctgctcacagtgttttatgaggttgcagtcaagatgttggctggggctgtagtcatctggagatttaactacggctggaggatccacttcaccatggttcactcacctggtgctggttgctggcaggaaatttcagctcttctcttatatggatctcttcacagattgcttgagtgtcctcaccgtatggtgactggcttcctttacagaaatcagttgaagggaatgggcaagtaagaaacagcaatgctttttatgacctagtcctgaagttccccaccattacttatgttcattggaagccagttgctaaggagagcctgcactcaaagattggggaaatagactttatctttcaaagtgttgaagaatttgcagacgtattttaaaaccaccacacaatccatcaacacatcatgtcggctctattcttgaaatagatccagaatttgaccacttttcaccatctccattgctattacccagatctaatcaacaccatcacttgcctggactagagatttcctcctcactgggctctctgcttctatctttagcccattgctatgatttggctgtgtccccacccaaaatctcatcttgaattataatcttcataatccccatgtgtcaaaggagagaccaggtggaggtaactgaa

SEQ ID NO: 14 - DNA, 1832 bp, Homo sapiens
gggttttgcgggtataattacattcaggatctcaggatactgcattatctgtgtgacccctaaatctgatgacaagtgtctgttttttgtttttgtttttgagacagagcctcgctctgtcacccaggctggagtgctgtggtgtgatctcggctcactgcaacctccgcctcccaggttcaagcaattctctgcctcagcctcccgagtaaatgtgattacaggcaggcgcctgccagcacacccagctgattttagtatttttagtagagatggggtttcaccatcttggccaggctggtcttgaattcctgacctcgtgatccacccacttcagcttcccaaagttctgggattacaggcgtgagccaccgcacccggctgacaagtgtccttctaagaaacacacagaggagaagacacagaagaggagagcaccatgtgatggtagacacagaaattggagttctacagccacaagccaaggaactcctggagccaccaggagatggaagatgcaaagaactgattttctctcagagcctctggagggagtgtggccctggtgacaccttgattttggacttctggcctacagaaccatgcacacaggaggacttcatttcccaggtctccttgcagtgaagttgaggccatgtgactggtcttgggccaatggaatgggtgcagaagggacacagcccatttctagactcagcctgaaatgtcctccataatccttactctttctcccttcactcactggctgcaggaagctgagaattatccttggacttacataaagcattttggactttatgtaagtaacaacctgttgtattaagctactaagattttacggttgtttgttaaatcagctaaccttaaacatcctaacaactacaaatagaatacctgttactgcatacataaaaatacaaaaattagctggatgtggtcccacctgtagtcccagctactcgggaggctgaggcaggagaattgcttgaacctgggaggcggaggttgtggtgagctgagatcgcaccactgcactgcagcctgggcaacagagcaggactctatctcaaaaaaaaaacacaataaacatttcttacctactgtagtttttgtgggtcaggaatctgggagcagcttagttggatgatttctgctcacagtgttttatgaggttgcagtcaagatgttggctggggctgtagtcatctggagatttaactacggctggaggatccacttcaccatggttcactcacctggtgctggttgctggcaggaaatttcagctcttctcttatatggatctcttcacagattgcttgagtgtcctcaccgtatggtgactggcttcctttacagaaatcagttgaagggaatgggcaagtaagaaacagcaatgctttttatgacctagtcctgaagttccccaccattacttatgttcattggaagccagttgctaaggagagcctgcactcaaagattggggaaatagactttatctttcaaagtgttgaagaatttgcagacgtattttaaaaccaccacacaatccatcaacacatcatgtcggctctattcttgaaatagatccagaatttgaccacttttcaccatctccattgctattacccagatctaatcaacaccatcacttgcctggactagagatttcctcctcactgggctctctgcttctatctttagcccattgctatgatttggctgtgtccccacccaaaatctcatcttgaattataatcttcataatccccatgtgtcaaaggagagaccaggtggaggtaactgaa

SEQ ID NO: 15 - DNA, 10394 bp, Homo sapiens
cgttgtttggcgtgtttttttttttgttttttgtcactgcctgcctgggtcctgcccgaggtctccatcctcggtttccctgtccttgccccgggccctgggagtgctctggaaggctgcgcagtattggaggggacagaatgaccttccggccttgagtccctggggagcagatggaccctactggaagtcagttggattcagatttctctcagcaagatactccttgcctgataattgaagattctcagcctgaaagccaggttctagaggatgattctggttctcacttcagtatgctatctcgacaccttcctaatctccagacgcacaaagaaaatcctgtgttggatgttgtgtccaatcctgaacaaacagctggagaagaacgaggagacggtaatagtgggttcaatgaacatttgaaagaaaacaaggttgcagaccctgtggattcttctaacttggacacatgtggttccatcagtcaggtcattgagcagttacctcagccaaacaggacaagcagtgttctgggaatgtcagtggaatctgctcctgctgtggaggaagagaagggagaagagttggaacagaaggagaaagagaaggaagaagatacttcaggcaatactacacattcccttggtgctgaagatactgcctcatcacagttgggttttggggttctggaactctcccagagccaggatgttgaggaaaatactgtgccatatgaagtggacaaagagcagctacaatcagtaaccaccaactctggttataccaggctgtctgatgtggatgctaatactgcaattaagcatgaagaacagtccaacgaagatatccccatagcagaacagtccagcaaggacatccctgtgacagcacagcccagtaaggatgtacatgttgtaaaagagcaaaatccaccacctgcaaggtcagaggacatgccttttagccccaaagcatctgttgctgctatggaagcaaaagaacagttgtctgcacaagaacttatggaaagtggactgcagattcagaagtcaccagagcctgaggttttgtcaactcaggaagacttgtttgaccagagcaataaaacagtatcttctgatggttgctctactccttcaagggaggaaggtgggtgttctttggcttccactcctgccaccactctgcatctcctgcagctctctggtcagaggtcccttgttcaggacagtctttccacgaattcttcagatcttgttgctccttctcctgatgctttccgatctactccttttatcgttcctagcagtcccacagagcaagaagggagacaagataagccaatggacacgtcagtgttatctgaagaaggaggagagccttttcagaagaaacttcaaagtggtgaaccagtggagttagaaaacccccctctcctgcctgagtccactgtatcaccacaagcctcaacaccaatatctcagagcacaccagtcttccctcctgggtcacttcctatcccatcccagcctcagttttctcatgacatttttattccttccccaagtctggaagaacaatcaaatgatgggaagaaagatggagatatgcatagttcatctttgacagttgagtgttctaaaacttcagagattgaaccaaagaattcccctgaggatcttgggctatctttgacaggggattcttgcaagttgatgctttctacaagtgaatatagtcagtccccaaagatggagagcttgagttctcacagaattgatgaagatggagaaaacacacagattgaggatacggaacccatgtctccagttctcaattctaaatttgttcctgctgaaaatgatagtatcctgatgaatccagcacaggatggtgaagtacaactgagtcagaatgatgacaaaacaaagggagatgatacagacaccagggatgacattagtattttagccactggttgcaagggcagagaagaaacggtagcagaagatgtttgtattgatctcacttgtgattcggggagtcaggcagttccgtcaccagctactcgatctgaggcactttctagtgtgttagatcaggaggaagctatggaaattaaagaacaccatccagaggaggggtcttcagggtctgaggtggaagaaatccctgagacaccttgtgaaagtcaaggagaggaactcaaagaagaaaatatggagagtgttccgttgcacctttctctgactgaaactcagtcccaagggttgtgtcttcaaaaggaaatgccaaaaaaagaatgctcagaagctatggaagttgaaaccagtgtgattagtattgattcccctcaaaagttggcaatacttgaccaagaattggaacataaggaacaggaagcttgggaagaagctacttcagaggactccagtgttgtcattgtagatgtgaaagagccatctcccagagttgatgtttcttgtgaacctttggagggagtggagaagtgctcagattcccagtcatgggaggatattgctccagaaatagaaccatgtgctgagaatagattagacaccaaggaagaaaagagtgtagaatatgaaggagatctgaaatcagggactgcagaaacagaacctgtagagcaagattcttcacagccttccttacctttagtgagagcagatgatcctttaagacttgaccaggagttgcagcagccccaaactcaggagaaaacaagtaattcattaacagaagactcaaaaatggctaatgcaaagcagctaagctcagatgcagaggcccagaagctggggaagccctctgcccatgcctcacaaagcttctgtgaaagttctagtgaaaccccatttcatttcactttgcctaaagaaggtgatatcatcccaccattgactggtgcaaccccacctcttattgggcacctaaaattggagcccaagagacacagtactcctattggtattagcaactatccagaaagcaccatagcaaccagtgatgtcatgtctgaaagcatggtggagacccatgatcccatacttgggagtggaaaaggggattctggggctgccccagacgtggatgataaattatgtctaagaatgaaactggttagtcctgagactgaggcgagtgaagagtctttgcagttcaacctggaaaagcctgcaactggtgaaagaaaaaatggatctactgctgttgctgagtctgttgccagtccccagaagaccatgtctgtgttgagctgtatctgtgaagccaggcaagagaatgaggctcgaagtgaggatccccccaccacacccatcagggggaacttgctccactttccaagttctcaaggagaagaggagaaagaaaaattggagggtgaccatacaatcaggcagagtcaacagcctatgaagcccattagtcctgtcaaggaccctgtttctcctgcttcccagaagatggtcatacaagggccatccagtcctcaaggagaggcaatggtgacagatgtgctagaagaccagaaagaaggacggagtactaataaggaaaatcctagtaaggccttgattgaaaggcccagccaaaataacataggaatccaaaccatggagtgttccttgagggtcccagaaactgtttcagcagcaacccagactataaagaatgtgtgtgagcaggggaccagtacagtggaccagaactttggaaagcaagatgccacagttcagactgagagggggagtggtgagaaaccagtcagtgctcctggggatgatacagagtcgctccatagccagggagaagaagagtttgatatgcctcagcctccacatggccatgtcttacatcgtcacatgagaacaatccgggaagtacgcacacttgtcactcgtgtcattacagatgtgtattatgtggatggaacagaagtagaaagaaaagtaactgaggagactgaagagccaattgtagagtgtcaggagtgtgaaactgaagtttccccttcacagactgggggctcctcaggtgacctgggggatatcagctccttctcctccaaggcatccagcttacaccgcacatcaagtgggacaagtctctcagctatgcacagcagtggaagctcagggaaaggagccggaccactcagagggaaaaccagcgggacagaacccgcagattttgccttacccagctcccgaggaggcccaggaaaactgagtcctagaaaaggggtcagtcagacagggacgccagtgtgtgaggaggatggtgatgcaggccttggcatcagacagggagggaaggctccagtcacgcctcgtgggcgtgggcgaaggggccgcccaccttctcggaccactggaaccagagaaacagctgtgcctggccccttgggcatagaggacatttcacctaacttgtcaccagatgataaatccttcagccgtgtcgtgccccgagtgccagactccaccagacgaacagatgtgggtgctggtgctttgcgtcgtagtgactctccagaaattcctttccaggctgctgctggcccttctgatggcttagatgcctcctctccaggaaatagctttgtagggctccgtgttgtagccaagtggtcatccaatggctacttttactctgggaaaatcacacgagatgtcggagctgggaagtataaattgctctttgatgatgggtacgaatgtgatgtgttgggcaaagacattctgttatgtgaccccatcccgctggacactgaagtgacggccctctcggaggatgagtatttcagtgcaggagtggtgaaaggacataggaaggagtctggggaactgtactacagcattgaaaaagaaggccaaagaaagtggtataagcgaatggctgtcatcctgtccttggagcaaggaaacagactgagagagcagtatgggcttggcccctatgaagcagtaacacctcttacaaaggcagcagatatcagcttagacaatttggtggaagggaagcggaaacggcgcagtaacgtcagctccccagccacccctactgcctccagtagcagcagcacaacccctacccgaaagatcacagaaagtcctcgtgcctccatgggagttctctcaggcaaaagaaaacttatcacttctgaagaggaacggtcccctgccaagcgaggtcgcaagtctgccacagtaaaacctggtgcagtaggggcaggagagtttgtgagcccctgtgagagtggagacaacaccggtgaaccctctgccctggaagagcagagagggcctttgcctctcaacaagaccttgtttctgggctacgcatttctccttaccatggccacaaccagtgacaagttggccagccgctccaaactgccagatggtcctacaggaagcagtgaagaagaggaggaatttttggaaattcctcctttcaacaagcagtatacagaatcccagcttcgagcaggagctggctatatccttgaagatttcaatgaagcccagtgtaacacagcttaccagtgtcttctaattgcggatcagcattgtcgaacccggaagtacttcctgtgccttgccagtgggattccttgtgtgtctcatgtctgggtccatgatagttgccatgccaaccagctccagaactaccgtaattatctgttgccagctgggtacagccttgaggagcaaagaattctggactggcaaccccgtgaaaatcctttccagaatctgaaggtactcttggtatcagaccaacagcagaacttcctggagctctggtctgagatcctcatgactggtggtgcagcctctgtgaagcagcaccattcaagtgcccataacaaagatattgctttaggggtatttgatgtggtggtgacggacccctcatgcccagcctcggtgctgaagtgtgctgaagcattgcagctgcctgtggtgtcacaagagtgggtgatccagtgcctcattgttggggagagaattggattcaagcagcatccaaaatataaacacgattatgtttctcactaaagatacttggtcttactggttttattccctgctatcgtggagattgtgttttaaccaggttttaaatgtgtcttgtgtgtaactggattccttgcatggatcttgtatatagttttatttgctgaacttttatgataaaataaatgttgaatctctttggttgtagtaactgggatttcttcatctgtttttttgagcttaatctcagaacaaatgacaagacatagtactttctctgagtctttcaacaggcttattcacttacggaggacagctcaccaaggaaattgaaaagttaagagtgaactttattctgtggcatcattcccaaaaggttattccagggtgtctaaaatgctatgcttgcagaaactcagtttaaggtaggtgaaggcccagattaacagttgtgccaaaagttgagtggaattgggcacagctctgtttcctgacagttaaaaaagacctcatgctctctctctgagctgagatcacagctcacctgtgggtactccccaactcttagagctaaagggagaacgaaaggaccaactgccatgaagggacagtgaccataagcttgatggaatgaccttccgtaagataaacatgggaagcacaagtgagaacacctggaaatgttacacgttctagtcaaagacccaatattattattattattattgtcacaatagctggaagcagttccttcccttcctctggcatcactgatccctgcatggcttctcattctctaaagcaggggtcaacaaggtttttttctgtaaagggtcaaagagtaaatatttcaggctttgtgggccatttgatccatcacaactactcgcctttgctgtgagggcatgaaagcaaccatagacaatgagtaaacaaatgggcacggctgtgtttcagtaaaactgtacaaaaacagacagcaggccatagtttgccagctcctgctccagagacagcagtggaaagggtgatctttagttgataatagcagggaataagttgtcagagcttcccagtgtgtgtagaatatgtagtgatgaaaaccagatgcagtgactataacctgatgccagaacactgcattctttttcagtttggagggcgttgttcagtgaatatttctttttacttacactgatatgaatattgattaccagtgatggctgggccatattaagataacttcaacccctatggtttgtgtaagatgggtaattgggcctgcaatcttcagtatttaaaaatctaacaacttgatctcaattttttcttaaggacctttttcttggagaataatacttttttttttttttttttttttgagacggaatttcgctcttgttgcccaggctggaatgcaatggcacaatctcagctcactgcagcgtctgcttcccaggttcaagcaattctcctgtctcagcctcctgagtagctgggattacaggcacatgccaccacacctggctaatttttgtatttttagtagaatcgaggtttcatcatgttggtcaggctggtctcaaactcctgacttcaggtgatccgcccgcctcggcctcccaaagtgctgggattacaggtgtgagccaccatgcccggcctaagaaatacttttaagtatattttcattagctagaattgcccaatctgtgtaggtataaattacttggtatagggagagagaaagcctatcttacctgttgctttcttacttggtggtaacatccagcagttagtctatttataaacataattactttttcacatatgaaccataaaatatttaactttctgctctatattgtttgtttaccgctgtatctcccacagcttgaacagtaccaaggtacgtagtaggtgctcaataaatgactattgaataaatgaacatatccaacaaatgttctcaatgtaaaggatcagagatgccacatgttctccttgatgggagagacccttccacatgggaatgatgggaaggagttgtactcctggatgttcagtaactgcttctaggagaaaaggtagagtcctatcactaagccgcagatatttatttgtgtgtggctagaatgggatgttttgaatcttctgttacaaccttgggaacgtggctgttatttcaatttatgagccagaaattttcacatcccgaaactacaaaagagaaaaagagccttattaagtgtcatgctttcccaagactaccttcaaagaaatatgaatcaggataacctgtgatctaaataatgtcatcttaaaactgaagagtttcttttgactcttctgctacaatagcttagaaaaaaatctgcttgcagacattttagagagaaaggacaatgaagtgattttctgaatgggaatgacagacctctgggaagccagctaccactgaatctcggtatcagttttttttaaagtttagagttagaaggggtggtcgcctcctttcacagatgcggaagctagggaccagcaaggcggggtgcccacggctgcacagctagttcatatcagaattgggagtggaaggcccactgcctcccagcatagcaatacataacctagcaaaggacttaacacctatctcactgtcaggttttttagtattttatgatgatgatgacttctactagaaaataacctccattaaaattattaaagatggtcacacctctatctctaagccttacttataaaatgagggtatttggactaaagtcttcttccagttctagaattctacaactcattaaaaagccaccttaaaaagtctactgagttacccaagggttgctcctacctgcccagagttccaccagcctgggtatagtatttgttataatctagtcgtaacagtagttgagccaaatctgagttgatctgatgattccgaacactggagagaatcttgaacaggagtgaagactggcggctaaagccctgcagagagaaggactcagctgtcattccacttcagctcaccaactctccatatggaggatgggggcggagggaggagtttcttggaaaagccttgttcaaaattctacagaaccacctggccttcccacattcctatttctattagttcctaaaatgacttgtaccaaatccatacatgcatgacttcctatgaaagtactctttcatcagtaggaatttagtagctggtttccagttaatgtattttgtcaagtactggggttggggagaacccgttttgattacaagcagataattatctcagtgagatgggggttagttcaaggaagtaaggaggggggaggatgtgaggaagttagaacaacccaatgcttatttgatgggctgaataaactattcaggactgaactatttttgagcactgtgaggtggcacagtaattacctgcttcaaaatcaactgataccaacatttttatctttgtatcttatctctgtacgtgtgtgtattgaggaaatgctttactgactcagaggaaagatcatgaattctccattggcaaaaccacctctgtcctttcggcaaggctgcatacttccaggcagacgcaccttcacgagaatgctcagctgggcggctccacgctcatccagtgggcctaggttctgactgaccagcgaacaaaaactgtgacagagatctaggatttcattcaggcagtgaaacacctacccgggaaacagagttggcattaggaaaggaaggaaggtacatccatgaagttaaagtgttaggagaacagtctgattaatagctgatctaattaatagctgacctcccaaatctgacaggatagacactgccacgtgcaaggcctgccagcccctcagacgcacaaaatgcgtaaaacaaatgcatcctttcctggctaagcgagtattactctcttagccctgcaccaaacctccaatctagccacatttaactcttcatttcttagacccgcagagtgtcttcctgcctctgagctgtgagtgttgttccctttgcccgggatgctcttgtttttaataccagttcaagtcccactctctcagtgaagcactcccttccccactatagcctttagtgaaccctcgtttcttgcttctttattatctgtactgttgtccacttggcaattgttcaggcctctgtgttgttactgatttttgtatgtatatatatatatatgtcttgtttttccaactagattgtgagctccttaagggcagagccatgaattatacctctttgtatccccagtgccttgcatacagtaagcactcaata

SEQ ID NO: 16 - DNA, 6837 bp, Homo sapiens
agcatcgagtcggccttgttgcctactggagtctccgcagagcccgggcgggagtagctggtggaccccgttgagctgccgaacttccgggactcccccgcgaccccttcccagcttcccgtccgctccgccgcagcgattgtctcggtgggttgattcggcacaaaccgcccgacccaggggccggtgcgcgtgtggaaggggaagcactcccctcgtggtcgcctggaggtgcgctggaggagggggtgacataaccagggactcgaggtccgccgtgggaatgatccacgaactgctcttggctctgagcgggtaccctgggtccattttcacctggaacaagcggagtggcctgcaggtatcgcaggacttccctttcctccaccccagtgagaccagtgtcctgaatcgactctgccggctcggcacagactatattcgcttcactgagttcattgaacagtacacgggccatgtgcaacagcaggatcaccatccatctcaacagggccaaggtgggttacatggaatctacctgcgggccttctgcacagggctggattctgttttgcagccttatcgccaagcactgcttgatttggaacaagagttcctgggtgatccccatctctccatatcacatgtcaactacttcctagaccagttccagcttctttttccctctgtgatggttgtagtagaacaaattaaaagtcaaaagattcatggttgtcaaatcctggaaacagtctacaaacacagctgtggggggttgcctcctgttcgaagtgcactggaaaaaatcctggccgtttgtcatggggtcatgtataaacagctctcagcctggatgctccatggactcctcttggaccagcatgaagaattctttatcaaacaggggccatcttctggtaatgtcagtgcccagccagaagaggacgaggaggatctgggcattgggggactgacaggaaaacaactgagagaactgcaggacttgcgcctgattgaggaagagaacatgctggcaccatctctgaagcagttttccctacgagtggagattttgccatcctacattccagtgagggttgctgaaaaaatcctatttgttggagaatctgtccagatgtttgagaatcaaaatgtgaacctgactagaaaaggatccattttgaaaaaccaggaagacacttttgctgcagagctgcaccgtctcaagcagcagccactcttcagcttggtggactttgaacaggtggtggatcgcattcgcagcactgtggctgagcatctctggaagttgatggtagaagaatccgatttactgggtcagctgaagatcattaaagacttttaccttctgggacgtggagaactgtttcaggccttcattgacacagctcaacacatgttgaaaacaccacccactgcagtaactgagcatgatgtgaatgtggcctttcaacagtcagcacacaaggtattgctagatgatgacaaccttctccctctgttgcacttgacaatcgagtatcacggaaaggagcacaaagcagatgctactcaggcaagagaagggccttctcgggaaacttctccccgggaagcccctgcatctggctgggcagccctaggtctttcctacaaagtacagtggccactacatattctcttcaccccagctgtcctggaaaagtacaatgttgtttttaagtacttactgagtgtgcgccgggtgcaagctgagctgcagcactgctgggccctacaaatgcagcgcaagcacctcaagtcgaaccagactgatgcaatcaagtggcgcctaagaaatcacatggcatttttggtggataatcttcagtactatctccaggtagatgtgttggagtctcagttctcccagctgcttcatcagatcaattctacccgagactttgaaagcatccgattggctcatgaccacttcctgagcaatttgctggctcaatcctttatcctattgaaacctgtgtttcactgcctgaatgaaatcctagatctctgtcacagtttttgtttgctggtcagtcagaacctaggcccactggatgagcgtggagccgcccagctgagcattctcgtgaagggctttagccgccagtcttcactcctgttcaagattctctccagtgttcggaatcatcagatcaactcagatttggctcaactactgttacgactagattataacaaatactatacccaggctggtggaactctgggcagtttcgggatgtgaaaatttctggctcataaattgaaataacagccacgttcccaaggttgtaacagaagattcaaaacatcccattctagccacacacaaataaatatctgcggcttagtgataggactctaccttttctcctagaagcagttactgaacatccaggagtacaactccttcccatcattcccatgtggaagggtctctcccatcaaggagaacatgtggcatctctgatcctttacattgagaacatttgttggatatgttcatttattcaatagtcatttattgagcacctactacgtaccttggtactgttcaagctgtgggagatacagcggtagacaaacaatatagagcagaaagttaaatattttatggttcatatgtgaaaaagtaattatgtttataaatagactaactgctggatgttaccaccaagtaagaaagcaacaggtaagataggctttctctctccctataccaagtaatttatacctacacagattgggcaattctagctaatgaaaatatacttaaaagtatttcttaggccgggcatggtggctcacacctgtaatcccagcactttgggaggccgaggcgggcggatcacctgaagtcaggagtttgagaccagcctgaccaacatgatgaaacctcgattctactaaaaatacaaaaattagccaggtgtggtggcatgtgcctgtaatcccagctactcaggaggctgagacaggagaattgcttgaacctgggaagcagacgctgcagtgagctgagattgtgccattgcattccagcctgggcaacaagagcgaaattccgtctcaaaaaaaaaaaaaaaaaaaaaagtattattctccaagaaaaaggtccttaagaaaaaattgagatcaagttgttagatttttaaatactgaagattgcaggcccaattacccatcttacacaaaccataggggttgaagttatcttaatatggcccagccatcactggtaatcaatattcatatcagtgtaagtaaaaagaaatattcactgaacaacgccctccaaactgaaaaagaatgcagtgttctggcatcaggttatagtcactgcatctggttttcatcactacatattctacacacactgggaagctctgacaacttattccctgctattatcaactaaagatcaccctttccactgctgtctctggagcaggagctggcaaactatggcctgctgtctgtttttgtacagttttactgaaacacagccgtgcccatttgtttactcattgtctatggttgctttcatgccctcacagcaaaggcgagtagttgtgatggatcaaatggcccacaaagcctgaaatatttactctttgaccctttacagaaaaaaaccttgttgacccctgctttagagaatgagaagccatgcagggatcagtgatgccagaggaagggaaggaactgcttccagctattgtgacaataataataataataatattgggtctttgactagaacgtgtaacatttccaggtgttctcacttgtgcttcccatgtttatcttacggaaggtcattccatcaagcttatggtcactgtcccttcatggcagttggtcctttcgttctccctttagctctaagagttggggagtacccacaggtgagctgtgatctcagctcagagagagagcatgaggtcttttttaactgtcaggaaacagagctgtgcccaattccactcaacttttggcacaactgttaatctgggccttcacctaccttaaactgagtttctgcaagcatagcattttagacaccctggaataaccttttgggaatgatgccacagaataaagttcactcttaacttttcaatttccttggtgagctgtcctccgtaagtgaataagcctgttgaaagactcagagaaagtactatgtcttgtcatttgttctgagattaagctcaaaaaaacagatgaagaaatcccagttactacaaccaaagagattcaacatttattttatcataaaagttcagcaaataaaactatatacaagatccatgcaaggaatccagttacacacaagacacatttaaaacctggttaaaacacaatctccacgatagcagggaataaaaccagtaagaccaagtatctttagtgagaaacataatcgtgtttatattttggatgctgcttgaatccaattctctccccaacaatgaggcactggatcacccactcttgtgacaccacaggcagctgcaatgcttcagcacacttcagcaccgaggctgggcatgaggggtccgtcaccaccacatcaaatacccctaaagcaatatctgcaaggagcaagggaaagtgaagaaggaaaggacactcaacttagccctccattagaaagagagatttgattctaaccaatacatcccactctgcacaaaccaaagccctattatgtcaaacacactgctactgatcatgaccaaaggcagagttataatcactatgtgctgaccttgtagaaatatttaacaaatatacgtccagtgcttcacttatgttgactcacctcttgaaggtggtacttttcttctctaagaaacatggatacggtcaacctattaggcctgagccttggaccacaaggcctaacacctacaggtctaaggagatccctggaacaaagacactacacacactctttcaggtacctttgttatgggcacttgaatggtgctgcttcacagaggctgcaccaccagtcatgaggatctcagaccagagctccaggaagttctgctgttggtctgataccaagagtaccttcagattctggaaaggattttcacggggttgcctatgaaggagacaggaaaggaccttagcatgacaagtaatatccaacaaactgcctttctgcaaagggactcatgtacatctgaatgctttcaaaaataaatgccccatcagacatagtgtctcaagcctgtaatcccagcactttgggaggctgtcgtggttggatctcttgggcctgggagttcgagaccagcctgggcaatgtggtgagaccccatctctacaaaagacaacaaaaaaattagctgggtgtggtggcgagtgcctgtagtcccagcagcttgggaggctgaggtagggggatcacttcagcctgggaggttgaggctgcagtaagtcgtcactgcgccactgtactccagcctaggtgacagagcaagacttcatcttaaaaaactaagccctatattagggtcccccttctcttccttctttctatgaatgatctgtattccttgcattcctggctttctaatttccatgtttgttctggggctgagaataatccaaatcatgctcctgagcctatatatttttaatgcttgcttaaaacttagttctctgactttacaggttgagaatattgaacctatatacaaatcttcacacatttgcaaaaggttcctagccaatgtaacctagggaaataaactagataaactcctgaagtcatttcaaacccactcaaatttatcccacagacattccaatttctagaaagctttactctctcacctagattctcttccctccaaagcttgctgtcctcctgcctatacaattctggatgggcttcaaatacttaccagtccagaattctttgctcctcaaggctgtacccagctggcaacagataattacggtagttctggagctggttggcatggcaactatcatggacccagacatgagacacacaaggaatcccactggcaaggcacaggaagtacttccgggttcgacaatgctgatccgcaattagaagacactggtaagctgtgttacactgcaagaaaagaagcagagccaatgggtttggtgacttctgtggaaagctcctaagcagcagccataatgagccatgaagagcagatctgaagactcccaactactacccaaaatgtgatttagtctatcctgcccaaggccactcttctcactggaaggcccaagtaatttccatagatgttctctctgcctcacctgcagcatactgaggacctaaatcctcaacggacaaccaaaacctatgaactcagcctttcaggctaaaaatcagcaaccctaataggggtttctactactaaacataaacatcaatcttcttttgtcccagcaacagaaccatagccattaactaacccaaggtcctaccttctcttccctatacacaacaaaaattctatttcatgcaaaaacattttggcagtttctcagttcctgaaatctctggctactttatccaggttccccaacccctcccaggcctcttctcaacacagcaagttggctcttatcattgccactatattaggttacacaaagaaactcctcacctgggcttcattgaaatcttcaaggatatagccagctcctgctcgaagctgggattctgtatactgcttgttgaaaggaggaatttccaaaaattctatattaaaaaaaaaaaccaagata

SEQ ID NO: 17 - DNA, 733 bp, Artificial sequence
  Probe
cacaatctccacgatagcagggaataaaaccagtaagaccaagtatctttagtgagaaacataatcgtgtttatattttggatgctgcttgaatccaattctctccccaacaatgaggcactggatcacccactcttgtgacaccacaggcagctgcaatgcttcagcacacttcagcaccgaggctgggcatgaggggtccgtcaccaccacatcaaatacccctaaagcaatatctgcaaggagcaagggaaagtgaagaaggaaaggacactcaacttagccctccattagaaagagagatttgattctaaccaatacatcccactctgcacaaaccaaagccctattatgtcaaacacactgctactgatcatgaccaaaggcagagttataatcactatgtgctgaccttgtagaaatatttaacaaatatacgtccagtgcttcacttatgttgactcacctcttgaaggtggtacttttcttctctaagaaacatggatacggtcaacctattaggcctgagccttggaccacaaggcctaacacctacaggtctaaggagatccctggaacaaagacactacacacactctttcaggtacctttgttatgggcacttgaatggtgctgcttcacagaggctgcaccaccagtcatgaggatctcagaccagagctccaggaagttctgctgttggtctgataccaagagtaccttcagattctgg

SEQ ID NO: 18 - DNA, 734 bp, Artificial sequence
  Probe
gctagaattgcccaatctgtgtaggtataaattacttggtatagggagagagaaagcctatcttacctgttgctttcttacttggtggtaacatccagcagttagtctatttataaacataattactttttcacatatgaaccataaaatatttaactttctgctctatattgtttgtctaccgctgtatctcccacagcttgaacagtaccaaggtacgtagtaggtgctcaataaatgactattgaataaatgaacatatccaacaaatgttctcaatgtaaaggatcagagatgccacatgttctccttgatgggagagacccttccacatgggaatgatgggaaggagttgtactcctggatgttcagtaactgcttctaggagaaaaggtagagtcctatcactaagccgcagatatttatttgtgtgtggctagaatgggatgttttgaatcttctgttacaaccttgggaacgtggctgttatttcaatttatgagccagaaattttcacatcccgaaactgcccagagttccaccagcctgggtatagtatttgttataatctagtcgtaacagtagttgagccaaatctgagttgatctgatgattccgaacactggagagaatcttgaacaggagtgaagactggcggctaaagcccttcacgagaatgctcagctgggcggctccacgctcatccagtgggcctaggttctgactgaccagca

SEQ ID NO: 19 - DNA, 2289 bp, Homo sapiens
tcgcggccgcgtcracgcgtggtagggggcccagagcaagccgaaggcaagcacgatggcgctcaccagccggcccacccgcgccccgtgccgcccggagccccagcgggcgccccgcagccgtgccagcgtcacgctgtagcagccgagcatcagccgaaaggaagcacgaaagcggtcagagtctccaggctcaggtgggcggcggcgtggaccggcgacgggtggcacagctggcatacgcggtccctccacaggtggcggtagacggcggccgggacggcgagcaacagggcggccagccagaccgccagcagcaggcggcgggccagggccgggctgcgcagccgaggcgccaggaaggggcgggtgactgcgaggcagcgctgcaggctgagcaggccggtgagcagcacgcttggcgtacatgctgagcgcgcacacgtagtacaccgccttgcagcccgcctggcccagcggccaggcctgccggtcaggaaggccacaaagagcggcgtgagcagcagcaccgcgccgtcggccagcgccaggtgcagcacaagcgtggccgccagcggtcgcccccgtgcaggctgccagcccgccaagctccacaccacgaagccgttgccaggcagccccagcagcgccgccagcagcaggaaggctgtgcctgtggcccgcgaagtcttccagctcagcagtgtctcgttccctgggggacggtagcagaccgacatccttctgggcctacaggacacagaaaaaaagtggggaagctgggggaccctacaaggatccttggcaggaaagcagggattgtgttcattttgagggtttcactgtcagtgagagtctcagcttccatgcaactgtccatcacggctgcaactgaaatcagagctgggacacagcgcaccagaagctaaagtcttgatgccatcaaaggacatcccctgccccattcacayattcacatctctgtcacgtccactaatcggcaaaaggagaaaagtgagagaagatgacctaagtgtgactgcagcaggcagctctggaaaatgaagccagagcagtgagccagcccctcctccgaccaaggaggaaggaaagagcagccccagcacaggagagaaccacccagcccagaagttccagggaaggaactctccggtccaccatggagtacctctcagctctgaaccccagtgacttactcaggtcagtatctaatataagctcggagtttggacggagggtctggacctcagctccaccaccccagcgacctttccgtgtctgtgatcacaagcggaccatccggaaaggcctgacagctgccacccgccaggagctgctagccaaagcattggagaccctactgctgaatggagtgctaaccctggtgctagaggaggatggaactgcagtggacagtgaggacttcttccagctgctggaggatgacacgtgcctgatggtgttgcagtctggtcagagctggagccctacaaggagtggagtgctgtcatatgggcctggacgggagagccccaagcacagcaaggacatcggccgattcacctttgacgtgtacaagcaaaaccctcgagacctctttggcagcctgaatgtcaaagccacattctacgggctctactctatgagttgtgactttcaaggacttggcccaaagaaagtactcagggagctccttcgttggacctccacactgctgcaaggcctgggccatatgttgctgggaatttcctccacccttcgtcatgcagtggagggggctgagcagtggcagcagaagggccgcctccattcctactaaggggctctgagcttctgcccccagaatcattccaaccgacccactgcaaagactatgacagcatcaaatttcaggacctgcagacagtacaggctagataacccacccaatttccccactgtcctctgatcccctcgtgacagaacctttcagcataacgcctcacatcccaagtctatacccttacctgaagaatgctgttctttcctagccacctttctagcctcccacttgccctgaaaggccaagatcaagatgtcccccaggcatcttgatcccagcctgactgctgctacatctaatcccctaccaatgcctcctgtccctaaactccccagcatactgatgacagccctctctgactttaccttgagatctgtcttcatacccttcccctcaaactaacaaaaacatttccaataaaaatatcaaatatttaccgtcaaccc

SEQ ID NO: 20 - DNA, 1511 bp, Homo sapiens
cacatttcatccttttacatggttcccatctaccctcacaacacatgtcatcaccaaagacacacatacaagctccaatggcttttgccaggcaattcttcctccaggaccccatctggcccctccctcatccctccccttggactttgcccttcttactggccaggcaggggggccagagtccaggcttgactcattcccaccttgtcctgggctgagatcccaggtttgtaacagaaaacaccactaaagccccagcacaggagagaaccacccagcccagaagttccagggaaggaactctccggtccaccatggagtacctctcagctctgaaccccagtgacttactcaggtcagtatctaatataagctcggagtttggacggagggtctggacctcagctccaccaccccagcgacctttccgtgtctgtgatcacaagcggaccatccggaaaggcctgacagctgccacccgccaggagctgctagccaaagcattggagaccctactgctgaatggagtgctaaccctggtgctagaggaggatggaactgcagtggacagtgaggacttcttccagctgctggaggatgacacgtgcctgatggtgttgcagtctggtcagagctggagccctacaaggagtggagtgctgtcatatggcctgggacgggagaggcccaagcacagcaaggacatcgcccgattcacctttgacgtgtacaagcaaaaccctcgagacctctttggcagcctgaatgtcaaagccacattctacgggctctactctatgagttgtgactttcaaggacttggcccaaagaaagtactcagggagctccttcgttggacctccacactgctgcaaggcctgggccatatgttgctgggaatttcctccacccttcgtcatgcagtggagggggctgagcagtggcagcagaagggccgcctccattcctactaaggggctctgagcttctgcccccagaatcattccaaccgacccactgcaaagactatgacagcatcaaatttcaggacctgcagacagtacaggctagataacccacccaatttccccactgtcctctgatcccctcgtgacagaacctttcagcataacgcctcacatcccaagtctatacccttacctgaagaatgctgttctttcctagccacctttctggcctcccacttgccctgaaaggccaagatcaagatgtcccccaggcatcttgatcccagcctgactgctgctacatctaatcccctaccaatgcctcctgtccctaaactccccagcatactgatgacagccctctctgactttaccttgagatctgtcttcatacccttcccctcaaactaacaaaaacatttccaataaaaatatcaaatatttaccactaagacttctgactccaatttaaaccaggaaagggatggggtggataccccattttgccctcccccatcaacacccagtcccagatccaaagcc

SEQ ID NO: 21 - DNA, 6530 bp, Homo sapiens
ttttgttagtttgaggggaagggtatgaagacagatctcaaggtaaagtcagagagggctgtcatcagtatgctggggagtttagggacaggaggcattggtaggggattagatgtagcagcagtcaggctgggatcaagatgcctgggggacatcttgatcttggcctttcagggcaagtgggaggccagaaaggtggctaggaaagaacagcattcttcaggtaagggtatagacttgggatgtgaggcgttatgctgaaaggttctgtcacgaggggatcagaggacagtggggaaattgggtgggttatctagcctgtactgtctgcaggtcctgaaatttgatgctgtcatagtctttgcagtgggtcggttggaatgattctgggggcagaagctcagagccccttagtaggaatggaggcggcccttctgctgccactgctcagccccctccactgcatgacgaagggtggaggaaattcccagcaacatatggcccaggccttgcagcagtgtggaggtccaacgaaggagctccctgaatggcagagacaagaggaaatcagatgatttggaaaacttgggaggaagccatcaagctgggagatgaggactttccacaagcaagagctaactaggggtaggtgggtgcaagaggacgaattatggggactatccaactgtaggggatggggcagtatgacatgttgatttctgacctgagtactttctttgggccaagtccttgaaagtcacaactcatagagtagagcccgtagaatgtggctttgacattcaggctgccaaagaggtctcgagggttttgcttgtacacgtcaaaggtgaatcgggcgatgtccttgctgtgcttgggcctctcccgtcccaggccatatgacagcactccactctgtaggacacccttgtcagtgcagtagatcctcataccagacacccaccactaatctccatcagcactgggtcagaccctccctcgcttggactttctgtccactgtgtgacatccttgacaattccacaactcctcctgcacctggtccccaggatcagggttaagctagagaggaagcccgggaaagctctaaaggacaggcattggaagcagccccagtataggcctcttacccttgtagggctccagctctgaccagactgcaacaccatcaggcacgtgtcatcctccagcagctggaagaagtcctcactgtccactgcagttccatcctcctctagcaccagggttagcactccattcagcagtagggtctccaatgcctgcccaatggcaagaagcaagaagggcaggtcttatcccatgccccttccctctttagctgcccaacatccatcagttggctctagacattggtcgatgtcccactttgactttccggcactttgatacctcctaaaggttgcagctctccgtgttcttcagtttttgggggatcctagctagaggctgacctttttcctctttgctcctaccatgtcattggcatctccccttgctcccctccaagtcacttctggtttggaattggaaagcaagccaggttctcacgaagtccacccttctgtcttatctacaatgctgcacctcacttcccacaccctcaagagttctccagaagtgttttcagtaatagtgtttaacctttttgagtccttactctgtgccaggtatgaggactttacctacattatcctcttactcctttcaacaaccctaggaggtgatgtattattattgcctttttatagttgaagaaactgaggttttggtaggttgaacaacttcccaaggtttgacaggcaggaagtggcagaatcagaatttgaacttgatttgtcacacaaatcacctttccatactagcttctgaattctgtccctcgaactctccctatctcctgctaacccctgctcccatagaaaagctcactcggtggaaaatgaacaaattgaccagagctcattaggcccactccgctgcttttagccctcagagggaggggcagctgtgtgacttcagccctctgctccatcatcacaagttgccactgttgtggagccccttggctacccctgctataggaaccgaggaacttggcctacttactttggctagcagctcctggcgggtggcagctgtcaggcctttccggatggtccgcttgtgatcacagacacggaaaggtcgctggggtggtggagctgaggtccagaccctccgtccaaactccgagcttatattagatactgacctggtagttgagaagaaaagtcaagaaggggcgaggaggggcttggtgagtgtaaagggcatgatgagggtagagtggctagagggctagggagggagagatctaggtttatcgattagggatgagggagagaccatggagtgcaggtgggggcgggtggctcaggagcttgacaagcccactgtggagtggggagcaggagaggaaggggtactggttagtctcctaggggctgagtggagtattgttgccctgcctatatcccctaaaggtggagggtagagcggagggttagcagtcacctgagtaagtcactggggttcagagctgagaggtactccatggtggaccggagagttccttccctggaacttctgggctgggtggttctctcctgtgctggggctttagtggtgttttctgttacaaacctgggatctcagcccaggacaaggtgggaatgagtcaagcctggactctggcccccctgcctggccagtaagaagggcaaagtccaaggggagggatgagggaggggccagatggggtcctggaggaagaattgcctggcaaaagccattggagcttgtatgtgtgtctttggtgatgacatgtgttgtgagggtagatgggaaccatgtaaaaggatgaaatgtgacttctggtgtttttttatttctatggagggaatttctggggacggtttctggctctcaggctctgagaagctgcagtttatgagtggctctgtgtgtgctgccacctactggagaagccataagctgcagctttaggaaaagggaacccggggcagagtgtggggaagtgggatggcagcatggcagggctttggaaaatgagaggtgagactgtgtccaggaagggtgtaaggagaggatggatcctgatacatggattcaggatcattagggtcctgtctgggacactggccttcctgcttacctgctctttccttcctccttggtcggaggaggggctggctcactgctctggcttcattttccagagctgcctgctgcagtcacacttaggtcatcttctctcacttttctccttttgccgattagtggacgtgacagagatgtgaatggggcagggatgtcctttgatggcatcaagactttagcttctggtgcgctgtgtcccagctctgatttcagttgcagccgtgatggacagttgcatggaagctgagactctcactgacagtgaaaccctcaaatgaacacaatccctgctttcctgccaaggatccttgtagggtcccccagcttccccactttttttctgtgtcctgacaaagaaacacagagtaacttgattgccctgtgacctggccagttgcatttcccctgcaggcttgagcccaagccagagccttgaaaaggtattcaggttgttgcccaaaacactgaaaaaaactggccctggccctgaaccaaataccttgaaccctcgtaaactccataccctgacccccttgttttggatatacccaggtagaacaactctctctcactgtctgttgtgaggatacgctgtagcccactcattaagtacattctcctaataaatgctttggactgatcaccctgccagtcttttgtcttgggcaatctatacttttctcagaggttcccaaggcctactgaagggacttaacatactcttaatggctttcctctctcttgttttaccttatgccctcacttcctgagttaacctcccaaatacaggatcacctgtacccaagcccttagctcaagaatacaggatcacctgtacccaagcccttagctcaagctctgctttggaagaacccaaactaagacagtgctcctggtgccctccccaagcaacctcaagttctggctgttacttgagcagaggcctttcttttcccttcccccagctctatccatctgccaggcccccctcaaatctcttcatttccaagttttgcttgacttttccaagaggagagggctgcttcttagtatgtccctactcatcctttcctttcttgtcttgtatcctggtgcagcctggtaatggggcctcttcatggttgtgtgtcatgactccctaaccattatgcctccatgcatcccctgttcctcctggaacctagcaccatgccttacatggaaaagctgtcattgacagcccggtgagagccctgagggtggagtgactggggcagggcctgaggcaagaggtgggaggaggtaggaggccaggggctcagccggaccaggagactggaaacaggcaaggataaggcaggtgggggactgagttgtttgggtcacctctgcaggccagagagaccaggcaacatacacactgcagaaggtgggctgggaggattggggccagagctgggggagggatgagaacagaagcaggaccaggattcagcagagtcctcctatttccttccaccaccagggaatcttactgccccacttcagcttgtgctgtttcctggcaaggcaggctctcacatgcctggacgcctgggtgcgttggtgatgggaaggagcagggtgagggaggggccccaggagaggcccaggatgagcctcatcttgtccctccccattcttgtcttaccctctgcaaatgtgataggcacaggacaggagtaggcacctcgcctactgctgcttaacctttcagcttctccaggcccccaatcctgcttgctcccagcttggtaagtagatctgtgcacgtccctttacaccccaccatccagttttgcccagatgtgctagaatggggctggacaaagaaggaggggccagactagaggagtggtggtagagatagtgacagcctggggtgatgactttatgcctgtttaccactgagctctgggaaggaggccaggagtggggcaggtcaactgactgggagcaggggatctgggttccaagaaggagttgtgtttgaggtggggtctgggtcctcgtggaagtcaggactcccaggcagaaaagaggcaggctgcagggaagtaaggaggaggcatggcaccttctcatcgggcatcacaggtggggttttgccccacccctgaacgccctctgtggcgccttccacccacctgtaggcccagaaggatgtcggtctgctaccgtcccccagggaacgagacactgctgagctggaagacttcgcgggccacaggcacagccttcctgctgctggcggcgctgctggggctgcctggcaacggcttcgtggtgtggagcttggcgggctggcggcctgcacgggggcgaccgctggcggccacgcttgtgctgcacctggcgctggccgacggcgcggtgctgctgctcacgccgctctttgtggccttcctgacccggcaggcctggccgctgggccaggcgggctgcaaggcggtgtactacgtgtgcgcgctcagcatgtacgccagcgtgctgctcaccggcctgctcagcctgcagcgctgcctcgcagtcacccgccccttcctggcgcctcggctgcgcagcccggccctggcccgccgcctgctgctggcggtctggctggccgccctgttgctcgccgtcccggccgccgtctaccgccacctgtggagggaccgcgtatgccagctgtgccacccgtcgccggtccacgccgccgcccacctgagcctggagactctgaccgctttcgtgcttcctttcgggctgatgctcggctgctacagcgtgacgctggcacggctgcggggcgcccgctggggctccgggcggcacggggcgcgggtgggccggctggtgagcgccatcgtgcttgccttcggcttgctctgggccccctaccacgcagtcaaccttctgcaggcggtcgcagcgctggctccaccggaaggggccttggcgaagctgggcggagccggccaggcggcgcgagcgggaactacggccttggccttcttcagttctagcgtcaacccggtgctctacgtcttcaccgctggagatctgctgccccgggcaggtccccgtttcctcacgcggctcttcgaaggctctggggaggcccgagggggcggccgctctagggaagggaccatggagctccgaactacccctcagctgaaagtggtggggcagggccgcggcaatggagacccggggggtgggatggagaaggacggtccggaatgggacctttgacagcagaccct

SEQ ID NO: 22 - DNA, 424 bp, Artificial sequence
  Probe
ggattagatgtagcagcagtcaggctgggatcaagatgcctgggggacatcttgatcttggcctttcagggcaagtgggaggctagaaaggtggctaggaaagaacagcattcttcaggtaagggtatagacttgggatgtgaggcgttatgctgaaaggttctgtcacgaggggatcagaggacagtggggaaattgggtgggttatctagcctgtactgtctgcaggtcctgaaatttgatgctgtcatagtctttgcagtgggtcggttggaatgattctgggggcagaagctcagagccccttagtaggaatggaggcggcccttctgctgccactgctcagccccctccactgcatgacgaagggtggaggaaattcccagcaacatatggcccaggccttgcagcagtgtggaggtcc

SEQ ID NO: 23 - DNA, 424 bp, Artificial sequence
  Probe
ggacctccacactgctgcaaggcctgggccatatgttgctgggaatttcctccacccttcgtcatgcagtggagggggctgagcagtggcagcagaagggccgcctccattcctactaaggggctctgagcttctgcccccagaatcattccaaccgacccactgcaaagactatgacagcatcaaatttcaggacctgcagacagtacaggctagataacccacccaatttccccactgtcctctgatcccctcgtgacagaacctttcagcataacgcctcacatcccaagtctatacccttacctgaagaatgctgttctttcctagccacctttctagcctcccacttgccctgaaaggccaagatcaagatgtcccccaggcatcttgatcccagcctgactgctgctacatctaatcc

SEQ ID NO: 24 - DNA, 7042 bp, Homo sapiens
aagaagaggtagcgagtggacgtgactgctctatcccgggcaaaagggatagaaccagaggtggggagtctgggcagtcggcgacccgcgaagacttgaggtgccgcagcggcatccggagtagcgccgggctccctccggggtgcagccgccgtcgggggaagggcgccacaggccgggaagacctcctccctttgtgtccagtagtggggtccaccggagggcggcccgtgggccgggcctcaccgcggcgctccgggactgtggggtcaggctgcgttgggtggacgcccacctcgccaaccttcggaggtccctgggggtcttcgtgcgccccggggctgcagagatccaggggaggcgcctgtgaggcccggacctgccccggggcgaagggtatgtggcgagacagagccctgcacccctaattcccggtggaaaactcctgttgccgtttccctccaccggcctggagtctcccagtcttgtcccggcagtgccgccctccccactaagacctaggcgcaaaggcttggctcatggttgacagctcagagagagaaagatctgagggaagatggatgcaaaagctcgaaattgtttgcttcaacatagagaagctctggaaaaggacatcaagacatcctacatcatggatcacatgattagtgatggatttttaacaatatcagaagaggaaaaagtaagaaatgagcccactcaacagcaaagagcagctatgctgattaaaatgatacttaaaaaagataatgattcctacgtatcattctacaatgctctactacatgaaggatataaagatcttgctgcccttctccatgatggcattcctgttgtctcttcttccagtgtaaggacagtcctgtgtgaaggtggagtaccacagaggccagttgtttttgtcacaaggaagaagctggtgaatgcaattcagcagaagctctccaaattgaaaggtgaaccaggatgggtcaccatacatggaatggcaggctgtgggaagtctgtattagctgcagaagctgttagagatcattcccttttagaaggttgtttcccagggggagtgcattgggtttcagttgggaaacaagacaaatctgggcttctgatgaaactgcagaatctttgcacacggttggatcaggatgagagtttttcccagaggcttccacttaatattgaagaggctaaagaccgtctccgcattctgatgcttcgcaaacacccaaggtctctcttgatcttggatgatgtttgggactcttgggtgttgaaagcttttgacagtcagtgtcagattcttcttacaaccagagacaagagtgttacagattcagtaatgggtcctaaatatgtagtccctgtggagagttccttaggaaaggaaaaaggacttgaaattttatccctttttgttaatatgaagaaggcagatttgccagaacaagctcatagtattataaaagaatgtaaaggctctccccttgtagtatctttaattggtgcacttttacgtgattttcccaatcgctgggagtactacctcaaacagcttcagaataagcagtttaagagaataaggaaatcttcgtcttatgattatgaggctctagatgaagccatgtctataagtgttgaaatgctcagagaagacatcaaagattattacacagatctttccatccttcagaaggacgttaaggtgcctacaaaggtgttatgtattctctgggacatggaaactgaagaagttgaagacatactgcaggagtttgtaaataagtctcttttattctgtgatcggaatggaaagtcgtttcgttattatttacatgatcttcaagtagattttcttacagagaagaattgcagccagcttcaggatctacataagaagataatcactcagtttcagagatatcaccagccgcatactctttcaccagatcaggaagactgtatgtattggtacaactttctggcctatcacatggccagtgccaagatgcacaaggaactttgtgctttaatgttttccctggattggattaaagcaaaaacagaacttgtaggccctgctcatctgattcatgaatttgtggaatacagacatatactagatgaaaaggattgtgcagtcagtgagaattttcaggagtttttatctttaaatggacaccttcttggacgacagccatttcctaatattgtacaactgggtctctgtgagccggaaacttcagaagtttatcagcaagctaagctgcaggccaagcaggaggtcgataatggaatgctttacctggaatggataaacaaaaaaaacatcacgaatctttcccgcttagttgtccgcccccacacagatgctgtttaccatgcctgcttttctgaggatggtcagagaatagcttcttgtggagctgataaaaccttacaggtgttcaaagctgaaacaggagagaaacttctagaaatcaaggctcatgaggatgaagtgctttgttgtgcattctctacagatgacagatttatagcaacctgctcagtggataaaaaagtgaagatttggaattctatgactggggaactagtacacacctatgatgagcactcagagcaagtcaattgctgccatttcaccaacagtagtcatcatcttctcttagccactgggtcaagtgactgcttcctcaaactttgggatttgaatcaaaaagaatgtcgaaataccatgtttggtcatacaaattcagtcaatcactgcagattttcaccagatgataagcttttggctagttgttcagctgatggaaccttaaagctttgggatgcgacatcagcaaatgagaggaaaagcattaatgtgaaacagttcttcctaaatttggaggaccctcaagaggatatggaagtgatagtgaagtgttgttcgtggtctgctgatggtgcaaggataatggtggcagcaaaaaataaaatctttttgtggaatacagactcacgttcaaaggtggctgattgcagaggacatttaagttgggttcatggtgtgatgttttctcctgatggatcatcatttttgacatcttctgatgaccagacaatcaggctctgggagacaaagaaagtatgtaagaactctgctgtaatgttaaagcaagaagtagatgttgtgtttcaagaaaatgaagtgatggtccttgcagttgaccatataagacgtctgcaactcattaatggaagaacaggtcagattgattatctgactgaagctcaagttagctgctgttgcttaagtccacatcttcagtacattgcatttggagatgaaaatggagccattgagattttagaacttgtaaacaatagaatcttccagtccaggtttcagcacaagaaaactgtatggcacatccagttcacagccgatgagaagactcttatttcaagttctgatgatgctgaaattcaggtatggaattggcaattggacaaatgtatctttctacgaggccatcaggaaacagtgaaagactttagactcttgaaaaattcaagactgctttcttggtcatttgatggaacagtgaaggtatggaatattattactggaaataaagaaaaagactttgtctgtcaccagggtacagtactttcttgtgacatttctcacgatgctaccaagttttcatctacctctgctgacaagactgcaaagatctggagttttgatctccttttgccacttcatgaattgaggggccacaacggctgtgtgcgctgctctgccttctctgtggacagtaccctgctggcaacgggagatgacaatggagaaatcaggatatggaatgtctcaaacggtgagcttcttcatttgtgtgctccgctttcagaagaaggagctgctacccatggaggctgggtgactgacctttgcttttctccagatggcaaaatgcttatctctgctggaggatatattaagtggtggaacgttgtcactggggaatcctcacagaccttctacacaaatggaaccaatcttaagaaaatacacgtgtcccctgacttcaaaacatatgtgactgtggataatcttggtattttatatattttacagactttagaataaaatagttaagcattaatgtagttgaactttttaaatttttgaattggaaaaaaattctaatgaaaccctgatatcaactttttataaagctcttaattgttgtgcagtattgcattcattacaaaagtgtttgtggttggatgaataatattaatgtagctttttcccaaatgaacatacctttaatcttgtttttcatgatcatcattaacagtttgtccttaggatgcaaatgaaaatgtgaatacataccttgttgtactgttggtaaaattctgtcttgatgcattcaaaatggttgacataattaatgagaagaatttggaagaaattggtattttaatactgtctgtatttattactgttatgcaggctgtgcctcagggtagcagtggcctgctttttgaaccacacttaccccaagggggttttgttctcctaaatacaatcttagaggttttttgcactctttaaatttgctttaaaaatattgtgtctgtgtgcatagtctgcagcatttcctttaattgactcaataagtgagtcttggatttagcaggcccccccaccttttttttttgtttttggagacagagtcttgctttgttgccaggctggagtgcagtggcgcgatctcggctcaccacaatcgctgcctcctgggttcaagcaattctcctgcctcagcctcccgagtagctgggactacaggtgtgcgcacatgccaggctaatttttgtatttttagtagagacggggtttcaccatgttggccgggatggtctcgatctcttgacctcatgatctacccgccttggcctcccaaagtgctgagattacaggcgtgagccaccgtgcctggccaggccccttctcttttaatggagacagggtcttgcactatcacccaggctggagtgcagtggcataatcatacctcattgcagcctcagactcctgggttcaagcaatcctcctgcctcagcctcccaagtagctgagactgcaggcacgagccaccacacccagctaatttttaagttttcttgtagagacagggtctcactatgttgtctaggctggtcttgaactcttggcctcaagtaatcctcctgcctcagcctcccaaagtgttgggattgcagatatgagccactggcctggccttcagcagttctttttgtgaagtaaaacttgtatgttggaaagagtagattttattggtctacccttttctcactgtagctgctggcagccctgtgccatatctggactctagttgtcagtatctgagttggacactattcctgctccctcttgtttcttacatatcagacttcttacttgaatgaaacctgatctttcctaatcctcacttttttcttttttaaaaagcagtttctccactgctaaatgttagtcattgaggtggggccaattttaatcataagccttaataagatttttctaagaaatgtgaaatagaacaattttcatctaattccatttacttttagatgaatggcattgtgaatgccattcttttaatgaatttcaagagaattctctggttttctgtgtaattccagatgagtcactgtaactctagaagattaaccttccagccaacctattttcctttcccttgtctctctcatcctcttttccttccttctttcctttctcttcttttatctccaaggttaatcaggaaaaatagcttttgacaggggaaaaaactcaataactagctatttttgacctcctgatcaggaactttagttgaagcgtaaatctaaagaaacattttctctgaaatatattattaagggcaatggagataaattaatagtagatgtggttcccagaaaatataatcaaaattcaaagattttttttgtttctgtaactggaactaaatcaaatgattactagtgttaatagtagataacttgtttttattgttggtgcatattagtataactgtggggtaggtcggggagagggtaagggaatagatcactcagatgtattttagataagctatttagcctttgatggaatcataaatacagtgaatacaatcctttgcattgttaaggaggttttttgtttttaaatggtgggtcaaggagctagtttacaggcttactgtgatttaagcaaatgtgaaaagtgaaaccttaattttatcaaaagaaatttctgtaaatggtatgtctccttagaatacccaaatcataattttatttgtacacactgttaggggctcatctcatgtaggcagagtataaagtattaccttttggaattaaaagccactgactgttataaagtataacaacacacatcaggttttaaaaagccttgaatggcccttgtcttaaaaagaaattaggagccaggtgcggtggcacgtgcctgtagtcccagctccttgggaggctgagacaggaggattccttgagccctggagtttgagtccagcctgggtgacatagcaagaccctgtcttaaaagaaaaatgggaagaaagacaaggtaacatgaagaaagaagagatacctagtatgatggagctgcaaatttcatggcagttcatgcagtcggtcaagaggaggattttgttttgtagtttgcagatgagcatttctaaagcattttcccttgctgtatttttttgtattataaattacattggacttcatatatataatttttttttacattatatgtctcttgtatgttttgaaactcttgtatttatgatatagcttatatgatttttttgccttggtatacattttaaaatatgaatttaaaaaatttttgtaaaaataaaattcacaaaattgttttgaaaaacaaaaaaaaaaaaaa

SEQ ID NO: 25 - DNA, 3019 bp, Artificial sequence
  Probe
tttttttttttttttttgaaaaacatttttggattgtttcattctttgcttgtcatttatctgttgattagaccactaaagtgaaggattcaagctaaatacatcaacctttctatttaggctttatcagctatatgtaaattcaattctatcaaaattttctgagtgcctcctcagtgtgtctctctgatggttcctgcccggtatggctggcatgaagaagatccacggacttgcgaatgctaacgcggggcttggggatgggtttggagggtttgttttcaaagctttctggaagtgtggaggagtgtcccccttttcttgcttgtagtgctagctggtaagcgacttcgaatgcctgtcccagggttaggatgatttcataggctaaattcacatcaaaggcagtaaacacatgacagtagtggtgattagacttcaaatcttttgtgatataggcaaatgttgagaggtcttctgggtcctgggcagcacaggagatattacgaatttcatgctcagcaattatgttcttatttgttgcatcaataaatttgactcctttatatgagacagaaagaataatagtagggaccttcttcatttgctctgtagacttctgacagttagcccgcatttttgcacaagcatcttgggttgattctgtccccctaagctcttttatcagcatagaacctaaataaaaagctttgtaatcacacgactggaagataagcttttctgggtgatgctgccagtactgtaccggggtagaggctgtggcttcattcggaggtcgcaaggtaatggaaggttctccccagtctcctgtctgagccatctgcctctccagttttgatcggggaatatcatcaaagtagttttcatttcttctcctccttgcatcgccctgcatgataatgtgaggaacgtctagggagccaccagtggtgtaagtgctttggctaagtgatggagacaactgaggaggagtgtgattaccactgggttccctgagggtgatggaccgagggggcttctgtgggggatcgtcgtgcagcctgtctcccagagatgccaaaatacgtttcctgtggccaatcaaattgatttttaaaacattaataagttcaacctcccagatttttttcaacaggtccatcgaagtgtagccattaattagaaaggctttggtgtagtcgcccagttcaatggaatccagccactcagctacagaggtgggatggtagccatcatgcccaatgggtctcatctttggaaggagctggattgcctgtagaattctttgtctgtgcccagaattaaggattccaatttccaacaaatcctgatcttccataacattgcttcccataaactgcacattgtcaaatccattagccatcaggtggttctcgtactgaggtagcccaatgctttccaaccattgtcccactgtttggacagggcatctgggtcttgtggtctcaccattcatctctttaagttcgttgttgattccaacatctatggaactcattattttgtcaatttcttcccattccgatgtgaaggatggtgttctttcagaattccctttagaactgtgttcagcagtggaagattcactccagttaactcttgatgttttctcattggaaggataggcaatgagatcagaatcagatttagagacactttttgacaaatgcatgtcgatcaaggctttaggcaatgaccttattctccccagagtacaggctctctccacaaatccccctgcgttcataacccactggtccccattccgagatccactcctggttgatcttgtgccaacaatggtatggttttcgagttggttgctttttttgtgaaaaattgttctactgaccactttgggtttaattttctttaccaaaggttctgagttattttctattggagactgcttgaaaggcaaaggactgtttgccaaactgacttcatcttgtcctttttcacattgttctcttttcccatagagatgaaatggattttcaggggactcacaggctggagaggatccatggagcaggcctgcaaattgcccaggatcatattcttttgggggatcattgtcatcctgtcgggagaggtcatctgtatggttagttccctcattcttgacttctgtggttcccacagaagaggtgggtggactagcaggaggactggcagtaaagcttgtgcaccctgtagatgtgttgatttcaaaatattcttggttgtgattcattcggtgaaaatccagagaagacacaatggatgttcgctgtttaggctggggtcgaatgacttttacaatatttttgagggcagtatcaggggatggaggtgaacaatcaggtgttgggcctgttgagctgtttctatggttactagttcctggagtagtaactgctacctcagaggcattatcagttcttggggaaggtgcccttgcaatttctaaggagcaaggtttctttgtaacagctgtgtccatgagatcacacagaaagttctcattttctgaaggaaatgtatccagagaagcagatggtacaatttccatagtgtaatttctcttctttggataggactcctgggcaagcatggggaagccaaggttcctacatccattacacggagttaatgcttcccaaagtcctgatggcccacacgtattttcatcatcatcctcttcttccacttctcctggtgacaaattgattgtagatgaggttcttacactctggcttccatttttcccaagttcttcctctgaaatcttgctcaaattatctaagtagtggtctgatatngtgtggcacaagtcttcaaacgaataatccttttcttgacagagttttatttcatccaagagttttgataattctccagtgacggtttcacttttggtcttttgggaaggagactcaacaggagatgaaatgtgtgtttcttgtgttgcatcttcctgtacaggctcttcgag

SEQ ID NO: 26 - DNA, 1752 bp, Artificial sequence
  Probe
agaacgcagaccagcccaagctgacagcttgagtatgccttcttctgctgcctggttttgggggctgtatgacgtactggtcggtagtaaagattaatatgtaagaaatgtggagctaggatcaagtcatactccacagcctgcctggcaaactatgttttacttctgactttgctctctcgctgagaacattaatctgtcaagctggcgggctcctttgatagcaactttcccaggggcatgatgtggcaatgccacctctcagcccaggactaccgctattaccccgtggacggctactccctgcttaaacgcttccctcttcatcctcttacaggacccagatgccctgtccaaacagtgggacaatggttggaaagcattgggctacctcagtacgagaaccacctgatggctaatggatttgacaatgtgcagtttatgggaagcaatgttatggaagatcaggatttgttggaaattggaatccttaattctgggcacagacaaagaattctacaggcaatccagctccttccaaagatgagacccattgggcatgatggctaccatcccacctctgtagctgagtggctggattccattgaactgggcgactacaccaaagcctttctaattaatggctacacttcgatggacctgttgaaaaaaatctgggaggttgaacttattaatgttttaaaaatcaatttgattggccacaggaaacgtattttggcatctctgggagacaggctgcacgacgatcccccacagaagccccctcggtccatcaccctcaggacaggagactggggagaaccttccattaccttgcgacctccgaatgaagccacagcctctaccccggtacagtactggcagcatcacccagaaaagcttatcttccagtcgtgtgattacaaagctttttatttaggttctatgctgataaaagagcttagggggacagaatcaacccaagatgcttgtgcaaaaatgcgggctaactgtcagaagtctacagagcaaatgaagaaggtccctactattattctttctgtctcatataaaggagtcaaatttattgatgcaacaaataagaacataattgctgagcatgaaattcgtaatatctcctgtgctgcccaggacccagaagacctctcaacatttgcctatatcacaaaagatttgaagtctaatcaccactactgtcatgtgtttactgcctttgatgtgaatttagcctatgaaatcatcctaaccctgggacaggcattcgaagtcgcttaccagctagcactacaagcaagaaaagggggacactcctccacacttccagaaagctttgaaaacaaaccctccaaacccatccccaagccccgcgttagcattcgcaagtccgtggatcttcttcatgccagccataccgggcaggaaccatcagagagacacactgaggaggcactcagaaaattttgatagaattgaatttacatatagctgataaagcctaaatagaaaggttgatgtatttagcttgaatccttcactttagtggtctaatcaacagataaatgacaagcaaagaatgaaacaatccaaaaatgtttttcaaaacaattttgtgaattttatttttacaaaaattttttaaattcatattttaaaatgtataccaaggcaaaaaaatcatataagctatatcataaatacaagagtttcaaaacatacaagagacatataatg

SEQ ID NO: 27 - DNA, 367 bp, Artificial sequence
  Probe
ccgcgttagcattcgcaagtccgtggatcttcttcatgccagccataccgggcaggaaccatcagagagacacactgaggaggcactcagaaaattttgatagaattgaatttacatatagctgataaagcctaaatagaaaggttgatgtatttagcttgaatccttcactttagtggtctaatcaacagataaatgacaagcaaagaatgaaacaatccaaaaatgtttttcaaaacaattttgtgaattttatttttacaaaaattttttaaattcatattttaaaatgtataccaaggcaaaaaaatcatataagctatatcataaatacaagagtttcaaaacatacaagagacatataatg

SEQ ID NO: 28 - DNA, 367 bp, Artificial sequence
  Probe
cattatatgtctcttgtatgttttgaaactcttgtatttatgatatagcttatatgatttttttgccttggtatacattttaaaatatgaatttaaaaaatttttgtaaaaataaaattcacaaaattgttttgaaaaacatttttggattgtttcattctttgcttgtcatttatctgttgattagaccactaaagtgaaggattcaagctaaatacatcaacctttctatttaggctttatcagctatatgtaaattcaattctatcaaaattttctgagtgcctcctcagtgtgtctctctgatggttcctgcccggtatggctggcatgaagaagatccacggacttgcgaatgctaacgcgg

SEQ ID NO: 29 - DNA, 2457 bp, Homo sapiens
cacgcagcaggatggcaagggctccgcttggggtcctgctcctcttggggcttctcggcaggggtgtggggaagaacgaggaactgcgtctttatcaccatctcttcaacaactatgacccaggaagccggccagtgcgggagcctgaggatactgtcaccatcagcctcaaggtcaccctgacgaatctcatctcactgaatgaaaaagaggagactctcaccactagcgtctggattggaatcgattggcaggattaccgactcaactacagcaaggacgactttgggggtatagaaaccctgcgagtcccttcagaactcgtgtggctgccagagattgtgctggaaaacaatattgatggccagttcggagtggcctacgacgccaacgtgctcgtctacgagggcggctccgtgacgtggctgcctccggccatctaccgcagcgtctgcgcagtggaggtcacctacttccccttcgattggcagaactgttcgcttattttccgctctcagacgtacaatgccgaagaggtggagttcacttttgccgtagacaacgacggcaagaccatcaacaagatcgacatcgacacagaggcctatactgagaacggcgagtgggccatcgacttctgcccgggggtgatccgccgccaccacggtggcgccaccgacggcccaggggagactgacgtcatctactcgctcatcatccgccggaagccgctcttctacgtcattaacatcatcgtgccctgtgtgctcatctcgggcctggtgctgctcgcctacttcctgccggcgcaggccggcggccagaaatgcacggtctccatcaacgtcctgctcgcccagaccgtcttcttgttcctcattgcccagaaaatcccagagacttctctgagcgtgccgctcctgggcaggttccttattttcgtcatggtggtcgccacgctcattgtcatgaattgcgtcatcgtgctcaacgtgtcccagcggacgcccaccacccacgccatgtccccgcggctgcgccacgttctcctggagctgctgccgcgcctcctgggctccccgccgccgcccgaggccccccgggccgcctcgcccccaaggcgggcgtcgtcggtgggcttattgctccgcgcggaggagctgatactgaaaaagccacggagcgagctcgtgtttgaggggcagaggcaccggcaggggacctggacggctgccttctgccagagcctgggcgccgccgcccccgaggtccgctgctgtgtggatgccgtgaacttcgtggccgagagcacgagagatcaggaggccaccggcgaggaagtgtccgactgggtgcgcatggggaatgcccttgacaacatctgcttctgggccgctctggtgctcttcagcgtgggctccagcctcatcttcctcggggcctacttcaaccgagtgcctgatctcccctacgcgccgtgtatccagccttagctcgcaccgacttcaatttcccacccatctccagtaggaaattgattttgaaaaagtaggctgccgccaccacggcattatgatcccttccccctgctgatcaatctgcagtttgtgaacttcacaagaatggtgtgtgcccgttccctggcgtgtgtaggcctggccgcagtccaggggtcagcaggaggaaagggttcacataggctctcaggtgccagtcttccagaaagcaaggactgcccttcattcagccttgctgacctcccagcctttctaaggctcagccccacgggactctggtggctgccagcttgtgagctatctatctatattcatttcatagccaaacaggagacccctttgcaggacttgcacacagggaggctgtagccaggaaaccctcttcttccctggtctggctctgctggagcgggtgggaaccaaacaccttcagtgctggtggccctcaggcccacaggtttaaggctgaggctgccctgacccttccacagtcatttcttctaggttttcttggcccagcactgcccatcccaccccatgaggctcactcattgcagatcccagcccaccctgcccctttcttccccaccctggaggctctctctgcctagtctacagtactgacagaaagcaaggacatgcggcctgcatggtgggagctggttgaattgtctttattaacaaacaggatatccaaggccactacattgaggaggggggaggggggagggaggagaagggttacttgctgctcacactatatacagatgcaagcaaggggcgtggagagtgagggctccctgctccctccctccaccggggaagggcatgggctagaagaggagaggggggtcgggaatggggggaatgttttggctgcggggtcccccctccattccctggagtttgggggaaggggaatcattaaagtgctttcaga

SEQ ID NO: 30 - DNA, 4863 bp, Homo sapiens
ggagatagcgcctgtcagtcggtgggtcggtcctcgcgccggccctccccctccccggtctccgggggaggcgcggtggagtccgcccccggggttctccgatgggggagaagcggcgacggcggcagtggagtaaccgagccggagcgtgagcggccccggtgccccgttccccacggaggccatgggcgacccagcccccgcccgcagcctggacgacatcgacctgtccgccctgcgggaccctgctgggatctttgagcttgtggaggtggtcggcaatggaacctacggacaggtgtacaagggtcggcatgtcaagacggggcagctggctgccatcaaggtcatggatgtcacggaggacgaggaggaagagatcaaacaggagatcaacatgctgaaaaagtactctcaccaccgcaacatcgccacctactacggagccttcatcaagaagagccccccgggaaacgatgaccagctctggctggtgatggagttctgtggtgctggttcagtgactgacctggtaaagaacacaaaaggcaacgccctgaaggaggactgtatcgcctatatctgcagggagatcctcaggggtctggcccatctccatgcccacaaggtgatccatcgagacatcaaggggcagaatgtgctgctgacagagaatgctgaggtcaagctagtggattttggggtgagtgctcagctggaccgcaccgtgggcagacggaacactttcattgggactccctactggatggctccagaggtcatcgcctgtgatgagaaccctgatgccacctatgattacaggagtgatatttggtctctaggaatcacagccatcgagatggcagagggagccccccctctgtgtgacatgcaccccatgcgagccctcttcctcattcctcggaaccctccgcccaggctcaagtccaagaagtggtctaagaagttcattgacttcattgacacatgtctcatcaagacttacctgagccgcccacccacggagcagctactgaagtttcccttcatccgggaccagcccacggagcggcaggtccgcatccagcttaaggaccacattgaccgatcccggaagaagcggggtgagaaagaggagacagaatatgagtacagcggcagcgaggaggaagatgacagccatggagaggaaggagagccaagctccatcatgaacgtgcctggagagtcgactctacgccgggagtttctccggctccagcaggaaaataagagcaactcagaggctttaaaacagcagcagcagctgcagcagcagcagcagcgagaccccgaggcacacatcaaacacctgctgcaccagcggcagcggcgcatagaggagcagaaggaggagcggcgccgcgtggaggagcaacagcggcgggagcgggagcagcggaagctgcaggagaaggagcagcagcggcggctggaggacatgcaggctctgcggcgggaggaggagcggcggcaggcggagcgcgagcaggaatacaagcggaagcagctggaggagcagcggcagtcagaacgtctccagaggcagctgcagcaggagcatgcctacctcaagtccctgcagcagcagcaacagcagcagcagcttcagaaacagcagcagcagcagctcctgcctggggacaggaagcccctgtaccattatggtcggggcatgaatcccgctgacaaaccagcctgggcccgagaggtagaagagagaacaaggatgaacaagcagcagaactctcccttggccaagagcaagccaggcagcacggggcctgagccccccatcccccaggcctccccagggcccccaggacccctttcccagactcctcctatgcagaggccggtggagccccaggagggaccgcacaagagcctggtggcacaccgggtcccactgaagccatatgcagcacctgtaccccgatcccagtccctgcaggaccagcccacccgaaacctggctgccttcccagcctcccatgaccccgaccctgccatccccgcacccactgccacgcccagtgcccgaggagctgtcatccgccagaattcagaccccacctctgaaggacctggccccagcccgaatcccccagcctgggtccgcccagataacgaggccccacccaaggtgcctcagaggacctcatctatcgccactgcccttaacaccagtggggccggagggtcccggccagcccaggcagtccgtgccagtaaccccgacctcaggaggagcgaccctggctgggaacgctcggacagcgtccttccagcctctcacgggcacctcccccaggctggctcactggagcggaaccgcgtgggagtctcctccaaaccggacagctcccctgtgctctcccctgggaataaagccaagcccgacgaccaccgctcacggccaggccggcccgcaagctataagcgagcaattggtgaggactttgtgttgctgaaagagcggactctggacgaggcccctcggcctcccaagaaggccatggactactcgtcgtccagcgaggaggtggaaagcagtgaggacgacgaggaggaaggcgaaggcgggccagcagaggggagcagagatacccctgggggccgcagcgatggggatacagacagcgtcagcaccatggtggtccacgacgtcgaggagatcaccgggacccagcccccatacgggggcggcaccatggtggtccagcgcacccctgaagaggagcggaacctgctgcatgctgacagcaatgggtacacaaacctgcctgacgtggtccagcccagccactcacccaccgagaacagcaaaggccaaagcccaccctcgaaggatgggagtggtgactaccagtctcgtgggctggtaaaggcccctggcaagagctcgttcacgatgtttgtggatctagggatctaccagcctggaggcagtggggacagcatccccatcacagccctagtgggtggagagggcactcggctcgaccagctgcagtacgacgtgaggaagggttctgtggtcaacgtgaatcccaccaacacccgggcccacagtgagacccctgagatccggaagtacaagaagcgattcaactccgagatcctctgtgcagccctttggggggtcaacctgctggtgggcacggagaacgggctgatgttgctggaccgaagtgggcagggcaaggtgtatggactcattgggcggcgacgcttccagcagatggatgtgctggaggggctcaacctgctcatcaccatctcagggaaaaggaacaaactgcgggtgtattacctgtcctggctccggaacaagattctgcacaatgacccagaagtggagaagaagcagggctggaccaccgtgggggacatggagggctgcgggcactaccgtgttgtgaaatacgagcggattaagttcctggtcatcgccctcaagagctccgtggaggtgtatgcctgggcccccaaaccctaccacaaattcatggccttcaagtcctttgccgacctcccccaccgccctctgctggtcgacctgacagtagaggaggggcagcggctcaaggtcatctatggctccagtgctggcttccatgctgtggatgtcgactcggggaacagctatgacatctacatccctgtgcacatccagagccagatcacgccccatgccatcatcttcctccccaacaccgacggcatggagatgctgctgtgctacgaggacgagggtgtctacgtcaacacgtacgggcgcatcattaaggatgtggtgctgcagtggggggagatgcctacttctgtggcctacatctgctccaaccagataatgggctggggtgagaaagccattgagatccgctctgtggagacgggccacctcgacggggtcttcatgcacaaacgagctcagaggctcaagttcctgtgtgagcggaatgacaaggtgttttttgcctcagtccgctctgggggcagcagccaagtttacttcatgactctgaaccgtaactgcatcatgaactggtgacggggccctgggctggggctgtcccacactggacccagctctccccctgcagccaggcttcccgggccgcccctctttcccctccctgggcttttgcttttactggtttgatttcactggagcctgctgggaacgtgacctctgacccctgatgctttcgtgatcacgtgaccatcctcttccccaacatgtcctcttcccaaaactgtgcctgtccccagcttctggggagggacacagcttccccttcccaggaattgagtgggcctagcccctccccccttttctccatttgagaggagagtgcttggggcttgaaccccttaccccactgctgctgactgggcagggccctggacccctttatttgcacgtcaggggagccggctccccccttgaatgtaccagaccctggggggggtcactgggccctagatttttggggggtcaccagccactccaggggcagggaccatttcttcattttctgaaagcactttaatgattccccttcccccaaactccagggaatggaggggggaccccgccagccaaaacattccccccattcccgacccccctctcctcttctagcccatgcccttccccggtggagggagggagcagggagccctcactctccacgccccttgcttgcatctgtatatagtgtgagcagcaagtaacccttctccctccccccccacccctcctcaatgtagtggccttggatatcctgtttgttaataaagacaattcaaccagct

SEQ ID NO: 31 - DNA, 283 bp, Artificial sequence
  Probe
agctggttgaattgtctttattaacaaacaggatatccaaggccactacattgaggaggggggaggggggagggaggagaagggttacttgctgctcacactatatacagatgcaagcaaggggcgtggagagtgagggctccctgctccctccctccaccggggaagggcatgggctagaagaggagaggggggtcgggaatggggggaatgttttggctgcggggtcccccctccattccctggagtttgggggaaggggaatcattaaagtgctttcaga

SEQ ID NO: 32 - DNA, 283 bp, Artificial sequence
  Probe
tctgaaagcactttaatgattccccttcccccaaactccagggaatggaggggggaccccgcagccaaaacattccccccattcccgacccccctctcctcttctagcccatgcccttccccggtggagggagggagcagggagccctcactctccacgccccttgcttgcatctgtatatagtgtgagcagcaagtaacccttctcctccctcccccctcccccctcctcaatgtagtggccttggatatcctgtttgttaataaagacaattcaaccagct

SEQ ID NO: 33 - DNA, 2714 bp, Homo sapiens
ggcacagggcgaggttttatacacctgaaagaagagaatgtcaagacgaagtagccgtttacaagctaagcagcagccccagcccagccagacggaatccccccaagaagcccagataatccaggccaagaagaggaaaactacccaggatgtcaaaaaaagaagagaggaggtcaccaagaaacatcagtatgaaattaggaattgttggccacctgtattatctggggggatcagtccttgcattatcattgaaacacctcacaaagaaataggaacaagtgatttctccagatttacaaattacagatttaaaaatctttttattaatccttcacctttgcctgatttaagctggggatgttcaaaagaagtctggctaaacatgttaaaaaaggagagcagatatgttcatgacaaacattttgaagttctgcattctgacttggaaccacagatgaggtccatacttctagactggcttttagaggtatgtgaagtatacacacttcatagggaaacattttatcttgcacaagacttttttgatagatttatgttgacacaaaaggatataaataaaaatatgcttcaactcattggaattacctcattattcattgcttccaaacttgaggaaatctatgctcctaaactccaagagtttgcttacgtcactgatggtgcttgcagtgaagaggatatcttaaggatggaactcattatattaaaggctttaaaatgggaactttgtcctgtaacaatcatctcctggctaaatctctttctccaagttgatgctcttaaagatgctcctaaagttcttctacctcagtattctcaggaaacattcattcaaatagctcagcttttagatctgtgtattctagccattgattcattagagttccagtacagaatactgactgctgctgccttgtgccattttacctccattgaagtggttaagaaagcctcaggtttggagtgggacagtatttcagaatgtgtagattggatggtaccttttgtcaatgtagtaaaaagtactagtccagtgaagctgaagacttttaagaagattcctatggaagacagacataatatccagacacatacaaactatttggctatgctggaggaagtaaattacataaacaccttcagaaaagggggacagttgtcaccagtgtgcaatggaggcattatgacaccaccgaagagcactgaaaaaccaccaggaaaacactaaagaagataactaagcaaacaagttggaattcaccaagattgggtagaactggtatcactgaactactaaagttttacagaaagtagtgctgtgattgattgccctagccaattcacaagttacactgccattctgattttaaaacttacaattggcactaaagaatacatttaattatttcctatgttagctgttaaagaaacagcaggacttgtttacaaagatgtcttcattcccaaggttactggatagaagccaaccacagtctataccatagcaatgtttttcctttaatccagtgttactgtgtttatcttgataaactaggaattttgtcactggagttttggactggataagtgctaccttaaagggtatactaagtgatacagtactttgaatctagttgttagattctcaaaattcctacactcttgactagtgcaatttggttcttgaaaattaaatttaaacttgtttacaaaggtttagttttgtaataaggtgactaatttatctatagctgctatagcaagctattataaaacttgaatttctacaaatggtgaaatttaatgttttttaaactagtttatttgccttgccataacacattttttaactaataaggcttagatgaacatggtgttcaacctgtgctctaaacagtgggagtaccaaagaaattataaacaagataaatgctgtggctccttcctaactggggctttcttgacatgtaggttgcttggtaataacctttttgtatatcacaatttgggtgaaaaacttaagtaccctttcaaactatttatatgaggaagtcactttactactctaagatatccctaaggaatttttttttttaatttagtgtgactaaggctttatttatgtttgtgaaactgttaaggtcctttctaaattcctccattgtgagataaggacagtgtcaaagtgataaagcttaacacttgacctaaacttctattttcttaaggaagaagagtattaaatatatactgactcctagaaatctatttattaaaaaaagacatgaaaacttgctgtacataggctagctatttctaaatattttaaattagcttttctaaaaaaaaaatccagcctcataaagtagattagaaaactagattgctagtttattttgttatcagatatgtgaatctcttctccctttgaagaaactatacatttattgttacggtatgaagtcttctgtatagtttgtttttaaactaatatttgtttcagtattttgtctgaaaagaaaacaccactaattgtgtacatatgtattatataaacttaaccttttaatactgtttatttttagcccattgtttaaaaaataaaagttaaaaaaatttaactgcttaaaagtaaagttttgccattgcttggagaaacttttttttccttctctgcgctgccagctgta

SEQ ID NO: 34 - DNA, 6773 bp, Homo sapiens
caagcatgtgatgttcttgtaccttcttctgatagtacatctcaacagttgactccatatagtcaagtccatatttgtttgagatctggcaactatcaggaggtaatacagattttcattgaagacaacttaaccttgagtttacctgtccagttccgacagtcagtcctaagagaactctttaagaaagctcaacagggaaatgaagctctagatgaaatctgttttaaagtttgtgcctgtaatacagtccgtgatatactggaaggcagaacaattagtgttcaatttaaccagctatttcttagaccaaataaagagaaaatagactttcttcttgaggtatgttcaagatcagtaaatttagaaaaagcttcagagtctttgaaaggaaacatggctgcttttctaaagaatgtgtgtctggggttggaagatctgcagtatgttttcatgatttcttcacatgagcttttcattacattgttgaaagatgaagaacgaaagctacttgttgatcagatgaggaagagatcccctagagtaaatctgtgcattaaacctgtaacttcattttatgatatcccagcttcagcaagtgtcaacattggtcagttagagcatcaacttatattgtcagtggatccttggaggattagacaaattttaattgaattacatggtatgacttcagagcgccagttctggacagtgtctaataagtgggaagtaccttctgtctatagtggtgttatcctgggaattaaagacaatttaacaagagatttggtttatattcttatggccaaaggtttgcactgcagtactgttaaggacttttcccatgctaaacagctctttgctgcttgtttggagttggtaacagagttctcaccgaagcttcgtcaggtcatgctgaatgagatgttgcttttggatattcatacacacgaagctgggacagggcaggcaggagagagaccgccatccgaccttataagtagagtacgaggctatctggaaatgaggcttcctgatattcctcttcgtcaagttatagctgaggaatgtgttgcctttatgttaaactggagagaaaatgaataccttacactccaagttcctgcatttttgcttcagagtaatccatatgtaaagcttggacagcttttagcagctacatgcaaagaacttccaggccctaaagaaagcagacggactgccaaagacctttgggaagttgttgttcaaatctgtagtgtgtccagtcagcacaaacgaggaaatgatggcagagttagtttaataaaacagagggaatctacgttaggtatcatgtatcggagtgaactgctttcttttatcaaaaaattacgagaaccactcgttttgactattattttatcactctttgtgaaacttcacaatgttcgggaggacattgtgaatgatattacagctgaacacatttctatttggccatcttccattcccaacctccagtctgtggactttgaagctgtggcaatcacagtgaaagagctagttcgatatacactcagtataaatccaaataaccattcttggttaattatccaggcagatatttactttgcaacgaatcagtattcagcagctcttcactattacctccaggcaggagctgtgtgttctgacttctttaacaaggctgtgccccctgatgtttatacagaccaggtaataaaacgaatgataaaatgttgttctttgctgaattgccacacacaggtggctattttatgtcagttcctcagagaaattgactacaaaacagcgtttaaatctctgcaagaacaaaacagtcatgatgctatggactcctactacgactacatatgggatgttaccattttggaatacttgacttatcttcatcataaaagaggagaaacagataaaagacaaattgcaatcaaagccatcggccagacagagttgaatgcaagcaatccagaagaagtgttacagctggcagcgcagagaaggaaaaaaaagtttctccaagcaatggcaaaactttacttttaagcagttaaatttttttaacttttattttttaaacaatgggctaaaaataaacagtattaaaaggttaagtttatataatacatatgtacacaattagtggtgttttcttttcagacaaaatactgaaacaaatattagtttaaaaacaaactatacagaagacttcataccgtaacaataaatgtatagtttcttcaaagggagaagagattcacatatctgataacaaaataaactagcaatctagttttctaatctactttatgaggctggattttttttttagaaaagctaatttaaaatatttagaaatagctagcctatgtacagcaagttttcatgtctttttttaataaatagatttctaggagtcagtatatatttaatactcttcttccttaagaaaatagaagtttaggtcaagtgttaagctttatcactttgacactgtccttatctcacaatggaggaatttagaaaggaccttaacagtttcacaaacataaataaagccttagtcacactaaattaaaaaaaaaaattccttagggatatcttagagtagtaaagtgacttcctcatataaatagtttgaaagggtacttaagtttttcacccaaattgtgatatacaaaaaggttattaccaagcaacctacatgtcaagaaagccccagttaggaaggagccacagcatttatcttgtttataatttctttggtactcccactgtttagagcacaggttgaacaccatgttcatctaagccttattagttaaaaaatgtgttatggcaaggcaaataaactagtttaaaaaacattaaatttcaccatttgtagaaattcaagttttataatagcttgctatagcagctatagataaattagtcaccttattacaaaactaaacctttgtaaacaagtttaaatttaattttcaagaaccaaattgcactagtcaagagtgtaggaattttgagaatctaacaactagattcaaagtactgtatcacttagtataccctttaaggtagcacttatccagtccaaaactccagtgacaaaattcctagtttatcaagataaacacagtaacactggattaaaggaaaaacattgctatggtatagactgtggttggcttctatccagtaaccttgggaatgaagacatctttgtaaacaagtcctgctgtttctttaacagctaacataggaaataattaaatgtattctttagtgccaattgtaagttttaaaatcagaatggcagtgtaacttgtgaattggctagggcaatcaatcacagcactactttctgtaaaactttagtagttcagtgataccagttctacccaatcttggtgaattccaacttgtttgcttagttatcttctttagtgttttcctggtggtttttcagtgctcttcggtggtgtcataatgcctccattgcacactggtgacaactgtcccccttttctgaaggtgtttatgtaatttacttcctcctatacatgggaagaaatcatgcactgatttcataaatcaaagtcaaaccagacttctgggtacttatttgagattatttaggcctaattttaatagtctttttatgtctttgcaagtgtgaagggtcatattctgaaagtttctgtaacgttatatattttttaaactctttatctagactgttggcattgatcttgagacacttcacaaatcttgctttgatttcaaagtaattttattaacttttctacattttgaaatcagtgtgccccttagaactttctttcccctgaaactgcctgaaggagtactctattcctaccatcagttttggtgacttactagattcagatagcaaagccaaaaaactcacaaaaaaacataccagcatagccaaatagtttgtatgtgtctggatattatgtctgtcttccataggaatcttcttaaaagtcttcagcttcactggactagtactttttactacattgacaaaaggtaccatccaatctacacattctgaaatactgtcccactccaaacctagatagatagaaaaaagttagaaaagcatgaaggttgtacatcagaaactatcttacatatgtctgatgtacttgttgctgtttttgagatattttaaaagaaaccaaatcataaccaagaagtttagcatgtcaaaacagattatcactctcaaactatttacatgactatgttgaagggaaaaaggacttcagaacttcttaaccagtaccttctacatatgaaattgaaatggtcaaatcccaaagaactcttaaagcagaactataatgttgattcatttcaactgtatttaaattccatttggtctttttgttgatacacattcaggattggaaagtacttctaacagaaagataattactgaacagctaattttttttttgccaaagttttaaatgcatgtttagcagaatgttaaagttcagagactgtagtcccattagaagttgtgaaaaggtaagaagacaacaaatagagagtcttacctgaggctttcttaaccacttcaatggaggtaaaatggcacaaggcagcagcagtcagtattctgtactggaactctaatgaatcaatggctagaatacacagatctaaaagctaacaggaaaaacaaaagtacaagcaatttaggagaaagatgagtactaaatgtctcttgctaaaaccttagggatctagagataaataagccaccacccggccaggcgcggtggctcacgcctgtaatcccagcactttgggaggccgaggcgggtggatcacaaggtcacgagatcgagaccatcctggctaacacggtgaaaccccgcctctactaaaaaatacaaaaaattagctgggcttggtggcacgcgcctgtagtcccagctactcgggaggctgaggcaggagaatggcgtgaacccgggatgcagagcttgcagtgagcagagatcgcgccactgcactccagcctgggcgaaagagcgagactccgtctcaaaaaaaaaaaaaacaagccaccaacctgaaggaagtagacaaggaaggactgttgcaatacagtgtgacatgtactagcaggaagggcacctaatccagattggaaaagatagtgatggcctcaaattgccataaatgggtcttaaaagataagggagccaggaagagtaggaggcagagaatgttctaggtatagggacattacttggaactcagttcacagttcagaactcctaaggtgaaaaataaataaggagtaccttcatttcttatcaagaaagatgaggggtggtggctagaaagaggcatggtctagattggatcacaaagggtctttaagaagtcagaattttataggctgattcttgaagctactggaagatttttaaatcaaagttccattttaagaaagataccttagaatgcagtgaagcagacagactagaagaaaacatgtttattaagcagtgagattagttaaaaggctgtataatctaggcaataagagctgaactagtagcagtggaatggtatagtgtaaaaggggtagatttcacagatttgagaagatacttgtgcagtggaattaaacttcaattctctttgtcctcattggtccagaaggtaggagaaatgggagaagagctgggaattggaagtgaaatattactgttatatacctctagaaagtccacattgtttatcggcttatcaaagatttaccatcactatcagaagggtatagctgcctaggacaatttgggatgctaggaattctggatgaaaaaattaagcttttaataaaaagttttataaaataaaccaatttcagtatacttagtggttatccaatttgagtattcataatgtgctagatttaagcaccactgcccacaaattttaacctaggtgacttaataattatccccaaatgtcttccatatgttagattttcacatcccacatagaataagagggtagattttcttcacttttgttatatggcagatacagcagccttaagattacttacgagaagtaagcaagaaagaatgggatctcctctttttttttttttttaattttttgagatggagtcttgctctgttgctcaggctggagtgcagtagtgcgatctcggctcactgcaacctccacctcccaggttccagcgattctcctgcctcagcctcccaagtaacatgttggctaggctgcctcagccgcccaaactcctgacctcaagtgatctgcctgcctgcctcagccgcccaaagtgctgagattagagacctgagccacagtgcccggccagatcctcctcctcctctacttacttactttgttaaatatgctagcctggaaaagtttactttgaatttatgttctaaaaaatttttttaacaaagtaattttaattctgatatttaacttgataggcactctgtgtatccaaatgtaaagacatcatacagaataattctatgccattataaagcttaaacacaactggcgaaaaaaatgcttttccccattttatatcaaaaagagatactttagtttggactcctaaagaatgaaagtactcagaaaagtgtaaggactttgtttttctagaaatattaagcaacataaacactggggacagaactttatgcgtc

SEQ ID NO: 35 - DNA, 1590 bp, Mus musculus
ctgagaaccagacatcaggatggcaggggctctgcttggtgccctgcttctcctgacactctttggcagaagccagggaaagaatgaagagcttagcctgtatcaccatctcttcgacaattatgatccagaatgccggccagttaggagacctgaggacactgtcaccatcaccctcaaggtcaccctaaccaacctcatctcactgaacgagaaagaagaaactctgaccaccagtgtctggattggcattgactggcacgactatcggctcaactacagcaaggacgattttgcaggtgtaggaatcctccgggtcccttcagaacatgtatggctgccagagattgttctagaaaacaatattgatgggcagtttggagtggcctacgacagcaatgttctagtctatgagggaggctatgtgagctggttgcccccagccatctaccgcagcacctgcgcagtggaggtcacctatttcccctttgactggcagaactgctctctcatttttcgctcccagacctacaatgctgaggaggtggagttcatctttgccgtggatgacgacggcaataccatcaacaagattgacattgacacggcagcttttaccgagaatggagaatgggccatagactactgcccaggcatgattcgccgctatgagggaggttccacagaaggtcctggagaaactgacgtcatctatacgctcatcatccgccggaagccgcttttttacgtcattaacatcattgtgccttgcgtgctcatttctggcttggtgctgctcgcttacttcctgcctgcgcaggctggtggccagaaatgcacggtctctatcaacgtcctgctagcccagactgtcttcttgtttctaattgcccagaaaattccagagacttctctgagcgtgccactgctgggcaggtatcttatattcgtcatggtggttgccacgctcattgtcatgaattgcgtcatcgtgctcaacgtatctttgaggacgccaacgactcatgctacatcccctcggctgcgccagattttattagagctgctgccgcgtctcctgggctcgagcccacccccagaggatccccgaactgcctcaccagcgaggcgtgcctcgtctgtgggcattctgctcagagcggaggagctcatcttgaaaaagccgcggagcgaactcgtgtttgagggtcagaggcatcggcacggaacttggaccgcagccctctgccagaacctgggtgctgcagccccagaaatccgctgctgtgtggatgctgtgaactttgtggctgagagcacaagagaccaggaagccactggagaggaactgtccgactgggtgcgtatggggaaggccctggacaatgtctgtttttgggcagctttggtgctcttcagcgttggttctactctcatcttccttgggggttacttcaaccaagttcctgatctcccttacccaccgtgcatccaaccatgagcctgcactggcacccacctctcccccaccccccaagaaagagattttgaaaacaggccgctgacaataaatctggtttgtgaacttgc

SEQ ID NO: 36 - DNA, 2227 bp, Mus musculus
tgtgagcagcaagtagcccttctccctcctgtatcctttctcaatgtagtggccttggatatatcccctttgttaataaagacaattcaaccagcttccaccattttgagatcctactattgttctctctcaatcctggagagatttgagagttgagaatgcagagggtagaggaaaggcattaggctctgtgaagttactgtgataatagagacgaagtaaggtggatgaataggccagggatcagtcctgacacggtaggaccctttgagaatagtttttaccagccccagcagggccaggccagacttctggcttcagtgtttctatatctgggtcttgtaaaaacctcattggctatcaactagataaacattctttaggttagaaggagccaagagcaaaattgaaccaattgcctccaagtgcctgaccaaaccacccacccatcttctacttccctgaggagttggacccacccacatgaccacacaacccctcgggcagttcacaaaccagatttattgtcagcggcctgttttcaaaatctctttcttggggggtgggggagaggtgggtgccagtgcaggctcatggttggatgcacggtgggtaagggagatcaggaacttggttgaagtaacccccaaggaagatgagagtagaaccaacgctgaagagcaccaaagctgcccaaaaacagacattgtccagggccttccccatacgcacccagtcggacagttcctgtgagagagagcttagcgagggaggagcctggagggcggggcatctagcactgctccgcctcaacctcccaacccacctctccagtggcttcctggtctcttgtgctctcagccacaaagttcacagcatccacacagcagcggatttctggggctgcagcacccaggttctggcagagggctgctgctaaggcaacagcaagcgctaggtcattaaaagagcgtcctaacggcgagtgtatgcctttgacccaagagcagtgcttaccggtccaagttccgtgccgatgcctctgaccctcaaacacgagttcgctccgcggctttttcaagatgagctcctccgctctgagcagaatgcccacagacgaggcacgcctcgctggtgaggcagttcggggatcctctgggggtgggctcgagcccaggagacgcggcagcagctctaataaaatctgcagccggggcagagagaggttccaagcccgcttcccacccctgggcagtactttctccaaccagcgcttacctggcgcagccgaggggatgtagcatgagtcgttggcgtcctcaaagatacgttgagcacgatgacgcaattcatgacaatgagcgtggcaaccaccatgacgaatataagatacctgatatacagaagcctgatgtcacagcaccccacaaacaaggcactagctgccctctacctcacaaataccacctcgcacagctggtggcgttacttcttgatcctcctcaacgatgccagtattgtcctggcccttctgcatataccatctgttgcggacatgaaggggattcccagcaatttggacaccctgctgtgggtctaccacttccacagctccaccgaggtgagggtattagaatggcagaatctggagaggtccccagctcttcctgctatggccctttccatgtgatcattccactcactacccttgctcctccaggtggccttacagcctccacttctatcttccctggaacttgctgtggccgcagctcacgaatatctggtgcaaaggttcagagagcttaagtcccaggaccccctggaatccgacaagtcgcccacccagaaggccaccctagggctggtgctaagagaagctgcagccagcatcatgagctttggagccaccttgttagaggtgctgctctgggaggctgagggatgggaataaaagggggagagggctaggccaacaaaagcaaggacctctagcccatatgccccaatgtagatctcggccctgtggctgcagcaggaggtgcagcgactggacggcggcaacgactgcccaggcccagccccagacactggggatcctggtagggcgctggcccgtgtagccctggccgcagggcaggggattcggcaagctggaacggcagctggcgcaagtgcccggtacctgatccagggggcgtggttgtacctgtgtggacgaggtttg

SEQ ID NO: 37 - DNA, 2472 bp, Homo sapiens
agcatcgagtcggccttgttgcctactggagtctccgcagagcccgggcgggagtagctggtggaccccgttgagctgccgaacttccgggactcccccgcgaccccttcccagcttcccgtccgctccgccgcagcgattgtctcggtgggttgattcggcacaaaccgcccgacccaggggccggtgcgcgtgtggaaggggaagcactcccctcgtggtcgcctggaggtgcgctggaggagggggtgacataaccagggactcgaggtccgccgtgggaatgatccacgaactgctcttggctctgagcgggtaccctgggtccattttcacctggaacaagcggagtggcctgcaggtatcgcaggacttccctttcctccaccccagtgagaccagtgtcctgaatcgactctgccggctcggcacagactatattcgcttcactgagttcattgaacagtacacgggccatgtgcaacagcaggatcaccatccatctcaacagggccaaggtgggttacatggaatctacctgcgggccttctgcacagggctggattctgttttgcagccttatcgccaagcactgcttgatttggaacaagagttcctgggtgatccccatctctccatatcacatgtcaactacttcctagaccagttccagcttctttttccctctgtgatggttgtagtagaacaaattaaaagtcaaaagattcatggttgtcaaatcctggaaacagtctacaaacacagctgtggggggttgcctcctgttcgaagtgcactggaaaaaatcctggccgtttgtcatggggtcatgtataaacagctctcagcctggatgctccatggactcctcttggaccagcatgaagaattctttatcaaacaggggccatcttctggtaatgtcagtgcccagccagaagaggacgaggaggatctgggcattgggggactgacaggaaaacaactgagagaactgcaggacttgcgcctgattgaggaagagaacatgctggcaccatctctgaagcagttttccctacgagtggagattttgccatcctacattccagtgagggttgctgaaaaaatcctatttgttggagaatctgtccagatgtttgagaatcaaaatgtgaacctgactagaaaaggatccattttgaaaaaccaggaagacacttttgctgcagagctgcaccgtctcaagcagcagccactcttcagcttggtggactttgaacaggtggtggatcgcattcgcagcactgtggctgagcatctctggaagttgatggtagaagaatccgatttactgggtcagctgaagatcattaaagacttttaccttctgggacgtggagaactgtttcaggccttcattgacacagctcaacacatgttgaaaacaccacccactgcagtaactgagcatgatgtgaatgtggcctttcaacagtcagcacacaaggtattgctagatgatgacaaccttctccctctgttgcacttgacaatcgagtatcacggaaaggagcacaaagcagatgctactcaggcaagagaagggccttctcgggaaacttctccccgggaagcccctgcatctggctgggcagccctaggtctttcctacaaagtacagtggccactacatattctcttcaccccagctgtcctggaaaaaaatagacaattttaaaaaccaaacagaatgggactgtcttctgcaagcctacctacaaacaggtacaatgttgtttttaagtacttactgagtgtgcgccgggtgcaagctgagctgcagcactgctgggccctacaaatgcagcgcaagcacctcaagtcgaaccagactgatgcaatcaagtggcgcctaagaaatcacatggcatttttggtggataatcttcagtactatctccaggtagatgtgttggagtctcagttctcccagctgcttcatcagatcaattctacccgagactttgaaagcatccgattggctcatgaccacttcctgagcaatttgctggctcaatcctttatcctattgaaacctgtgtttcactgcctgaatgaaatcctagatctctgtcacagtttttgttcgctggtcagtcagaacctaggcccactggatgagcgtggagccgcccagctgagcattctcgtgaagggctttagccgccagtcttcactcctgttcaagattctctccagtgttcggaatcatcagatcaactcagatttggctcaactactgttacgactagattataacaaatactatacccaggctggtggaactctgggcagtttcgggatgtgaaaatttctggctcataaattgaaataacagccacgttcccaaggttgtaacagaagattcaaaacatcccattctagccacacacaaataaatatctgcggcttaaaaaaaaaaaaaaaaaa

SEQ ID NO: 38 - DNA, 4165 bp, Homo sapiens
agcatcgagtcggccttgttgcctactggagtctccgcagagcccgggcgggagtagctggtggaccccgttgagctgccgaacttccgggactcccccgcgaccccttcccagcttcccgtccgctccgccgcagcgattgtctcggtgggttgattcggcacaaaccgcccgacccaggggccggtgcgcgtgtggaaggggaagcactcccctcgtggtcgcctggaggtgcgctggaggagggggtgacataaccagggactcgaggtccgccgtgggaatgatccacgaactgctcttggctctgagcgggtaccctgggtccattttcacctggaacaagcggagtggcctgcaggtatcgcaggacttccctttcctccaccccagtgagaccagtgtcctgaatcgactctgccggctcggcacagactatattcgcttcactgagttcattgaacagtacacgggccatgtgcaacagcaggatcaccatccatctcaacagggccaaggtgggttacatggaatctacctgcgggccttctgcacagggctggattctgttttgcagccttatcgccaagcactgcttgatttggaacaagagttcctgggtgatccccatctctccatatcacatgtcaactacttcctagaccagttccagcttctttttccctctgtgatggttgtagtagaacaaattaaaagtcaaaagattcatggttgtcaaatcctggaaacagtctacaaacacagctgtggggggttgcctcctgttcgaagtgcactggaaaaaatcctggccgtttgtcatggggtcatgtataaacagctctcagcctggatgctccatggactcctcttggaccagcatgaagaattctttatcaaacaggggccatcttctggtaatgtcagtgcccagccagaagaggacgaggaggatctgggcattgggggactgacaggaaaacaactgagagaactgcaggacttgcgcctgattgaggaagagaacatgctggcaccatctctgaagcagttttccctacgagtggagattttgccatcctacattccagtgagggttgctgaaaaaatcctatttgttggagaatctgtccagatgtttgagaatcaaaatgtgaacctgactagaaaaggatccattttgaaaaaccaggaagacacttttgctgcagagctgcaccgtctcaagcagcagccactcttcagcttggtggactttgaacaggtggtggatcgcattcgcagcactgtggctgagcatctctggaagttgatggtagaagaatccgatttactgggtcagctgaagatcattaaagacttttaccttctgggacgtggagaactgtttcaggccttcattgacacagctcaacacatgttgaaaacaccacccactgcagtaactgagcatgatgtgaatgtggcctttcaacagtcagcacacaaggtattgctagatgatgacaaccttctccctctgttgcacttgacaatcgagtatcacggaaaggagcacaaagcagatgctactcaggcaagagaagggccttctcgggaaacttctccccgggaagcccctgcatctggctgggcagccctaggtctttcctacaaagtacagtggccactacatattctcttcaccccagctgtcctggaaaagtacaatgttgtttttaagtacttactgagtgtgcgccgggtgcaagctgagctgcagcactgctgggccctacaaatgcagcgcaagcacctcaagtcgaaccagactgatgcaatcaagtggcgcctaagaaatcacatggcatttttggtggataatcttcagtactatctccaggtagatgtgttggagtctcagttctcccagctgcttcatcagatcaattctacccgagactttgaaagcatccgattggctcatgaccacttcctgagcaatttgctggctcaatcctttatcctattgaaacctgtgtttcactgcctgaatgaaatcctagatctctgtcacagtttttgtttgctggtcagtcagaacctaggcccactggatgagcgtggagccgcccagctgagcattctcgtgaagggctttagccgccagtcttcactcctgttcaagattctctccagtgttcggaatcatcagatcaactcagatttggctcaactactgttacgactagattataacaaatactatacccaggctggtggaactctgggcagtttcgggatgtgaaaatttctggctcataaattgaaataacagccacgttcccaaggttgtaacagaagattcaaaacatcccattctagccacacacaaataaatatctgcggcttagtgataggactctaccttttctcctagaagcagttactgaacatccaggagtacaactccttcccatcattcccatgtggaagggtctctcccatcaaggagaacatgtggcatctctgatcctttacattgagaacatttgttggatatgttcatttattcaatagtcatttattgagcacctactacgtaccttggtactgttcaagctgtgggagatacagcggtagacaaacaatatagagcagaaagttaaatattttatggttcatatgtgaaaaagtaattatgtttataaatagactaactgctggatgttaccaccaagtaagaaagcaacaggtaagataggctttctctctccctataccaagtaatttatacctacacagattgggcaattctagctaatgaaaatatacttaaaagtatttcttaggccgggcatggtggctcacacctgtaatcccagcactttgggaggccgaggcgggcggatcacctgaagtcaggagtttgagaccagcctgaccaacatgatgaaacctcgattctactaaaaatacaaaaattagccaggtgtggtggcatgtgcctgtaatcccagctactcaggaggctgagacaggagaattgcttgaacctgggaagcagacgctgcagtgagctgagattgtgccattgcattccagcctgggcaacaagagcgaaattccgtctcaaaaaaaaaaaaaaaaaaaaaagtattattctccaagaaaaaggtccttaagaaaaaattgagatcaagttgttagatttttaaatactgaagattgcaggcccaattacccatcttacacaaaccataggggttgaagttatcttaatatggcccagccatcactggtaatcaatattcatatcagtgtaagtaaaaagaaatattcactgaacaacgccctccaaactgaaaaagaatgcagtgttctggcatcaggttatagtcactgcatctggttttcatcactacatattctacacacactgggaagctctgacaacttattccctgctattatcaactaaagatcaccctttccactgctgtctctggagcaggagctggcaaactatggcctgctgtctgtttttgtacagttttactgaaacacagccgtgcccatttgtttactcattgtctatggttgctttcatgccctcacagcaaaggcgagtagttgtgatggatcaaatggcccacaaagcctgaaatatttactctttgaccctttacagaaaaaaaccttgttgacccctgctttagagaatgagaagccatgcagggatcagtgatgccagaggaagggaaggaactgcttccagctattgtgacaataataataataataatattgggtctttgactagaacgtgtaacatttccaggtgttctcacttgtgcttcccatgtttatcttacggaaggtcattccatcaagcttatggtcactgtcccttcatggcagttggtcctttcgttctccctttagctctaagagttggggagtacccacaggtgagctgtgatctcagctcagagagagagcatgaggtcttttttaactgtcaggaaacagagctgtgcccaattccactcaacttttggcacaactgttaatctgggccttcacctaccttaaactgagtttctgcaagcatagcattttagacaccctggaataaccttttgggaatgatgccacagaataaagttcactcttaacttttcaa

SEQ ID NO: 39 - DNA, 27 bp, Artificial sequence
  Synthetic oligonucleotide
ggagagaaccacccagcccagaagttc

SEQ ID NO: 40 - DNA, 23 bp, Artificial sequence
  Synthetic oligonucleotide
aggaatggaggcggcccttctgc

SEQ ID NO: 41 - DNA, 23 bp, Artificial sequence
  Synthetic oligonucleotide
cggaggagctcatcttgaaaaag

SEQ ID NO: 42 - DNA, 24 bp, Artificial sequence
  Synthetic oligonucleotide
gatcaggaacttggttgaagtaac

SEQ ID NO: 43 - DNA, 25 bp, Artificial sequence
  Synthetic oligonucleotide
tgtgagcagcaagtaacccttctcc

SEQ ID NO: 44 - DNA, 793 bp, Artificial sequence
  Probe
acagagttgaatgcaagcaatccagaagaagtgttacagctggcagcgcagagaaggaaaaaaaagtttctccaagcaatggcaaaactttacttttaagcagttaaatttttttaacttttattttttaaacaatgggctaaaaataaacagtattaaaaggttaagtttatataatacatatgtacacaattagtggtgttttcttttcagacaaaatactgaaacaaatattagtttaaaaacaaactatacagaagacttcataccgtaacaataaatgtatagtttcttcaaagggagaagagattcacatatctgataacaaaataaactagcaatctagttttctaatctactttatgaggctggattttttttttagaaaagctaatttaaaatatttagaaatagctagcctatgtacagcaagttttcatgtctttttttaataaatagatttctaggagtcagtatatatttaatactcttcttccttaagaaaatagaagtttaggtcaagtgttaagctttatcactttgacactgtccttatctcacaatggaggaatttagaaaggaccttaacagtttcacaaacataaataaagccttagtcacactaaattaaaaaaaaaaattccttagggatatcttagagtagtaaagtgacttcctcatataaatagtttgaaagggtacttaagtttttcacccaaattgtgatatacaaaaaggttattaccaagcaacctacatgtcaagaaagccccagttaggaaggagccacagc